(19) United States
(12) Patent Application Publication
(10) Pub. No.: US 2026/0078148 A1
(43) Pub. Date: Mar. 19, 2026

(54) AMATOXIN ANTIBODY-DRUG CONJUGATES AND USES THEREOF

(71) Applicant: Heidelberg Pharma Research GmbH, Ladenburg (DE)

(72) Inventors: Charlotte Fenton McDonagh, Winchester, MA (US); Rajiv Panwar, Acton, MA (US); Torsten Hechler, Ladenburg (DE); Michael Kulke, Ladenburg (DE); Ganapathy N. Sarma, San Ramon, CA (US); Andreas Pahl, Ladenburg (DE); Christoph Mueller, Ladenburg (DE); Werner Simon, Ladenburg (DE); Christian Lutz, Ladenburg (DE); Francesca Gallo, Ladenburg (DE)

(73) Assignee: Heidelberg Pharma Research GmbH, Ladenburg (DE)

(21) Appl. No.: 19/400,588

(22) Filed: Nov. 25, 2025

Related U.S. Application Data
(63) Continuation of application No. 17/605,766.
(60) Provisional application No. 62/838,290, filed on Apr. 24, 2019.

(51) Int. Cl. C07K 7/64; A61K 45/06; A61K 47/68; A61P 35/00; C07K 16/28

(52) U.S. Cl. CPC C07K 7/64; A61K 45/06; A61K 47/6831; A61K 47/6867; A61P 35/00; C07K 16/2803; C07K 2317/565; C07K 2319/55

(57) ABSTRACT

Amatoxins, as well as antibody-drug conjugates (ADCs) and compositions have amatoxin and can be used for cancer therapy, to prepare a patient for hematopoietic stem cell transplant therapy and to improve the engraftment of hematopoietic stem cell transplants by selectively depleting endogenous hematopoietic stem cells prior to the transplant procedure. Methods and compositions treat various hematopoietic diseases, metabolic disorders, cancers, and autoimmune diseases and prevent graft-versus-host disease (GVHD).

### CROSS-REFERENCE TO RELATED APPLICATIONS

This application is a continuation of U.S. patent application Ser. No. 17/605,766, filed on Oct. 22, 2021, which is the National Stage entry under § 371 of International Application No. PCT/EP2020/061555, filed on Apr. 24, 2020, and which claims the benefit to U.S. Patent Application No. 62/838,290, filed on Apr. 24, 2019, each of which are incorporated by reference in their entirety.

### REFERENCE TO A SEQUENCE LISTING

The present application is accompanied by an XML file as a computer readable form containing the sequence listing entitled, “2025-11-24 SeqList-006211USCONTO1-as-filed”, created on Nov. 24, 2025, with a file size of 752,725 bytes, the content of which is hereby incorporated by reference in its entirety.

### BACKGROUND OF THE INVENTION

### Field of the Invention

The present invention relates to amatoxins, Antibody-Drug Conjugates (ADCs) comprising an amatoxin, compositions comprising such ADCs, and methods for using the same.

### Description of Related Art

Monoclonal antibodies (mAb) can be conjugated to a therapeutic agent to form an antibody drug conjugate (ADC). ADCs can exhibit increased efficacy, as compared to an unconjugated antibody. The linkage of the antibody to the drug can be direct, or indirect via a linker. An important aspect of successful therapeutic ADCs is that the ADC be not only effective but well-tolerated. Often the cytotoxin impacts both efficacy and tolerability.

ADC have been proposed as therapeutic agents for the treatment of cancer. The use of ADCs for the local delivery of cytotoxic or cytostatic agents, i.e. drugs to kill or inhibit tumor cells in the treatment of cancer theoretically allows targeted delivery of the drug moiety to tumors, and intracellular accumulation therein, where systemic administration of these unconjugated drug agents may result in unacceptable levels of toxicity, also to normal cells.

ADCs have also been proposed as a therapeutic regimen for preparing patients for transplant and stem cell therapy. By conditioning a patient with a cell-specific ADC, stem cells or immune cells can be selectively depleted while leaving the patient's remaining immune system largely intact. For example, Palchaudhuri et al. (2016) Nat. Biotechnol. 34, 738-745 describes the use of a single dose of an anti-CD45 ADC where an anti-CD45 antibody was conjugated to saporin, and its ability to enable engraftment of donor cells and for treatment in a sickle-cell anemia model. Unlike irradiation, the CD45-SAP ADC was reported to have avoided neutropenia and anemia, and provided for rapid recovery of T and B cells with minimal overall toxicity. There remains a need for toxins that can be used for non-genotoxic targeted ADC-conditioning where the toxin is potent to the target cells while minimizing patient side effects.

### SUMMARY OF THE INVENTION

The present invention provides amatoxins that can be used in antibody drug conjugates (ADC), e.g., for delivery of the amatoxin to a target cell. The present invention further provides particular modifications of antibodies that can be used in antibody drug conjugates for the delivery of the amatoxin to a target cell. The present invention further relates to combinations of said amatoxins and antibodies which have increased efficacy, enhanced tolerability in vivo, and therefore advantageous therapeutic windows.

The invention includes but is not limited to the following embodiments:

1. An antibody-drug conjugate (ADC), comprising:
an antibody, or an antigen-binding fragment thereof, conjugated to an amatoxin, or a derivative or analogue thereof, via a linker, wherein the antibody, or the antigen binding fragment thereof, comprises an Fc region comprising at least two mutations consisting of L234A and L235A (according to EU index),
wherein said Fc region further comprises a mutation consisting of D265C (according to EU index), and
wherein said antibody, or antigen binding fragment thereof, specifically binds to an antigen expressed on the cell surface of a cancer cell, and
wherein said antibody, or antigen binding fragment thereof, specifically binds to prostate-specific membrane antigen (PSMA), Her2 antigen, CD37, or CD123.

2. The antibody-drug conjugate (ADC) according to embodiment 1, wherein said antibody, or antigen binding fragment thereof, specifically binds to PSMA and comprises a CDRH1 according to SEQ ID NO. 378, a CDRH2 according to SEQ ID NO. 379, a CDRH3 according to SEQ ID NO. 380, a CDRL1 according to SEQ ID NO. 381, a CDRL2 according to SEQ ID NO. 382, and a CDRL3 according to SEQ ID NO. 383.

3. The antibody-drug conjugate (ADC) according to embodiment 2, wherein said antibody, or antigen binding fragment thereof, comprises a heavy chain variable region according to SEQ ID NO. 375 and a light chain variable region according to SEQ ID NO. 377.

4. The antibody-drug conjugate (ADC) according to embodiment 3, wherein said antibody comprises a heavy chain according to SEQ ID NO. 372, SEQ ID NO. 373, or SEQ ID NO. 374, and a light chain according to SEQ ID NO. 376, or an antigen binding fragment thereof.

5. The antibody-drug conjugate (ADC) according to embodiment 1, wherein said antibody, or antigen binding fragment thereof, is conjugated to any compound which is selected from the group consisting of HDP30.2060, HDP30.2115, HDP30.2347, HDP30.1699, HDP30.2371, HDP30.0880:

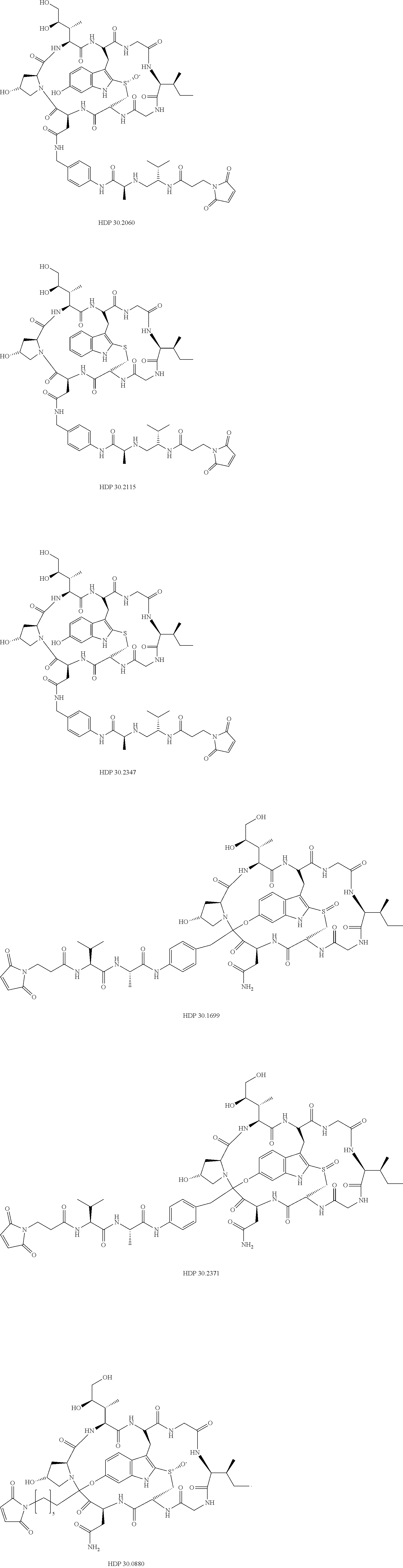

6. An antibody-drug conjugate (ADC) according to embodiment 5, wherein the drug antibody ratio (DAR) is about 1, 2, 3, or 4, preferably wherein the DAR is 2.

7. A method, comprising:
treating cancer in a patient with the antibody-drug conjugate (ADC) of embodiment 6.

8. A pharmaceutical composition, comprising:
The antibody-drug conjugate (ADC) according to embodiment 1, and
at least one pharmaceutically acceptable carrier.

9. A method, comprising:
treating of B lymphocyte-associated malignancies, or B cell-mediated autoimmune diseases selected from the group consisting of non-Hodgkin's lymphoma, follicular lymphoma, diffuse large B cell non-Hodgkin's lymphoma, chronic lymphocytic leukemia, rheumatoid arthritis, granulomatosis with polyangiitis and microscopic polyangiitis and pemphigus vulgaris with the pharmaceutical composition according to embodiment 8.

### BRIEF DESCRIPTION OF THE DRAWINGS

FIG. 1A shows the structure of formula IV. “Ab” represents an antibody. FIG. 1A represents Conjugate A, referred to in the examples. Conjugate A is also referred to as ADC A.

FIG. 1B shows the structure of formula VI. “Ab” represents an antibody. FIG. 1B represents Conjugate B, referred to in the examples. Conjugate B is also referred to as ADC B.

FIG. 1C shows the structure of formula IIa. “Ab” represents an antibody. FIG. 1C represents Conjugate C, referred to in the examples. Conjugate C is also referred to as ADC C.

FIG. 2A graphically depicts results from in vitro cytoxicity assays using Kasumi-1 cells in the presence of ADC with pre-incubation of ADC in media to evaluate serum stability of conjugates.

FIG. 2B graphically depicts results from in vitro cytoxicity assays using Kasumi-1 cells in the presence of ADC with pre-incubation of ADC in 50% human serum to evaluate serum stability of conjugates.

FIG. 3 graphically depicts results from in vitro cytoxicity assays using Kasumi-1 cells in the presence of ADC with iteration on the time of incubation with titrated ADC samples to evaluate differences in the kinetics of cytotoxicity between cleavable and non-cleavable conjugates.

FIG. 4A graphically depicts results from in vitro cell killing assays using Kasumi-1 cells. The ADC tested was anti-CD117 ADC C.

FIG. 4B graphically depicts results from in vitro cell killing assays using CD34+ cells. The ADC tested was anti-CD117 ADC C.

FIG. 5 graphically depicts the ability of anti-CD117 ADC C to potently deplete human CD34+ in the bone marrow of humanized NSG mice.

FIG. 6 are Kaplan Meier plots reflecting the survival of Kasumi-1 implanted humanized NSG mice treated with the indicated doses of conjugates or controls.

FIG. 7 graphically depicts the efficacy of Conjugate C as evaluated in male cynomolgus monkeys. 2.0 mg/kg dose (LALA) 30.2867 batch issues may have resulted in a decrease in HSC sensitivity FIG. 8 graphically depicts tolerability of ADCs containing either Conjugate A or Conjugate C in male cynomolgus monkeys.

FIG. 9 graphically depicts pharmacokinetic analysis of conjugate A and conjugate C administered to male cynomolgus monkeys.

FIG. 10 graphically depicts that anti-CD2 and CD5 ADC C are able to deplete T cells.

FIG. 11 graphically depicts that while anti-CD2 ADCs A and C are saturated by Day 5, some cells still express CD5.

FIG. 12A graphically depicts results showing anti-CD45 ADCs A or C in in vitro cell killing assays.

FIG. 12B graphically depicts results showing anti CD45 ADCs A or B in in vitro cell killing assays.

FIG. 13 graphically depicts results showing anti-CD45 ADCs A or C in in vitro cell killing assays.

FIG. 14 graphically depicts results of anti-CD45 ADCs A or C in in vivo cell depletion experiments.

FIG. 15 graphically depicts results from administration of anti-CD45 ADCs A or C to mice at a number of dose amounts. Levels of peripheral lymphocytes, HSCs, and lymphocytes are shown. All ADCs were administered at 1 mg/kg.

FIG. 16 graphically depicts results showing anti-CD137 ADCs A and C in a T cell killing assay.

FIG. 17 graphically depicts cell line serum stability of anti-CD137 ADCs A and C over 48 hours.

FIG. 18 depicts the structures of amanitin-linker constructs HDP30.2867, 30.0880, 30.2371, and 30.1699, respectively.

FIG. 19 depicts the structures of amanitin-linker constructs HDP30.2115, 30.2060, and 30.2347, respectively.

FIG. 20A graphically depicts the cytotoxic activity in vitro of ADC compounds T-D265C-30.2867 and T-D265C-30.0880, respectively, on SKBR-3 cell lines in 96h-BrdU assay.

FIG. 20B graphically depicts the cytotoxic activity in vitro of ADC compounds T-D265C-30.2867 and T-D265C-30.0880, respectively, on NCI-N87 cell lines in 96h-BrdU assay.

FIG. 20C graphically depicts the cytotoxic activity in vitro of ADC compounds T-D265C-30.2867 and T-D265C-30.0880, respectively, on BT474 cell lines in 96h-BrdU assay.

FIG. 20D graphically depicts the cytotoxic activity in vitro of ADC compounds T-D265C-30.2867 and T-D265C-30.0880, respectively, on JIMT-1 cell lines in 96h-BrdU assay.

FIG. 21A graphically depicts results of SDS-PAGE/Western Blot analysis of the stability of ADC compounds T-D265C-30.2867 and T-D265C-30.0880, respectively, after incubation for 0, 4 and 10 days, respectively, in human plasma.

FIG. 21B graphically depicts results of SDS-PAGE/Western Blot analysis of the stability of ADC compounds T-D265C-30.2867 and T-D265C-30.0880, respectively, after incubation for 0, 4 and 10 days, respectively, in mouse plasma.

FIG. 21C graphically depicts results of SDS-PAGE/Western Blot analysis of the stability of ADC compounds T-D265C-30.2867 and T-D265C-30.0880, respectively, after incubation for 0, 4 and 10 days, respectively, in cynomolgus plasma.

FIG. 21D graphically depicts results of SDS-PAGE/Western Blot analysis of the stability of ADC compounds T-D265C-30.2867 and T-D265C-30.0880, respectively, after incubation for 0, 4 and 10 days, respectively, in PBS (control).

FIG. 22A graphically depicts the cytotoxic activity in vitro of ADC compound T-D265C-30.2867 on SBR-3 cells after incubation for 0, 4 and 10 days, respectively, in human plasma.

FIG. 22B graphically depicts the cytotoxic activity in vitro of ADC compound T-D265C-30.2867 on SBR-3 cells after incubation for 0, 4 and 10 days, respectively, in mouse plasma.

FIG. 22C graphically depicts the cytotoxic activity in vitro of ADC compound T-D265C-30.2867 on SBR-3 cells after incubation for 0, 4 and 10 days, respectively, in cynomolgus plasma.

FIG. 22D graphically depicts the cytotoxic activity in vitro of ADC compound T-D265C-30.2867 on SBR-3 cells after incubation for 0, 4 and 10 days, respectively, in PBS (control).

FIG. 23A graphically depicts the cytotoxic activity in vitro of ADC compound T-D265C-30.2867 on NCI-N87 cells after incubation for 0, 4 and 10 days, respectively, in human plasma.

FIG. 23B graphically depicts the cytotoxic activity in vitro of ADC compound T-D265C-30.2867 on NCI-N87 cells after incubation for 0, 4 and 10 days, respectively, in mouse plasma.

FIG. 23C graphically depicts the cytotoxic activity in vitro of ADC compound T-D265C-30.2867 on NCI-N87 cells after incubation for 0, 4 and 10 days, respectively, in cynomolgus plasma.

FIG. 23D graphically depicts the cytotoxic activity in vitro of ADC compound T-D265C-30.2867 on NCI-N87 cells after incubation for 0, 4 and 10 days, respectively, in PBS (control).

FIG. 24A graphically depicts the cytotoxic activity in vitro of ADC compound T-D265C-30.2867 on JIMT-1 cells after incubation for 0, 4 and 10 days, respectively, in human plasma.

FIG. 24B graphically depicts the cytotoxic activity in vitro of ADC compound T-D265C-30.2867 on JIMT-1 cells after incubation for 0, 4 and 10 days, respectively, in mouse plasma.

FIG. 24C graphically depicts the cytotoxic activity in vitro of ADC compound T-D265C-30.2867 on JIMT-1 cells after incubation for 0, 4 and 10 days, respectively, in cynomolgus plasma.

FIG. 24D graphically depicts the cytotoxic activity in vitro of ADC compound T-D265C-30.2867 on JIMT-1 cells after incubation for 0, 4 and 10 days, respectively, in PBS (control).

FIG. 25 graphically depicts results of cytotoxic efficacy analysis of Anti-Her2-ADCs in JIMT-1-cell xenograft tumor mouse models in vivo using ADC compounds T-D265C-30.2867, T-D265C-30.0880 and T-D265C-30.1699.

FIG. 26 graphically depicts results of cytotoxic efficacy analysis of Anti-Her2-ADCs in NCI-N87-cell xenograft tumor mouse models in vivo using ADC compounds T-D265C-30.2867, T-D265C-30.0880 and T-D265C-30.1699.

FIG. 27A graphically depicts the cytotoxic activity in vitro of ADC compounds h3/F11-D265C-Var16-30.2867 and h3/F11-D265C-Var16-30.0880, respectively, on LNCap cell lines in 96h-BrdU assay.

FIG. 27B graphically depicts the cytotoxic activity in vitro of ADC compounds h3/F11-D265C-Var16-30.2867 and h3/F11-D265C-Var16-30.0880, respectively, on 22RV1 cell lines in 96h-BrdU assay.

FIG. 27C graphically depicts the cytotoxic activity in vitro of ADC compounds h3/F11-D265C-Var16-30.2867 and h3/F11-D265C-Var16-30.0880, respectively, on PC3 cell lines in 96h-BrdU assay.

FIG. 28 graphically depicts results of cytotoxic efficacy analysis of Anti-PSMA-ADCs in LNCap-cell xenograft tumor mouse models in vivo using ADC compounds h3/F11-D265C-Var16-30.2867, h3/F11-D265C-Var16-30.0880 and h3/F11-D265C-Var16-30.2060.

FIG. 29A graphically depicts results of a tolerability study in monkeys using the Anti-Digoxigenin ADC compound DIG-D265C-30.2867 at different doses between 1 mg/kg and 20 mg/kg. Results for assessment of LDH parameters are shown.

FIG. 29B graphically depicts results of a tolerability study in monkeys using the Anti-Digoxigenin ADC compound DIG-D265C-30.2867 at different doses between 1 mg/kg and 20 mg/kg. Results for assessment of AST parameters are shown.

FIG. 29C graphically depicts results of a tolerability study in monkeys using the Anti-Digoxigenin ADC compound DIG-D265C-30.2867 at different doses between 1 mg/kg and 20 mg/kg. Results for assessment of ALT parameters are shown.

FIG. 30A graphically depicts results of a tolerability study in monkeys using DIG-D265C-30.2867. Pharmacokinetic data for the ADC in serum are shown.

FIG. 30B graphically depicts results of a tolerability study in monkeys using DIG-D265C-30.2867. Pharmacokinetic data for the ADC in serum are shown.

FIG. 31A graphically depicts results of a tolerability study in monkeys using DIG-D265C-30.2867. Pharmacokinetic data for the amatoxin in serum are shown.

FIG. 31B graphically depicts results of a tolerability study in monkeys using DIG-D265C-30.2867. Pharmacokinetic data for the amatoxin in serum are shown.

FIG. 32A graphically depicts the cytotoxic activity in vitro of ADC compounds comprising an anti-Her2 antibody with a D265C mutation (T-D265C) conjugated to structurally different amanitin derivatives on JIMT-1 cells.

FIG. 32B graphically depicts the cytotoxic activity in vitro of ADC compounds comprising an anti-Her2 antibody with a D265C mutation (T-D265C) conjugated to structurally different amanitin derivatives on JIMT-1 cells.

FIG. 33A graphically depicts the cytotoxic activity in vitro of ADC compounds comprising an anti-Her2 antibody with a D265C mutation (T-D265C) conjugated to structurally different amanitin derivatives on NCI-N87 cells.

FIG. 33B graphically depicts the cytotoxic activity in vitro of ADC compounds comprising an anti-Her2 antibody with a D265C mutation (T-D265C) conjugated to structurally different amanitin derivatives on NCI-N87 cells.

FIG. 34A graphically depicts the cytotoxic activity in vitro of ADC compounds comprising an anti-Her2 antibody with a D265C mutation (T-D265C) conjugated to structurally different amanitin derivatives on SKBR-3 cells.

FIG. 34B graphically depicts the cytotoxic activity in vitro of ADC compounds comprising an anti-Her2 antibody with a D265C mutation (T-D265C) conjugated to structurally different amanitin derivatives on SKBR-3 cells.

FIG. 35A graphically depicts the cytotoxic activity in vitro of ADC compounds comprising an anti-PSMA antibody with a D265C mutation (h3/F11-D265C-Var16) conjugated to structurally different amanitin derivatives on LNCap cells.

FIG. 35B graphically depicts the cytotoxic activity in vitro of ADC compounds comprising an anti-PSMA antibody with a D265C mutation (h3/F11-D265C-Var16) conjugated to structurally different amanitin derivatives on LNCap cells.

FIG. 36A graphically depicts the cytotoxic activity in vitro of ADC compounds comprising an anti-PSMA antibody with a D265C mutation (h3/F11-D265C-Var16) conjugated to structurally different amanitin derivatives on 22RV1 cells.

FIG. 36B graphically depicts the cytotoxic activity in vitro of ADC compounds comprising an anti-PSMA antibody with a D265C mutation (h3/F11-D265C-Var16) conjugated to structurally different amanitin derivatives on 22RV1 cells.

FIG. 37 graphically depicts results of cytotoxic efficacy analysis of various Anti-PSMA-ADCs comprising structurally different amanitin derivatives in an LNCap-cell xenograft tumor mouse model in vivo. Means+/−SEM for all groups at maximum tolerated dose (MTD) ½ and ¼ are shown.

FIG. 38 graphically depicts results of cytotoxic efficacy analysis of various Anti-PSMA-ADCs comprising structurally different amanitin derivatives in an LNCap-cell xenograft tumor mouse model in vivo. Means+/−SEM for all groups at maximum tolerated dose (MTD) ½ are shown.

FIG. 39 graphically depicts results of cytotoxic efficacy analysis of various Anti-PSMA-ADCs comprising structurally different amanitin derivatives in an LNCap-cell xenograft tumor mouse model in vivo. Means+/−SEM for all groups at maximum tolerated dose (MTD) ¼ are shown.

FIG. 40 graphically depicts results of cytotoxic efficacy analysis of various Anti-PSMA-ADCs comprising structurally different amanitin derivatives in an LNCap-cell xenograft tumor mouse model in vivo. Means+/−SEM for ADCs with linker attachment at amino acid 1 of the amatoxin at maximum tolerated dose (MTD) ½ and ¼ are shown.

FIG. 41 graphically depicts results of cytotoxic efficacy analysis of various Anti-PSMA-ADCs comprising structurally different amanitin derivatives in an LNCap-cell xenograft tumor mouse model in vivo. Means+/−SEM for ADCs with linker attachment at amino acid 4 of the amatoxin at maximum tolerated dose (MTD) ½ and ¼ are shown.

FIG. 42 graphically depicts results of cytotoxic efficacy analysis of various Anti-PSMA-ADCs comprising structurally different amanitin derivatives in an LNCap-cell xenograft tumor mouse model in vivo. Means+/−SEM for ADCs with cleavable linkers at maximum tolerated dose (MTD) ¼ are shown.

FIG. 43 graphically depicts results of cytotoxic efficacy analysis of anti-Her2 ADCs comprising triple L234A/L235A/D265C mutations and the amatoxin-linker construct HDP30.2060 and HDP30.2867, respectively (T-LALA-D265C-30.2060, T-LALA-D265C-30.2867, respectively) on Her2-positive NCI-N87 cells in a CDX mouse model.

FIG. 44 graphically depicts results of cytotoxic efficacy analysis of anti-PSMA ADCs comprising triple L234A/L235A/D265C mutations and the amatoxin-linker construct HDP30.2060 and HDP30.2867, respectively (h3/F11-LALA-D265C-Var-30.2060, h3/F11-LALA-D265C-Var-30.2867, respectively) on PSMA-positive LNCap cells in a CDX mouse model.

FIG. 45A graphically depicts results of a 96-hour BrdU assay for assessment of the cytotoxic potential in vitro of Her2-specific ADCs comprising an antibody carrying L234A/L235A mutations and/or the D265C mutation, or control mutations, on Her2-positive SKBR-3 cells.

FIG. 45B graphically depicts results of a 96-hour BrdU assay for assessment of the cytotoxic potential in vitro of Her2-specific ADCs comprising an antibody carrying L234A/L235A mutations and/or the D265C mutation, or control mutations, on Her2-positive SKBR-3 cells.

FIG. 45C graphically depicts results of a 96-hour BrdU assay for assessment of the cytotoxic potential in vitro of Her2-specific ADCs comprising an antibody carrying L234A/L235A mutations and/or the D265C mutation, or control mutations, on Her2-positive SKBR-3 cells.

FIG. 45D graphically depicts results of a 96-hour BrdU assay for assessment of the cytotoxic potential in vitro of Her2-specific ADCs comprising an antibody carrying L234A/L235A mutations and/or the D265C mutation, or control mutations, on Her2-positive SKBR-3 cells.

FIG. 46A graphically depicts results of a 96-hour CTG assay for assessment of the cytotoxic potential in vitro of Her2-specific ADCs comprising an antibody carrying L234A/L235A mutations and/or the D265C mutation, or control mutations, on Her2-negative THP-1 cells.

FIG. 46B graphically depicts results of a 96-hour CTG assay for assessment of the cytotoxic potential in vitro of Her2-specific ADCs comprising an antibody carrying L234A/L235A mutations and/or the D265C mutation, or control mutations, on Her2-negative THP-1 cells.

FIG. 46C graphically depicts results of a 96-hour CTG assay for assessment of the cytotoxic potential in vitro of Her2-specific ADCs comprising an antibody carrying L234A/L235A mutations and/or the D265C mutation, or control mutations, on Her2-negative THP-1 cells.

FIG. 46D graphically depicts results of a 96-hour CTG assay for assessment of the cytotoxic potential in vitro of Her2-specific ADCs comprising an antibody carrying L234A/L235A mutations and/or the D265C mutation, or control mutations, on Her2-negative THP-1 cells.

FIG. 47A graphically depicts results of a 120-hour CTG assay for assessment of the cytotoxic potential in vitro of Her2-specific ADCs comprising an antibody carrying L234A/L235A mutations and/or the D265C mutation, or control mutations, on Her2-negative THP-1 cells.

FIG. 47B graphically depicts results of a 120-hour CTG assay for assessment of the cytotoxic potential in vitro of Her2-specific ADCs comprising an antibody carrying L234A/L235A mutations and/or the D265C mutation, or control mutations, on Her2-negative THP-1 cells.

FIG. 47C graphically depicts results of a 120-hour CTG assay for assessment of the cytotoxic potential in vitro of Her2-specific ADCs comprising an antibody carrying L234A/L235A mutations and/or the D265C mutation, or control mutations, on Her2-negative THP-1 cells.

FIG. 47D graphically depicts results of a 120-hour CTG assay for assessment of the cytotoxic potential in vitro of Her2-specific ADCs comprising an antibody carrying L234A/L235A mutations and/or the D265C mutation, or control mutations, on Her2-negative THP-1 cells.

### DETAILED DESCRIPTION OF THE INVENTION

For clarity of disclosure, and not by way of limitation, the detailed description of the invention is divided into the subsections which follow.

The present invention relates to an amatoxin, or derivative or analogue thereof, comprising the structure of formula (An) or formula (Bn)

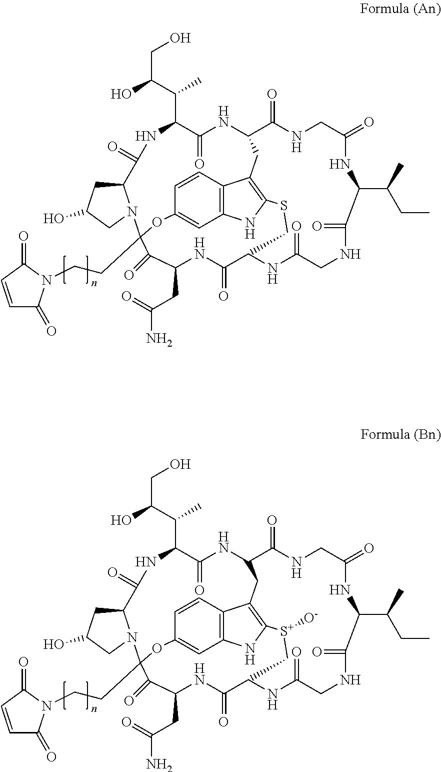

wherein n is 2, 3, 4, 5, 6, 7, 8 or 9.

In one embodiment, the invention relates to an amatoxin, or derivative or analogue thereof, comprising the structure of formula (A)

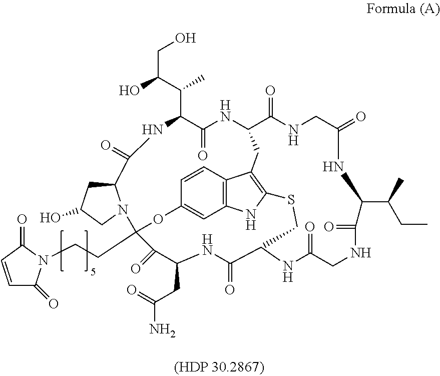

or an enantiomer or diastereomer thereof.

In a further embodiment, the invention relates to an amatoxin, or derivative or analogue thereof, comprising the structure of formula (B)

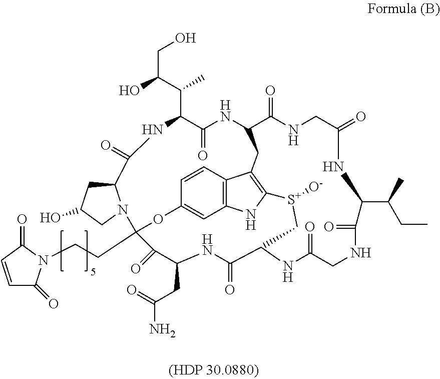

or an enantiomer or diastereomer thereof.

Said amatoxins, or derivatives or analogues thereof, can be used in the preparation of an antibody-drug conjugate (ADC).

ADCs according to the present invention have been shown by the inventors to have a particularly high plasma stability and tolerability, and therefore, an improved therapeutic window.

The present invention further relates to an antibody-drug conjugate (ADC) comprising an antibody, or an antigen-binding fragment thereof, conjugated to an amatoxin via a linker, the ADC having the structure of formula (I):

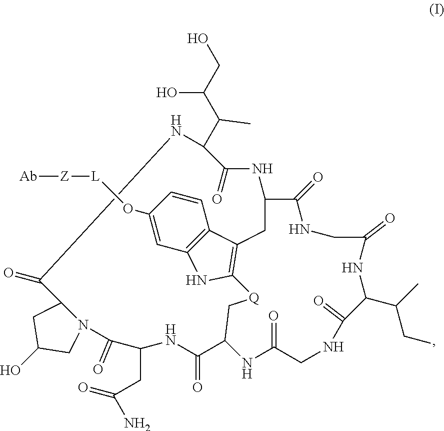

or a stereoisomer thereof,
wherein:
Q is S or a sulfoxid group;
L is a non-cleavable linker;
Z is a chemical moiety formed by a coupling reaction between a reactive substituent
present on L and a reactive substituent present within the antibody, or antigen-binding fragment thereof, and
Ab is the antibody, or the antigen binding fragment thereof.

Said ADC may have the structure of formula (Ia):

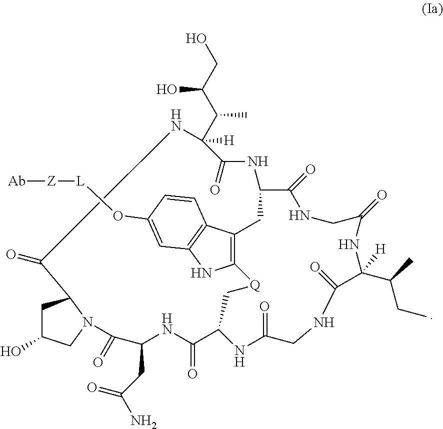

Said ADC may also have the structure of formula (Ib):

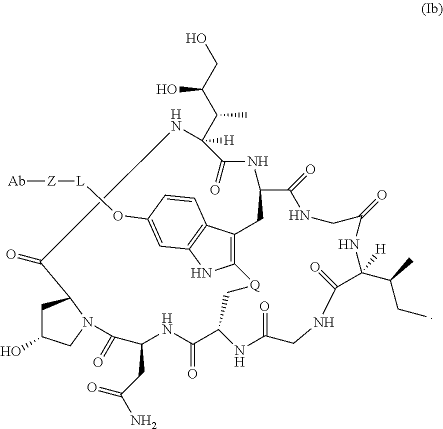

In said ADCs, L can comprise one or more of a bond, —(C═O)—, a —C(O)NH— group, an —OC(O)NH— group, C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, heteroarylene, a —(CH₂CH₂O)ₚ— group where p is an integer from 1-6, or a solubility enhancing group;
wherein each C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, or heteroarylene may optionally be substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro;
or each C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, or heteroarylene may optionally be interrupted by one or more heteroatoms selected from O, S and N.

Said solubility enhancing group may have the formula —Oₐ—C(O)NH—SO₂—NR¹—, wherein:
a is 0 or 1; and
R¹ is selected from the group consisting of hydrogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, each of which may be optionally substituted or optionally interrupted by one or more heteroatoms selected from O, S and NR³, wherein R³ is independently selected from the group consisting of hydrogen and C₁-C₄ alkyl.

In said ADCs, L may comprise a —(CH₂)ₙ— unit, where n is an integer from 2-6. Preferably L is —(CH₂)ₙ—, where n is 6.

In said ADCs, Ab, Z, and L, taken together as Ab-Z-L, can be represented by the formula:

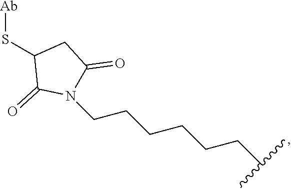

wherein S is the sulfur atom of a cysteine residue present in the antibody, or the antigen-binding fragment thereof.

In one embodiment, the present invention relates to an antibody-drug conjugate (ADC) comprising an antibody, or an antigen-binding fragment thereof, conjugated to an amatoxin via a linker, the ADC having the structure of formula (I):

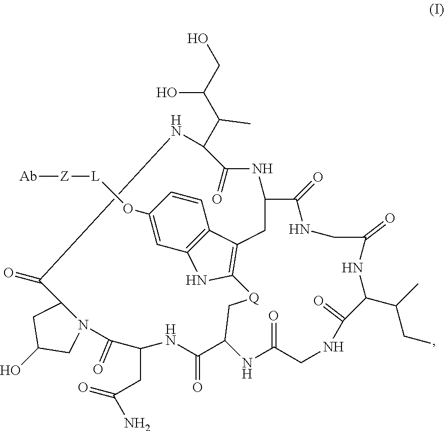

or a stereoisomer thereof,
wherein:
   Q is S or a sulfoxid group;
   L is a cleavable linker;
   Z is a chemical moiety formed by a coupling reaction between a reactive substituent present on L and a reactive substituent present within the antibody, or antigen-binding fragment thereof, and
   Ab is the antibody, or the antigen binding fragment thereof.

Said ADC can have the structure of formula (Ia):

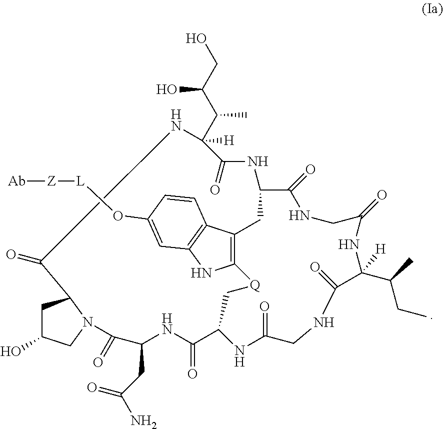

Said ADC can have the structure of formula (Ib):

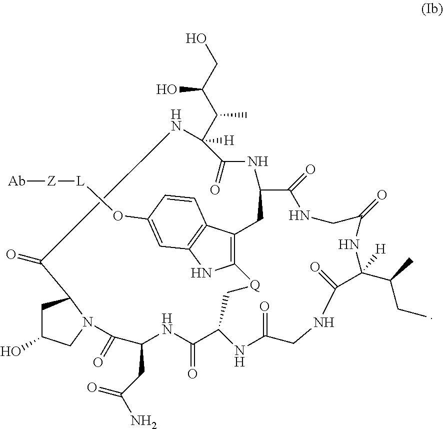

In said ADCs, L comprises one or more of a hydrazine, a disulfide, a thioether, an amino acid, a peptide consisting of up to 10 amino acids, a p-aminobenzyl (PAB) group, a heterocyclic self-immolative group, C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, heteroaryl, a —(C═O)— group, a —C(O)NH— group, an —OC(O)NH— group, a —(CH₂CH₂O)ₚ— group where p is an integer from 1-6, or a solubility enhancing group;
wherein each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, or heteroaryl group may be optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro;
or each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, or heteroaryl group may optionally be interrupted by one or more heteroatoms selected from O, S and N.

Said solubility enhancing group may have the formula —Oₐ—C(O)NH—SO₂—NR¹—, wherein:
a is 0 or 1; and
R¹ is selected from the group consisting of hydrogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, each of which may be optionally substituted or optionally interrupted by one or more heteroatoms selected from O, S and NR³, wherein R³ is independently selected from the group consisting of hydrogen and C₁-C₄ alkyl.

According to one embodiment of the present invention, L comprises a peptide selected from the group consisting of Phe-Lys, Val-Lys, Phe-Ala, Phe-Cit, Val-Ala, Val-Cit, and Val-Arg. L may further comprise a PAB group.

In one embodiment, L is represented by the formula:

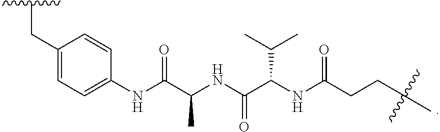 [SMILES: *CCC(=O)N[C@H](C(=O)N[C@@H](C)C(=O)Nc1ccc(C*)cc1)C(C)C]

According to a preferred embodiment, the invention relates to an ADC comprising an antibody conjugated to an amatoxin, the ADC having a structure according to formula (II):

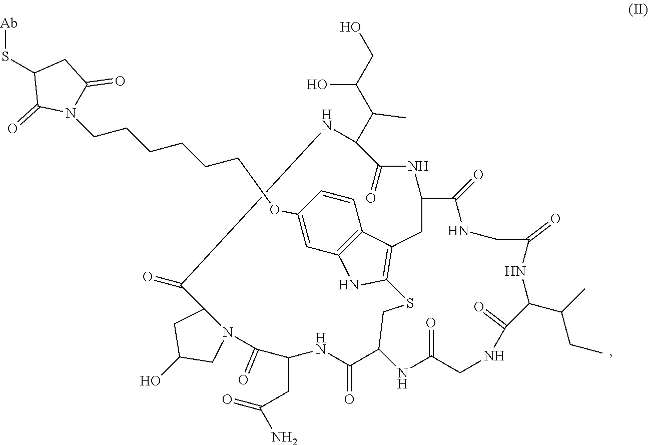

or a stereoisomer thereof.

Said ADC can have the structure of formula (IIa):

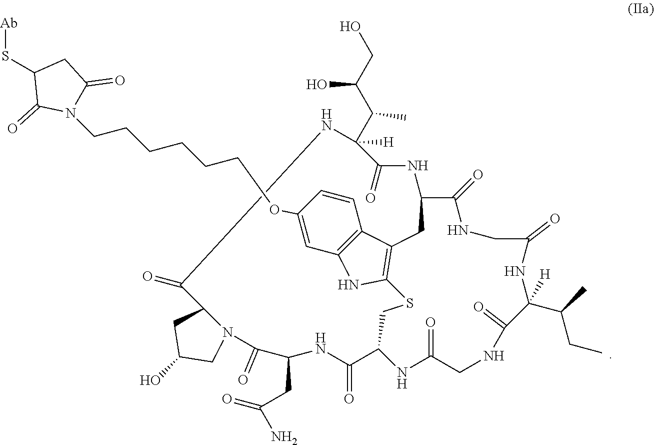

Said ADC can have the structure of formula (IIb):

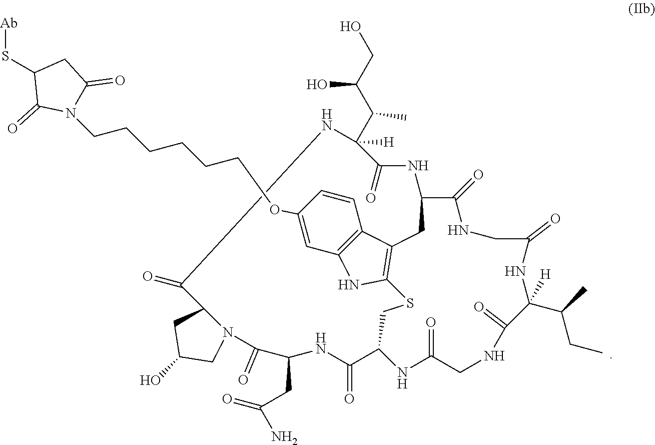

According to a preferred embodiment of the invention, the antibody, or the antigen binding fragment thereof, specifically binds to an antigen expressed on the cell surface of a cancer cell, or a human stem cell, in particular a hematopoietic stem cell (HSC), or a T cell.

According to a further preferred embodiment of the invention, the antibody, or the antigen binding fragment thereof, specifically binds to human Her2, PSMA, CD37, or CD123.

According to another preferred embodiment of the present invention, the antibody, or the antigen binding fragment thereof, comprises an Fc region comprising at least one mutation selected from the group consisting of D265C, D265A, A118C, H435A, L234A, or L235A (according to EU index).

According to another preferred embodiment of the present invention, the antibody, or antigen binding fragment thereof, specifically binds to PSMA and comprises a CDRH1 according to SEQ ID NO. 378, a CDRH2 according to SEQ ID NO. 379, a CDRH3 according to SEQ ID NO. 380, a CDRL1 according to SEQ ID NO. 381, a CDRL2 according to SEQ ID NO. 382, and a CDRL3 according to SEQ ID NO. 383.

According to another preferred embodiment of the present invention, the antibody, or antigen binding fragment thereof, comprises a heavy chain variable region according to SEQ ID NO. 375 and a light chain variable region according to SEQ ID NO. 377.

According to another preferred embodiment of the present invention, said antibody comprises a heavy chain according to SEQ ID NO. 371, SEQ ID NO. 372, SEQ ID NO. 373, or SEQ ID NO. 374, and a light chain according to SEQ ID NO. 376, or an antigen binding fragment thereof.

The invention further relates to an antibody-drug conjugate (ADC) comprising an antibody, or an antigen-binding fragment thereof, conjugated to an amatoxin, or a derivative or analogue thereof, via a linker, wherein the antibody, or the antigen binding fragment thereof, comprises an Fc region comprising at least two mutations consisting of L234A and L235A (according to EU index).

According to a particularly preferred embodiment, said Fc region is further comprising a mutation consisting of D265C (according to EU index).

According to a preferred embodiment of the present invention, said antibody, or antigen binding fragment thereof, specifically binds to an antigen expressed on the cell surface of a cancer cell, preferably of a human cancer cell.

According to another preferred embodiment of the present invention, said antibody, or antigen binding fragment thereof, specifically binds to prostate-specific membrane antigen (PSMA), preferably human PSMA, or to Her2 antigen, CD37, or CD123.

A preferred embodiment relates to an antibody-drug conjugate (ADC), wherein said antibody, or antigen binding fragment thereof, specifically binds to PSMA and comprises a CDRH1 according to SEQ ID NO. 378, a CDRH2 according to SEQ ID NO. 379, a CDRH3 according to SEQ ID NO. 380, a CDRL1 according to SEQ ID NO. 381, a CDRL2 according to SEQ ID NO. 382, and a CDRL3 according to SEQ ID NO. 383.

According to a preferred embodiment of the present invention, said antibody specifically binding to PSMA, or antigen binding fragment thereof, comprises a heavy chain variable region according to SEQ ID NO. 375 and a light chain variable region according to SEQ ID NO. 377.

According to another preferred embodiment of the present invention, said antibody comprises a heavy chain according to SEQ ID NO. 372, SEQ ID NO. 373, or SEQ ID NO. 374, and a light chain according to SEQ ID NO. 376, or an antigen binding fragment thereof.

In further preferred embodiments, the antibody-drug conjugate (ADC) comprises an antibody, or antigen binding fragment thereof, which is conjugated to any compound which is selected from the group consisting of HDP30.2060, HDP30.2115, HDP30.2347, HDP30.1699, HDP30.2371, HDP30.0880, and HDP30.2867.

The invention further relates to an antibody-drug conjugate (ADC) comprising an antibody, or an antigen-binding fragment thereof, which specifically binds to human PSMA, and comprises a heavy chain having an amino acid sequence according to SEQ ID NO. 374 and a light chain having an amino acid sequence according to SEQ ID NO. 376, and which is conjugated to compound HDP30.2060. Most preferrably, in said ADC the compound HDP30.2060 is linked directly to the sulfur atom of cysteine D265C of said antibody (EU numbering).

The invention further relates to an antibody-drug conjugate (ADC) comprising an antibody, or an antigen-binding fragment thereof, which specifically binds to human PSMA, and comprises a heavy chain having an amino acid sequence according to SEQ ID NO. 374 and a light chain having an amino acid sequence according to SEQ ID NO. 376, and which is conjugated to compound HDP30.2115. Most preferrably, in said ADC the compound HDP30.2115 is linked directly to the sulfur atom of cysteine D265C of said antibody.

The invention further relates to an antibody-drug conjugate (ADC) comprising an antibody, or an antigen-binding fragment thereof, which specifically binds to human PSMA, and comprises a heavy chain having an amino acid sequence according to SEQ ID NO. 374 and a light chain having an amino acid sequence according to SEQ ID NO. 376, and which is conjugated to compound HDP30.2347. Most preferrably, in said ADC the compound HDP30.2347 is linked directly to the sulfur atom of cysteine D265C of said antibody.

The invention further relates to an antibody-drug conjugate (ADC) comprising an antibody, or an antigen-binding fragment thereof, which specifically binds to human PSMA, and comprises a heavy chain having an amino acid sequence according to SEQ ID NO. 374 and a light chain having an amino acid sequence according to SEQ ID NO. 376, and which is conjugated to compound HDP30.1699. Most preferrably, in said ADC the compound HDP30.1699 is linked directly to the sulfur atom of cysteine D265C of said antibody.

The invention further relates to an antibody-drug conjugate (ADC) comprising an antibody, or an antigen-binding fragment thereof, which specifically binds to human PSMA, and comprises a heavy chain having an amino acid sequence according to SEQ ID NO. 374 and a light chain having an amino acid sequence according to SEQ ID NO. 376, and which is conjugated to compound HDP30.2371. Most preferrably, in said ADC the compound HDP30.2371 is linked directly to the sulfur atom of cysteine D265C of said antibody.

The invention further relates to an antibody-drug conjugate (ADC) comprising an antibody, or an antigen-binding fragment thereof, which specifically binds to human PSMA, and comprises a heavy chain having an amino acid sequence according to SEQ ID NO. 374 and a light chain having an amino acid sequence according to SEQ ID NO. 376, and which is conjugated to compound HDP30.0880. Most preferrably, in said ADC the compound HDP30.0880 is linked directly to the sulfur atom of cysteine D265C of said antibody.

The invention further relates to an antibody-drug conjugate (ADC) comprising an antibody, or an antigen-binding fragment thereof, which specifically binds to human PSMA, and comprises a heavy chain having an amino acid sequence according to SEQ ID NO. 374 and a light chain having an amino acid sequence according to SEQ ID NO. 376, and which is conjugated to compound HDP30.2867. Most preferrably, in said ADC the compound HDP30.2867 is linked directly to the sulfur atom of cysteine D265C of said antibody.

In said antibody-drug conjugates (ADCs) according to the present invention, the drug antibody ratio (DAR) is about 1, 2, 3, or 4, preferably the DAR is 2.

A further aspect of the present invention relates to said ADCs for use in the treatment of cancer in a patient, particularly wherein the cancer is selected from the group consisting of breast cancer, pancreatic cancer, cholangiocarcinoma, colorectal cancer, lung cancer, prostate cancer, ovarian cancer, prostate cancer, stomach cancer, kidney cancer, malignant melanoma, hematological cancer, leukemia, and malignant lymphoma.

The invention further relates to the use of any of said ADCs for treatment of cancer in a patient, particularly wherein the cancer is selected from the group consisting of breast cancer, pancreatic cancer, cholangiocarcinoma, colorectal cancer, lung cancer, prostate cancer, ovarian cancer, prostate cancer, stomach cancer, kidney cancer, malignant melanoma, hematological cancer, leukemia, and malignant lymphoma.

According to another preferred embodiment of the present invention, said antibody-drug conjugate (ADC) comprises an antibody, or antigen binding fragment thereof, that specifically binds to an antigen expressed on the cell surface of a hematopoietic stem cell (HSC), preferably of a human HSC.

A further embodiment of the present invention relates to a method of depleting a population of cells in a human subject, said method comprising administering the said ADC to the subject, wherein the ADC comprises an antibody, or an antigen-binding fragment thereof, that specifically binds to an extracellular antigen expressed by cells in the population of cells.

A further embodiment of the present invention relates to a method of conditioning a human subject for cell transplantation, said method comprising administering said ADC to the human subject such that the endogenous stem or endogenous immune cells in the human subject are depleted, wherein the ADC specifically binds to an extracellular antigen expressed by the endogenous stem or endogenous immune cells.

A further embodiment of the present invention relates to said method, further comprising administering to the human subject allogenic stem cells or allogeneic immune cells.

A further embodiment of the present invention relates to said method, wherein the ADC specifically binds to an extracellular antigen expressed on an immune cell, and wherein the subject has or is at risk of developing graft versus host disease (GVHD).

The invention further relates to a pharmaceutical composition comprising any of said ADCs, or combinations thereof, and at least a pharmaceutically acceptable carrier.

### Definitions

Unless stated otherwise, the following terms and phrases as used herein are intended to have the following meanings.

The term “acyl” as used herein refers to —C(═O)R, wherein R is hydrogen (“aldehyde”), C₁-C₁₂ alkyl, C₂-C₁₂ alkenyl, C₂-C₁₂ alkynyl, C₃-C₇ carbocyclyl, C₆-C₂₀ aryl, 5-10 membered heteroaryl, or 5-10 membered heterocyclyl, as defined herein. Non-limiting examples include formyl, acetyl, propanoyl, benzoyl, and acryloyl.

The term “C₁-C₁₂ alkyl” as used herein refers to a straight chain or branched, saturated hydrocarbon having from 1 to 12 carbon atoms. Representative C₁-C₁₂ alkyl groups include, but are not limited to, -methyl, -ethyl, -n-propyl, -n-butyl, -n-pentyl, and -n-hexyl; while branched C₁-C₁₂ alkyls include, but are not limited to, -isopropyl, -sec-butyl, -isobutyl, -tert-butyl, -isopentyl, and 2-methylbutyl. A C₁-C₁₂ alkyl group can be unsubstituted or substituted.

The term “alkenyl” as used herein refers to C₂-C₁₂ hydrocarbon containing normal, secondary, or tertiary carbon atoms with at least one site of unsaturation, i.e., a carbon-carbon, sp² double bond. Examples include, but are not limited to: ethylene or vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, and the like. An alkenyl group can be unsubstituted or substituted.

“Alkynyl” as used herein refers to a C₂-C₁₂ hydrocarbon containing normal, secondary, or tertiary carbon atoms with at least one site of unsaturation, i.e., a carbon-carbon, sp triple bond. Examples include, but are not limited to acetylenic and propargyl. An alkynyl group can be unsubstituted or substituted.

“Aryl” as used herein refers to a C₆-C₂₀ carbocyclic aromatic group. Examples of aryl groups include, but are not limited to, phenyl, naphthyl and anthracenyl. An aryl group can be unsubstituted or substituted.

“Arylalkyl” as used herein refers to an acyclic alkyl radical in which one of the hydrogen atoms bonded to a carbon atom, typically a terminal or sp³ carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. The arylalkyl group comprises 6 to 20 carbon atoms, e.g. the alkyl moiety, including alkanyl, alkenyl or alkynyl groups, of the arylalkyl group is 1 to 6 carbon atoms and the aryl moiety is 5 to 14 carbon atoms. An alkaryl group can be unsubstituted or substituted.

“Cycloalkyl” as used herein refers to a saturated carbocyclic radical, which may be mono- or bicyclic. Cycloalkyl groups include a ring having 3 to 7 carbon atoms as a monocycle or 7 to 12 carbon atoms as a bicycle. Examples of monocyclic cycloalkyl groups include cyclopropyl, cyclobutyl, cyclopentyl, cyclohexyl, cycloheptyl, and cyclooctyl. A cycloalkyl group can be unsubstituted or substituted.

“Cycloalkenyl” as used herein refers to an unsaturated carbocyclic radical, which may be mono- or bicyclic. Cycloalkenyl groups include a ring having 3 to 6 carbon atoms as a monocycle or 7 to 12 carbon atoms as a bicycle. Examples of monocyclic cycloalkenyl groups include 1-cyclopent-1-enyl, 1-cyclopent-2-enyl, 1-cyclopent-3-enyl, 1-cyclohex-1-enyl, 1-cyclohex-2-enyl, and 1-cyclohex-3-enyl. A cycloalkenyl group can be unsubstituted or substituted.

“Heteroaralkyl” as used herein refers to an acyclic alkyl radical in which one of the hydrogen atoms bonded to a carbon atom, typically a terminal or sp³ carbon atom, is replaced with a heteroaryl radical. Typical heteroarylalkyl groups include, but are not limited to, 2-benzimidazolylmethyl, 2-furylethyl, and the like. The heteroarylalkyl group comprises 6 to 20 carbon atoms, e.g. the alkyl moiety, including alkanyl, alkenyl or alkynyl groups, of the heteroarylalkyl group is 1 to 6 carbon atoms and the heteroaryl moiety is 5 to 14 carbon atoms and 1 to 3 heteroatoms selected from N, O, P, and S. The heteroaryl moiety of the heteroarylalkyl group may be a monocycle having 3 to 7 ring members (2 to 6 carbon atoms or a bicycle having 7 to 10 ring members (4 to 9 carbon atoms and 1 to 3 heteroatoms selected from N, O, P, and S), for example: a bicyclo[4,5], [5,5], [5,6], or [6,6] system.

“Heteroaryl” and “heterocycloalkyl” as used herein refer to an aromatic or non-aromatic ring system, respectively, in which one or more ring atoms is a heteroatom, e.g. nitrogen, oxygen, and sulfur. The heteroaryl or heterocycloalkyl radical comprises 2 to 20 carbon atoms and 1 to 3 heteroatoms selected from N, O, P, and S. A heteroaryl or heterocycloalkyl may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo[4,5], [5,5], [5,6], or [6,6] system. Heteroaryl and heterocycloalkyl can be unsubstituted or substituted.

Heteroaryl and heterocycloalkyl groups are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566.

Examples of heteroaryl groups include by way of example and not limitation pyridyl, thiazolyl, tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, benzotriazolyl, benzisoxazolyl, and isatinoyl.

Examples of heterocycloalkyls include by way of example and not limitation dihydroypyridyl, tetrahydropyridyl (piperidyl), tetrahydrothiophenyl, piperidinyl, 4-piperidonyl, pyrrolidinyl, 2-pyrrolidonyl, tetrahydrofuranyl, tetrahydropyranyl, bis-tetrahydropyranyl, tetrahydroquinolinyl, tetrahydroisoquinolinyl, decahydroquinolinyl, octahydroisoquinolinyl, piperazinyl, quinuclidinyl, and morpholinyl.

By way of example and not limitation, carbon bonded heteroaryls and heterocycloalkyls are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heteroaryls and heterocycloalkyls are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or beta-carboline. Still more typically, nitrogen bonded heterocycles include 1-aziridyl, 1-azetedyl, 1-pyrrolyl, 1-imidazolyl, 1-pyrazolyl, and 1-piperidinyl.

“Substituted” as used herein and as applied to any of the above alkyl, alkenyl, alkynyl, aryl, arylalkyl, cycloalkyl, heteroaryl, heterocyclyl, and the like, means that one or more hydrogen atoms are each independently replaced with a substituent. Unless otherwise constrained by the definition of the individual substituent, the foregoing chemical moieties, such as “alkyl”, “alkylene”, “heteroalkyl”, “heteroalkylene”, “alkenyl”, “alkenylene”, “heteroalkenyl”, “heteroalkenylene”, “alkynyl”, “alkynylene”, “heteroalkynyl”, “heteroalkynylene”, “cycloalkyl”, “cycloalkylene”, “heterocyclolalkyl”, heterocycloalkylene”, “aryl,” “arylene”, “heteroaryl”, and “heteroarylene” groups can optionally be substituted. Typical substituents include, but are not limited to, —X, —R, —OH, —OR, —SH, —SR, NH₂, —NHR, —N(R)₂, —N⁺(R)₃, —CX₃, —CN, —OCN, —SCN, —NCO, —NCS, —NO, —NO₂, —N₃, —NC(═O)H, —NC(═O)R, —C(═O)H, —C(═O)R, —C(═O)NH₂, —C(═O)N(R)₂, —SO₃—, —SO₃H, —S(═O)₂R, —OS(═O)₂OR, —S(═O)₂NH₂, —S(═O)₂N(R)₂, —S(═O)R, —OP(═O)(OH)₂, —OP(═O)(OR)₂, —P(═O)(OR)₂, —PO₃, —PO₃H₂, —C(═O)X, —C(═S)R, —CO₂H, —CO₂R, —CO₂—, —C(═S)OR, —C(═O)SR, —C(═S)SR, —C(═O)NH₂, —C(═O)N(R)₂, —C(═S)NH₂, —C(═S)N(R)₂, —C(═NH)NH₂, and —C(═NR)N(R)₂; wherein each X is independently selected for each occasion from F, Cl, Br, and I; and each R is independently selected for each occasion from C₁-C₁₂ alkyl, C₆-C₂₀ aryl, C₃-C₁₄ heterocycloalkyl or heteroaryl, protecting group and prodrug moiety. Wherever a group is described as “optionally substituted,” that group can be substituted with one or more of the above substituents, independently for each occasion.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene,” “alkenylene,” “arylene,” “heterocycloalkylene,” and the like.

Wherever a substituent is depicted as a di-radical (i.e., has two points of attachment to the rest of the molecule), it is to be understood that the substituent can be attached in any directional configuration unless otherwise indicated.

“Isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers,” or sometimes “optical isomers.”

A carbon atom bonded to four non-identical substituents is termed a “chiral center.” “Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116). A mixture containing equal amounts of individual enantiomeric forms of opposite chirality is termed a “racemic mixture.”

The compounds disclosed in this description and in the embodiments may comprise one or more asymmetric centers, and different diastereomers and/or enantiomers of each of the compounds may exist. The description of any compound in this description and in the embodiments is meant to include all enantiomers, diastereomers, and mixtures thereof, unless stated otherwise. In addition, the description of any compound in this description and in the embodiments is meant to include both the individual enantiomers, as well as any mixture, racemic or otherwise, of the enantiomers, unless stated otherwise. When the structure of a compound is depicted as a specific enantiomer, it is to be understood that the invention of the present application is not limited to that specific enantiomer. Accordingly, enantiomers, optical isomers, and diastereomers of each of the structural formulae of the present disclosure are contemplated herein. In the present specification, the structural formula of the compound represents a certain isomer for convenience in some cases, but the present disclosure includes all isomers, such as geometrical isomers, optical isomers based on an asymmetrical carbon, stereoisomers, tautomers, and the like, it being understood that not all isomers may have the same level of activity. The compounds may occur in different tautomeric forms. The compounds according to the disclosure are meant to include all tautomeric forms, unless stated otherwise. When the structure of a compound is depicted as a specific tautomer, it is to be understood that the invention of the present application is not limited to that specific tautomer.

The compounds of any formula described herein include the compounds themselves, as well as their salts, and their solvates, if applicable. A salt, for example, can be formed between an anion and a positively charged group (e.g., amino) on a compound of the disclosure. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate). The term “pharmaceutically acceptable anion” refers to an anion suitable for forming a pharmaceutically acceptable salt. Likewise, a salt can also be formed between a cation and a negatively charged group (e.g., carboxylate) on a compound of the disclosure. Suitable cations include sodium ion, potassium ion, magnesium ion, calcium ion, and an ammonium cation such as tetramethylammonium ion. Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. The compounds of the disclosure also include those salts containing quaternary nitrogen atoms.

Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Additionally, the compounds of the present disclosure, for example, the salts of the compounds, can exist in either hydrated or unhydrated (the anhydrous) form or as solvates with other solvent molecules. Non-limiting examples of hydrates include monohydrates, dihydrates, etc. Non-limiting examples of solvates include ethanol solvates, acetone solvates, etc. “Solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O. A hydrate refers to, for example, a mono-hydrate, a di-hydrate, a tri-hydrate, etc.

In addition, a crystal polymorphism may be present for the compounds or salts thereof represented by the formulae disclosed herein. It is noted that any crystal form, crystal form mixture, or anhydride or hydrate thereof, is included in the scope of the present disclosure.

As used herein, the term “about” refers to a value that is within 10% above or below the value being described. For example, the term “about 5 nM” indicates a range of from 4.5 nM to 5.5 nM.

As used herein, the term “amatoxin” refers to a member of the amatoxin family of peptides produced by Amanita phalloides mushrooms, or a variant or derivative thereof, such as a variant or derivative thereof capable of inhibiting RNA polymerase II activity. Suitable amatoxins and derivatives thereof are further described herein below. As described herein, amatoxins may be conjugated to an antibody, or antigen-binding fragment thereof, for instance, by way of a linker moiety (L) (thus forming a conjugate (i.e., ADC)). Exemplary methods of amatoxin conjugation and linkers useful for such processes are described below.

In the context of the present invention the term “amatoxin” includes all cyclic peptides composed of 8 amino acids as isolated from the genus Amanita and described in Wieland, T. and Faulstich H. (Wieland T, Faulstich H., CRC Crit Rev Biochem. 5 (1978) 185-260), further all chemical derivatives thereof, further all semisynthetic analogs thereof, further all synthetic analogs thereof built from building blocks according to the master structure of the natural compounds (cyclic, 8 amino acids), further all synthetic or semisynthetic analogs containing non-hydroxylated amino acids instead of the hydroxylated amino acids, further all synthetic or semisynthetic analogs, in which the sulfoxide moiety is replaced by a sulfone, thioether, or by atoms different from sulfur, e.g., a carbon atom as in a carbanalog of amanitin.

As used herein, a “derivative” of a compound refers to a species having a chemical structure that is similar to the compound, yet containing at least one chemical group not present in the compound and/or deficient of at least one chemical group that is present in the compound. The compound to which the derivative is compared is known as the “parent” compound. Typically, a “derivative” may be produced from the parent compound in one or more chemical reaction steps.

As used herein, an “analogue” of a compound is structurally related but not identical to the compound and exhibits at least one activity of the compound. The compound to which the analogue is compared is known as the “parent” compound. The afore-mentioned activities include, without limitation: binding activity to another compound; inhibitory activity, e.g. enzyme inhibitory activity; toxic effects; activating activity, e.g. enzyme-activating activity. It is not required that the analogue exhibits such an activity to the same extent as the parent compound. A compound is regarded as an analogue within the context of the present application, if it exhibits the relevant activity to a degree of at least 1% (more preferably at least 5%, more preferably at least 10%, more preferably at least 20%, more preferably at least 30%, more preferably at least 40%, and more preferably at least 50%) of the activity of the parent compound. Thus, an “analogue of an amatoxin”, as it is used herein, refers to a compound that is structurally related to any one of □-amanitin, β-amanitin, γ-amanitin, ε-amanitin, amanin, amaninamide, amanullin, and amanullinic acid and that exhibits at least 1% (more preferably at least 5%, more preferably at least 10%, more preferably at least 20%, more preferably at least 30%, more preferably at least 40%, and more preferably at least 50%) of the inhibitory activity against mammalian RNA polymerase II as compared to at least one of H-amanitin, β-amanitin, γ-amanitin, ε-amanitin, amanin, amaninamide, amanullin, and amanullinic acid. An “analogue of an amatoxin” suitable for use in the present invention may even exhibit a greater inhibitory activity against mammalian RNA polymerase II than any one of □-amanitin, β-amanitin, γ-amanitin, ε-amanitin, amanin, amaninamide, amanullin, or amanullinic acid. The inhibitory activity might be measured by determining the concentration at which 50% inhibition occurs (IC₅₀ value). The inhibitory activity against mammalian RNA polymerase II can be determined indirectly by measuring the inhibitory activity on cell proliferation.

A “semisynthetic analogue” refers to an analogue that has been obtained by chemical synthesis using compounds from natural sources (e.g. plant materials, bacterial cultures, fungal cultures or cell cultures) as starting material. Typically, a “semisynthetic analogue” of the present invention has been synthesized starting from a compound isolated from a mushroom of the Amanitaceae family. In contrast, a “synthetic analogue” refers to an analogue synthesized by so-called total synthesis from small (typically petrochemical) building blocks. Usually, this total synthesis is carried out without the aid of biological processes.

According to some embodiments of the present invention, the amatoxin can be selected from the group consisting of D-amanitin, D-amanitin, amanin, amaninamide and analogues, derivatives and salts thereof.

Functionally, amatoxins are defined as peptides or depsipeptides that inhibit mammalian RNA polymerase II. Preferred amatoxins are those with a functional group (e.g. a carboxylic group, an amino group, a hydroxy group, a thiol or a thiol-capturing group) that can be reacted with linker molecules or target-binding moieties as defined below.

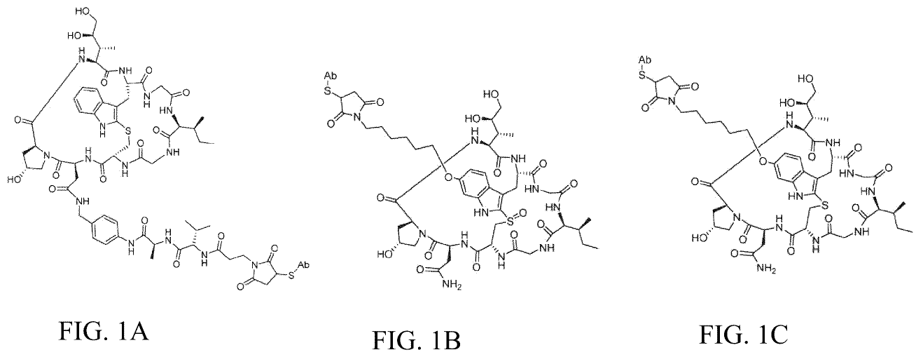

In the context of the present invention, the term “amanitins” particularly refers to bicyclic structure that are based on an aspartic acid or asparagine residue in position 1, a proline residue, particularly a hydroxyproline residue in position 2, an isoleucine, hydroxyisoleucine or dihydroxyisoleucine in position 3, a tryptophan or hydroxytryptophan residue in position 4, glycine residues in positions 5 and 7, an isoleucine residue in position 6, and a cysteine residue in position 8, particularly a derivative of cysteine that is oxidized to a sulfoxide or sulfone derivative (for the numbering and representative examples of amanitins, see FIG. 1A to FIG. 1C), and furthermore includes all chemical derivatives thereof, further all semisynthetic analogues thereof, further all synthetic analogues thereof built from building blocks according to the master structure of the natural compounds (cyclic, 8 amino acids), further all synthetic or semisynthetic analogues containing non-hydroxylated amino acids instead of the hydroxylated amino acids, further all synthetic or semisynthetic analogues, in each case wherein any such derivative or analogue is functionally active by inhibiting mammalian RNA polymerase II

As used herein, the term “antibody” refers to an immunoglobulin molecule that specifically binds to, or is immunologically reactive with, a particular antigen, and includes monoclonal, genetically engineered, and otherwise modified forms of antibodies, including but not limited to chimeric antibodies, humanized antibodies, heteroconjugate antibodies (e.g., bi-tri- and quad-specific antibodies, diabodies, triabodies, and tetrabodies), and antigen binding fragments of antibodies, including, for example, Fab′, F(ab′)₂, Fab, Fv, rlgG, and scFv fragments. Unless otherwise indicated, the term “monoclonal antibody” (mAb) is meant to include both intact molecules, as well as antibody fragments (including, for example, Fab and F(ab′)₂ fragments) that are capable of specifically binding to a target protein. As used herein, the Fab and F(ab′)₂ fragments refer to antibody fragments that lack the Fc fragment of an intact antibody. Examples of these antibody fragments are described herein.

As used herein, the term “monoclonal antibody” refers to an antibody that is derived from a single clone, including any eukaryotic, prokaryotic, or phage clone, and not the method by which it is produced.

The antibodies of the present invention are generally isolated or recombinant. “Isolated,” when used herein refers to a polypeptide, e.g., an antibody, that has been separated and/or recovered from a cell or cell culture from which it was expressed. Thus, an “isolated antibody,” refers to an antibody which is substantially free of other antibodies having different antigenic specificities. For instance, an isolated antibody that specifically binds to CD117 is substantially free of antibodies that specifically bind antigens other than CD117.

The term “antigen-binding fragment,” as used herein, refers a fragment of an antibody that retains the ability to specifically bind to a target antigen. The antigen-binding function of an antibody can be performed by fragments of a full-length antibody. The antibody fragment can be, for example, a Fab, F(ab′)₂, scFv, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment containing two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb including V_{H} and V_{L} domains; (vi) a dAb fragment that consists of a V_{H} domain (see, e.g., Ward et al., Nature 341:544-546, 1989); (vii) a dAb which consists of a V_{H} or a V_{L} domain; (viii) an isolated complementarity determining region (CDR); and (ix) a combination of two or more (e.g., two, three, four, five, or six) isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single chain Fv (scFv); see, for example, Bird et al., Science 242:423-426, 1988 and Huston et al., Proc. Natl. Acad. Sci. USA 85:5879-5883, 1988). Antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art.

As used herein, the term “anti-CD117 antibody” or “an antibody that binds to CD117” refers to an antibody that is capable of binding CD117, e.g., human CD117, with sufficient affinity such that the antibody is useful as a diagnostic and/or therapeutic agent in targeting CD117. The amino acid sequences of the two main isoforms of human CD117 are provided in SEQ ID NO: 145 (isoform 1) and SEQ ID NO: 146 (isoform 2). An “anti-CD117 ADC” refers to an ADC wherein the antibody is an anti-CD117 antibody.

As used herein, the term “anti-CD45 antibody” or “an antibody that binds to CD45” refers to an antibody that is capable of binding CD45, e.g., human CD117, with sufficient affinity such that the antibody is useful as a diagnostic and/or therapeutic agent in targeting CD45. An “anti-CD45 ADC” refers to an ADC wherein the antibody is an anti-CD45 antibody.

As used herein, the term “anti-CD137 antibody” or “an antibody that binds to CD137” refers to an antibody that is capable of binding CD137, e.g., human CD137, with sufficient affinity such that the antibody is useful as a diagnostic and/or therapeutic agent in targeting CD137. An “anti-CD137 ADC” refers to an ADC wherein the antibody is an anti-CD137 antibody.

As used herein, the term “anti-CD2 antibody” or “an antibody that binds to CD2” refers to an antibody that is capable of binding CD2, e.g., human CD2, with sufficient affinity such that the antibody is useful as a diagnostic and/or therapeutic agent in targeting CD2. An “anti-CD2 ADC” refers to an ADC wherein the antibody is an anti-CD2 antibody.

As used herein, the term “anti-CD5 antibody” or “an antibody that binds to CD5” refers to an antibody that is capable of binding CD5, e.g., human CD5, with sufficient affinity such that the antibody is useful as a diagnostic and/or therapeutic agent in targeting CD5. An “anti-CD5 ADC” refers to an ADC wherein the antibody is an anti-CD5 antibody.

As used herein, the term “bispecific antibody” refers to, for example, a monoclonal, often a human or humanized antibody that is capable of binding at least two different antigens. For instance, one of the binding specificities can be directed towards a hematopoietic stem cell surface antigen, CD117 (e.g., GNNK+CD117), and the other can specifically bind a different hematopoietic stem cell surface antigen or another cell surface protein, such as a receptor or receptor subunit involved in a signal transduction pathway that potentiates cell growth, among others.

As used herein, the term “complementarity determining region” (CDR) refers to a hypervariable region found both in the light chain and the heavy chain variable domains of an antibody. The more highly conserved portions of variable domains are referred to as framework regions (FRs). The amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The antibodies described herein may contain modifications in these hybrid hypervariable positions. The variable domains of native heavy and light chains each contain four framework regions that primarily adopt a β-sheet configuration, connected by three CDRs, which form loops that connect, and in some cases form part of, the β-sheet structure. The CDRs in each chain are held together in close proximity by the framework regions in the order FR1-CDR1-FR2-CDR2-FR3-CDR3-FR4 and, with the CDRs from the other antibody chains, contribute to the formation of the target binding site of antibodies (see Kabat et al., Sequences of Proteins of Immunological Interest, National Institute of Health, Bethesda, MD., 1987). In certain embodiments, numbering of immunoglobulin amino acid residues is performed according to the immunoglobulin amino acid residue numbering system of Kabat et al., unless otherwise indicated (although any antibody numbering scheme, including, but not limited to IMGT and Chothia, can be utilized).

As used herein, the terms “condition” and “conditioning” refer to processes by which a patient is prepared for receipt of a transplant, e.g., a transplant containing hematopoietic stem cells. Such procedures promote the engraftment of a hematopoietic stem cell transplant (for instance, as inferred from a sustained increase in the quantity of viable hematopoietic stem cells within a blood sample isolated from a patient following a conditioning procedure and subsequent hematopoietic stem cell transplantation. According to the methods described herein, a patient may be conditioned for hematopoietic stem cell transplant therapy by administration to the patient of an ADC capable of binding an antigen expressed by hematopoietic stem cells, such as CD117 (e.g., GNNK+CD117). Administration of an ADC capable of binding an HSC antigen to a patient in need of hematopoietic stem cell transplant therapy can promote the engraftment of a hematopoietic stem cell graft, for example, by selectively depleting endogenous hematopoietic stem cells, thereby creating a vacancy filled by an exogenous hematopoietic stem cell transplant.

As used herein, the term “conjugate” refers to a compound formed by the chemical bonding of a reactive functional group of one molecule, such as an antibody or antigen-binding fragment thereof, with an appropriately reactive functional group of another molecule, such as a cytotoxin described herein. Conjugates may include a linker between the two molecules bound to one another. Examples of linkers that can be used for the formation of a conjugate include peptide-containing linkers, such as those that contain naturally occurring or non-naturally occurring amino acids, such as D-amino acids. Linkers can be prepared using a variety of strategies described herein and known in the art. Depending on the reactive components therein, a linker may be cleaved, for example, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012). Notably, the term “conjugate” (when referring to a compound) is also referred to interchangeably herein as a “drug conjugate”, “antibody drug conjugate” or “ADC”.

As used herein, the term “coupling reaction” refers to a chemical reaction in which two or more substituents suitable for reaction with one another react so as to form a chemical moiety that joins (e.g., covalently) the molecular fragments bound to each substituent. Coupling reactions include those in which a reactive substituent bound to a fragment that is a cytotoxin, such as a cytotoxin known in the art or described herein, reacts with a suitably reactive substituent bound to a fragment that is an antibody, or antigen-binding fragment thereof, such as an antibody, antigen-binding fragment thereof, or specific anti-CD117 antibody that binds CD117 (such as GNNK+CD117) known in the art or described herein. Examples of suitably reactive substituents include a nucleophile/electrophile pair (e.g., a thiol/haloalkyl pair, an amine/carbonyl pair, or a thiol/α,β-unsaturated carbonyl pair, among others), a diene/dienophile pair (e.g., an azide/alkyne pair, among others), and the like. Coupling reactions include, without limitation, thiol alkylation, hydroxyl alkylation, amine alkylation, amine condensation, amidation, esterification, disulfide formation, cycloaddition (e.g., [4+2] Diels-Alder cycloaddition, [3+2] Huisgen cycloaddition, among others), nucleophilic aromatic substitution, electrophilic aromatic substitution, and other reactive modalities known in the art or described herein.

As used herein, “CRU (competitive repopulating unit)” refers to a unit of measure of long-term engrafting stem cells, which can be detected after in-vivo transplantation.

As used herein, the term “donor” refers to a human or animal from which one or more cells are isolated prior to administration of the cells, or progeny thereof, into a recipient. The one or more cells may be, for example, a population of hematopoietic stem cells.

As used herein, the term “diabody” refers to a bivalent antibody containing two polypeptide chains, in which each polypeptide chain includes V_{H} and V_{L} domains joined by a linker that is too short (e.g., a linker composed of five amino acids) to allow for intramolecular association of V_{H} and V_{L} domains on the same peptide chain. This configuration forces each domain to pair with a complementary domain on another polypeptide chain so as to form a homodimeric structure. Accordingly, the term “triabody” refers to trivalent antibodies containing three peptide chains, each of which contains one V_{H} domain and one V_{L} domain joined by a linker that is exceedingly short (e.g., a linker composed of 1-2 amino acids) to permit intramolecular association of V_{H} and V_{L} domains within the same peptide chain. In order to fold into their native structures, peptides configured in this way typically trimerize so as to position the V_{H} and V_{L} domains of neighboring peptide chains spatially proximal to one another (see, for example, Holliger et al., Proc. Natl. Acad. Sci. USA 90:6444-48, 1993).

As used herein, “drug-to-antibody ratio” or “DAR” refers to the number of drugs, e.g., amatoxin, attached to the antibody of a conjugate. The DAR of an ADC can range from 1 to 8, although higher loads are also possible depending on the number of linkage sites on an antibody. In certain embodiments, the conjugate has a DAR of 1, 2, 3, 4, 5, 6, 7, or 8.

As used herein, the term “endogenous” describes a substance, such as a molecule, cell, tissue, or organ (e.g., a hematopoietic stem cell or a cell of hematopoietic lineage, such as a megakaryocyte, thrombocyte, platelet, erythrocyte, mast cell, myeloblast, basophil, neutrophil, eosinophil, microglial cell, granulocyte, monocyte, osteoclast, antigen-presenting cell, macrophage, dendritic cell, natural killer cell, T-lymphocyte, or B-lymphocyte) that is found naturally in a particular organism, such as a human patient.

As used herein, the term “engraftment potential” is used to refer to the ability of hematopoietic stem and progenitor cells to repopulate a tissue, whether such cells are naturally circulating or are provided by transplantation. The term encompasses all events surrounding or leading up to engraftment, such as tissue homing of cells and colonization of cells within the tissue of interest. The engraftment efficiency or rate of engraftment can be evaluated or quantified using any clinically acceptable parameter as known to those of skill in the art and can include, for example, assessment of competitive repopulating units (CRU); incorporation or expression of a marker in tissue(s) into which stem cells have homed, colonized, or become engrafted; or by evaluation of the progress of a subject through disease progression, survival of hematopoietic stem and progenitor cells, or survival of a recipient. Engraftment can also be determined by measuring white blood cell counts in peripheral blood during a post-transplant period. Engraftment can also be assessed by measuring recovery of marrow cells by donor cells in a bone marrow aspirate sample.

As used herein, the term “exogenous” describes a substance, such as a molecule, cell, tissue, or organ (e.g., a hematopoietic stem cell or a cell of hematopoietic lineage, such as a megakaryocyte, thrombocyte, platelet, erythrocyte, mast cell, myeloblast, basophil, neutrophil, eosinophil, microglial cell, granulocyte, monocyte, osteoclast, antigen-presenting cell, macrophage, dendritic cell, natural killer cell, T-lymphocyte, or B-lymphocyte) that is not found naturally in a particular organism, such as a human patient. Exogenous substances include those that are provided from an external source to an organism or to cultured matter extracted therefrom.

The terms “Fc”, “Fc region,” and “Fc domain,” as used herein refer to the portion of an IgG antibody that correlates to a crystallizable fragment obtained by papain digestion of an IgG molecule. The Fc region comprises the C-terminal half of two heavy chains of an IgG molecule that are linked by disulfide bonds. It has no antigen binding activity but contains the carbohydrate moiety and binding sites for complement and Fc receptors, including the FcRn receptor. An Fc region contains the second constant domain CH2 (e.g., residues at EU positions 231-340 of IgG1) and the third constant domain CH3 (e.g., residues at EU positions 341-447 of human IgG1). As used herein, the Fc region includes the “lower hinge region” (e.g., residues at EU positions 233-239 of IgG1). Fc can refer to this region in isolation, or this region in the context of an antibody, antibody fragment, or Fc fusion protein. Polymorphisms have been observed at a number of positions in Fc domains, including but not limited to EU positions 270, 272, 312, 315, 356, and 358, and thus slight differences between the sequences presented in the instant application and sequences known in the art can exist. Thus, a “wild type IgG Fc domain” or “WT IgG Fc domain” refers to any naturally occurring IgG Fc region (i.e., any allele). The sequences of the heavy chains of human IgG1, IgG2, IgG3 and IgG4 can be found in a number of sequence databases, for example, at the Uniprot database (www.uniprot.org) under accession numbers P01857 (IGHG1_HUMAN), P01859 (IGHG2_HUMAN), P01860 (IGHG3_HUMAN), and P01861 (IGHG1_HUMAN), respectively. An example of a “WT” Fc region is provided in SEQ ID NO: 122 (which provides a heavy chain constant region containing an Fc region).

The terms “modified Fc region” or “variant Fc region” as used herein refers to an IgG Fc domain comprising one or more amino acid substitutions, deletions, insertions or modifications introduced at any position within the Fc region.

The terms “full length antibody” and “intact antibody” are used herein interchangeably to refer to an antibody in its substantially intact form, and not an antibody fragment as defined herein. In one embodiment, an ADC described herein comprises an intact antibody. Thus, for an IgG antibody, an intact antibody comprises two heavy chains each comprising a variable region, a constant region and an Fc region, and two light chains each comprising a variable region and a constant region. More specifically, an intact IgG comprises two light chains each comprising a light chain variable region (VL) and a light chain constant region (CL), and comprises two heavy chains each comprising a heavy chain variable region (VH) and three heavy chain constant regions (CH1, CH2, and CH3). CH2 and CH3 represent the Fc region of the heavy chain.

As used herein, the term “framework region” or “FW region” includes amino acid residues that are adjacent to the CDRs of an antibody or antigen-binding fragment thereof. FW region residues may be present in, for example, human antibodies, humanized antibodies, monoclonal antibodies, antibody fragments, Fab fragments, single chain antibody fragments, scFv fragments, antibody domains, and bispecific antibodies, among others.

As used herein, the term “hematopoietic stem cells” (“HSCs”) refers to immature blood cells having the capacity to self-renew and to differentiate into mature blood cells containing diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Such cells may include CD34⁺ cells. CD34⁺ cells are immature cells that express the CD34 cell surface marker. In humans, CD34+ cells are believed to include a subpopulation of cells with the stem cell properties defined above, whereas in mice, HSCs are CD34−. In addition, HSCs also refer to long term repopulating HSCs (LT-HSC) and short term repopulating HSCs (ST-HSC). LT-HSCs and ST-HSCs are differentiated, based on functional potential and on cell surface marker expression. For example, human HSCs are CD34+, CD38-CD45RA−, CD90+, CD49F+, and lin− (negative for mature lineage markers including CD2, CD3, CD4, CD7, CD8, CD10, CD11B, CD19, CD20, CD56, CD235A). In mice, bone marrow LT-HSCs are CD34−, SCA-1+, C-kit+, CD135−, Slamfl/CD150+, CD48−, and lin− (negative for mature lineage markers including Ter119, CD11b, Gr1, CD3, CD4, CD8, B220, IL7ra), whereas ST-HSCs are CD34+, SCA-1+, C-kit+, CD135−, Slamfl/CD150+, and lin− (negative for mature lineage markers including Ter19, CD11b, Gr1, CD3, CD4, CD8, B220, IL7ra). In addition, ST-HSCs are less quiescent and more proliferative than LT-HSCs under homeostatic conditions. However, LT-HSC have greater self-renewal potential (i.e., they survive throughout adulthood, and can be serially transplanted through successive recipients), whereas ST-HSCs have limited self-renewal (i.e., they survive for only a limited period of time, and do not possess serial transplantation potential). Any of these HSCs can be used in the methods described herein. ST-HSCs are particularly useful because they are highly proliferative and thus, can more quickly give rise to differentiated progeny.

As used herein, the term “hematopoietic stem cell functional potential” refers to the functional properties of hematopoietic stem cells which include 1) multi-potency (which refers to the ability to differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal (which refers to the ability of hematopoietic stem cells to give rise to daughter cells that have equivalent potential as the mother cell, and further that this ability can repeatedly occur throughout the lifetime of an individual without exhaustion), and 3) the ability of hematopoietic stem cells or progeny thereof to be reintroduced into a transplant recipient whereupon they home to the hematopoietic stem cell niche and re-establish productive and sustained hematopoiesis.

As used herein, the term “human antibody” is intended to include antibodies having variable and constant regions derived from human germline immunoglobulin sequences. A human antibody may include amino acid residues not encoded by human germline immunoglobulin sequences (e.g., mutations introduced by random or site-specific mutagenesis in vitro or during gene rearrangement or by somatic mutation in vivo). However, the term “human antibody”, as used herein, is not intended to include antibodies in which CDR sequences derived from the germline of another mammalian species, such as a mouse, have been grafted onto human framework sequences. A human antibody can be produced in a human cell (for example, by recombinant expression) or by a non-human animal or a prokaryotic or eukaryotic cell that is capable of expressing functionally rearranged human immunoglobulin (such as heavy chain and/or light chain) genes. When a human antibody is a single chain antibody, it can include a linker peptide that is not found in native human antibodies. For example, an Fv can contain a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. Human antibodies can also be produced using transgenic mice that are incapable of expressing functional endogenous immunoglobulins, but which can express human immunoglobulin genes (see, for example, PCT Publication Nos. WO 1998/24893; WO 1992/01047; WO 1996/34096; WO 1996/33735; U.S. Pat. Nos. 5,413,923; 5,625,126; 5,633,425; 5,569,825; 5,661,016; 5,545,806; 5,814,318; 5,885,793; 5,916,771; and 5,939,598).

A “humanized” antibody refers to an antibody that contains minimal sequences derived from non-human immunoglobulin. Thus, “humanized” forms of non-human (e.g., murine) antibodies are chimeric antibodies that contain minimal sequence derived from the non-human antibody. All or substantially all of the FW regions may also be those of a human immunoglobulin sequence. The humanized antibody can also contain at least a portion of an immunoglobulin constant region (Fc), typically that of a human immunoglobulin consensus sequence. Methods of antibody humanization are known in the art and have been described, for example, in Riechmann et al., Nature 332:323-7, 1988; U.S. Pat. Nos. 5,530,101; 5,585,089; 5,693,761; 5,693,762; and 6,180,370.

As used herein, the term “antibody-like protein” refers to a protein that has been engineered (e.g. by mutagenesis of Ig loops) to specifically bind to a target molecule. Typically, such an antibody-like protein comprises at least one variable peptide loop attached at both ends to a protein scaffold. This double structural constraint greatly increases the binding affinity of the antibody-like protein to levels comparable to that of an antibody. The length of the variable peptide loop typically consists of 10 to 20 amino acids. The scaffold protein may be any protein having good solubility properties. Preferably, the scaffold protein is a small globular protein. Antibody-like proteins include without limitation affibodies, anticalins, and designed ankyrin repeat proteins (Binz et al., 2005). Antibody-like proteins can be derived from large libraries of mutants, e.g. by panning from large phage display libraries, and can be isolated in analogy to regular antibodies. Also, antibody-like binding proteins can be obtained by combinatorial mutagenesis of surface-exposed residues in globular proteins.

As used herein, the term “Fab” relates to an IgG fragment comprising the antigen binding region, said fragment being composed of one constant and one variable domain from each heavy and light chain of the antibody.

As used herein, the term “F(ab)₂” relates to an IgG fragment consisting of two Fab fragments connected to one another by disulfide bonds.

As used herein, the term “scFv” relates to a single-chain variable fragment being a fusion of the variable regions of the heavy and light chains of immunoglobulins, linked together with a short linker, usually comprising serine (S) and/or glycine (G) residues. This chimeric molecule retains the specificity of the original immunoglobulin, despite removal of the constant regions and the introduction of a linker peptide.

According to one aspect of the present invention, the present invention relates to said conjugate or pharmaceutical composition as described for use in the treatment of cancer.

According to one aspect of the present invention, the present invention relates to said conjugate or pharmaceutical composition as described for use in the treatment of B lymphocyte-associated malignancies or B cell-mediated autoimmune diseases, in particular for use in the treatment of non-Hodgkin's lymphoma, follicular lymphoma, diffuse large B cell non-Hodgkin's lymphoma, chronic lymphocytic leukaemia, rheumatoid arthritis, granulomatosis with polyangiitis and microscopic polyangiitis and pemphigus vulgaris.

As used herein, patients that are “in need of” a hematopoietic stem cell transplant include patients that exhibit a defect or deficiency in one or more blood cell types, as well as patients having a stem cell disorder, autoimmune disease, cancer, or other pathology described herein. Hematopoietic stem cells generally exhibit 1) multi-potency, and can thus differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal, and can thus give rise to daughter cells that have equivalent potential as the mother cell, and 3) the ability to be reintroduced into a transplant recipient whereupon they home to the hematopoietic stem cell niche and re-establish productive and sustained hematopoiesis. Hematopoietic stem cells can thus be administered to a patient defective or deficient in one or more cell types of the hematopoietic lineage in order to re-constitute the defective or deficient population of cells in vivo. For example, the patient may be suffering from cancer, and the deficiency may be caused by administration of a chemotherapeutic agent or other medicament that depletes, either selectively or non-specifically, the cancerous cell population. Additionally or alternatively, the patient may be suffering from a hemoglobinopathy (e.g., a non-malignant hemoglobinopathy), such as sickle cell anemia, thalassemia, Fanconi anemia, aplastic anemia, and Wiskott-Aldrich syndrome. The subject may be one that is suffering from adenosine deaminase severe combined immunodeficiency (ADA SCID), HIV/AIDS, metachromatic leukodystrophy, Diamond-Blackfan anemia, and Schwachman-Diamond syndrome. The subject may have or be affected by an inherited blood disorder (e.g., sickle cell anemia) or an autoimmune disorder. Additionally or alternatively, the subject may have or be affected by a malignancy, such as neuroblastoma or a hematologic cancer. For instance, the subject may have a leukemia, lymphoma, or myeloma. In some embodiments, the subject has acute myeloid leukemia, acute lymphoid leukemia, chronic myeloid leukemia, chronic lymphoid leukemia, multiple myeloma, diffuse large B-cell lymphoma, or non-Hodgkin's lymphoma. In some embodiments, the subject has myelodysplastic syndrome. In some embodiments, the subject has an autoimmune disease, such as scleroderma, multiple sclerosis, ulcerative colitis, Crohn's disease, Type 1 diabetes, or another autoimmune pathology described herein. In some embodiments, the subject is in need of chimeric antigen receptor T-cell (CART) therapy. In some embodiments, the subject has or is otherwise affected by a metabolic storage disorder. The subject may suffer or otherwise be affected by a metabolic disorder selected from the group consisting of glycogen storage diseases, mucopolysaccharidoses, Gaucher's Disease, Hurlers Disease, sphingolipidoses, metachromatic leukodystrophy, or any other diseases or disorders which may benefit from the treatments and therapies disclosed herein and including, without limitation, severe combined immunodeficiency, Wiscott-Aldrich syndrome, hyper immunoglobulin M (IgM) syndrome, Chediak-Higashi disease, hereditary lymphohistiocytosis, osteopetrosis, osteogenesis imperfecta, storage diseases, thalassemia major, sickle cell disease, systemic sclerosis, systemic lupus erythematosus, multiple sclerosis, juvenile rheumatoid arthritis and those diseases, or disorders described in “Bone Marrow Transplantation for Non-Malignant Disease,” ASH Education Book, 1:319-338 (2000), the disclosure of which is incorporated herein by reference in its entirety as it pertains to pathologies that may be treated by administration of hematopoietic stem cell transplant therapy. Additionally or alternatively, a patient “in need of” a hematopoietic stem cell transplant may one that is or is not suffering from one of the foregoing pathologies, but nonetheless exhibits a reduced level (e.g., as compared to that of an otherwise healthy subject) of one or more endogenous cell types within the hematopoietic lineage, such as megakaryocytes, thrombocytes, platelets, erythrocytes, mast cells, myeloblasts, basophils, neutrophils, eosinophils, microglia, granulocytes, monocytes, osteoclasts, antigen-presenting cells, macrophages, dendritic cells, natural killer cells, T-lymphocytes, and B-lymphocytes. One of skill in the art can readily determine whether one's level of one or more of the foregoing cell types, or other blood cell type, is reduced with respect to an otherwise healthy subject, for instance, by way of flow cytometry and fluorescence activated cell sorting (FACS) methods, among other procedures, known in the art.

As used herein a “neutral antibody” refers to an antibody, or an antigen binding fragment thereof, that is not capable of significantly neutralizing, blocking, inhibiting, abrogating, reducing or interfering with the activities of a particular or specified target (e.g., CD117), including the binding of receptors to ligands or the interactions of enzymes with substrates. In one embodiment, a neutral anti-CD117 antibody, or fragment thereof, is an anti-CD117 antibody that does not substantially inhibit SCF-dependent cell proliferation and does not cross block SCF binding to CD117. An example of a neutral antibody is Ab67 (or an antibody having the binding regions of Ab67). In contrast, an “antagonist” anti-CD117 antibody inhibits SCF-dependent proliferation and is able to cross block SCF binding to CD117. An example of an antagonist antibody is Ab55 (or an antibody having the binding regions of Ab55).

As used herein, the term “recipient” refers to a patient that receives a transplant, such as a transplant containing a population of hematopoietic stem cells. The transplanted cells administered to a recipient may be, e.g., autologous, syngeneic, or allogeneic cells.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) taken from a subject.

As used herein, the term “scFv” refers to a single chain Fv antibody in which the variable domains of the heavy chain and the light chain from an antibody have been joined to form one chain. scFv fragments contain a single polypeptide chain that includes the variable region of an antibody light chain (V_{L}) (e.g., CDR-L1, CDR-L2, and/or CDR-L3) and the variable region of an antibody heavy chain (V_{H}) (e.g., CDR-H1, CDR-H2, and/or CDR-H3) separated by a linker. The linker that joins the V_{L} and VH regions of a scFv fragment can be a peptide linker composed of proteinogenic amino acids. Alternative linkers can be used to so as to increase the resistance of the scFv fragment to proteolytic degradation (for example, linkers containing D-amino acids), in order to enhance the solubility of the scFv fragment (for example, hydrophilic linkers such as polyethylene glycol-containing linkers or polypeptides containing repeating glycine and serine residues), to improve the biophysical stability of the molecule (for example, a linker containing cysteine residues that form intramolecular or intermolecular disulfide bonds), or to attenuate the immunogenicity of the scFv fragment (for example, linkers containing glycosylation sites). It will also be understood by one of ordinary skill in the art that the variable regions of the scFv molecules described herein can be modified such that they vary in amino acid sequence from the antibody molecule from which they were derived. For example, nucleotide or amino acid substitutions leading to conservative substitutions or changes at amino acid residues can be made (e.g., in CDR and/or framework residues) so as to preserve or enhance the ability of the scFv to bind to the antigen recognized by the corresponding antibody.

The terms “specific binding” or “specifically binding”, as used herein, refers to the ability of an antibody to recognize and bind to a specific protein structure (epitope) rather than to proteins generally. If an antibody is specific for epitope “A”, the presence of a molecule containing epitope A (or free, unlabeled A), in a reaction containing labeled “A” and the antibody, will reduce the amount of labeled A bound to the antibody. By way of example, an antibody “binds specifically” to a target if the antibody, when labeled, can be competed away from its target by the corresponding non-labeled antibody. In one embodiment, an antibody specifically binds to a target, e.g., CD117, CD45, CD2, CD5, CD137, CD134, or CD252, if the antibody has a K_{D} for the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, or less (less meaning a number that is less than 10⁻¹², e.g. 10⁻¹³) In one embodiment, the term “specific binding to CD117” or “specifically binds to CD117,” as used herein, refers to an antibody or that binds to CD117 and has a dissociation constant (K_{D}) of 1.0×10⁻⁷ M or less, as determined by surface plasmon resonance. In one embodiment, K_{D}(M) is determined according to standard bio-layer interferometery (BLI). In one embodiment, K_{off} (1/s) is determined according to standard bio-layer interferometery (BLI). It shall be understood, however, that the antibody may be capable of specifically binding to two or more antigens which are related in sequence. For example, in one embodiment, an antibody can specifically bind to both human and a non-human (e.g., mouse or non-human primate) orthologs of CD117, CD45, CD2, CD5, CD137, CD134, or CD252.

As used herein, the terms “subject” and “patient” refer to an organism, such as a human, that receives treatment for a particular disease or condition as described herein. For instance, a patient, such as a human patient, may receive treatment prior to hematopoietic stem cell transplant therapy in order to promote the engraftment of exogenous hematopoietic stem cells.

As used herein, the phrase “substantially cleared from the blood” refers to a point in time following administration of a therapeutic agent, e.g., an ADC comprising an amatoxin, to a patient when the concentration of the therapeutic agent in a blood sample isolated from the patient is such that the therapeutic agent is not detectable by conventional means (for instance, such that the therapeutic agent is not detectable above the noise threshold of the device or assay used to detect the therapeutic agent). A variety of techniques known in the art can be used to detect antibodies, or antibody fragments, such as ELISA-based detection assays known in the art or described herein. Additional assays that can be used to detect antibodies, or antibody fragments, include immunoprecipitation techniques and immunoblot assays, among others known in the art.

As used herein, the phrase “stem cell disorder” broadly refers to any disease, disorder, or condition that may be treated or cured by conditioning a subject's target tissues, and/or by ablating an endogenous stem cell population in a target tissue (e.g., ablating an endogenous hematopoietic stem or progenitor cell population from a subject's bone marrow tissue) and/or by engrafting or transplanting stem cells in a subject's target tissues. For example, Type I diabetes has been shown to be cured by hematopoietic stem cell transplant and may benefit from conditioning in accordance with the compositions and methods described herein. Additional disorders that can be treated using the compositions and methods described herein include, without limitation, sickle cell anemia, thalassemias, Fanconi anemia, aplastic anemia, Wiskott-Aldrich syndrome, ADA SCID, HIV/AIDS, metachromatic leukodystrophy, Diamond-Blackfan anemia, and Schwachman-Diamond syndrome. Additional diseases that may be treated using the patient conditioning and/or hematopoietic stem cell transplant methods described herein include inherited blood disorders (e.g., sickle cell anemia) and autoimmune disorders, such as scleroderma, multiple sclerosis, ulcerative colitis, and Crohn's disease. Additional diseases that may be treated using the conditioning and/or transplantation methods described herein include a malignancy, such as a neuroblastoma or a hematologic cancer, such as leukemia, lymphoma, and myeloma. For instance, the cancer may be acute myeloid leukemia, acute lymphoid leukemia, chronic myeloid leukemia, chronic lymphoid leukemia, multiple myeloma, diffuse large B-cell lymphoma, or non-Hodgkin's lymphoma. Additional diseases treatable using the conditioning and/or transplantation methods described herein include myelodysplastic syndrome. In some embodiments, the subject has or is otherwise affected by a metabolic storage disorder. For example, the subject may suffer or otherwise be affected by a metabolic disorder selected from the group consisting of glycogen storage diseases, mucopolysaccharidoses, Gaucher's Disease, Hurlers Disease, sphingolipidoses, metachromatic leukodystrophy, or any other diseases or disorders which may benefit from the treatments and therapies disclosed herein and including, without limitation, severe combined immunodeficiency, Wiscott-Aldrich syndrome, hyper immunoglobulin M (IgM) syndrome, Chediak-Higashi disease, hereditary lymphohistiocytosis, osteopetrosis, osteogenesis imperfecta, storage diseases, thalassemia major, sickle cell disease, systemic sclerosis, systemic lupus erythematosus, multiple sclerosis, juvenile rheumatoid arthritis and those diseases, or disorders described in “Bone Marrow Transplantation for Non-Malignant Disease,” ASH Education Book, 1:319-338 (2000), the disclosure of which is incorporated herein by reference in its entirety as it pertains to pathologies that may be treated by administration of hematopoietic stem cell transplant therapy.

As used herein, the term “transfection” refers to any of a wide variety of techniques commonly used for the introduction of exogenous DNA into a prokaryotic or eukaryotic host cell, such as electroporation, lipofection, calcium-phosphate precipitation, DEAE-dextran transfection and the like.

As used herein, the terms “treat” or “treatment” refers to reducing the severity and/or frequency of disease symptoms, eliminating disease symptoms and/or the underlying cause of said symptoms, reducing the frequency or likelihood of disease symptoms and/or their underlying cause, and improving or remediating damage caused, directly or indirectly, by disease. Beneficial or desired clinical results include, but are not limited to, promoting the engraftment of exogenous hematopoietic cells in a patient following antibody conditioning therapy as described herein and subsequent hematopoietic stem cell transplant therapy. Additional beneficial results include an increase in the cell count or relative concentration of hematopoietic stem cells in a patient in need of a hematopoietic stem cell transplant following conditioning therapy and subsequent administration of an exogenous hematopoietic stem cell graft to the patient. Beneficial results of therapy described herein may also include an increase in the cell count or relative concentration of one or more cells of hematopoietic lineage, such as a megakaryocyte, thrombocyte, platelet, erythrocyte, mast cell, myeloblast, basophil, neutrophil, eosinophil, microglial cell, granulocyte, monocyte, osteoclast, antigen-presenting cell, macrophage, dendritic cell, natural killer cell, T-lymphocyte, or B-lymphocyte, following conditioning therapy and subsequent hematopoietic stem cell transplant therapy. Additional beneficial results may include the reduction in quantity of a disease-causing cell population, such as a population of cancer cells (e.g., CD117+ leukemic cells) or autoimmune cells (e.g., CD117+ autoimmune lymphocytes, such as a CD117+ T-cell that expresses a T-cell receptor that cross-reacts with a self-antigen). Insofar as the methods of the present invention are directed to preventing disorders, it is understood that the term “prevent” does not require that the disease state be completely thwarted. Rather, as used herein, the term preventing refers to the ability of the skilled artisan to identify a population that is susceptible to disorders, such that administration of the compounds of the present invention may occur prior to onset of a disease. The term does not imply that the disease state is completely avoided.

As used herein, the terms “variant” and “derivative” are used interchangeably and refer to naturally-occurring, synthetic, and semi-synthetic analogues of a compound, peptide, protein, or other substance described herein. A variant or derivative of a compound, peptide, protein, or other substance described herein may retain or improve upon the biological activity of the original material.

As used herein, the term “vector” includes a nucleic acid vector, such as a plasmid, a DNA vector, a plasmid, a RNA vector, virus, or other suitable replicon. Expression vectors described herein may contain a polynucleotide sequence as well as, for example, additional sequence elements used for the expression of proteins and/or the integration of these polynucleotide sequences into the genome of a mammalian cell. Certain vectors that can be used for the expression of antibodies and antibody fragments of the invention include plasmids that contain regulatory sequences, such as promoter and enhancer regions, which direct gene transcription. Other useful vectors for expression of antibodies and antibody fragments contain polynucleotide sequences that enhance the rate of translation of these genes or improve the stability or nuclear export of the mRNA that results from gene transcription. These sequence elements may include, for example, 5′ and 3′ untranslated regions and a polyadenylation signal site in order to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, and nourseothricin.

### Antibody-Drug Conjugates (ADCs)

Antibodies, and antigen-binding fragments thereof, as described herein can be conjugated (linked) to a cytotoxic molecule (i.e., a cytotoxin), thus forming an antibody-drug conjugate (ADC). As used herein, the terms “cytotoxin”, “cytotoxic moiety”, and “drug” are used interchangeably.

In particular, the ADCs as disclosed herein include an antibody (including an antigen-binding fragment thereof) conjugated to an amatoxin, e.g., amatoxin set forth in Formula (V), wherein the cytotoxic moiety, when not conjugated to an antibody, has a cytotoxic or cytostatic effect. In various embodiments, the cytotoxic moiety exhibits reduced or no cytotoxicity when bound in a conjugate, but resumes cytotoxicity after cleavage from the linker. In various embodiments, the cytotoxic moiety maintains cytotoxicity without cleavage from the linker. In some embodiments, the cytotoxic molecule is conjugated to a cell internalizing antibody, or antigen-binding fragment thereof as disclosed herein, such that following the cellular uptake of the antibody, or fragment thereof, the cytotoxin may access its intracellular target and, e.g., mediate hematopoietic cell death. ADCs of the present disclosure therefore may be of the general formula

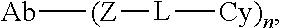

wherein an antibody or antigen-binding fragment thereof (Ab) is conjugated (covalently linked) to linker (L), through a chemical moiety (Z), to a cytotoxic moiety (Cy).

Accordingly, the antibody or antigen-binding fragment thereof may be conjugated to a number of drug moieties as indicated by integer n, which represents the average number of cytotoxins per antibody, which may range, e.g., from about 1 to about 20. Any number of cytotoxins can be conjugated to the antibody, e.g., 1, 2, 3, 4, 5, 6, 7, or 8. In some embodiments, n is from 1 to 4. In some embodiments, n is 1. The average number of drug moieties per antibody in preparations of ADC from conjugation reactions may be characterized by conventional means such as mass spectroscopy, ELISA assay, and HPLC. The quantitative distribution of ADC in terms of n may also be determined. In some instances, separation, purification, and characterization of homogeneous ADC where n is a certain value from ADC with other drug loadings may be achieved by means such as reverse phase HPLC or electrophoresis.

For some antibody-drug conjugates, n may be limited by the number of attachment sites on the antibody. For example, where the attachment is a cysteine thiol, an antibody may have only one or several cysteine thiol groups, or may have only one or several sufficiently reactive thiol groups through which a linker may be attached. Generally, antibodies do not contain many free and reactive cysteine thiol groups which may be linked to a drug moiety; primarily, cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups. In certain embodiments, higher drug loading, e.g. n>5, may cause aggregation, insolubility, toxicity, or loss of cellular permeability of certain antibody-drug conjugates.

In certain embodiments, fewer than the theoretical maximum of drug moieties are conjugated to an antibody during a conjugation reaction. An antibody may contain, for example, lysine residues that do not react with the drug-linker intermediate or linker reagent, as discussed below. Only the most reactive lysine groups may react with an amine-reactive linker reagent. In certain embodiments, an antibody is subjected to denaturing conditions to reveal reactive nucleophilic groups such as lysine or cysteine.

The loading (drug/antibody ratio) of an ADC may be controlled in different ways, e.g., by: (i) limiting the molar excess of drug-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number and/or position of linker-drug attachments.

### Cytotoxins

The cytotoxin of the antibody-drug conjugates described herein is an amatoxin or derivative thereof. Amatoxins are potent and selective inhibitors of RNA polymerase II and thereby also inhibit the transcription and protein biosynthesis of the affected cells. As used herein, the term “amatoxin” refers to a member of the amatoxin family of peptides produced by Amanitaphalloides mushrooms, or a variant or derivative thereof, such as a variant or derivative thereof capable of inhibiting RNA polymerase II activity. Amatoxins are rigid bicyclic octapeptides having the basic sequence Ile-Trp-Gly-Ile-Gly-Cys-Asn(or Asp)-Pro, crosslinked by an attachment between the Cys sulfur and position 2 of the Trp indole ring, forming a tryptathionine. Depending on the particular amatoxin, certain amino acid substituents are varied by post-translational modification (i.e. Pro to Hyp; Ile to DHIle; and Trp to 5-OH Trp).

Amatoxins may be isolated from a variety of mushroom species (e.g., Amanita phalloides, Galerina marginata, Lepiota brunneo-incarnata) or may be prepared semi-synthetically or synthetically. Different mushroom species contain varying amounts of different amatoxin family members. A member of this family, α-amanitin, is known to be an extremely potent inhibitor of eukaryotic RNA polymerase II and to a lesser degree, RNA polymerase III, thereby inhibiting transcription and protein biosynthesis. Wieland, Int. J. Pept. Protein Res. 1983, 22(3):257-276.

Structures of the various naturally occurring amatoxins are represented by formula (III) and accompanying Table 1, and are disclosed in, e.g., Zanotti et al., Int. J. Peptide Protein Res. 30, 1987, 450-459, which is incorporated by reference herein in its entirety.

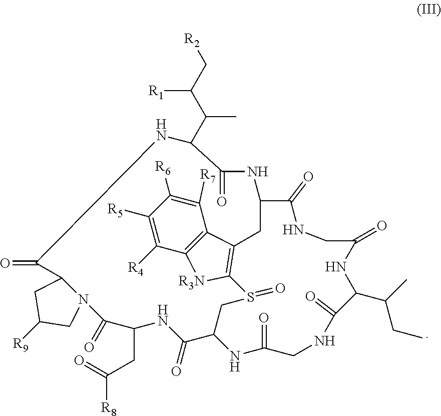

TABLE 1
Amatoxin structures

| Name | R₁ | R₂ | R₃, R₄ | R₅ | R₆, R₇ | R₈ | R₉ |
|---|---|---|---|---|---|---|---|
| α-amanitin | OH | OH | H | OH | H | NH₂ | OH |
| β-amanitin | OH | OH | H | OH | H | OH | OH |
| γ-amanitin | OH | H | H | OH | H | NH₂ | OH |
| ε-amanitin | OH | H | H | OH | H | OH | OH |
| Amanin | OH | OH | H | H | H | OH | OH |
| Amaninamide | OH | OH | H | H | H | NH₂ | OH |
| Amanullin | H | H | H | OH | H | NH₂ | OH |
| Amanullinic acid | H | H | H | OH | H | OH | OH |
| Proamanullin | H | H | H | OH | H | NH₂ | H |

Antibodies, or antigen-binding fragments thereof, that recognize and bind to an antigen expressed on the cell surface of a human stem cell or a T cell can be conjugated to an amatoxin, such as an α-amanitin or a derivative thereof, as described in, for example, U.S. Pat. Nos. 9,233,173 and 9,399,681 and US Patent Application Publication Nos. 2016/0089450, 2016/0002298, 2015/0218220, 2014/0294865, the disclosure of each of which is incorporated herein by reference as it pertains, for example, to amatoxins, such as α-amanitin, as well as covalent linkers that can be used for covalent conjugation. Exemplary methods of amatoxin conjugation and linkers useful for such processes are described herein. Exemplary linker-containing amatoxins useful for conjugation to an antibody, or antigen-binding fragment, in accordance with the compositions and methods are also described herein.

As used herein, the term “amatoxin derivative” or “amanitin derivative” refers to an amatoxin that has been chemically modified at one or more positions relative to a naturally occurring amatoxin, such as α-amanitin, β-amanitin, γ-amanitin, ε-amanitin, amanin, amaninamide, amanullin, amanullinic acid, or proamanullin. In each instance, the derivative may be obtained by chemical modification of a naturally occurring compound (“semi-synthetic”), or may be obtained from an entirely synthetic source. Synthetic routes to various amatoxin derivatives are disclosed in, for example, U.S. Pat. No. 9,676,702 and in Perrin et al., J. Am. Chem. Soc. 2018, 140, p. 6513-6517, each of which is incorporated by reference herein in their entirety with respect to synthetic methods for preparing and derivatizing amatoxins.

In some embodiments, the amatoxin or derivative thereof is represented by formula (V):

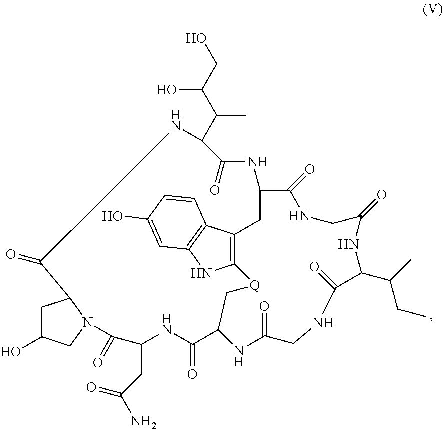

or an enantiomer or diastereomer thereof.

In some embodiments, Q is S. In some embodiments, Q is a sulfoxid group.

In one embodiment, the amatoxin or derivative thereof is represented by formula (Va):

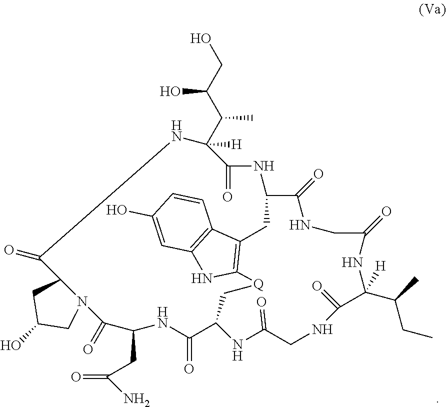

In some embodiments, Q is S. In some embodiments, Q is a sulfoxid group.

In this particular embodiment, the amatoxin or derivative thereof is represented by formula (Vb):

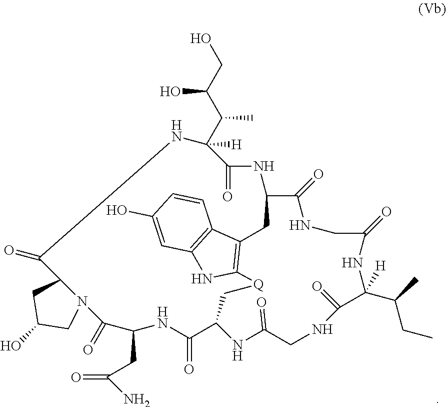

In some embodiments, Q is S. In some embodiments, Q is a sulfoxid group.

Additional amatoxins that may be used for conjugation to an antibody, or antigen-binding fragment thereof, in accordance with the compositions and methods described herein are described, for example, in WO 2016/142049; WO 2016/071856; WO 2017/149077; WO 2018/115466; and WO 2017/046658, the disclosures of which are incorporated herein by reference in their entirety.

### Linkers

The term “Linker” as used herein means a divalent chemical moiety comprising a covalent bond or a chain of atoms that covalently attaches an antibody or fragment thereof (Ab) to an amatoxin as described herein, e.g., an amatoxin of formulae (IV), (IVa), (IVb), (V), (Va), or (Vb), to form an antibody-drug conjugate (ADC).

Covalent attachment of the antibody and the amatoxin requires the linker to have two reactive functional groups, i.e. bivalency in a reactive sense. Bivalent linker reagents which are useful to attach two or more functional or biologically active moieties, such as peptides, nucleic acids, drugs, toxins, antibodies, haptens, and reporter groups are known, and methods have been described their resulting conjugates (Hermanson, G. T. (1996) Bioconjugate Techniques; Academic Press: New York, p. 234-242).

Accordingly, present linkers have two reactive termini, one for conjugation to an antibody and the other for conjugation to an amatoxin. The antibody conjugation reactive terminus of the linker (reactive moiety, defined herein as Z′) is typically a chemical moiety that is capable of conjugation to the antibody through, e.g., a cysteine thiol or lysine amine group on the antibody, and so is typically a thiol-reactive group such as a Michael acceptor (as in maleimide), a leaving group, such as a chloro, bromo, iodo, or an R-sulfanyl group, or an amine-reactive group such as a carboxyl group. Conjugation of the linker to the antibody is described more fully herein below.

The amatoxin conjugation reactive terminus of the linker is typically a chemical moiety that is capable of conjugation to the amatoxin through formation of a bond with a reactive substituent within the amatoxin molecule. Non-limiting examples include, for example, formation of an amide bond with a basic amine or carboxyl group on the amatoxin, via a carboxyl or basic amine group on the linker, respectively, or formation of an ether or the like, via alkylation of an OH group on the amatoxin via e.g., a leaving group on the linker.

When the term “linker” is used in describing the linker in conjugated form, one or both of the reactive termini will be absent (such as reactive moiety Z′, having been converted to chemical moiety Z, as described herein below) or incomplete (such as being only the carbonyl of the carboxylic acid) because of the formation of the bonds between the linker and/or the amatoxin, and between the linker and/or the antibody or antigen-binding fragment thereof. Such conjugation reactions are described further herein below.

A variety of linkers can be used to conjugate the antibodies, antigen-binding fragments, and ligands described to a cytotoxic molecule. Generally, linkers suitable for the present disclosure may be substantially stable in circulation, but allow for release of the amatoxin within or in close proximity to the target cells. In some embodiments, certain linkers suitable for the present disclosure may be categorized as “cleavable” or “non-cleavable”. Generally, cleavable linkers contain one or more functional groups that is cleaved in response to a physiological environment. For example, a cleavable linker may contain an enzymatic substrate (e.g., valine-alanine) that degrades in the presence of an intracellular enzyme (e.g., cathepsin B), an acid-cleavable group (e.g., a hydrozone) that degrades in the acidic environment of a cellular compartment, or a reducible group (e.g., a disulfide) that degrades in an intracellular reducing environment. By contrast, generally, non-cleavable linkers are released from the ADC during degradation (e.g., lysosomal degradation) of the antibody moiety of the ADC inside the target cell.

### Non-Cleavable Linkers

Non-cleavable linkers suitable for use herein further may include one or more groups selected from a bond, —(C═O)—, C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, heteroarylene, and combinations thereof, each of which may be optionally substituted, and/or may include one or more heteroatoms (e.g., S, N, or O) in place of one or more carbon atoms. Non-limiting examples of such groups include (CH₂)ₚ, (C═O)(CH₂)ₚ, and polyethyleneglycol (PEG; (CH₂CH₂O)ₚ), units, wherein p is an integer from 1-6, independently selected for each occasion.

In some embodiments, the linker L comprises one or more of a bond, —(C═O)—, a —C(O)NH— group, an —OC(O)NH— group, C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, heteroarylene, a —(CH₂CH₂O)ₚ— group where p is an integer from 1-6, or a solubility enhancing group;
wherein each C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, or heteroarylene may optionally be substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro;

In some embodiments, each C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, or heteroarylene may optionally be interrupted by one or more heteroatoms selected from O, S and N.

In some embodiments, each C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, or heteroarylene may optionally be interrupted by one or more heteroatoms selected from O, S and N and may be optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

In some embodiments, the linker L comprises a solubility enhancing group of the formula —Oₐ—C(O)NH—SO₂—N(R¹)—, wherein:
a is 0 or 1;
R¹ is selected from the group consisting of hydrogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, each of which may be optionally substituted or optionally interrupted by one or more heteroatoms selected from O, S and NR³, wherein R³ is independently selected from the group consisting of hydrogen and C₁-C₄ alkyl. Such solubility enhancing groups are described in, for example, U.S. Pat. No. 9,636,421 and U.S. Patent Application Publication No. 2017/0298145, the disclosures of each of which are incorporated herein by reference in their entirety.

In some embodiments, the solubility enhancing group of the formula —Oₐ—C(O)NH—SO₂—N(R¹)— further comprises a C₁-C₆ alkylene or a —(CH₂CH₂O)ₚ— group, where p is an integer from 1-6. Non-limiting examples of such solubility enhancing groups include those depicted in Table 2, above.

TABLE 2
Exemplary solubility enhancing groups

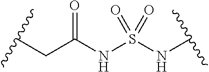 [SMILES: *CC(=O)NS(=O)(=O)N*]

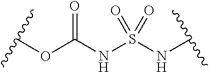 [SMILES: *NS(=O)(=O)NC(=O)O*]

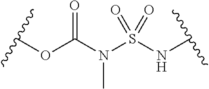 [SMILES: *NS(=O)(=O)N(C)C(=O)O*]

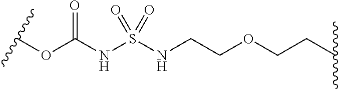 [SMILES: *CCOCCNS(=O)(=O)NC(=O)O*]

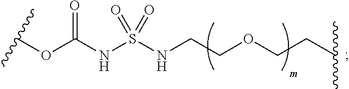

m = 1-6

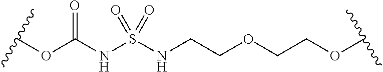 [SMILES: *OCCOCCNS(=O)(=O)NC(=O)O*]

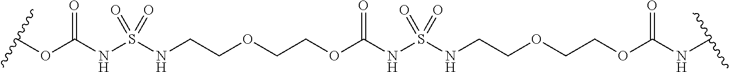 [SMILES: *NC(=O)OCCOCCNS(=O)(=O)NC(=O)OCCOCCNS(=O)(=O)NC(=O)O*]

In some embodiments, the non-cleavable linker comprises a —(CH₂)ₙ— unit, where n is an integer from, 2-12, e.g., 2-6. In some embodiments, the non-cleavable linker comprises a —(CH₂)ₙ— where n is 1, 2, 3, 4, 5, or 6. In some embodiments, the non-cleavable linker is —(CH₂)ₙ— where n is 6, represented by the formula:

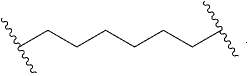 [SMILES: *CCCCCC*]

### Cleavable Linkers

In some embodiments, the linker conjugating the antibody or antigen binding fragment thereof and the amatoxin is cleavable under intracellular conditions, such that cleavage of the linker releases the drug unit from the antibody in the intracellular environment. Cleavable linkers are designed to exploit the differences in local environments, e.g., extracellular and intracellular environments, including, for example, pH, reduction potential or enzyme concentration, to trigger the release of the amatoxin in the target cell. Generally, cleavable linkers are relatively stable in circulation, but are particularly susceptible to cleavage in the intracellular environment through one or more mechanisms (e.g., including, but not limited to, activity of proteases, peptidases, and glucuronidases). Cleavable linkers used herein are substantially stable in circulating plasma and/or outside the target cell and may be cleaved at some efficacious rate inside the target cell or in close proximity to the target cell.

Suitable cleavable linkers include those that may be cleaved, for instance, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012, the disclosure of which is incorporated herein by reference as it pertains to linkers suitable for covalent conjugation). Suitable cleavable linkers may include, for example, chemical moieties such as a hydrazine, a disulfide, a thioether or a dipeptide.

Linkers hydrolyzable under acidic conditions include, for example, hydrazones, semicarbazones, thiosemicarbazones, cis-aconitic amides, orthoesters, acetals, ketals, or the like. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661, the disclosure of each of which is incorporated herein by reference in its entirety as it pertains to linkers suitable for covalent conjugation. Such linkers are relatively stable under neutral pH conditions, such as those in the blood, but are unstable at below pH 5.5 or 5.0, the approximate pH of the lysosome.

Linkers cleavable under reducing conditions include, for example, a disulfide. A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935, the disclosure of each of which is incorporated herein by reference in its entirety as it pertains to linkers suitable for covalent conjugation.

Linkers susceptible to enzymatic hydrolysis can be, e.g., a peptide-containing linker that is cleaved by an intracellular peptidase or protease enzyme, including, but not limited to, a lysosomal or endosomal protease. One advantage of using intracellular proteolytic release of the therapeutic agent is that the agent is typically attenuated when conjugated and the serum stabilities of the conjugates are typically high. In some embodiments, the peptidyl linker is at least two amino acids long or at least three amino acids long. Exemplary amino acid linkers include a dipeptide, a tripeptide, a tetrapeptide or a pentapeptide. Examples of suitable peptides include those containing amino acids such as Valine, Alanine, Citrulline (Cit), Phenylalanine, Lysine, Leucine, and Glycine. Amino acid residues which comprise an amino acid linker component include those occurring naturally, as well as minor amino acids and non-naturally occurring amino acid analogs, such as citrulline. Exemplary dipeptides include valine-citrulline (vc or val-cit) and alanine-phenylalanine (af or ala-phe). Exemplary tripeptides include glycine-valine-citrulline (gly-val-cit) and glycine-glycine-glycine (gly-gly-gly). In some embodiments, the linker includes a dipeptide such as Val-Cit, Ala-Val, or Phe-Lys, Val-Lys, Ala-Lys, Phe-Cit, Leu-Cit, Ile-Cit, Phe-Arg, or Trp-Cit. Linkers containing dipeptides such as Val-Cit or Phe-Lys are disclosed in, for example, U.S. Pat. No. 6,214,345, the disclosure of which is incorporated herein by reference in its entirety as it pertains to linkers suitable for covalent conjugation. In some embodiments, the linker comprises a dipeptide selected from Val-Ala and Val-Cit.

Linkers suitable for conjugating the antibodies, antigen-binding fragments, described herein to a cytotoxic molecule include those capable of releasing an amatoxin by a 1,6-elimination process. Chemical moieties capable of this elimination process include the p-aminobenzyl (PAB) group, 6-maleimidohexanoic acid, pH-sensitive carbonates, and other reagents as described in Jain et al., Pharm. Res. 32:3526-3540, 2015, the disclosure of which is incorporated herein by reference in its entirety as it pertains to linkers suitable for covalent conjugation.

In some embodiments, the linker includes a “self-immolative” group such as the afore-mentioned PAB or PABC (para-aminobenzyloxycarbonyl), which are disclosed in, for example, Carl et al., J. Med. Chem. (1981) 24:479-480; Chakravarty et al (1983) J. Med. Chem. 26:638-644; U.S. Pat. No. 6,214,345; US20030130189; US20030096743; U.S. Pat. No. 6,759,509; US20040052793; U.S. Pat. Nos. 6,218,519; 6,835,807; 6,268,488; US20040018194; WO98/13059; US20040052793; U.S. Pat. Nos. 6,677,435; 5,621,002; US20040121940; WO2004/032828). Other such chemical moieties capable of this process (“self-immolative linkers”) include methylene carbamates and heteroaryl groups such as aminothiazoles, aminoimidazoles, aminopyrimidines, and the like. Linkers containing such heterocyclic self-immolative groups are disclosed in, for example, U.S. Patent Publication Nos. 20160303254 and 20150079114, and U.S. Pat. No. 7,754,681; Hay et al. (1999) Bioorg. Med. Chem. Lett. 9:2237; US 2005/0256030; de Groot et al (2001) J. Org. Chem. 66:8815-8830; and U.S. Pat. No. 7,223,837. In some embodiments, a dipeptide is used in combination with a self-immolative linker.

In some embodiments, the linker L comprises one or more of a hydrazine, a disulfide, a thioether, an amino acid, a peptide consisting of up to 10 amino acids, ap-aminobenzyl (PAB) group, a heterocyclic self-immolative group, C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, heteroaryl, a —(C═O)— group, a —C(O)NH— group, an —OC(O)NH— group, a —(CH₂CH₂O)ₚ— group where p is an integer from 1-6, or a solubility enhancing group;
wherein each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, or heteroaryl group may be optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

In some embodiments, each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, or heteroaryl group may optionally be interrupted by one or more heteroatoms selected from O, S and N.

In some embodiments, each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, or heteroaryl group may optionally be interrupted by one or more heteroatoms selected from O, S and N and may be optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

One of skill in the art will recognize that one or more of the groups listed may be present in the form of a bivalent (diradical) species, e.g., C₁-C₆ alkylene and the like.

In some embodiments, the linker L comprises comprises a solubility enhancing group of the formula —Oₐ—C(O)NH—SO₂—N(R′)—, wherein:
a is 0 or 1;
R¹ is selected from the group consisting of hydrogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, each of which may be optionally substituted or optionally interrupted by one or more heteroatoms selected from O, S and NR³, wherein R³ is independently selected from the group consisting of hydrogen and C₁-C₄ alkyl. Such solubility enhancing groups are described in, for example, U.S. Pat. No. 9,636,421 and U.S. Patent Application Publication No. 2017/0298145, the disclosures of each of which are incorporated herein by reference in their entirety.

In some embodiments, the solubility enhancing group of the formula —Oₐ—C(O)NH—SO₂—N(R¹)— further comprises a C₁-C₆ alkylene or a —(CH₂CH₂O)ₚ— group, where p is an integer from 1-6. Non-limiting examples of such solubility enhancing groups include those depicted in Table 2, above.

In some embodiments, the linker includes a p-aminobenzyl group (PAB). In one embodiment, the p-aminobenzyl group is disposed between the cytotoxic drug and a protease cleavage site in the linker. In one embodiment, the p-aminobenzyl group is part of a p-aminobenzyloxycarbonyl unit. In one embodiment, the p-aminobenzyl group is part of a p-aminobenzylamido unit.

In some embodiments, the linker comprises a dipeptide selected from the group consisting of Phe-Lys, Val-Lys, Phe-Ala, Phe-Cit, Val-Ala, Val-Cit, and Val-Arg. In some embodiments, the linker comprises one or more of PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB.

In some embodiments, the linker comprises a combination of one or more of a peptide, oligosaccharide, —(CH₂)ₚ—, —(CH₂CH₂O)ₚ—, PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB.

In some embodiments, the linker comprises a —(C═O)(CH₂)ₚ— unit, wherein p is an integer from 1-6.

In some embodiments, the linker comprises PAB-Ala-Val-propionyl, represented by the formula:

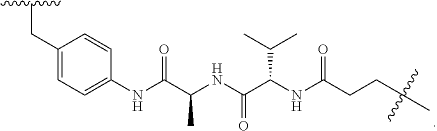 [SMILES: *CCC(=O)N[C@H](C(=O)N[C@@H](C)C(=O)Nc1ccc(C*)cc1)C(C)C]

In some embodiments, the linker comprises PAB-Cit-Val-propionyl, represented by the formula:

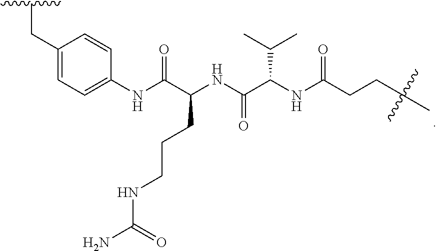 [SMILES: *CCC(=O)N[C@H](C(=O)N[C@@H](CCCNC(N)=O)C(=O)Nc1ccc(C*)cc1)C(C)C]

Such PAB-dipeptide-propionyl linkers are disclosed in, e.g., International Patent Application Publication No. WO2017/149077, which is incorporated by reference herein in its entirety. Further, the cytotoxins disclosed in WO2017/149077 are incorporated by reference herein.

It will be recognized by one of skill in the art that any one or more of the chemical groups, moieties and features disclosed herein may be combined in multiple ways to form linkers useful for conjugation of the antibodies and amatoxins as disclosed herein. Further linkers useful in conjunction with the compositions and methods described herein, are described, for example, in U.S. Patent Application Publication No. 2015/0218220, the disclosure of which is incorporated herein by reference in its entirety.

### Linker-Amatoxin and Linker-Antibody Conjugation

In certain embodiments, the linker is reacted with an amatoxin or derivative thereof according to any of formulae (V), (Va), or (Vb) under appropriate conditions to form a linker-amatoxin conjugate. In certain embodiments, reactive groups are used on the amatoxin or linker to form a covalent attachment.

The amatoxin-linker conjugate is subsequently reacted with the antibody, derivatized antibody, or antigen-binding fragment thereof, under appropriate conditions to form the ADC. Alternatively, the linker may first be reacted with the antibody, derivatized antibody or antigen-binding fragment thereof, to form a linker-antibody conjugate, and then reacted with the amatoxin to form the ADC. Such conjugation reactions will now be described more fully.

A number of different reactions are available for covalent attachment of linkers or amatoxin-linker conjugates to the antibody or antigen-binding fragment thereof. Suitable attachment points on the antibody molecule include, but are not limited to, the amine groups of lysine, the free carboxylic acid groups of glutamic acid and aspartic acid, the sulfhydryl groups of cysteine, and the various moieties of aromatic amino acids. For instance, non-specific covalent attachment may be undertaken using a carbodiimide reaction to link a carboxy (or amino) group on a linker to an amino (or carboxy) group on an antibody moiety. Additionally, bifunctional agents such as dialdehydes or imidoesters may also be used to link the amino group on a linker to an amino group on an antibody moiety. Also available for attachment of amatoxins to antibody moieties is the Schiff base reaction. This method involves the periodate oxidation of a glycol or hydroxy group on either the antibody or linker, thus forming an aldehyde which is then reacted with the linker or antibody, respectively. Covalent bond formation occurs via formation of a Schiff base between the aldehyde and an amino group. Isothiocyanates may also be used as coupling agents for covalently attaching amatoxins or antibody moieties to linkers. Other techniques are known to the skilled artisan and within the scope of the present disclosure.

Linkers useful in for conjugation to the antibodies or antigen-binding fragments as described herein include, without limitation, linkers containing a chemical moiety Z, formed by a coupling reaction between the antibody and a reactive chemical moiety (referred to herein as a reactive substituent, Z′) on the linker as depicted in Table 3, below. Wavy lines designate points of attachment to the antibody or antigen-binding fragment, and the cytotoxic molecule, respectively.

TABLE 3
Exemplary chemical moieties Z formed by coupling reactions in the formation of antibody-drug conjugates.

| Exemplary Coupli [symbol] | |
|---|---|
| Reactions | Chemical Moiety Z Formed by Coupling Reactions |
| [3 + 2] Cycloaddition | 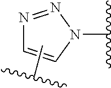 |
| [3 + 2] Cycloaddition | 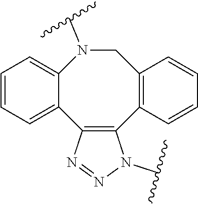 [SMILES: *N1Cc2ccccc2-c2c(nnn2*)-c2ccccc21] |
| [3 + 2] Cycloaddition, Esterification | 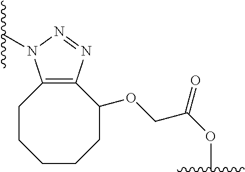 [SMILES: *OC(=O)COC1CCCCCc2c1nnn2*] |
| [3 + 2] Cycloaddition, Esterification | 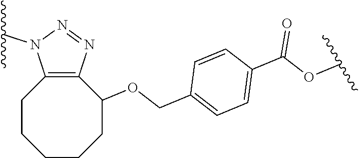 [SMILES: *OC(=O)c1ccc(COC2CCCCCc3c2nnn3*)cc1] |
| [3 + 2] Cycloaddition, Esterification | 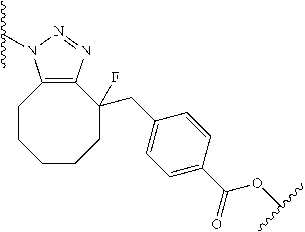 [SMILES: *OC(=O)c1ccc(CC2(F)CCCCCc3c2nnn3*)cc1] |
| [3 + 2] Cycloaddition, Esterification | 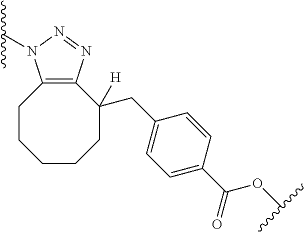 [SMILES: *OC(=O)c1ccc(CC2CCCCCc3c2nnn3*)cc1] |
| [3 + 2] Cycloaddition, Esterification | 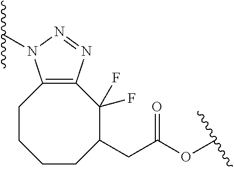 [SMILES: *OC(=O)CC1CCCCc2c(nnn2*)C1(F)F] |
| [3 + 2] Cycloaddition, Esterification | 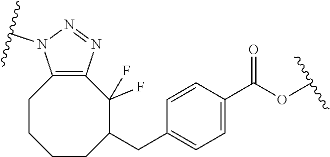 [SMILES: *OC(=O)c1ccc(CC2CCCCc3c(nnn3*)C2(F)F)cc1] |
| [3 + 2] Cycloaddition, Esterification | 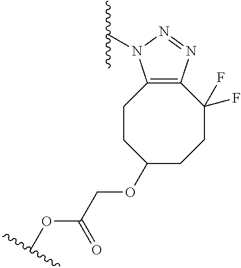 [SMILES: *OC(=O)COC1CCc2c(nnn2*)C(F)(F)CC1] |
| [3 + 2] Cycloaddition, Esterification | 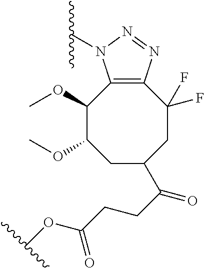 [SMILES: *OC(=O)CCC(=O)C1C[C@H](OC)[C@@H](OC)c2c(nnn2*)C(F)(F)C1] |
| [3 + 2] Cycloaddition, Esterification | 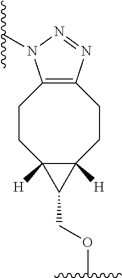 [SMILES: *OC[C@@H]1[C@@H]2CCc3c(nnn3*)CC[C@@H]21] |
| [3 + 2] Cycloaddition, Esterification | 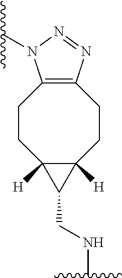 [SMILES: *NC[C@@H]1[C@@H]2CCc3c(nnn3*)CC[C@@H]21] |
| [3 + 2] Cycloaddition, Esterification | 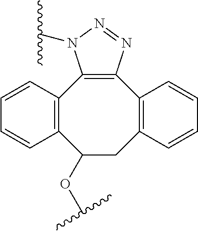 [SMILES: *OC1Cc2ccccc2-c2nnn(*)c2-c2ccccc21] |
| [3 + 2] Cycloaddition, Esterification | 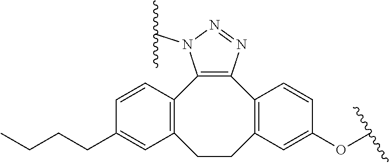 [SMILES: *Oc1ccc2c(c1)CCc1cc(CCCC)ccc1-c1c-2nnn1*] |
| [3 + 2] Cycloaddition | 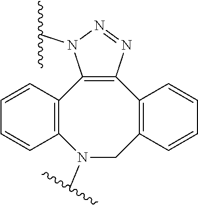 [SMILES: *N1Cc2ccccc2-c2nnn(*)c2-c2ccccc21] |
| Michael addition | 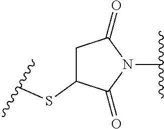 [SMILES: *SC1CC(=O)N(*)C1=O] |
| Michael addition | 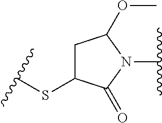 [SMILES: *SC1CC(OC)N(*)C1=O] |
| Imine condensation, Amidation | 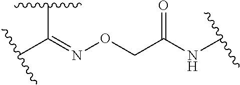 [SMILES: *NC(=O)CON=C(*)*] |
| Imine condensation | 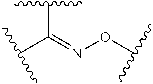 [SMILES: *ON=C(*)*] |
| Disulfide formation | 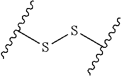 [SMILES: *SS*] |
| Thiol alkylation | 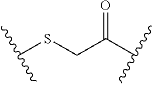 [SMILES: *SCC(*)=O] |
| Condensation, Michael addition | 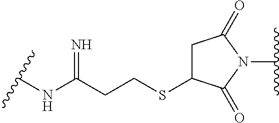 [SMILES: *NC(=N)CCSC1CC(=O)N(*)C1=O] |

[symbol]

indicates data missing or illegible when filed

One of skill in the art will recognize that a reactive substituent Z′ attached to the linker and a reactive substituent on the antibody or antigen-binding fragment thereof, are engaged in the covalent coupling reaction to produce the chemical moiety Z, and will recognize the reactive substituent Z′. Therefore, antibody-drug conjugates useful in conjunction with the methods described herein may be formed by the reaction of an antibody, or antigen-binding fragment thereof, with a linker or amatoxin-linker conjugate, as described herein, the linker or amatoxin-linker conjugate including a reactive substituent Z′, suitable for reaction with a reactive substituent on the antibody, or antigen-binding fragment thereof, to form the chemical moiety Z.

As depicted in Table 3, examples of suitably reactive substituents Z′ on the linker and reactive substituents on the antibody or antigen-binding fragment thereof include a nucleophile/electrophile pair (e.g., a thiol/haloalkyl pair, an amine/carbonyl pair, or a thiol/α,β-unsaturated carbonyl pair, and the like), a diene/dienophile pair (e.g., an azide/alkyne pair, or a diene/α,β-unsaturated carbonyl pair, among others), and the like. Coupling reactions between the reactive substitutents to form the chemical moiety Z include, without limitation, thiol alkylation, hydroxyl alkylation, amine alkylation, amine or hydroxylamine condensation, hydrazine formation, amidation, esterification, disulfide formation, cycloaddition (e.g., [4+2] Diels-Alder cycloaddition, [3+2] Huisgen cycloaddition, among others), nucleophilic aromatic substitution, electrophilic aromatic substitution, and other reactive modalities known in the art or described herein. In some embodiments, the reactive substituent Z′ is an electrophilic functional group suitable for reaction with a nucleophilic functional group on the antibody, or antigen-binding fragment thereof.

Reactive substituents that may be present within an antibody, or antigen-binding fragment thereof, as disclosed herein include, without limitation, nucleophilic groups such as (i)N-terminal amine groups, (ii) side chain amine groups, e.g. lysine, (iii) side chain thiol groups, e.g. cysteine, and (iv) sugar hydroxyl or amino groups where the antibody is glycosylated. Reactive substituents that may be present within an antibody, or antigen-binding fragment thereof, as disclosed herein include, without limitation, hydroxyl moieties of serine, threonine, and tyrosine residues; amino moieties of lysine residues; carboxyl moieties of aspartic acid and glutamic acid residues; and thiol moieties of cysteine residues, as well as propargyl, azido, haloaryl (e.g., fluoroaryl), haloheteroaryl (e.g., fluoroheteroaryl), haloalkyl, and haloheteroalkyl moieties of non-naturally occurring amino acids. In some embodiments, the reactive substituents present within an antibody, or antigen-binding fragment thereof as disclosed herein include, are amine or thiol moieties. Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by introducing one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

In some embodiments, the reactive substituent Z′ attached to the linker is a nucleophilic group which is reactive with an electrophilic group present on an antibody. Useful electrophilic groups on an antibody include, but are not limited to, aldehyde and ketone carbonyl groups. A nucleophilic group (e.g., a) heteroatom of can react with an electrophilic group on an antibody and form a covalent bond to the antibody. Useful nucleophilic groups include, but are not limited to, hydrazide, oxime, amino, hydroxyl, hydrazine, thiosemicarbazone, hydrazine carboxylate, and arylhydrazide.

In some embodiments, chemical moiety Z is the product of a reaction between reactive nucleophilic substituents present within the antibodies, or antigen-binding fragments thereof, such as amine and thiol moieties, and a reactive electrophilic substituent Z′ attached to the linker. For instance, Z′ may be a Michael acceptor (e.g., maleimide), activated ester, electron-deficient carbonyl compound, or an aldehyde, among others.

Several representative and non-limiting examples of reactive substituents Z′ and the resulting chemical moieties Z are provided in Table 4.

TABLE 4
Complementary reactive substituents and chemical moieties

| | Functional | | |
|---|---|---|---|
| | Group | | |
| | on Antibody | Z′ group | Z group |
| Naturally Occurring | 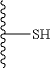 [SMILES: *S] | 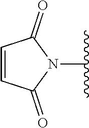 [SMILES: *N1C(=O)C=CC1=O] | 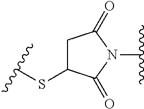 [SMILES: *SC1CC(=O)N(*)C1=O] |
| | | 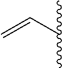 [SMILES: *C=C] | 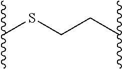 [SMILES: *CCS*] |
| | 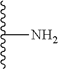 [SMILES: *N] | 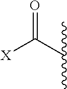 | 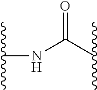 [SMILES: *NC(*)=O] |
| Synthet- ically Intro- duced | 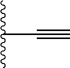 [SMILES: *C#C] | 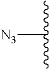 [SMILES: *N=[N+]=[N-]] | 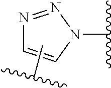 |
| | 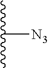 [SMILES: *N=[N+]=[N-]] | 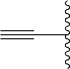 [SMILES: *C#C] | 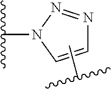 |
| | | 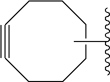 | 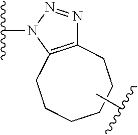 |
| | 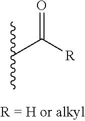 | 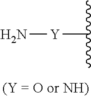 | 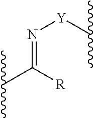 |

For instance, linkers suitable for the synthesis of linker-antibody conjugates and ADCs include, without limitation, reactive substituents Z′ attached to the linker, such as a maleimide or haloalkyl group. These may be attached to the linker by, for example, reagents such as succinimidyl 4-(N-maleimidomethyl)-cyclohexane-L-carboxylate (SMCC), N-succinimidyl iodoacetate (SIA), sulfo-SMCC, m-maleimidobenzoyl-N-hydroxysuccinimidyl ester (MBS), sulfo-MBS, and succinimidyl iodoacetate, among others described, in for instance, Liu et al., 18:690-697, 1979, the disclosure of which is incorporated herein by reference as it pertains to linkers for chemical conjugation.

In some embodiments, the reactive substituent Z′ attached to linker L is a maleimide, azide, or alkyne. An example of a maleimide-containing linker is the non-cleavable maleimidocaproyl-based linker, which is particularly useful for the conjugation of microtubule-disrupting agents such as auristatins. Such linkers are described by Doronina et al., Bioconjugate Chem. 17:14-24, 2006, the disclosure of which is incorporated herein by reference as it pertains to linkers for chemical conjugation.

In some embodiments, the reactive substituent Z′ is —(C═O)— or —NH(C═O)—, such that the linker may be joined to the antibody, or antigen-binding fragment thereof, by an amide or urea moiety, respectively, resulting from reaction of the —(C═O)— or —NH(C═O)— group with an amino group of the antibody or antigen-binding fragment thereof.

In some embodiments, the reactive substituent Z′ is an N-maleimidyl group, halogenated N-alkylamido group, sulfonyloxy N-alkylamido group, carbonate group, sulfonyl halide group, thiol group or derivative thereof, alkynyl group comprising an internal carbon-carbon triple bond, (hetero)cycloalkynyl group, bicyclo[6.1.0]non-4-yn-9-yl group, alkenyl group comprising an internal carbon-carbon double bond, cycloalkenyl group, tetrazinyl group, azido group, phosphine group, nitrile oxide group, nitrone group, nitrile imine group, diazo group, ketone group, (O-alkyl)hydroxylamino group, hydrazine group, halogenated N-maleimidyl group, 1,1-bis(sulfonylmethyl)methylcarbonyl group or elimination derivatives thereof, carbonyl halide group, or an allenamide group, each of which may be optionally substituted. In some embodiments, the reactive substiuent comprises a cycloalkene group, a cycloalkyne group, or an optionally substituted (hetero)cycloalkynyl group.

In some embodiments, the chemical moiety Z is selected from Table 3 or Table 4. In some embodiments, the chemical moiety Z is:

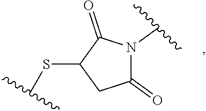

where S is a sulfur atom which represents the reactive substituent present within an antibody, or antigen-binding fragment thereof, that specifically binds to an antigen expressed on the cell surface of a human stem cell or a T cell (e.g., from the —SH group of a cysteine residue).

In some embodiments, the linker-reactive substituent group, taken together as L-Z′, prior to conjugation with the antibody or antigen binding fragment thereof, has the structure:

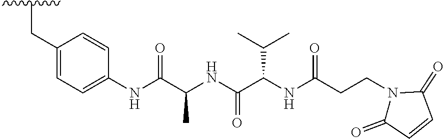 [SMILES: *Cc1ccc(NC(=O)[C@H](C)NC(=O)[C@@H](NC(=O)CCN2C(=O)C=CC2=O)C(C)C)cc1]

where the wavy line indicates the point of attachment to a substuent on the amatoxin. This linker-reactive substituent group L-Z′ may alternatively be referred to as N-beta-maleimidopropyl-Val-Ala-para-aminobenzyl (BMP-Val-Ala-PAB). The wavy line at the linker terminus indicates the point of attachment to the amatoxin. In some embodiments, the linker L and the chemical moiety Z, after conjugation to the antibody, taken together as L-Z-Ab, has the structure:

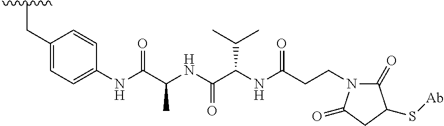

where S is a sulfur atom which represents the reactive substituent present within an antibody, or antigen-binding fragment thereof, that specifically binds to an antigen expressed on the cell surface of a human stem cell or a T cell (e.g., from the —SH group of a cysteine residue. The wavy line at the linker terminus indicates the point of attachment to the amatoxin.

In some embodiments, the linker-reactive substituent group, taken together as L-Z′, prior to conjugation with the antibody or antigen binding fragment thereof, has the structure:

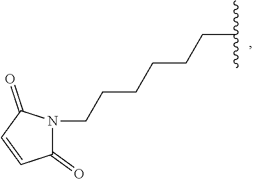 [SMILES: *CCCCCCN1C(=O)C=CC1=O]

This linker-reactive substituent group may alternatively be referred to as 1-n-hexyl-maleimide, which is a non-cleavable linker. The wavy line at the linker terminus indicates the point of attachment to the amatoxin.

In some embodiments, the linker L and the chemical moiety Z, after conjugation to the antibody, taken together as L-Z-Ab, has the structure:

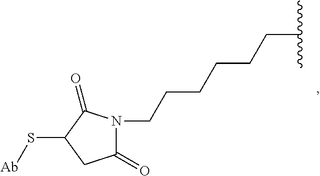

where S is a sulfur atom which represents the reactive substituent present within an antibody, or antigen-binding fragment thereof, that specifically binds to an antigen expressed on the cell surface of a cell, e.g., a tumor cell or a human stem cell or a T cell (e.g., from the —SH group of a cysteine residue). The wavy line at the linker terminus indicates the point of attachment to the amatoxin. One of skill in the art will recognize that in this embodiment, the linker-reactive substituent group structure L-Z′, prior to conjugation with the antibody or antigen binding fragment thereof, includes a maleimide as the group Z′. The foregoing linker moieties and amatoxin-linker conjugates, among others useful in conjunction with the compositions and methods described herein, are described, for example, in U.S. Patent Application Publication No. 2015/0218220 and Patent Application Publication No. WO2017/149077, the disclosure of each of which is incorporated herein by reference in its entirety.

In one aspect, the cytotoxin of the ADC as disclosed herein is an amatoxin or derivative thereof as represented by any of formulae (V), (Va), or (Vb). One of skill in the art will recognize that such amatoxins present multiple possibilities for attachment points to the linker.

In some embodiments, the amatoxin has the structure of formula (V), and the linker is attached by an ether bond to the OH group of the hydroxyl tryptophan residue. In such embodiments, the ADC may be represented by formula (I):

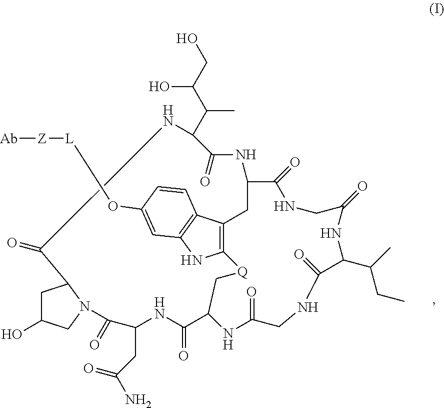

or a stereoisomer thereof,
wherein:
   Q is S;
   L is a linker;
   Z is a chemical moiety formed by a coupling reaction between a reactive substituent present on L and a reactive substituent present within an antibody, or antigen-binding fragment thereof; and
   Ab is an antibody,
   each as disclosed herein.

In some embodiments, the amatoxin has the structure of formula (Va), and the linker is attached by an ether bond to the OH group of the hydroxyl tryptophan residue. In such embodiments, the ADC may be represented by formula (Ia):

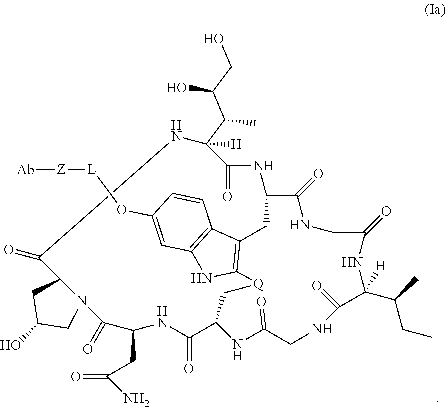

In some embodiments, the amatoxin has the structure of formula (Vb), and the linker is attached by an ether bond to the OH group of the hydroxyl tryptophan residue. In such embodiments, the ADC may be represented by formula (Ib):

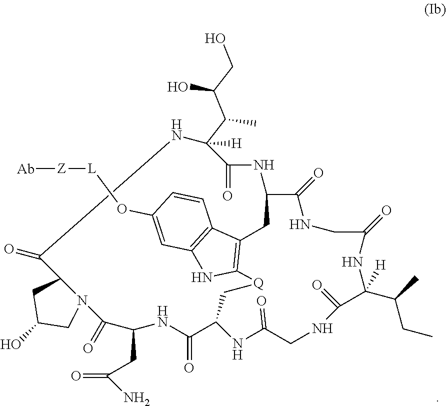

In some embodiments, the linker L of the ADC of formula (I), (Ia), or (Ib) is a non-cleavable linker. In some embodiments, the non-cleavable linker L comprises one or more of a bond, —(C═O)—, a —C(O)NH— group, an —OC(O)NH— group, C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, heteroarylene, a —(CH₂CH₂O)ₚ— group where p is an integer from 1-6, or a solubility enhancing group;
wherein each C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, or heteroarylene may optionally be substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro; or
each C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, or heteroarylene may optionally be interrupted by one or more heteroatoms selected from O, S and N.

In some embodiments, the non-cleavable linker L comprises a solubility enhancing group of the formula —Oₐ—C(O)NH—SO₂—NR¹—, wherein:
a is 0 or 1; and
R¹ is selected from the group consisting of hydrogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, each of which may be optionally substituted, optionally interrupted, or both, by one or more heteroatoms selected from O, S and NR³, wherein R³ is independently selected from the group consisting of hydrogen and C₁-C₄ alkyl.

In some embodiments, the non-cleavable linker L comprises a —(CH₂)ₙ— unit, where n is an integer from 2-6. In some embodiments, the non-cleavable linker L is —(CH₂)ₙ—, where n is 6.

In some embodiments, Ab, Z, and the non-cleavable linker L, taken together as Ab-Z-L, is represented by the formula:

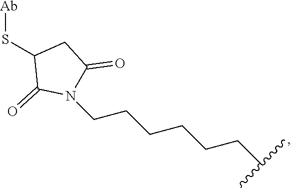

where S is a sulfur atom which represents the reactive substituent present within the antibody, or antigen-binding fragment thereof, that specifically binds to an antigen expressed on a cell, e.g., surface of a human stem cell or a T cell (e.g., from the —SH group of a cysteine residue). The wavy line at the linker terminus indicates the point of attachment to the amatoxin.

In some embodiments, the ADC according to formula (I) is represented by formula (II):

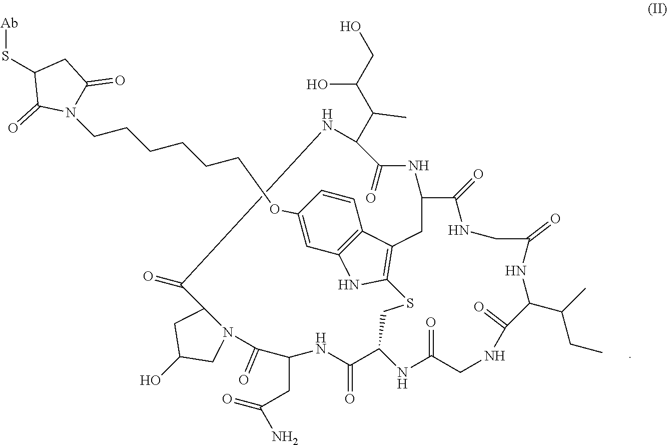

a stereoisomer thereof, or a pharmaceutically acceptable salt thereof.

In some embodiments, the ADC according to formula (II) is represented by formula (IIa):

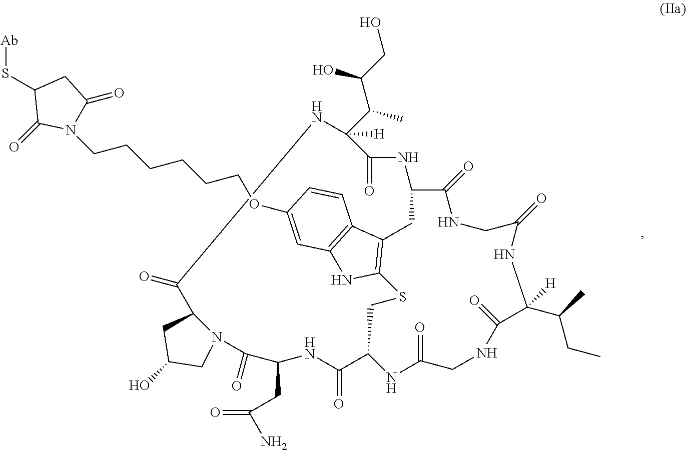

a stereoisomer thereof, or a pharmaceutically acceptable salt thereof.

In some embodiments, the ADC according to formula (II) is represented by formula (IIb):

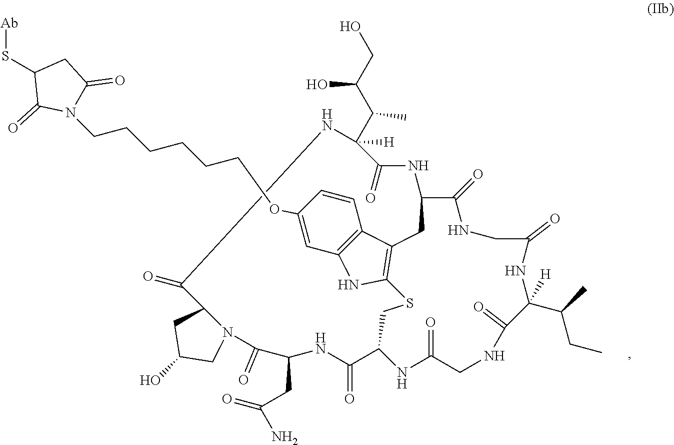

a stereoisomer thereof, or a pharmaceutically acceptable salt thereof.

Surprisingly, according to the present disclosure, it has been found that ADCs comprising an amatoxin and a non-cleavable linker conjugating the amatoxin to the antibody moiety of the ADC have improved tolerability as compared to an ADC comprising an amatoxin and a cleavable linker. For example, in some embodiments, the improved tolerability may be increased therapeutic index. In some embodiments, the improved tolerability may be a smaller elevation, or absence of elevation, of one or more blood liver enzyme levels (e.g., AST, ALT, ADH, or total bilirubin) at a particular dose of the ADC comprising a non-cleavable linker as compared to an ADC comprising a cleavable linker.

In some embodiments, the linker L of the ADC of formula (I), (Ia), or (Ib) is a cleavable linker. In some embodiments, the cleavable linker L comprises one or more of a hydrazine, a disulfide, a thioether, an amino acid, a peptide consisting of up to 10 amino acids, a p-aminobenzyl (PAB) group, a heterocyclic self-immolative group, C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, heteroaryl, a —(C═O)— group, a —C(O)NH— group, an —OC(O)NH— group, a —(CH₂CH₂O)ₚ— group where p is an integer from 1-6, or a solubility enhancing group;
wherein each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, or heteroaryl group may be optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro;
or each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, or heteroaryl group may optionally be interrupted by one or more heteroatoms selected from O, S and N.

In some embodiments, the cleavable linker L comprises a solubility enhancing group of the formula —Oₐ—C(O)NH—SO₂—NR¹—, wherein:
a is 0 or 1; and
R¹ is selected from the group consisting of hydrogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, each of which may be optionally substituted or optionally interrupted by one or more heteroatoms selected from O, S and NR³, wherein R³ is independently selected from the group consisting of hydrogen and C₁-C₄ alkyl.

In some embodiments, the cleavable linker L comprises a peptide selected from the group consisting of Phe-Lys, Val-Lys, Phe-Ala, Phe-Cit, Val-Ala, Val-Cit, and Val-Arg.

In some embodiments, the cleavable linker L further comprises a PAB group.

In some embodiments, the cleavable linker L is represented by the formula:

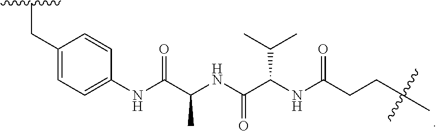 [SMILES: *CCC(=O)N[C@H](C(=O)N[C@@H](C)C(=O)Nc1ccc(C*)cc1)C(C)C]

### Preparation of Antibody-Drug Conjugates

In the ADCs of formula Ab-(Z-L-Cy)ₙ as disclosed herein, such as an ADC of any of formulae (I), (Ia), or (Tb), (II), (IIa), or (IIb), an antibody or antigen binding fragment thereof (Ab) is conjugated to one or more cytotoxic drug moieties (Cy; e.g., an amatoxin), for example, from about 1 to about 20 cytotoxic moieties per antibody, through a linker L and a chemical moiety Z, each as disclosed herein. In some embodiments, n is 1. In some embodiments, n is about 1 to about 5, about 1 to about 4, about 1 to about 3, or about 3 to about 5. In some embodiments, n is about 1, about 2, about 3, or about 4.

The ADCs of the present disclosure may be prepared by several routes, employing organic chemistry reactions, conditions, and reagents known to those skilled in the art, including: (1) reaction of a reactive substituent of an antibody or antigen binding fragment thereof with a bivalent linker reagent to form Ab-Z-L as described herein above, followed by reaction with a cytotoxic moiety Cy; or (2) reaction of a reactive substituent of a cytotoxic moiety with a bivalent linker reagent to form Cy-L-Z′, followed by reaction with a reactive substituent of an antibody or antigen binding fragment thereof as described herein above, to form an ADC of formula Ab-(Z-L-Cy)ₙ. Additional methods for preparing ADC are described herein.

In one embodiment, the antibody or antigen binding fragment thereof can have one or more carbohydrate groups that can be chemically modified to have one or more sulfhydryl groups. The ADC is then formed by conjugation through the sulfhydryl group's sulfur atom as described herein above.

In another embodiment, the antibody can have one or more carbohydrate groups that can be oxidized to provide an aldehyde (—CHO) group (see, for e.g., Laguzza, et al., J. Med. Chem. 1989, 32(3), 548-55). The ADC is then formed by conjugation through the corresponding aldehyde as described herein above. Other protocols for the modification of proteins for the attachment or association of cytotoxins are described in Coligan et al., Current Protocols in Protein Science, vol. 2, John Wiley & Sons (2002), incorporated herein by reference.

Methods for the conjugation of linker-drug moieties to cell-targeted proteins such as antibodies, immunoglobulins or fragments thereof are found, for example, in U.S. Pat. Nos. 5,208,020; 6,441,163; WO2005037992; WO2005081711; and WO2006/034488, all of which are hereby expressly incorporated by reference in their entirety.

Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

In some embodiments, the ADC of formula (IIa) may be prepared by conjugation of a thiol group on the antibody to the amatoxin-linker conjugate Cy-L-Z′, represented by the structure:

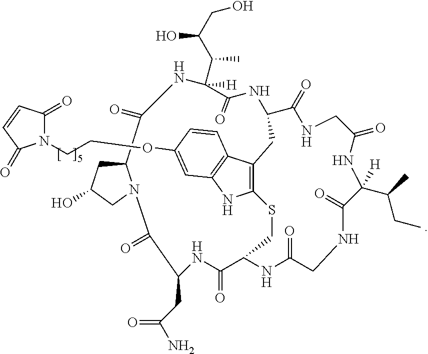

This amatoxin-linker conjugate may be prepared according to Schemes 1 to 3, starting from commercially available 6-hydroxytryptophan (1). Compound 1 may be protected with tert-butyloxycarbonyl anhydride ((BOC)₂O). Hexahydropyrroloindole 3 may be produced as a mixture of cis and trans isomers upon irradiation of protected hydroxytryptophan 2 in the presence of oxygen and a sensitizer (Rose Bengal). Preparation of compound 3 (as well as procedure for preparation of compounds 2 to Va) are provided in International Patent Application Publication No. WO2019/030173, the disclosure of which is incorporated by reference herein in its entirety. Commercial 9-fluorenylmethoxycarbonyl (FMOC) protected 4-hydroxyproline (4) may be alkylated with allyl bromide and subsequently attached to tetrahydropyranyl (THP) polystyrene resin under mild acidic conditions (pyridinium para-toluenesulfonate; PPTS) to give the resin bound allyl ester 5, which may be deprotected with palladium tetrakistriphenylphosphine palladium and dimethyl barbituric acid to give intermediate 6. Protected amino acid 7 may be prepared according to the method reported in International Patent Application Publication No. WO2014/009025, the disclosure of which is incorporated herein in its entirety. Protected amine 7 may then be coupled to resin bound hydroxyproline 6 to afford peptide 8.

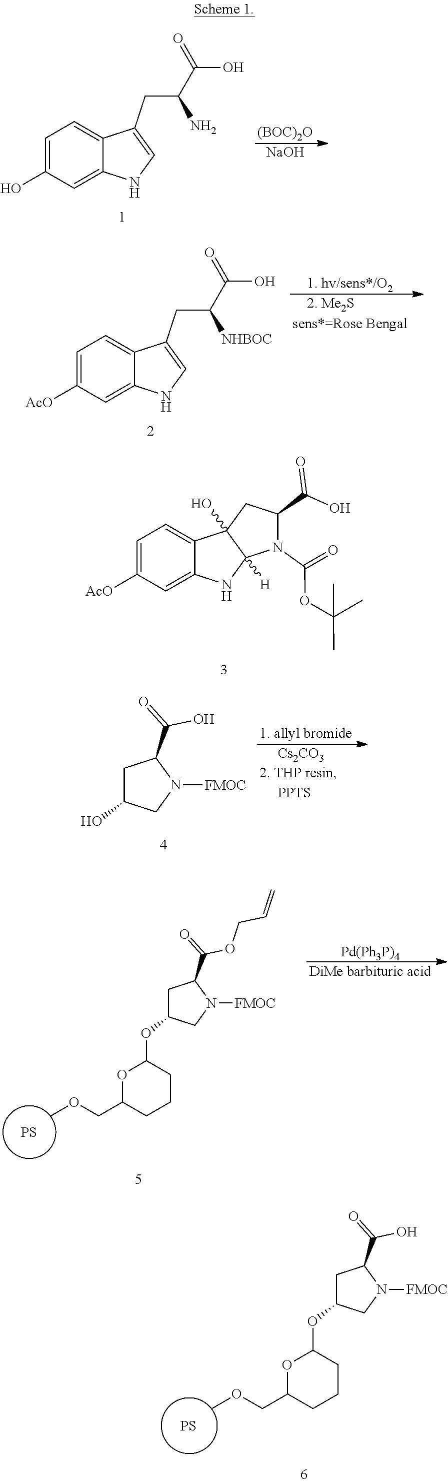

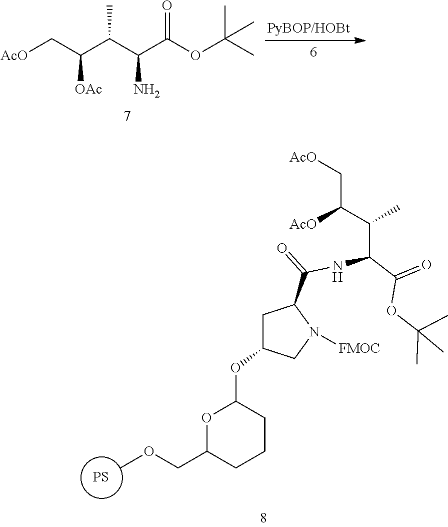

Peptide 8 may then be subjected to multiple coupling and deprotection reactions to provide monocyclic intermediate 9 (Scheme 2). Amino acids FmocAsn(Trt)OH, FmocCys(OTrt)OH, FmocGlyOH, FmocIleOH, FmocGlyOH, and hexahydropyrroloindole 3 may be utilized in sequential solid phase coupling reactions to yield intermediate 9. Each amino acid may be coupled using PyBOP/HOBT in dichloromethane and dimethylformamide (DMF) in the presence of diisopropylethylamine (DIEA). Deprotection may be performed with 20% piperidine in DMF.

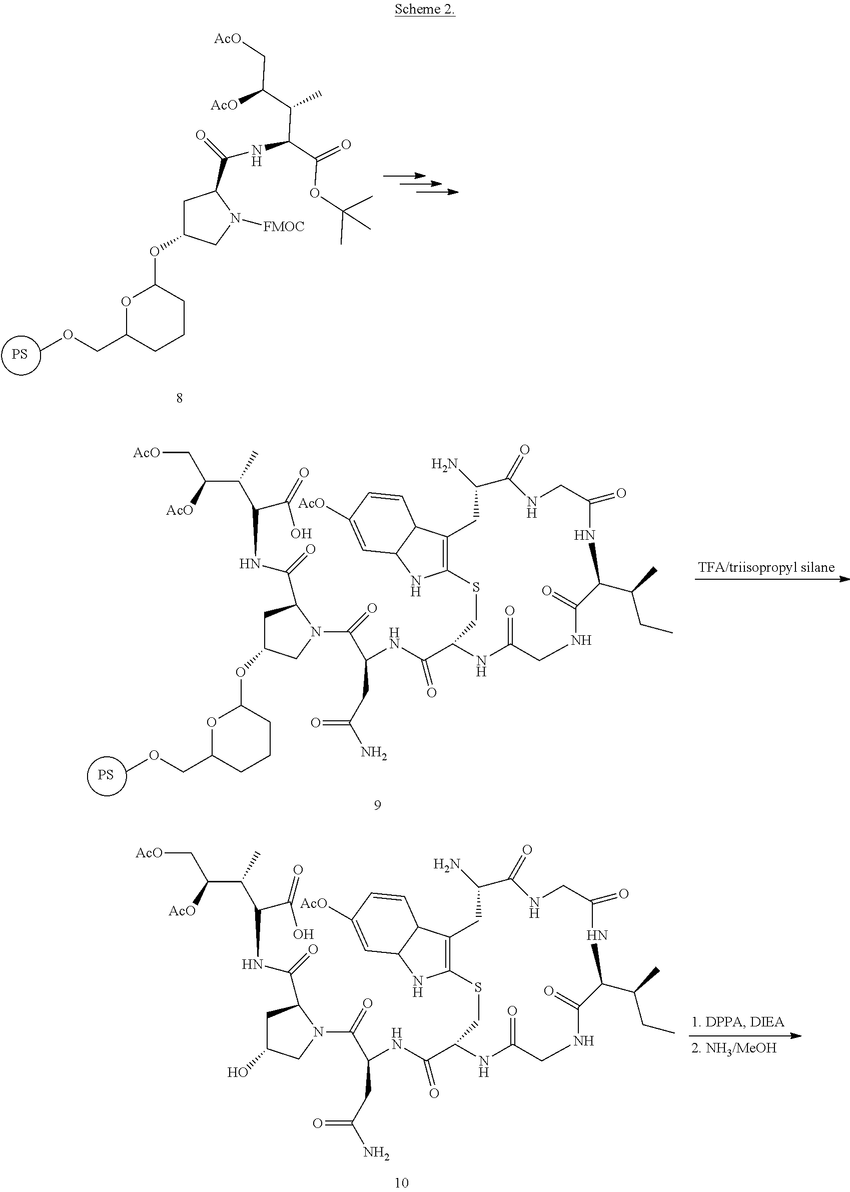

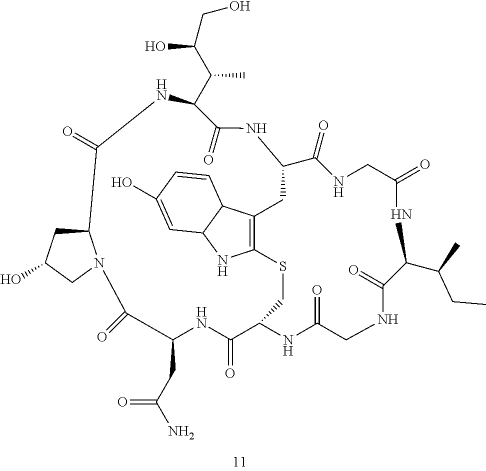 [SMILES: CC[C@H](C)[C@@H]1NC(=O)CNC(=O)[C@@H]2CC3=C(NC4C=C(O)C=CC34)SC[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

Following the final coupling reaction, intermediate 9 may be cleaved from the solid phase support resin with trifluoroacetic acid in the presence of triisopropylsilane to give peptide 10. Treatment of 10 with diphenylphosphorylazide (DPPA) and DIEA induced macrocyclization, and deprotection with ammoniacal methanol provided the amatoxin derivative 11 (i.e., formula Va, where Q=S).

The maleimidohexyl amatoxin conjugate 14 may be prepared from compound 11 according to Scheme 3. Diels-Alder adduct 12 may be prepared from maleimide and 2,5-dimethylfuran and then alkylated with 1,6-dibromohexane to give protected linker 13. Compound 11 may be alkylated with compound 13 in dimethyl sulfoxide (DMSO) in the presence of sodium hydroxide, followed by heating to 100° C. in DMSO to afford the amatoxin-linker conjugate 14. Procedures for preparing compounds 12 and 13, as well as O-alkylation of a related amatoxin (α-amanitin) have been previously reported in U.S. Patent Application Publication No. 2018/0043033, the disclosure of which is incorporated by reference herein in its entirety.

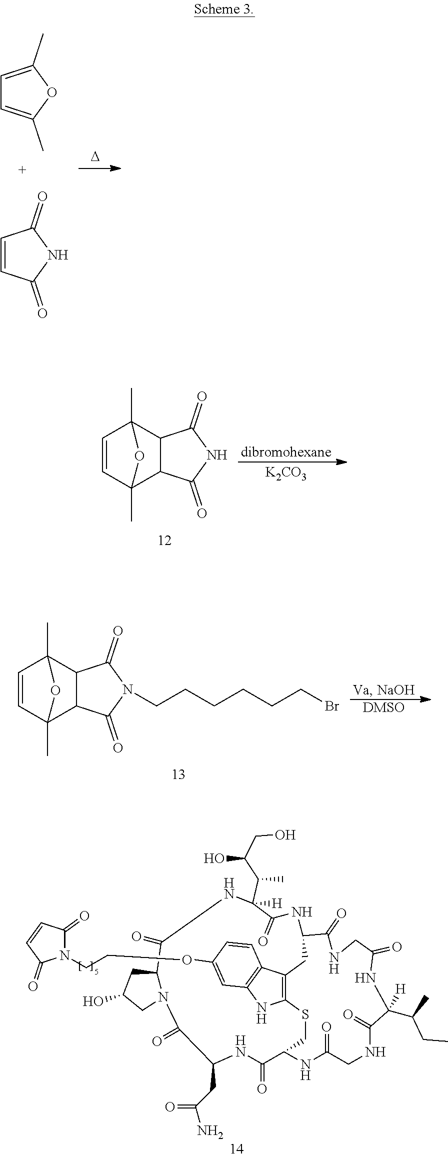

### Antibodies

The ADC compositions and methods disclosed herein comprise an agent to facilitate the selective delivery of such ADCs to a population of cells in the target tissues (e.g., cancer or tumor cells, or hematopoietic stem cells of the bone marrow stem cell niche). The cell target specificity of the ADC is determined by an antigen binding protein such as an antibody, or antigen binding portion thereof.

In one embodiment, the invention includes ADCs comprising antibodies, and antigen-binding fragments thereof, that specifically bind to human CD45, CD49d (VLA-4), CD49f(VLA-6), CD51, CD84, CD90, CD117, CD133, CD134, CD184 (CXCR4), HLA-DR, CD11a, CD18, CD34, CD41/61, CD43, CD58, CD71, CD97, CD162, CD166, CD205 and CD361, CD13, CD33, CD34, CD44, CD4, CD59, CD84/CD150, CD90/Thy1, CD93, CD105/Endoglin, CD123/IL-3R, CD126/IL-6R, CD133, CD135/Flt3 receptor, CD166/ALCAM, Prominin 2, Erythropoietin R, Endothelial Cell-Selective Adhesion Molecule, CD244, Tie1, Tie2, MPL, G-CSFR, CSF3R, IL-1R, gp130, Leukemia inhibitory factor Receptor, oncostatin M receptor, Embigin and IL-18R. Other examples of antigens that can be bound by the ADCs disclosed herein include, but are not limited to, CD7, CDw12, CD13, CD15, CD19, CD21, CD22, CD29, CD30, CD33, CD34, CD36, CD38, CD40, CD41, CD42a, CD42b, CD42c, CD42d, CD43, CD45RA, CD45RB, CD45RC, CD45RO, CD48, CD49b, CD49d, CD49e, CD49f, CD50, CD53, CD55, CD64a, CD68, CD71, CD72, CD73, CD81, CD82, CD85A, CD85K, CD90, CD99, CD104, CD105, CD109, CD110, CD111, CD112, CD114, CD115, CD123, CD124, CD126, CD127, CD130, CD131, CD133, CD135, CD138, CD151, CD157, CD162, CD164, CD168, CD172a, CD173, CD174, CD175, CD175s, CD176, CD183, CD191, CD200, CD201, CD205, CD217, CD220, CD221, CD222, CD223, CD224, CD225, CD226, CD227, CD228, CD229, CD230, CD235a, CD235b, CD236, CD236R, CD238, CD240, CD242, CD243, CD277, CD292, CDw293, CD295, CD298, CD309, CD318, CD324, CD325, CD338, CD344, CD349 or CD350. Other examples of antigens that can be bound by the ADCs disclosed herein include, but are not limited to, CD11a, CD18, CD37, CD47, CD52, CD58, CD62L, CD69, CD74, CD97, CD103, CD132, CD156a, CD179a, CD79b, CD184, CD232, CD244, CD252, CD302, CD305, CD317 and CD361.

In certain embodiments, an antibody, or antigen binding fragment thereof, in an ADC described herein has a certain dissociation rate which is particularly advantageous when used as a part of a conjugate. For example, an anti-CD117 antibody has, in certain embodiments, an off rate constant (Koff) for human CD117 and/or rhesus CD117 of 1×10⁻² to 1×10⁻³, 1×10⁻³ to 1×10⁻⁴, 1×10⁻⁵ to 1×10⁻⁶, 1×10⁻⁶ to 1×10⁻⁷ or 1×10⁻⁷ to 1×10⁻⁸, as measured by bio-layer interferometry (BLI). In some embodiments, the antibody or antigen-binding fragment thereof binds a cell surface antigen (e.g., human CD117 and/or rhesus CD117) with a K_{D} of about 100 nM or less, about 90 nM or less, about 80 nM or less, about 70 nM or less, about 60 nM or less, about 50 nM or less, about 40 nM or less, about 30 nM or less, about 20 nM or less, about 10 nM or less, about 8 nM or less, about 6 nM or less, about 4 nM or less, about 2 nM or less, about 1 nM or less as determined by a Bio-Layer Interferometry (BLI) assay.

### Anti-CD117 Antibodies

In one embodiment, the present invention includes ADCs comprising antibodies, and antigen-binding fragments thereof, that specifically bind to CD117, such as GNNK+CD117. Such ADCs may be used as therapeutic agents to, for example, (i) treat cancers and autoimmune diseases characterized by CD117+ cells and (ii) promote the engraftment of transplanted hematopoietic stem cells in a patient in need of transplant therapy. These therapeutic activities can be caused, for instance, by the binding of isolated anti-CD117 antibodies, antigen-binding fragments thereof, that bind to CD117 (e.g., GNNK+CD117) expressed on the surface of a cell, such as a cancer cell, autoimmune cell, or hematopoietic stem cell and subsequently inducing cell death. The depletion of endogenous hematopoietic stem cells can provide a niche toward which transplanted hematopoietic stem cells can home, and subsequently establish productive hematopoiesis. In this way, transplanted hematopoietic stem cells may successfully engraft in a patient, such as human patient suffering from a stem cell disorder described herein.

Antibodies and antigen-binding fragments capable of binding human CD117 (also referred to as c-Kit, mRNA NCBI Reference Sequence: NM_000222.2, Protein NCBI Reference Sequence: NP_000213.1), including those capable of binding GNNK+CD117, can be used in conjunction with the compositions and methods described herein in order to condition a patient for hematopoietic stem cell transplant therapy. Polymorphisms affecting the coding region or extracellular domain of CD117 in a significant percentage of the population are not currently well-known in non-oncology indications. There are at least four isoforms of CD117 that have been identified, with the potential of additional isoforms expressed in tumor cells. Two of the CD117 isoforms are located on the intracellular domain of the protein, and two are present in the external juxtamembrane region. The two extracellular isoforms, GNNK+ and GNNK−, differ in the presence (GNNK+) or absence (GNNK−) of a 4 amino acid sequence. These isoforms are reported to have the same affinity for the ligand (SCF), but ligand binding to the GNNK− isoform was reported to increase internalization and degradation. The GNNK+ isoform can be used as an immunogen in order to generate antibodies capable of binding CD117, as antibodies generated against this isoform will be inclusive of the GNNK+ and GNNK− proteins. The amino acid sequences of human CD117 isoforms 1 and 2 are described in SEQ ID Nos: 145 and 146, respectively. In certain embodiments, anti-human CD117 (hCD117) antibodies disclosed herein are able to bind to both isoform 1 and isoform 2 of human CD117.

As described below, a yeast library screen of human antibodies was performed to identify novel anti-CD117 antibodies, and fragments thereof, having diagnostic and therapeutic use. Antibody 54 (Ab54), Antibody 55 (Ab55), Antibody 56 (Ab56), Antibody 57 (Ab57), Antibody 58 (Ab58), Antibody 61 (Ab61), Antibody 66 (Ab66), Antibody 67 (Ab67), Antibody 68 (Ab68), and Antibody 69 (Ab69) were human antibodies that were identified in this screen. These antibodies cross react with human CD117 and rhesus CD117. Further, these antibodies disclosed herein are able to bind to both isoforms of human CD117, i.e., isoform 1 (SEQ ID NO: 145) and isoform 2 (SEQ ID NO: 146).

The amino acid sequences for the various binding regions of anti-CD117 antibodies, including Ab54, Ab55, Ab56, Ab57, Ab58, Ab61, Ab66, Ab67, Ab68, and Ab69 are described in the Sequence Table below. Included in the invention are ADCs comprising human anti-CD117 antibodies comprising the CDRs as set forth in the Sequence Table below, as well as human anti-CD117 antibodies comprising the variable regions set forth in the Sequence Table below.

In one embodiment, the invention provides an ADC comprising an anti-CD117 antibody, or antigen-binding fragment thereof, comprising binding regions, e.g., CDRs, variable regions, corresponding to those of Antibody 55. The heavy chain variable region (VH) amino acid sequence of Antibody 55 (i.e., Ab55) is set forth in SEQ ID NO: 19 (see Sequence Table). The VH CDR domain amino acid sequences of Antibody 55 are set forth in SEQ ID NO: 21 (VH CDR1); SEQ ID NO: 22 (VH CDR2), and SEQ ID NO: 23 (VH CDR3). The light chain variable region (VL) amino acid sequence of Antibody 55 is described in SEQ ID NO: 20 (see Sequence Table). The VL CDR domain amino acid sequences of Antibody 55 are set forth in SEQ ID NO: 24 (VL CDR1); SEQ ID NO: 25 (VL CDR2), and SEQ ID NO: 26 (VL CDR3). The heavy chain constant region of Antibody 55 is set forth in SEQ ID NO: 122. The light chain constant region of Antibody 55 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 21, 22, and 23, and a light chain variable region CDR set as set forth in SEQ ID Nos: 24, 25, and 26. In other embodiments, an anti-CD117 antibody, or antigen-binding portion thereof, comprises a variable light chain comprising the amino acid residues set forth in SEQ ID NO: 20, and a heavy chain variable region as set forth in SEQ ID NO: 19.

In one embodiment, the invention provides an an ADC comprising an anti-CD117 antibody, or antigen-binding fragment thereof, comprising binding regions, e.g., CDRs, variable regions, corresponding to those of Antibody 54. The heavy chain variable region (VH) amino acid sequence of Antibody 54 (i.e., Ab54) is set forth in SEQ ID NO: 29 (see Sequence Table). The VH CDR domain amino acid sequences of Antibody 54 are set forth in SEQ ID NO: 31 (VH CDR1); SEQ ID NO: 32 (VH CDR2), and SEQ ID NO: 33 (VH CDR3). The light chain variable region (VL) amino acid sequence of Antibody 54 is described in SEQ ID NO: 30 (see Sequence Table). The VL CDR domain amino acid sequences of Antibody 54 are set forth in SEQ ID NO: 34 (VL CDR1); SEQ ID NO: 35 (VL CDR2), and SEQ ID NO: 36 (VL CDR3). The heavy chain constant region of Antibody 54 is set forth in SEQ ID NO: 122. The light chain constant region of Antibody 54 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 31, 32, and 33, and a light chain variable region CDR set as set forth in SEQ ID Nos: 34, 35, and 36. In other embodiments, an anti-CD117 antibody, or antigen-binding portion thereof, comprises a variable light chain comprising the amino acid residues set forth in SEQ ID NO: 30, and a heavy chain variable region as set forth in SEQ ID NO: 29.

In one embodiment, the invention provides an ADC comprising an anti-CD117 antibody, or antigen-binding fragment thereof, comprising binding regions, e.g., CDRs, variable regions, corresponding to those of Antibody 56. The heavy chain variable region (VH) amino acid sequence of Antibody 56 (i.e., Ab56) is set forth in SEQ ID NO: 39 (see Sequence Table). The VH CDR domain amino acid sequences of Antibody 56 are set forth in SEQ ID NO: 41 (VH CDR1); SEQ ID NO: 42 (VH CDR2), and SEQ ID NO: 43 (VH CDR3). The light chain variable region (VL) amino acid sequence of Antibody 56 is described in SEQ ID NO: 40 (see Sequence Table). The VL CDR domain amino acid sequences of Antibody 56 are set forth in SEQ ID NO: 44 (VL CDR1); SEQ ID NO: 45 (VL CDR2), and SEQ ID NO: 46 (VL CDR3). The heavy chain constant region of Antibody 56 is set forth in SEQ ID NO: 122. The light chain constant region of Antibody 56 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 41, 42, and 43, and a light chain variable region CDR set as set forth in SEQ ID Nos: 44, 45, and 46. In other embodiments, an anti-CD117 antibody, or antigen-binding portion thereof, comprises a variable light chain comprising the amino acid residues set forth in SEQ ID NO: 40, and a heavy chain variable region as set forth in SEQ ID NO: 39.

In one embodiment, the invention provides an ADC comprising an anti-CD117 antibody, or antigen-binding fragment thereof, comprising binding regions, e.g., CDRs, variable regions, corresponding to those of Antibody 57. The heavy chain variable region (VH) amino acid sequence of Antibody 57 (i.e., Ab57) is set forth in SEQ ID NO: 49 (see Sequence Table). The VH CDR domain amino acid sequences of Antibody 57 are set forth in SEQ ID NO: 51 (VH CDR1); SEQ ID NO: 52 (VH CDR2), and SEQ ID NO: 53 (VH CDR3). The light chain variable region (VL) amino acid sequence of Antibody 57 is described in SEQ ID NO: 50 (see Sequence Table). The VL CDR domain amino acid sequences of Antibody 57 are set forth in SEQ ID NO: 54 (VL CDR1); SEQ ID NO: 55 (VL CDR2), and SEQ ID NO: 56 (VL CDR3). The heavy chain constant region of Antibody 57 is set forth in SEQ ID NO: 122. The light chain constant region of Antibody 57 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 51, 52, and 53, and a light chain variable region CDR set as set forth in SEQ ID Nos: 54, 55, and 56. In other embodiments, an anti-CD117 antibody, or antigen-binding portion thereof, comprises a variable light chain comprising the amino acid residues set forth in SEQ ID NO: 50, and a heavy chain variable region as set forth in SEQ ID NO: 49.

In one embodiment, the invention provides an ADC comprising an anti-CD117 antibody, or antigen-binding fragment thereof, comprising binding regions, e.g., CDRs, variable regions, corresponding to those of Antibody 58. The heavy chain variable region (VH) amino acid sequence of Antibody 58 (i.e., Ab58) is set forth in SEQ ID NO: 59 (see Sequence Table). The VH CDR domain amino acid sequences of Antibody 58 are set forth in SEQ ID NO: 61 (VH CDR1); SEQ ID NO: 62 (VH CDR2), and SEQ ID NO: 63 (VH CDR3). The light chain variable region (VL) amino acid sequence of Antibody 58 is described in SEQ ID NO: 60 (see Sequence Table). The VL CDR domain amino acid sequences of Antibody 58 are set forth in SEQ ID NO: 64 (VL CDR1); SEQ ID NO: 65 (VL CDR2), and SEQ ID NO: 66 (VL CDR3). The heavy chain constant region of Antibody 58 is set forth in SEQ ID NO: 122. The light chain constant region of Antibody 58 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 61, 62, and 63, and a light chain variable region CDR set as set forth in SEQ ID Nos: 64, 65, and 66. In other embodiments, an anti-CD117 antibody, or antigen-binding portion thereof, comprises a variable light chain comprising the amino acid residues set forth in SEQ ID NO: 60, and a heavy chain variable region as set forth in SEQ ID NO: 59.

In one embodiment, the invention provides an ADC comprising an anti-CD117 antibody, or antigen-binding fragment thereof, comprising binding regions, e.g., CDRs, variable regions, corresponding to those of Antibody 61. The heavy chain variable region (VH) amino acid sequence of Antibody 61 (i.e., Ab61) is set forth in SEQ ID NO: 69 (see Sequence Table). The VH CDR domain amino acid sequences of Antibody 61 are set forth in SEQ ID NO: 71 (VH CDR1); SEQ ID NO: 72 (VH CDR2), and SEQ ID NO: 73 (VH CDR3). The light chain variable region (VL) amino acid sequence of Antibody 61 is described in SEQ ID NO: 70 (see Sequence Table). The VL CDR domain amino acid sequences of Antibody 61 are set forth in SEQ ID NO: 74 (VL CDR1); SEQ ID NO: 75 (VL CDR2), and SEQ ID NO: 76 (VL CDR3). The heavy chain constant region of Antibody 61 is set forth in SEQ ID NO: 122. The light chain constant region of Antibody 61 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 71, 72, and 73, and a light chain variable region CDR set as set forth in SEQ ID Nos: 74, 75, and 76. In other embodiments, an anti-CD117 antibody, or antigen-binding portion thereof, comprises a variable light chain comprising the amino acid residues set forth in SEQ ID NO: 70, and a heavy chain variable region as set forth in SEQ ID NO: 69.

In one embodiment, the invention provides an ADC comprising an anti-CD117 antibody, or antigen-binding fragment thereof, comprising binding regions, e.g., CDRs, variable regions, corresponding to those of Antibody 66. The heavy chain variable region (VH) amino acid sequence of Antibody 66 (i.e., Ab66) is set forth in SEQ ID NO: 79 (see Sequence Table). The VH CDR domain amino acid sequences of Antibody 66 are set forth in SEQ ID NO: 81 (VH CDR1); SEQ ID NO: 82 (VH CDR2), and SEQ ID NO: 83 (VH CDR3). The light chain variable region (VL) amino acid sequence of Antibody 66 is described in SEQ ID NO: 80 (see Sequence Table). The VL CDR domain amino acid sequences of Antibody 66 are set forth in SEQ ID NO: 84 (VL CDR1); SEQ ID NO: 85 (VL CDR2), and SEQ ID NO: 86 (VL CDR3). The heavy chain constant region of Antibody 66 is set forth in SEQ ID NO: 122. The light chain constant region of Antibody 66 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 81, 82, and 83, and a light chain variable region CDR set as set forth in SEQ ID Nos: 84, 85, and 86. In other embodiments, an anti-CD117 antibody, or antigen-binding portion thereof, comprises a variable light chain comprising the amino acid residues set forth in SEQ ID NO: 80, and a heavy chain variable region as set forth in SEQ ID NO: 79.

In one embodiment, the invention provides an ADC comprising an anti-CD117 antibody, or antigen-binding fragment thereof, comprising binding regions, e.g., CDRs, variable regions, corresponding to those of Antibody 67. The heavy chain variable region (VH) amino acid sequence of Antibody 67 is set forth in SEQ ID NO: 9 (see Table 2). The VH CDR domain amino acid sequences of Antibody 67 are set forth in SEQ ID NO 11 (VH CDR1); SEQ ID NO: 12 (VH CDR2), and SEQ ID NO: 13 (VH CDR3). The light chain variable region (VL) amino acid sequence of Antibody 67 is described in SEQ ID NO: 10 (see Table 2). The VL CDR domain amino acid sequences of Antibody 67 are set forth in SEQ ID NO 14 (VL CDR1); SEQ ID NO: 15 (VL CDR2), and SEQ ID NO: 16 (VL CDR3). The full length heavy chain (HC) of Antibody 67 is set forth in SEQ ID NO: 110, and the full length heavy chain constant region of Antibody 67 is set forth in SEQ ID NO: 122. The light chain (LC) of Antibody 67 is set forth in SEQ ID NO: 109. The light chain constant region of Antibody 67 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 11, 12, and 13, and a light chain variable region CDR set as set forth in SEQ ID Nos: 14, 15, and 16. In other embodiments, an anti-CD117 antibody, or antigen-binding portion thereof, comprises a variable heavy chain comprising the amino acid residues set forth in SEQ ID NO: 9, and a heavy chain variable region as set forth in SEQ ID NO: 10. In further embodiments, an anti-CD117 antibody comprises a heavy chain comprising SEQ ID NO: 110 and a light chain comprising SEQ ID NO: 109.

In one embodiment, the invention provides an ADC comprising an anti-CD117 antibody, or antigen-binding fragment thereof, comprising binding regions, e.g., CDRs, variable regions, corresponding to those of Antibody 68. The heavy chain variable region (VH) amino acid sequence of Antibody 68 (i.e., Ab68) is set forth in SEQ ID NO: 89 (see Sequence Table). The VH CDR domain amino acid sequences of Antibody 68 are set forth in SEQ ID NO: 91 (VH CDR1); SEQ ID NO: 92 (VH CDR2), and SEQ ID NO: 93 (VH CDR3). The light chain variable region (VL) amino acid sequence of Antibody 68 is described in SEQ ID NO: 90 (see Sequence Table). The VL CDR domain amino acid sequences of Antibody 68 are set forth in SEQ ID NO: 94 (VL CDR1); SEQ ID NO: 95 (VL CDR2), and SEQ ID NO: 96 (VL CDR3). The heavy chain constant region of Antibody 68 is set forth in SEQ ID NO: 122. The light chain constant region of Antibody 68 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 91, 92, and 93, and a light chain variable region CDR set as set forth in SEQ ID Nos: 94, 95, and 96. In other embodiments, an anti-CD117 antibody, or antigen-binding portion thereof, comprises a variable light chain comprising the amino acid residues set forth in SEQ ID NO: 90, and a heavy chain variable region as set forth in SEQ ID NO: 89.

In one embodiment, the invention provides an ADC comprising an anti-CD117 antibody, or antigen-binding fragment thereof, comprising binding regions, e.g., CDRs, variable regions, corresponding to those of Antibody 69. The heavy chain variable region (VH) amino acid sequence of Antibody 69 (i.e., Ab69) is set forth in SEQ ID NO: 99 (see Sequence Table). The VH CDR domain amino acid sequences of Antibody 69 are set forth in SEQ ID NO: 101 (VH CDR1); SEQ ID NO: 102 (VH CDR2), and SEQ ID NO: 103 (VH CDR3). The light chain variable region (VL) amino acid sequence of Antibody 69 is described in SEQ ID NO: 100 (see Sequence Table). The VL CDR domain amino acid sequences of Antibody 69 are set forth in SEQ ID NO: 104 (VL CDR1); SEQ ID NO: 105 (VL CDR2), and SEQ ID NO: 106 (VL CDR3). The heavy chain constant region of Antibody 69 is set forth in SEQ ID NO: 122. The light chain constant region of Antibody 69 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 101, 102, and 103, and a light chain variable region CDR set as set forth in SEQ ID Nos: 104, 105, and 106. In other embodiments, an anti-CD117 antibody, or antigen-binding portion thereof, comprises a variable light chain comprising the amino acid residues set forth in SEQ ID NO: 100, and a heavy chain variable region as set forth in SEQ ID NO: 99.

Further, the amino acid sequences for the various binding regions of the anti-CD117 antibodies Ab77, Ab79, Ab81, Ab85, Ab86, Ab87, Ab88, and Ab89 are described in the Sequence Table provided below. Anti-CD117 antibodies having these sequences can also be used in the ADCs described herein.

In one embodiment, the invention provides an ADC comprising an anti-CD117 antibody, or antigen-binding fragment thereof, comprising binding regions, e.g., CDRs, variable regions, corresponding to those of Antibody 77. The heavy chain variable region (VH) amino acid sequence of Antibody 77 (i.e., Ab77) is set forth in SEQ ID NO: 147 (see Sequence Table). The VH CDR domain amino acid sequences of Antibody 77 are set forth in SEQ ID NO: 263 (VH CDR1); SEQ ID NO: 2 (VH CDR2), and SEQ ID NO: 3 (VH CDR3). The light chain variable region (VL) amino acid sequence of Antibody 77 is described in SEQ ID NO: 231 (see Sequence Table). The VL CDR domain amino acid sequences of Antibody 77 are set forth in SEQ ID NO: 264 (VL CDR1); SEQ ID NO: 265 (VL CDR2), and SEQ ID NO: 266 (VL CDR3). The heavy chain constant region of Antibody 77 is set forth in SEQ ID NO: 269. The light chain constant region of Antibody 77 is set forth in SEQ ID NO: 283. Thus, in certain embodiments, an anti-CD117 antibody, or antigen-binding portion thereof, comprises a variable heavy chain CDR set (CDR1, CDR2, and CDR3) as set forth in SEQ ID Nos: 263, 2, and 3, and a light chain variable region CDR set as set forth in SEQ ID Nos: 264, 265, and 266. In other embodiments, an anti-CD117 antibody, or antigen-binding portion thereof, comprises a variable light chain comprising the amino acid residues set forth in SEQ ID NO: 231, and a heavy chain variable region as set forth in SEQ ID NO: 147.

In one embodiment, the invention provides an ADC comprising an anti-CD117 antibody, or antigen-binding fragment thereof, comprising binding regions, e.g., CDRs, variable regions, corresponding to those of Antibody 79. The heavy chain variable region (VH) amino acid sequence of Antibody 79 (i.e., Ab79) is set forth in SEQ ID NO: 147 (see Sequence Table). The VH CDR domain amino acid sequences of Antibody 79 are set forth in SEQ ID NO: 263 (VH CDR1); SEQ ID NO: 2 (VH CDR2), and SEQ ID NO: 3 (VH CDR3). The light chain variable region (VL) amino acid sequence of Antibody 79 is described in SEQ ID NO: 233 (see Sequence Table). The VL CDR domain amino acid sequences of Antibody 79 are set forth in SEQ ID NO: 267 (VL CDR1); SEQ ID NO: 265 (VL CDR2), and SEQ ID NO: 266 (VL CDR3). The heavy chain constant region of Antibody 79 is set forth in SEQ ID NO: 269. The light chain constant region of Antibody 79 is set forth in SEQ ID NO: 283. Thus, in certain embodiments, an anti-CD117 antibody, or antigen-binding portion thereof, comprises a variable heavy chain CDR set (CDR1, CDR2, and CDR3) as set forth in SEQ ID Nos: 263, 2, and 3, and a light chain variable region CDR set as set forth in SEQ ID Nos: 267, 265, and 266. In other embodiments, an anti-CD117 antibody, or antigen-binding portion thereof, comprises a variable light chain comprising the amino acid residues set forth in SEQ ID NO: 233, and a heavy chain variable region as set forth in SEQ ID NO: 147.

In one embodiment, the invention provides an ADC comprising an anti-CD117 antibody, or antigen-binding fragment thereof, comprising binding regions, e.g., CDRs, variable regions, corresponding to those of Antibody 81. The heavy chain variable region (VH) amino acid sequence of Antibody 81 (i.e., Ab81) is set forth in SEQ ID NO: 147 (see Sequence Table). The VH CDR domain amino acid sequences of Antibody 81 are set forth in SEQ ID NO: 263 (VH CDR1); SEQ ID NO: 2 (VH CDR2), and SEQ ID NO: 3 (VH CDR3). The light chain variable region (VL) amino acid sequence of Antibody 81 is described in SEQ ID NO: 235 (see Sequence Table). The VL CDR domain amino acid sequences of Antibody 81 are set forth in SEQ ID NO: 264 (VL CDR1); SEQ ID NO: 268 (VL CDR2), and SEQ ID NO: 266 (VL CDR3). The heavy chain constant region of Antibody 81 is set forth in SEQ ID NO: 269. The light chain constant region of Antibody 81 is set forth in SEQ ID NO: 283. Thus, in certain embodiments, an anti-CD117 antibody, or antigen-binding portion thereof, comprises a variable heavy chain CDR set (CDR1, CDR2, and CDR3) as set forth in SEQ ID Nos: 263, 2, and 3, and a light chain variable region CDR set as set forth in SEQ ID Nos: 264, 268, and 266. In other embodiments, an anti-CD117 antibody, or antigen-binding portion thereof, comprises a variable light chain comprising the amino acid residues set forth in SEQ ID NO: 235, and a heavy chain variable region as set forth in SEQ ID NO: 147.

In one embodiment, the invention provides an ADC comprising an anti-CD117 antibody, or antigen-binding fragment thereof, comprising binding regions, e.g., CDRs, variable regions, corresponding to those of Antibody 85. The heavy chain variable region (VH) amino acid sequence of Antibody 85 (i.e., Ab86) is set forth in SEQ ID NO: 243 (see Sequence Table). The VH CDR domain amino acid sequences of Antibody 85 are set forth in SEQ ID NO: 245 (VH CDR1); SEQ ID NO: 246 (VH CDR2), and SEQ ID NO: 247 (VH CDR3). The light chain variable region (VL) amino acid sequence of Antibody 85 is described in SEQ ID NO: 242 (see Sequence Table). The VL CDR domain amino acid sequences of Antibody 85 are set forth in SEQ ID NO: 248 (VL CDR1); SEQ ID NO: 249 (VL CDR2), and SEQ ID NO: 250 (VL CDR3). The heavy chain constant region of Antibody 85 is set forth in SEQ ID NO: 269. The light chain constant region of Antibody 85 is set forth in SEQ ID NO: 283. Thus, in certain embodiments, an anti-CD117 antibody, or antigen-binding portion thereof, comprises a variable heavy chain CDR set (CDR1, CDR2, and CDR3) as set forth in SEQ ID Nos: 245, 246, and 247, and a light chain variable region CDR set as set forth in SEQ ID Nos: 248, 249, and 250. In other embodiments, an anti-CD117 antibody, or antigen-binding portion thereof, comprises a variable light chain comprising the amino acid residues set forth in SEQ ID NO: 244, and a heavy chain variable region as set forth in SEQ ID NO: 243.

In one embodiment, the invention provides an ADC comprising an anti-CD117 antibody, or antigen-binding fragment thereof, comprising binding regions, e.g., CDRs, variable regions, corresponding to those of Antibody 86. The heavy chain variable region (VH) amino acid sequence of Antibody 86 (i.e., Ab86) is set forth in SEQ ID NO: 251 (see Sequence Table). The VH CDR domain amino acid sequences of Antibody 86 are set forth in SEQ ID NO: 245 (VH CDR1); SEQ ID NO: 253 (VH CDR2), and SEQ ID NO: 3 (VH CDR3). The light chain variable region (VL) amino acid sequence of Antibody 86 is described in SEQ ID NO: 252 (see Sequence Table). The VL CDR domain amino acid sequences of Antibody 86 are set forth in SEQ ID NO: 254 (VL CDR1); SEQ ID NO: 249 (VL CDR2), and SEQ ID NO: 255 (VL CDR3). The heavy chain constant region of Antibody 86 is set forth in SEQ ID NO: 269. The light chain constant region of Antibody 86 is set forth in SEQ ID NO: 283. Thus, in certain embodiments, an anti-CD117 antibody, or antigen-binding portion thereof, comprises a variable heavy chain CDR set (CDR1, CDR2, and CDR3) as set forth in SEQ ID Nos: 245, 253, and 3, and a light chain variable region CDR set as set forth in SEQ ID Nos: 254, 249, and 255. In other embodiments, an anti-CD117 antibody, or antigen-binding portion thereof, comprises a variable light chain comprising the amino acid residues set forth in SEQ ID NO: 252, and a heavy chain variable region as set forth in SEQ ID NO: 251.

In one embodiment, the invention provides an ADC comprising an anti-CD117 antibody, or antigen-binding fragment thereof, comprising binding regions, e.g., CDRs, variable regions, corresponding to those of Antibody 87. The heavy chain variable region (VH) amino acid sequence of Antibody 87 (i.e., Ab87) is set forth in SEQ ID NO: 243 (see Sequence Table). The VH CDR domain amino acid sequences of Antibody 87 are set forth in SEQ ID NO: 245 (VH CDR1); SEQ ID NO: 246 (VH CDR2), and SEQ ID NO: 247 (VH CDR3). The light chain variable region (VL) amino acid sequence of Antibody 87 is described in SEQ ID NO: 256 (see Sequence Table). The VL CDR domain amino acid sequences of Antibody 87 are set forth in SEQ ID NO: 257 (VL CDR1); SEQ ID NO: 5 (VL CDR2), and SEQ ID NO: 255 (VL CDR3). The heavy chain constant region of Antibody 87 is set forth in SEQ ID NO: 269. The light chain constant region of Antibody 87 is set forth in SEQ ID NO: 283. Thus, in certain embodiments, an anti-CD117 antibody, or antigen-binding portion thereof, comprises a variable heavy chain CDR set (CDR1, CDR2, and CDR3) as set forth in SEQ ID Nos: 245, 246, and 247, and a light chain variable region CDR set as set forth in SEQ ID Nos: 257, 5, and 255. In other embodiments, an anti-CD117 antibody, or antigen-binding portion thereof, comprises a variable light chain comprising the amino acid residues set forth in SEQ ID NO: 256, and a heavy chain variable region as set forth in SEQ ID NO: 243.

In one embodiment, the invention provides an ADC comprising an anti-CD117 antibody, or antigen-binding fragment thereof, comprising binding regions, e.g., CDRs, variable regions, corresponding to those of Antibody 88. The heavy chain variable region (VH) amino acid sequence of Antibody 88 (i.e., Ab88) is set forth in SEQ ID NO: 258 (see Sequence Table). The VH CDR domain amino acid sequences of Antibody 88 are set forth in SEQ ID NO: 245 (VH CDR1); SEQ ID NO: 259 (VH CDR2), and SEQ ID NO: 3 (VH CDR3). The light chain variable region (VL) amino acid sequence of Antibody 88 is described in SEQ ID NO: 256 (see Sequence Table). The VL CDR domain amino acid sequences of Antibody 88 are set forth in SEQ ID NO: 257 (VL CDR1); SEQ ID NO: 5 (VL CDR2), and SEQ ID NO: 255 (VL CDR3). The heavy chain constant region of Antibody 88 is set forth in SEQ ID NO: 269. The light chain constant region of Antibody 88 is set forth in SEQ ID NO: 283. Thus, in certain embodiments, an anti-CD117 antibody, or antigen-binding portion thereof, comprises a variable heavy chain CDR set (CDR1, CDR2, and CDR3) as set forth in SEQ ID Nos: 245, 259, and 3, and a light chain variable region CDR set as set forth in SEQ ID Nos: 257, 5, and 255. In other embodiments, an anti-CD117 antibody, or antigen-binding portion thereof, comprises a variable light chain comprising the amino acid residues set forth in SEQ ID NO: 256, and a heavy chain variable region as set forth in SEQ ID NO: 258.

In one embodiment, the invention provides an ADC comprising an anti-CD117 antibody, or antigen-binding fragment thereof, comprising binding regions, e.g., CDRs, variable regions, corresponding to those of Antibody 89. The heavy chain variable region (VH) amino acid sequence of Antibody 89 (i.e., Ab89) is set forth in SEQ ID NO: 260 (see Sequence Table). The VH CDR domain amino acid sequences of Antibody 89 are set forth in SEQ ID NO: 245 (VH CDR1); SEQ ID NO: 2 (VH CDR2), and SEQ ID NO: 3 (VH CDR3). The light chain variable region (VL) amino acid sequence of Antibody 89 is described in SEQ ID NO: 252 (see Sequence Table). The VL CDR domain amino acid sequences of Antibody 89 are set forth in SEQ ID NO: 254 (VL CDR1); SEQ ID NO: 249 (VL CDR2), and SEQ ID NO: 255 (VL CDR3). The heavy chain constant region of Antibody 89 is set forth in SEQ ID NO: 269. The light chain constant region of Antibody 89 is set forth in SEQ ID NO: 283. Thus, in certain embodiments, an anti-CD117 antibody, or antigen-binding portion thereof, comprises a variable heavy chain CDR set (CDR1, CDR2, and CDR3) as set forth in SEQ ID Nos: 245, 2, and 3, and a light chain variable region CDR set as set forth in SEQ ID Nos: 254, 249, and 255. In other embodiments, an anti-CD117 antibody, or antigen-binding portion thereof, comprises a variable light chain comprising the amino acid residues set forth in SEQ ID NO: 252, and a heavy chain variable region as set forth in SEQ ID NO: 260.

In one embodiment, the invention provides an ADC comprising an anti-CD117 antibody, or antigen-binding fragment thereof, comprising binding regions, e.g., CDRs, variable regions, corresponding to those of Antibody 249. The heavy chain variable region (VH) amino acid sequence of Antibody 249 (i.e., Ab249) is set forth in SEQ ID NO: 238 (see Sequence Table). The VH CDR domain amino acid sequences of Antibody 249 are set forth in SEQ ID NO: 286 (VH CDR1); SEQ ID NO: 2 (VH CDR2), and SEQ ID NO: 287 (VH CDR3). The light chain variable region (VL) amino acid sequence of Antibody 249 is described in SEQ ID NO: 242 (see Sequence Table). The VL CDR domain amino acid sequences of Antibody 249 are set forth in SEQ ID NO: 288 (VL CDR1); SEQ ID NO: 249 (VL CDR2), and SEQ ID NO: 289 (VL CDR3). The heavy chain constant region of Antibody 249 is set forth in SEQ ID NO: 269. The light chain constant region of Antibody 249 is set forth in SEQ ID NO: 283. Thus, in certain embodiments, an anti-CD117 antibody, or antigen-binding portion thereof, comprises a variable heavy chain CDR set (CDR1, CDR2, and CDR3) as set forth in SEQ ID Nos: 286, 2, and 287, and a light chain variable region CDR set as set forth in SEQ ID Nos: 288, 249, and 289. In other embodiments, an anti-CD117 antibody, or antigen-binding portion thereof, comprises a variable light chain comprising the amino acid residues set forth in SEQ ID NO: 242, and a heavy chain variable region as set forth in SEQ ID NO: 238.

Further, included in the disclosure is anti-CD117 antibody drug conjugates (ADCs) comprising binding regions (heavy and light chain CDRs or variable regions) as set forth in SEQ ID Nos: 147 to 168. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 148. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 149. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 150. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 151. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 152. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 153. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 154. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 155. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 156. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 157. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 158. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 159. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 160. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 161. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 162. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 163. In one embodiment, the anti-CD117 antibody, or antigen binding portion thereof, comprises a heavy chain variable region as set forth in the amino acid sequence of SEQ ID NO: 164, and a light chain variable region as set forth in the amino acid sequence of SEQ ID NO: 165. In one embodiment, the anti-CD117 antibody, or antigen binding portion thereof, comprises a heavy chain variable region as set forth in the amino acid sequence of SEQ ID NO: 166, and a light chain variable region as set forth in the amino acid sequence of SEQ ID NO: 167. In one embodiment, the anti-CD117 antibody, or antigen binding portion thereof, comprises a heavy chain variable region as set forth in the amino acid sequence of SEQ ID NO: 168, and a light chain variable region as set forth in the amino acid sequence of SEQ ID NO: 169. In one embodiment, the anti-CD117 antibody, or antigen binding portion thereof, comprises a heavy chain variable region as set forth in the amino acid sequence of SEQ ID NO: 170, and a light chain variable region as set forth in the amino acid sequence of SEQ ID NO: 171. In one embodiment, the anti-CD117 antibody, or antigen binding portion thereof, comprises a heavy chain variable region as set forth in the amino acid sequence of SEQ ID NO: 172, and a light chain variable region as set forth in the amino acid sequence of SEQ ID NO: 173. In one embodiment, the anti-CD117 antibody, or antigen binding portion thereof, comprises a heavy chain variable region as set forth in the amino acid sequence of SEQ ID NO: 174, and a light chain variable region as set forth in the amino acid sequence of SEQ ID NO: 175. In one embodiment, the anti-CD117 antibody, or antigen binding portion thereof, comprises a heavy chain variable region as set forth in the amino acid sequence of SEQ ID NO: 176, and a light chain variable region as set forth in the amino acid sequence of SEQ ID NO: 177. In one embodiment, the anti-CD117 antibody, or antigen binding portion thereof, comprises a heavy chain variable region as set forth in the amino acid sequence of SEQ ID NO: 178, and a light chain variable region as set forth in the amino acid sequence of SEQ ID NO: 179. In one embodiment, the anti-CD117 antibody, or antigen binding portion thereof, comprises a heavy chain variable region as set forth in the amino acid sequence of SEQ ID NO: 180, and a light chain variable region as set forth in the amino acid sequence of SEQ ID NO: 181. In one embodiment, the anti-CD117 antibody, or antigen binding portion thereof, comprises a heavy chain variable region as set forth in the amino acid sequence of SEQ ID NO: 172, and a light chain variable region as set forth in the amino acid sequence of SEQ ID NO: 182. In one embodiment, the anti-CD117 antibody, or antigen binding portion thereof, comprises a heavy chain variable region as set forth in the amino acid sequence of SEQ ID NO: 183, and a light chain variable region as set forth in the amino acid sequence of SEQ ID NO: 184. In one embodiment, the anti-CD117 antibody, or antigen binding portion thereof, comprises a heavy chain variable region as set forth in the amino acid sequence of SEQ ID NO: 185, and a light chain variable region as set forth in the amino acid sequence of SEQ ID NO: 186. In one embodiment, the anti-CD117 antibody, or antigen binding portion thereof, comprises a heavy chain variable region as set forth in the amino acid sequence of SEQ ID NO: 187, and a light chain variable region as set forth in the amino acid sequence of SEQ ID NO: 188. In one embodiment, the anti-CD117 antibody, or antigen binding portion thereof, comprises a heavy chain variable region as set forth in the amino acid sequence of SEQ ID NO: 189, and a light chain variable region as set forth in the amino acid sequence of SEQ ID NO: 190. In one embodiment, the anti-CD117 antibody, or antigen binding portion thereof, comprises a heavy chain variable region as set forth in the amino acid sequence of SEQ ID NO: 191, and a light chain variable region as set forth in the amino acid sequence of SEQ ID NO: 192. In one embodiment, the anti-CD117 antibody, or antigen binding portion thereof, comprises a heavy chain variable region as set forth in the amino acid sequence of SEQ ID NO: 193, and a light chain variable region as set forth in the amino acid sequence of SEQ ID NO: 194. In one embodiment, the anti-CD117 antibody, or antigen binding portion thereof, comprises a heavy chain variable region as set forth in the amino acid sequence of SEQ ID NO: 195, and a light chain variable region as set forth in the amino acid sequence of SEQ ID NO: 196. In one embodiment, the anti-CD117 antibody, or antigen binding portion thereof, comprises a heavy chain variable region as set forth in the amino acid sequence of SEQ ID NO: 197, and a light chain variable region as set forth in the amino acid sequence of SEQ ID NO: 198. In one embodiment, the anti-CD117 antibody, or antigen binding portion thereof, comprises a heavy chain variable region as set forth in the amino acid sequence of SEQ ID NO: 199, and a light chain variable region as set forth in the amino acid sequence of SEQ ID NO: 200. In one embodiment, the anti-CD117 antibody, or antigen binding portion thereof, comprises a heavy chain variable region as set forth in the amino acid sequence of SEQ ID NO: 201, and a light chain variable region as set forth in the amino acid sequence of SEQ ID NO: 190. In one embodiment, the anti-CD117 antibody, or antigen binding portion thereof, comprises a heavy chain variable region as set forth in the amino acid sequence of SEQ ID NO: 202, and a light chain variable region as set forth in the amino acid sequence of SEQ ID NO: 203. In one embodiment, the anti-CD117 antibody, or antigen binding portion thereof, comprises a heavy chain variable region as set forth in the amino acid sequence of SEQ ID NO: 204, and a light chain variable region as set forth in the amino acid sequence of SEQ ID NO: 205. In one embodiment, the anti-CD117 antibody, or antigen binding portion thereof, comprises a heavy chain variable region as set forth in the amino acid sequence of SEQ ID NO: 206, and a light chain variable region as set forth in the amino acid sequence of SEQ ID NO: 207. In one embodiment, the anti-CD117 antibody, or antigen binding portion thereof, comprises a heavy chain variable region as set forth in the amino acid sequence of SEQ ID NO: 208, and a light chain variable region as set forth in the amino acid sequence of SEQ ID NO: 209. In one embodiment, the anti-CD117 antibody, or antigen binding portion thereof, comprises a heavy chain variable region as set forth in the amino acid sequence of SEQ ID NO: 210, and a light chain variable region as set forth in the amino acid sequence of SEQ ID NO: 211. In one embodiment, the anti-CD117 antibody, or antigen binding portion thereof, comprises a heavy chain variable region as set forth in the amino acid sequence of SEQ ID NO: 212, and a light chain variable region as set forth in the amino acid sequence of SEQ ID NO: 213. In one embodiment, the anti-CD117 antibody, or antigen binding portion thereof, comprises a heavy chain variable region as set forth in the amino acid sequence of SEQ ID NO: 214, and a light chain variable region as set forth in the amino acid sequence of SEQ ID NO: 215. In one embodiment, the anti-CD117 antibody, or antigen binding portion thereof, comprises a heavy chain variable region as set forth in the amino acid sequence of SEQ ID NO: 216, and a light chain variable region as set forth in the amino acid sequence of SEQ ID NO: 217. In one embodiment, the anti-CD117 antibody, or antigen binding portion thereof, comprises a heavy chain variable region as set forth in the amino acid sequence of SEQ ID NO: 218, and a light chain variable region as set forth in the amino acid sequence of SEQ ID NO: 219. In one embodiment, the anti-CD117 antibody, or antigen binding portion thereof, comprises a heavy chain variable region as set forth in the amino acid sequence of SEQ ID NO: 220, and a light chain variable region as set forth in the amino acid sequence of SEQ ID NO: 221. In one embodiment, the anti-CD117 antibody, or antigen binding portion thereof, comprises a heavy chain variable region as set forth in the amino acid sequence of SEQ ID NO: 222, and a light chain variable region as set forth in the amino acid sequence of SEQ ID NO: 223. In one embodiment, the anti-CD117 antibody, or antigen binding portion thereof, comprises a heavy chain variable region as set forth in the amino acid sequence of SEQ ID NO: 224, and a light chain variable region as set forth in the amino acid sequence of SEQ ID NO: 225. In one embodiment, the anti-CD117 antibody, or antigen binding portion thereof, comprises a heavy chain variable region as set forth in the amino acid sequence of SEQ ID NO: 226, and a light chain variable region as set forth in the amino acid sequence of SEQ ID NO: 227. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 228. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 229. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 230. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 231. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 232. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 233. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 234. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 235. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 236.

In one embodiment, the anti-CD117 antibody, or antigen binding portion thereof, of an ADC described herein comprises a heavy chain variable region as set forth in the amino acid sequence of SEQ ID NO: 147, and a light chain variable region as set forth in the amino acid sequence of SEQ ID NO: 237. In one embodiment, the anti-CD117 antibody, or antigen binding portion thereof, comprises a heavy chain variable region as set forth in the amino acid sequence of SEQ ID NO: 243, and a light chain variable region as set forth in the amino acid sequence of SEQ ID NO: 244. In one embodiment, the anti-CD117 antibody, or antigen binding portion thereof, comprises a heavy chain variable region as set forth in the amino acid sequence of SEQ ID NO: 251, and a light chain variable region as set forth in the amino acid sequence of SEQ ID NO: 252. In one embodiment, the anti-CD117 antibody, or antigen binding portion thereof, comprises a heavy chain variable region as set forth in the amino acid sequence of SEQ ID NO: 243, and a light chain variable region as set forth in the amino acid sequence of SEQ ID NO: 256. In one embodiment, the anti-CD117 antibody, or antigen binding portion thereof, comprises a heavy chain variable region as set forth in the amino acid sequence of SEQ ID NO: 258, and a light chain variable region as set forth in the amino acid sequence of SEQ ID NO: 256. In one embodiment, the anti-CD117 antibody, or antigen binding portion thereof, comprises a heavy chain variable region as set forth in the amino acid sequence of SEQ ID NO: 260, and a light chain variable region as set forth in the amino acid sequence of SEQ ID NO: 252. In one embodiment, the anti-CD117 antibody, or antigen binding portion thereof, comprises a heavy chain variable region as set forth in the amino acid sequence of SEQ ID NO: 238, and a light chain variable region as set forth in the amino acid sequence of SEQ ID NO: 239. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 239. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 240. In one embodiment, the anti-CD117 antibody, or antigen binding portion thereof, comprises a heavy chain variable region as set forth in the amino acid sequence of SEQ ID NO: 238, and a light chain variable region as set forth in the amino acid sequence of SEQ ID NO: 241. In one embodiment, the anti-CD117 antibody, or antigen binding portion thereof, comprises a heavy chain variable region as set forth in the amino acid sequence of SEQ ID NO: 238, and a light chain variable region as set forth in the amino acid sequence of SEQ ID NO: 242.

Certain of the anti-CD117 antibodies described herein are neutral antibodies, in that the antibodies do not substantially inhibit CD117 activity on a CD117 expressing cell. Neutral antibodies can be identified using, for example, an in in vitro stem cell factor (SCF)-dependent cell proliferation assay. In an SCF dependent cell proliferation assay, a neutral CD117 antibody will not kill CD34+ cells that are dependent on SCF to divide, as a neutral antibody will not block SCF from binding to CD117 such as to inhibit CD117 activity.

Neutral antibodies can be used for diagnostic purposes, given their ability to specifically bind to human CD117, but are also effective for killing CD117 expressing cells when conjugated to a cytotoxin, such as those described herein. Typically, antibodies used in conjugates have agonistic or antagonistic activity that is unique to the antibody. Described herein, however, is a unique approach to conjugates, especially in the context wherein the conjugate is being used as a conditioning agent prior to a stem cell transplantation. While antagonistic antibodies alone or in combination with a cytotoxin as a conjugate can be effective given the killing ability of the antibody alone in addition to the cytotoxin, conditioning with a conjugate comprising a neutral anti-CD117 antibody presents an alternative strategy where the activity of the antibody is secondary to the effect of the cytotoxin, but the internalizing and affinity characteristics, e.g., dissociation rate, of the antibody are important for effective delivery of the cytotoxin.

Examples of neutral anti-CD117 antibodies include Ab58, Ab61, Ab66, Ab67, Ab68, and Ab69. A comparison of the amino acid sequences of the CDRs of neutral, anti-CD117 antibody CDRs reveals consensus sequences among two groups of neutral antibodies identified. Ab58 and Ab61 share the same light chain CDRs and HC CDR3, with slight variations in the HC CDR1 and HC CDR2. Consensus sequences for the HC CDR1 and CDR2 are described in SEQ ID Nos: 133 and 134. Ab66, Ab67, Ab68, and Ab69 are also neutral antibodies. While Ab66, Ab67, Ab68, and Ab69 share the same light chain CDRs and the same HC CDR3, these antibodies have variability within their HC CDR1 and HC CDR2 regions. Consensus sequences for these antibodies in the HC CDR1 and HC CDR2 regions are provided in SEQ ID Nos: 139 and 140, respectively.

Antagonist antibodies are also provided herein, including Ab54, Ab55, Ab56, and Ab57. While Ab54, Ab55, Ab56, and Ab57 share the same light chain CDRs and the same HC CDR3, these antibodies have variability within their HC CDR1 and HC CDR2 regions. Consensus sequences for these antibodies in the HC CDR1 and HC CDR2 regions are provided in SEQ ID Nos: 127 and 128, respectively.

In one embodiment, the anti-CD117 antibody, or antigen binding fragment thereof, comprises variable regions having an amino acid sequence that is at least 95%, 96%, 97% or 99% identical to the SEQ ID Nos disclosed herein. Alternatively, the anti-CD117 antibody, or antigen binding fragment thereof, comprises CDRs comprising the SEQ ID Nos disclosed herein with framework regions of the variable regions described herein having an amino acid sequence that is at least 95%, 96%, 97% or 99% identical to the SEQ ID Nos disclosed herein.

The anti-CD117 antibodies described herein can be in the form of full-length antibodies, bispecific antibodies, dual variable domain antibodies, multiple chain or single chain antibodies, and/or binding fragments that specifically bind human CD117, including but not limited to Fab, Fab′, (Fab′)2, Fv), scFv (single chain Fv), surrobodies (including surrogate light chain construct), single domain antibodies, camelized antibodies and the like. They also can be of, or derived from, any isotype, including, for example, IgA (e.g., IgAQ1 or IgA2), IgD, IgE, IgG (e.g. IgG1, IgG2, IgG3 or IgG4), or IgM. In some embodiments, the anti-CD117 antibody is an IgG (e.g. IgG1, IgG2, IgG3 or IgG4).

Antibodies for use in conjunction with the methods described herein include variants of those antibodies described above, such as antibody fragments that contain or lack an Fc domain, as well as humanized variants of non-human antibodies described herein and antibody-like protein scaffolds (e.g., ¹⁰Fn3 domains) containing one or more, or all, of the CDRs or equivalent regions thereof of an antibody, or antibody fragment, described herein. Exemplary antigen-binding fragments of the foregoing antibodies include a dual-variable immunoglobulin domain, a single-chain Fv molecule (scFv), a diabody, a triabody, a nanobody, an antibody-like protein scaffold, a Fv fragment, a Fab fragment, a F(ab′)₂ molecule, and a tandem di-scFv, among others.

In one embodiment, anti-CD117 antibodies comprising one or more radiolabeled amino acids are provided. A radiolabeled anti-CD117 antibody may be used for both diagnostic and therapeutic purposes (conjugation to radiolabeled molecules is another possible feature). Nonlimiting examples of labels for polypeptides include, but are not limited to 3H, 14C, 15N, 35S, 90Y, 99Tc, and 125I, 131I, and 186Re. Methods for preparing radiolabeled amino acids and related peptide derivatives are known in the art (see for instance Junghans et al., in Cancer Chemotherapy and Biotherapy 655-686 (2d edition, Chafner and Longo, eds., Lippincott Raven (1996)) and U.S. Pat. Nos. 4,681,581, 4,735,210, 5,101,827, 5,102,990 (U.S. RE35,500), U.S. Pat. Nos. 5,648,471 and 5,697,902. For example, a radioisotope may be conjugated by a chloramine T method.

### Anti-CD45 Antibodies

In one embodiment, the present invention includes ADCs comprising antibodies, and antigen-binding fragments thereof, that specifically bind to a CD45 polypeptide, e.g., a human CD45 polypeptide, and uses thereof. In an exemplary embodiment, the antibody, or antigen-binding fragment thereof, that specifically binds to a CD45 polypeptide comprises a heavy chain variable region and a light chain variable region.

CD45 is a hematopoietic cell-specific transmembrane protein tyrosine phosphatase essential for T and B cell antigen receptor-mediated signaling. CD45 includes a large extracellular domain, and a phosphatase containing cytosolic domain. CD45 may act as both a positive and negative regulator depending on the nature of the stimulus and the cell type involved. Although there are a large number of permutations possible in the CD45 gene, only six isoforms are traditionally identified in humans. The isoforms are RA, RO, RB, RAB, RBC and RABC (Hermiston et al. 2003 “CD45: a critical regulator of signaling thresholds in immune cells.” Annu Rev Immunol. 2:107-137.). CD45RA is expressed on naïve T cells, and CD45RO is expressed on activated and memory T cells, some B cell subsets, activated monocytes/macrophages, and granulocytes. CD45RB is expressed on peripheral B cells, naïve T cells, thymocytes, weakly on macrophages, and dendritic cells.

In certain embodiments, the anti-CD45 antibody is selected from apamistamab (also known 90Y-BC8, Iomab-B, BC8; as described in, e.g., US20170326259, WO2017155937, and Orozco et al. Blood. 127.3 (2016): 352-359.) or BC8-B10 (as described, e.g., in Li et al. PloS one 13.10 (2018): e0205135.), each of which is incorporated by reference. Other anti-CD45 antibodies have been described, for example, in WO2003048327, WO2016016442, US20170226209, US20160152733, U.S. Pat. No. 9,701,756; US20110076270, or U.S. Pat. No. 7,825,222, each of which is incorporated by reference.

### Anti-CD137 Antibodies

The present invention includes ADCs comprising antibodies, and antigen-binding fragments thereof, that specifically bind to a CD137 polypeptide, e.g., a human CD137 polypeptide, and uses thereof. In an exemplary embodiment, the antibody, or antigen-binding fragment thereof, that specifically binds to a CD137 polypeptide comprises a heavy chain variable region and a light chain variable region.

T cells have been shown to express CD137, as this antigen is a transmembrane TNF receptor superfamily of costimulatory molecules and is expressed on a variety of hematopoietic cells and promotes T cell activation and regulates proliferation and survival of T cells (see, e.g., Cannons et al., J. Immunol. 167:1313-1324, 2001, the disclosure of which is incorporated herein by reference as it pertains to the expression of CD137 by T cells). CD137 is alternatively named tumor necrosis factor receptor superfamily member 9 (TNFRSF9), 4-1BB, or induced by lymphocyte activation (ILA).

In certain embodiments, the anti-CD137 antibody is selected from ADG106 (as described in, e.g., US20190055314, WO2019037711, WO2019036855); AGEN2373 (as described in, e.g., WO2018191502, US20180344870); ATOR-1017 (as described in, e.g., WO2018091740, US20180118841) PE0166 (as described in, e.g., Song et al. AACR 2019, Abstract 2397/21), urelumab (also known as BMS-663513; as described in, e.g., WO2004010947, WO2005035584, US20090068192, U.S. Pat. Nos. 7,659,384, 8,475,790, 8,137,667, US20100183621, U.S. Pat. No. 8,716,452, US20120141494, U.S. Pat. No. 9,382,328, US20140193422, WO2016029073, US20160368998, WO2017181034, US20190062445, Chin et al. Nature communications. 9.1 (2018): 4679; Segal et al. Clinical Cancer Research. 23.8 (2017): 1929-1936.); and utomilumab (also known as PF-05082566, MOR-7480; as described in, e.g., WO2012032433, US20120237498, US20140178368, WO2012145183, WO2015119923, WO2015179236, US20160152722, US20190031765, WO2017130076, Chin et al. Nature communications. 9.1 (2018): 4679; Segal et al. Clinical Cancer Research. 24.8 (2018): 1816-1823; Fisher et al. Cancer Immunology, Immunotherapy. 61.10 (2012): 1721-1733), each of which is incorporated by reference.

Other anti-CD137 antibodies have been described, for example, in WO2018134787, WO2019020774, WO2017077085, US20180327504, US20190099488, US2019006045, US20190015508, WO2019014328, US20190071510, WO2018127787, US20180258177, U.S. Ser. No. 10/174,122, WO2016110584, WO2018017761, WO2018098370, US20130149301, WO2019027754, WO2018156740, US20160244528, WO2016134358, U.S. Ser. No. 10/233,251, US20170226215, US20160083474, WO2017049452, US20180282422, WO2015188047, WO2010132389, US20120076722, US20110177104, WO2011031063, US20080305113, US20080008716, U.S. Pat. No. 7,829,088, US20090041763, WO2006126835, Söderström et al. Circulation J. 81.12 (2017): 1945-1952; Makkouk, et al. Annals of Oncology 28.2 (2016): 415-420; Martinez-Forero et al. J. of Immunology. 190.12 (2013): 6694-6706; Dubrot et al. Cancer immunology, immunotherapy. 59.8 (2010): 1223-1233; each of which is incorporated by reference.

In another embodiment, the antibody, or antigen-binding fragment thereof, comprises a heavy chain variable region that comprises an amino acid sequence having at least 95% identity to an anti-CD137 antibody herein, e.g., at least 95%, 96%, 97%, 98%, 99%, or 100% identity to an anti-CD137 antibody herein. In certain embodiments, an antibody comprises a modified heavy chain (HC) variable region comprising an HC variable domain of an anti-CD137 antibody herein, or a variant thereof, which variant (i) differs from the anti-CD137 antibody in 1, 2, 3, 4 or 5 amino acids substitutions, additions or deletions; (ii) differs from the anti-CD137 antibody in at most 5, 4, 3, 2, or 1 amino acids substitutions, additions or deletions; (iii) differs from the anti-CD137 antibody in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to the anti-CD137 antibody, wherein in any of (i)-(iv), an amino acid substitution may be a conservative amino acid substitution or a non-conservative amino acid substitution; and wherein the modified heavy chain variable region can have an enhanced biological activity relative to the heavy chain variable region of the anti-CD137 antibody, while retaining the CD137 binding specificity of the antibody.

In one embodiment, an anti-CD137 antibody that may be used in the methods and compositions (including ADCs) described herein is the murine anti-CD137 antibody BBK2 (Thermo Fisher; MS621PABX) or an anti-CD137 antibody comprising antigen binding regions corresponding to the BBK2 antibody. The BBK2 antibody (which may also be referred to as a BBK-2 antibody or an anti-4-1BB antibody), is a mouse monoclonal antibody (IgG1, kappa) that binds to the ectodomain of human 4-1BB recombinant protein (4-1BB is also known as CD137). In certain embodiments, the methods and compositions of the disclosure include an anti-CD137 antibody comprising the binding regions (e.g., the CDRs) of the BBK2 antibody. In another embodiment, the methods and compositions of the disclosure comprise an antibody that competitively inhibits the binding of the BBK2 antibody to its epitope on CD137. In certain embodiments, the anti-CD137 antibody is humanized BBK2 or chimeric BBK2.

In one embodiment, the methods and compositions described herein include a chimeric anti-CD137 (ch-BBK2) antibody comprising the variable heavy and light chain regions of BBK2. In certain embodiments, the chimeric BBK2 antibody is an IgG1 antibody comprising human constant regions. The heavy chain amino acid sequence of ch-BBK2 is described in SEQ ID NO: 290, and the light chain amino acid sequence of ch-BBK2 is described in SEQ ID NO: 291. The CDR regions (CDR1, CDR2, and CDR3) of each of the heavy and light chain sequences are described in bold below. The CDR regions of BBK2 can be defined according to Kabat numbering. CDRs as defined by Kabat numbering are described below for each of the heavy and light chain sequences (described in bold below). The variable regions of BBK2 are italicized.

(ch-BBK2 heavy chain; SEQ ID NO: 290)
QVQLQQPGAELVRPGASVKLSCKASGYTFTSYWINWVKQRPGQGLEWIG
NIYPSDSYTNYNQKFKDKATLTVDKSSNTVYMQLNSPTSEDSAVYYCTR
NGVEGYPHYYAMEYWGQGTSVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNH
YTQKSLSLSPGK
(ch-BBK2 light chain; SEQ ID NO: 291)
DIQMTQTTSALSASLGDRVTIGCRASQDLSNHLYWYQQKPDGTVKLLIY
YTSRLHSGVPSRFSGSGSGTDYSLTIRNLEQEDVATYFCQQGYTLPYTF
GGGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

Thus, in one embodiment, the VH CDR amino acid sequences of anti-CD137 antibody BBK2 (including ch-BBK2) are as follows: SYWIN (VH CDR1; SEQ ID NO: 292); NIYPSDSYTNYNQKFKD (VH CDR2; SEQ ID NO: 293) and NGVEGYPHYYAMEY (VH CDR3; SEQ ID NO: 294), and the VL CDR amino acid sequences of anti-CD137 antibody BBK2 (including ch-BBK2) are as follows: RASQDLSNHLY (VL CDR1; SEQ ID NO: 295); YTSRLHS (VL CDR2; SEQ ID NO: 296) and QQGYTLPYT (VL CDR3; SEQ ID NO: 297).

The heavy chain variable region of BBK2 is set forth in SEQ ID NO: 298 as QVQLQQPGAELVRPGASVKLSCKASGYTFTSYWINWVKQRPGQGLEWIGNIYPSDS YTNYNQKFKDKATLTVDKSSNTVYMQLNSPTSEDSAVYYCTRNGVEGYPHYYAME YWGQGTSVTVSS. The light chain variable region of BBK2 is set forth in SEQ ID NO: 299 as DIQMTQTTSALSASLGDRVTIGCRASQDLSNHLYWYQQKPDGTVKLLIYYTSRLHSG VPSRFSGSGSGTDYSLTIRNLEQEDVATYFCQQGYTLPYTFGGGTKLEIK. Anti-CD137 antibodies (including anti-CD137 ADCs) can comprise the heavy and light chain variable region amino acid sequences as set forth in SEQ ID Nos: 298 and 299, respectively.

In one embodiment, the anti-CD137 antibody, e.g., a chimeric (ch-BBK2) antibody or a humanized BBK2 antibody, comprises a heavy chain variable region comprising a CDR1 comprising the amino acid sequence of SEQ ID NO: 292, a CDR2 comprising the amino acid sequence of SEQ ID NO: 293, and a CDR3 comprising the amino acid sequence of SEQ ID NO: 294; and comprises a light chain variable region comprising a CDR1 comprising the amino acid sequence of SEQ ID NO: 295, a CDR2 comprising the amino acid sequence of SEQ ID NO: 296, and a CDR3 comprising the amino acid sequence of SEQ ID NO: 297.

In one embodiment, the anti-CD137 antibody, e.g., a chimeric (ch-BBK2) antibody or a humanized BBK2 antibody, comprises a heavy chain variable region comprising a CDR1 comprising the amino acid sequence of SEQ ID NO: 292, a CDR2 comprising the amino acid sequence of SEQ ID NO: 293, and a CDR3 comprising the amino acid sequence of SEQ ID NO: 294; and comprises a light chain variable region comprising a CDR1 comprising the amino acid sequence of SEQ ID NO: 295, a CDR2 comprising the amino acid sequence of SEQ ID NO: 296, and a CDR3 comprising the amino acid sequence of SEQ ID NO: 297.

Thus, BBK2, humanized BBK2, or chimeric BBK2 antibodies can be used in the anti-CD137 ADCs and methods described herein. Each of these antibodies can be conjugated to any of the cytotoxin described below using methods known in the art and those described herein.

### Anti-CD5 Antibodies

In certain embodiments, compositions and methods described herein include an ADC comprising an antibody, or fragment thereof, that specifically binds to human CD5. Human CD5 is also referred to as LEU1 or T1. Human CD5 is a type-I transmembrane glycoprotein found on the surface of thymocytes, T lymphocytes and a subset of B lymphocytes. Two isoforms of human CD5 have been identified. Isoform 1 contains 438 amino acids and is described in Jones. et al. (1988) Nature 323 (6086), 346-349 and below (NCBI Reference Sequence: NP_001333385.1):

(SEQ ID NO: 363)
MVCSQSWGRS SKQWEDPSQASKVCQRLNCG VPLSLGPFLV
TYTPQSSIICYGQL GSFSNCSHSRNDMCHS
LGLTCLEPQKTTPPTTRPPPTTTPEPTAPP RLQLVAQSGG
QHCAGVVEFYSGSLGGTISY EAQDKTQDLE NFLCNNLQCG
SFLKHLPETE AGRAQDPGEP REHQPLPIQWKIQNSSCTSL
EHCFRKIKPQ KSGRVLALLC SGFQPKVQSR LVGGSSICEG
TVEVRQGAQWAALCDSSSAR SSLRWEEVCR
EQQCGSVNSY RVLDAGDPTS RGLFCPHQKL
SQCHELWERNSYCKKVFVTCQDPNPAGLAAGTVASIILAL
VLLVVLLVVC GPLAYKKLVK KFRQKKQRQWIGPTGMNQNM
SFHRNHTATV RSHAENPTAS HVDNEYSQPP RNSHLSAYPA
LEGALHRSSMQPDNSSDSDY DLHGAQRL

T cells have been shown to express CD5, which is a cell adhesion molecule and has been implicated both in the proliferative response of activated T cells and in T cell helper function. It has also been shown to function as a receptor, delivering co-stimulatory signals to T cells by interacting with CD72, a cell surface protein exclusive to B cells. Antibodies, or antigen-binding fragments thereof, that bind CD5 may suppress T cell activation and T cell-mediated immune responses against hematopoietic stem cell grafts, for example, by inhibiting the interaction between CD5 and CD72. Antibodies, and antigen-binding fragments thereof, that bind CD5 can also be used to kill CD5+ T cells directly, for instance, by conjugating the antibody, or antigen-binding fragment thereof, to a cytotoxin (such as a cytotoxin described herein or known in the art) or by using an unconjugated antibody, or antigen-binding fragment thereof, capable of recruiting complement proteins to the T cell.

Additionally, subsets of activated B cells have been shown to express CD5, and this expression pattern is particularly common among autoreactive B cells (Werner-Favre et al., European Journal of Immunology 19:1209-1231 (1989), the disclosure of which is incorporated herein by reference in its entirety). CD5 has also been shown to be expressed by subsets of NK cells; particularly among patients that have multiple myeloma have been shown to harbor populations of low density CD5+(CD5LOW+) NK cells, and this surface antigen has been implicated in NK cell activation (Ishiyama et al., Anticancer Research 14:725-730 (1994), the disclosure of which is incorporated herein by reference in its entirety). Antibodies, or antigen-binding fragments thereof, that specifically bind CD5 can thus be used to attenuate the activation of B cells and NK cells. Antibodies, or antigen-binding fragments thereof, that bind CD5 can also be used to kill CD5+ B cells and NK cells directly, for instance, by conjugating the antibody, or antigen-binding fragment thereof, to a cytotoxin (such as a cytotoxin described herein or known in the art) or by using an unconjugated antibody, or antigen-binding fragment thereof, capable of recruiting complement proteins to the B cell or NK cell.

The present invention encompasses ADCs comprising antibodies, and antigen-binding fragments thereof, that specifically bind to a CD5 polypeptide, e.g., a human CD5 polypeptide, and uses thereof. In an exemplary embodiment, the antibody, or antigen-binding fragment thereof, that specifically binds to a CD5 polypeptide comprises a heavy chain variable region and a light chain variable region.

In one embodiment, an ADC comprises an antibody comprising the heavy chain variable region comprises one or more complementarity determining regions (CDRs). In one embodiment, the heavy chain variable region comprises a VH CDR1 comprising the amino acid sequence of SEQ ID NO: 341. In one embodiment, the heavy chain variable region comprises a VH CDR2 comprising the amino acid sequence of SEQ ID NO: 342. In one embodiment, the heavy chain variable region comprises a VH CDR3 comprising the amino acid sequence of SEQ ID NO: 343. In one embodiment, the heavy chain variable region comprises one or more VH CDRs selected from the group consisting of SEQ ID NO: 341, SEQ ID NO: 342, and SEQ ID NO: 343. In one embodiment, the heavy chain variable region comprises two or more VH CDRs selected from the group consisting of SEQ ID NO: 341, SEQ ID NO: 342, and SEQ ID NO: 343. In one embodiment, the heavy chain variable region comprises a VH CDR1 comprising SEQ ID NO: 341, a VH CDR2 comprising SEQ ID NO: 342, and a VH CDR3 comprising SEQ ID NO: 343.

In one embodiment, an ADC comprises an antibody comprising the light chain variable region comprises one or more complementarity determining regions (CDRs). In one embodiment, the light chain variable region comprises a VL CDR1 comprising the amino acid sequence of SEQ ID NO: 344. In one embodiment, the light chain variable region comprises a VL CDR2 comprising the amino acid sequence of SEQ ID NO: 345. In one embodiment, the light chain variable region comprises a VL CDR3 comprising the amino acid sequence of SEQ ID NO: 346. In one embodiment, the light chain variable region comprises one or more VL CDRs selected from the group consisting of SEQ ID NO: 344, SEQ ID NO: 345, and SEQ ID NO: 346. In one embodiment, the light chain variable region comprises two or more VL CDRs selected from the group consisting of SEQ ID NO: 344, SEQ ID NO: 345, and SEQ ID NO: 346. In one embodiment, the light chain variable region comprises a VL CDR1 comprising SEQ ID NO: 344, a VL CDR2 comprising SEQ ID NO: 345, and a VL CDR3 comprising SEQ ID NO: 346.

In an exemplary embodiment, the antibody, or antigen-binding fragment thereof, comprises a heavy chain variable region that comprises a VH CDR1 comprising SEQ ID NO: 341, a VH CDR2 comprising SEQ ID NO: 342, and a VH CDR3 comprising SEQ ID NO: 343, and a light chain variable region that comprises a VL CDR1 comprising SEQ ID NO: 344, a VL CDR2 comprising SEQ ID NO: 345, and a VL CDR3 comprising SEQ ID NO: 346.

In certain embodiments, one or more of the CDRs (i.e., one or more heavy chain CDRs having SEQ ID NOs: 341-343, and/or one or more light chain CDRs having SEQ ID NOs: 344-346) can comprise a conservative amino acid substitution (or 2, 3, 4, or 5 amino acid substitutions) while retaining the CD5 specificity of the antibody (i.e., specificity similar to an antibody, or antigen-binding fragment thereof, comprising heavy chain CDRs of SEQ ID NOs: 341 to 343, and light chain CDRs of SEQ ID NOs:344 to 346).

In certain embodiments, the anti-CD5 antibody, or antigen binding fragment thereof, is murine antibody 5D7, or a humanized version thereof. Murine antibody 5D7 binds to human CD5 and is described in US Patent Publication No. 20008/0245027, the contents of which relating to the antibody sequences disclosed therein are incorporated by reference herein. SEQ ID Nos: 353 to 358 described in the sequence summary table correspond to the CDRs of murine anti-CD5 antibody 5D7. A humanized version of anti-CD5 antibody 5D7 is described in SEQ ID NO: 359 (humanized heavy chain variable region) and SEQ ID NO: 360 (humanized light chain variable region). In one embodiment, the ADCs and uses thereof described herein include an antibody comprising the CDRs set forth in SEQ ID Nos: 353 to 358. In one embodiment, the ADCs and uses thereof described herein include an antibody comprising the heavy and light chain variable regions as set forth in SEQ ID Nos: 359 and 360, respectively.

In one embodiment, the antibody, or antigen-binding fragment thereof, comprises a heavy chain variable region that comprises the amino acid sequence set forth in SEQ ID NO: 359. In another embodiment, the antibody, or antigen-binding fragment thereof, comprises a heavy chain variable region that comprises an amino acid sequence having at least 95% identity to SEQ ID NO: 359, e.g., at least 95%, 96%, 97%, 98%, 99%, or 100% identity to SEQ ID NO: 359. In certain embodiments, an antibody comprises a modified heavy chain (HC) variable region comprising an HC variable domain comprising SEQ ID NO: 359, or a variant of SEQ ID NO: 359, which variant (i) differs from SEQ ID NO: 359 in 1, 2, 3, 4 or 5 amino acids substitutions, additions or deletions; (ii) differs from SEQ ID NO: 359 in at most 5, 4, 3, 2, or 1 amino acids substitutions, additions or deletions; (iii) differs from SEQ ID NO: 359 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO: 359, wherein in any of (i)-(iv), an amino acid substitution may be a conservative amino acid substitution or a non-conservative amino acid substitution; and wherein the modified heavy chain variable region can have an enhanced biological activity relative to the heavy chain variable region of SEQ ID NO: 359, while retaining the CD5 binding specificity of the antibody, i.e. has a binding specificity similar to an antibody, or antigen-binding fragment thereof, comprising SEQ ID NO: 359.

In one embodiment, the antibody, or antigen-binding fragment thereof, comprises a light chain variable region that comprises the amino acid sequence set forth in SEQ ID NO: 360. In another embodiment, the antibody, or antigen-binding fragment thereof, comprises a light chain variable region that comprises an amino acid sequence having at least 95% identity to SEQ ID NO: 360, e.g., at least 95%, 96%, 97%, 98%, 99%, or 100% identity to SEQ ID NO: 360. In certain embodiments, an antibody comprises a modified light chain (LC) variable region comprising an LC variable domain comprising SEQ ID NO: 360, or a variant of SEQ ID NO: 360, which variant (i) differs from SEQ ID NO: 360 in 1, 2, 3, 4 or 5 amino acids substitutions, additions or deletions; (ii) differs from SEQ ID NO: 360 in at most 5, 4, 3, 2, or 1 amino acids substitutions, additions or deletions; (iii) differs from SEQ ID NO: 360 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO: 360, wherein in any of (i)-(iv), an amino acid substitution may be a conservative amino acid substitution or a non-conservative amino acid substitution; and wherein the modified light chain variable region can have an enhanced biological activity relative to the light chain variable region of SEQ ID NO: 360, while retaining the CD5 binding specificity of the antibody, i.e., has a binding specificity similar to an antibody, or antigen-binding fragment thereof, comprising SEQ ID NO: 360.

In an exemplary embodiment, the antibody, or antigen-binding fragment thereof, comprises a heavy chain variable region that comprises an amino acid sequence having at least 95% identity to SEQ ID NO: 359, e.g., at least 95%, 96%, 97%, 98%, 99%, or 100% identity to SEQ ID NO: 359, and a light chain variable region that comprises an amino acid sequence having at least 95% identity to SEQ ID NO: 360, e.g., at least 95%, 96%, 97%, 98%, 99%, or 100% identity to SEQ ID NO: 360. In one embodiment, the antibody, or antigen-binding fragment thereof, comprises a heavy chain variable region that comprises SEQ ID NO: 359, and a light chain variable region that comprises SEQ ID NO: 360.

In another embodiment, the anti-CD5 antibody, or antigen-binding fragment thereof, can contain a heavy chain variable region that comprises a VH CDR1 comprising the amino acid sequence of SEQ ID NO: 353. In one embodiment, the heavy chain variable region comprises a VH CDR2 comprising the amino acid sequence of SEQ ID NO: 354. In one embodiment, the heavy chain variable region comprises a VH CDR3 comprising the amino acid sequence of SEQ ID NO: 355. In one embodiment, the heavy chain variable region comprises one or more VH CDRs selected from the group consisting of SEQ ID NO: 353, SEQ ID NO: 354, and SEQ ID NO: 355. In one embodiment, the heavy chain variable region comprises two or more VH CDRs selected from the group consisting of SEQ ID NO: 353, SEQ ID NO: 354, and SEQ ID NO: 355. In one embodiment, the heavy chain variable region comprises a VH CDR1 comprising SEQ ID NO: 353, a VH CDR2 comprising SEQ ID NO: 354, and a VH CDR3 comprising SEQ ID NO: 355.

In one embodiment, the light chain variable region comprises one or more complementarity determining regions (CDRs). In one embodiment, the light chain variable region comprises a VL CDR1 comprising the amino acid sequence of SEQ ID NO: 356. In one embodiment, the light chain variable region comprises a VL CDR2 comprising the amino acid sequence of SEQ ID NO: 357. In one embodiment, the light chain variable region comprises a VL CDR3 comprising the amino acid sequence of SEQ ID NO: 358. In one embodiment, the light chain variable region comprises one or more VL CDRs selected from the group consisting of SEQ ID NO: 356, SEQ ID NO: 357, and SEQ ID NO: 358. In one embodiment, the light chain variable region comprises two or more VL CDRs selected from the group consisting of SEQ ID NO: 356, SEQ ID NO: 357, and SEQ ID NO: 358. In one embodiment, the light chain variable region comprises a VL CDR1 comprising SEQ ID NO: 356, a VL CDR2 comprising SEQ ID NO: 357, and a VL CDR3 comprising SEQ ID NO: 358.

In an exemplary embodiment, the antibody, or antigen-binding fragment thereof, comprises a heavy chain variable region that comprises a VH CDR1 comprising SEQ ID NO: 353, a VH CDR2 comprising SEQ ID NO: 354, and a VH CDR3 comprising SEQ ID NO: 355, and a light chain variable region that comprises a VL CDR1 comprising SEQ ID NO: 356, a VL CDR2 comprising SEQ ID NO: 357, and a VL CDR3 comprising SEQ ID NO: 358.

In certain embodiments, one or more of the CDRs (i.e., one or more heavy chain CDRs having SEQ ID NOs: 353-355, and/or one or more light chain CDRs having SEQ ID NOs: 356-358) can comprise a conservative amino acid substitution (or 2, 3, 4, or 5 amino acid substitutions) while retaining the CD5 specificity of the antibody (i.e., specificity similar to an antibody, or antigen-binding fragment thereof, comprising heavy chain CDRs of SEQ ID NOs: 353 to 355, and light chain CDRs of SEQ ID NOs:356 to 358).

Antibodies and antigen-binding fragments thereof capable of binding CD5 antigen can be identified using techniques known in the art and described herein, such as by immunization, computational modeling techniques, and in vitro selection methods, such as the phage display and cell-based display platforms described below.

Anti-CD5 antibodies that can be used in conjunction with the compositions and methods described herein include those that have one or both of the following variable regions, or an amino acid sequence having at least 85% sequence identity thereto (e.g., an amino acid sequence having 85%, 90%, 95%, 97%, 98%, 99%, or more, sequence identity thereto):

a V_{L} having the amino acid sequence
(SEQ ID NO: 325)
DIQMTQSPSSMSASLGDRVTITCRASQDINSYLSWFQQKPGKSPKT
LIYRANRL
VDGVPSRFSGSGSGTDYTLTISSLQYEDFGIYYCQQYDESPWTFGG
GTKLEIK;
and
a V_{H} having the amino acid sequence
(SEQ ID NO: 326)
QIQLVQSGPGLKKPGGSVRISCAASGYTFTNYGMNWVKQAPGKGLR
WMGWI
NTHTGEPTYADDFKGRFTFSLDTSKSTAYLQINSLRAEDTATYFCT
RRGYDWY FDVWGQGTTVTVSS.

Antibodies and antigen-binding fragments thereof containing the foregoing V_{L} and V_{H} sequences are described, e.g., in U.S. Pat. No. 5,869,619, the disclosure of which is incorporated herein by reference as it pertains to anti-CD5 antibodies and antigen-binding fragments thereof, such as the he1 antibody. In some embodiments, the anti-CD5 antibody or antigen-binding fragment thereof includes the V_{L} and VH chains of SEQ ID NO: 325 and SEQ ID NO: 326. In some embodiments, the anti-CD5 antibody or antigen-binding fragment thereof includes the CDRs contained in the V_{L} and V_{H} chains of SEQ ID NO: 325 and SEQ ID NO: 326. In some embodiments, the anti-CD5 antibody or antigen-binding fragment thereof includes the CDRs contained in the V_{L} and V_{H} chains of SEQ ID NO: 325 and SEQ ID NO: 326 and the remainder of the V_{L} and V_{H} sequences have at least 85% sequence identity (e.g., 85%, 90%, 95%, 97%, 98%, 99%, or greater sequence identity) to the V_{L} and V_{H} sequences of SEQ ID NO: 325 and SEQ ID NO: 326.

In some embodiments, the anti-CD5 antibody or antigen-binding fragment thereof includes the following CDRs:

```
a CDR-H1 having the amino acid sequence
  (SEQ ID NO: 327)
GYTFTNY;
a CDR-H2 having the amino acid sequence
  (SEQ ID NO: 328)
NTHTGE;
a CDR-H3 having the amino acid sequence
  (SEQ ID NO: 329)
RGYDWYFDV;
a CDR-L1 having the amino acid sequence
  (SEQ ID NO: 330)
RASQDINSYLS;
a CDR-L2 having the amino acid sequence
(SEQ ID NO: 331)
RANRLVD;
and
a CDR-L3 having the amino acid sequence
(SEQ ID NO: 332)
QQYDESPWT.
```

Additional anti-CD5 antibodies that can be used in conjunction with the compositions and methods described herein include those that have one or both of the following variable regions, or an amino acid sequence having at least 85% sequence identity thereto (e.g., an amino acid sequence having 85%, 90%, 95%, 97%, 98%, 99%, or more, sequence identity thereto):

a VL having the amino acid sequence
(SEQ ID NO: 333)
DIQMTQSPSSLSASVGDRVTITCRASQDINSYLSWFQQKPGKAPKT
LIYRANRL ESGVPSRFSGSGSGTDYTLTIS
SLQYEDFGIYYCQQYDESPWTFGGGTKLEIK;
and
a VH having the amino acid sequence
(SEQ ID NO: 334)
EIQLVQSGGGLVKPGGSVRISCAASGYTFTNYGMNWVRQAPGKGLE
WMGWI
NTHYGEPTYADSFKGTRTFSLDDSKNTAYLQINSLRAEDTAVYFCT
RRGYDW YFDVWGQGGTTVTVSS

Antibodies and antigen-binding fragments thereof containing the foregoing V_{L} and V_{H} sequences are described, e.g., in U.S. Pat. No. 5,869,619, the disclosure of which is incorporated herein by reference as it pertains to anti-CD5 antibodies and antigen-binding fragments thereof, such as the he3 antibody. In some embodiments, the anti-CD5 antibody or antigen-binding fragment thereof includes the CDRs contained in the V_{L} and V_{H} chains of the antibody that includes the V_{L} and V_{H} chains of SEQ ID NO:327 and SEQ ID NO: 328. In some embodiments, the anti-CD5 antibody or antigen-binding fragment thereof includes the CDRs contained in the V_{L} and V_{H} chains of SEQ ID NO:327 and SEQ ID NO: 328 and the remainder of the V_{L} and V_{H} sequences have at least 85% sequence identity (e.g., 85%, 90%, 95%, 97%, 98%, 99%, or greater sequence identity) to the V_{L} and V_{H} sequences of SEQ ID NO:327 and SEQ ID NO: 328.

In some embodiments, the anti-CD5 antibody or antigen-binding fragment thereof includes the following CDRs:

```
a CDR-H1 having the amino acid sequence
  (SEQ ID NO: 335)
GYTFTNY;
a CDR-H2 having the amino acid sequence
  (SEQ ID NO: 336)
NTHYGE;
a CDR-H3 having the amino acid sequence
  (SEQ ID NO: 337)
RRGYDWYFDV;
a CDR-L1 having the amino acid sequence
  (SEQ ID NO: 338)
RASQDINSYLS;
a CDR-L2 having the amino acid sequence
  (SEQ ID NO: 339)
RANRLES;
and
a CDR-L3 having the amino acid sequence
(SEQ ID NO: 340)
QQYDESPWT.
```

Antibodies and antigen-binding fragments thereof containing the foregoing CDR sequences are described, e.g., in U.S. Pat. No. 5,869,619, the disclosure of which is incorporated herein by reference as it pertains to anti-CD5 antibodies and antigen-binding fragments thereof.

Other anti-CD5 antibodies that can be used in conjunction with the compositions and methods described herein include, for instance, anti-CD5 antibodies that are described in U.S. Pat. Nos. 5,821,123; 5,766,886; 5,770,196; 7,153,932; 5,621,083; 6,649,742; 6,146,631; 5,756,699; 5,744,580; 6,376,217; 5,837,491; and 6,146,850, the disclosures of each of which are incorporated herein by reference as they pertain to anti-CD5 antibodies and antigen-binding fragments thereof.

Other anti-CD5 antibodies that can be used in conjunction with the compositions and methods described herein include, for instance, those produced by the hybridoma cell line deposited as ATCC CRL 8000 (anti-CD5 murine antibody OKT1). Such antibodies are described in U.S. Pat. Nos. 4,515,894; 4,657,760; and 4,363,799, the disclosures of each of which are incorporated herein by reference as they pertains to anti-CD5 antibodies and antigen-binding fragments thereof.

Anti-CD5 antibodies that can be used in conjunction with the compositions and methods described herein include those that have one or more, or all, of the following CDRs:

```
a CDR-H1 having the amino acid sequence
  (SEQ ID NO: 341)
GYSITSGYY;
a CDR-H2 having the amino acid sequence
  (SEQ ID NO: 342)
ISYSGFT;
a CDR-H3 having the amino acid sequence
  (SEQ ID NO: 343)
AGDRTGSWFAY;
a CDR-L1 having the amino acid sequence
  (SEQ ID NO: 344)
QDISNY;
a CDR-L2 having the amino acid sequence
  (SEQ ID NO: 345)
ATS;
and
a CDR-L3 having the amino acid sequence
(SEQ ID NO: 346)
LQYASYPFT
```

Antibodies and antigen-binding fragments thereof containing the foregoing CDR sequences are described, e.g., in U.S. Pat. No. 8,679,500, the disclosure of which is incorporated herein by reference as it pertains to anti-CD5 antibodies and antigen-binding fragments thereof.

Anti-CD5 antibodies that can be used in conjunction with the compositions and methods described herein include those that have one or more, or all, of the following CDRs:

```
a CDR-H1 having the amino acid sequence
  (SEQ ID NO: 347)
GYIFTNYG;
a CDR-H2 having the amino acid sequence
  (SEQ ID NO: 348)
INTYNGEP;
a CDR-H3 having the amino acid sequence
  (SEQ ID NO: 349)
ARGDYYGYEDY;
a CDR-L1 having the amino acid sequence
  (SEQ ID NO: 350)
QGISNY;
a CDR-L2 having the amino acid sequence
  (SEQ ID NO: 351)
YTS;
and
a CDR-L3 having the amino acid sequence
(SEQ ID NO: 352)
QQYSKLPWT.
```

Antibodies and antigen-binding fragments thereof containing the foregoing CDR sequences are described, e.g., in U.S. Pat. No. 8,679,500.

Anti-CD5 antibodies that can be used in conjunction with the compositions and methods described herein include those that have one or more, or all, of the following CDRs:

```
a CDR-H1 having the amino acid sequence
  (SEQ ID NO: 353)
FSLSTSGMG;
a CDR-H2 having the amino acid sequence
  (SEQ ID NO: 354)
WWDDD;
a CDR-H3 having the amino acid sequence
  (SEQ ID NO: 355)
RRATGTGFDY;
a CDR-L1 having the amino acid sequence
  (SEQ ID NO: 356)
QDVGTA;
a CDR-L2 having the amino acid sequence
  (SEQ ID NO: 357)
WTSTRHT;
and
a CDR-L3 having the amino acid sequence
(SEQ ID NO: 358)
YNSYNT
```

Antibodies and antigen-binding fragments thereof containing the foregoing CDR sequences are described, e.g., in US Patent Application Publication No. 2008/0254027, the disclosure of which is incorporated herein by reference as it pertains to anti-CD5 antibodies and antigen-binding fragments thereof.

Other anti-CD5 antibodies that can be used in conjunction with the compositions and methods described herein include, for instance, anti-CD5 antibodies that are described in PCT Application Publication No. WO1992/014491, such as the anti-CD5 antibodies produced by hybridoma cell line deposited at the Institut Pasteur under No. 1-1025 on Jan. 10, 1991. The disclosure of PCT Application Publication No. WO1992/014491is incorporated herein by reference as it pertains to anti-CD5 antibodies and antigen-binding fragments thereof.

Other anti-CD5 antibodies that can be used in conjunction with the compositions and methods described herein include, for instance, anti-CD5 antibodies that are described in U.S. Pat. Nos. 6,010,902 and 7,192,736, US Patent Application Publication Nos. 2011/0250203 and 2017/0129128, and PCT Application Publication Nos. WO2016/172606; WO1994/023747; and WO1996/041608; the disclosures of each of which are incorporated herein by reference as they pertain to anti-CD5 antibodies and antigen binding fragments thereof.

In some embodiments, the anti-CD5 antibodies that can be used in conjunction with the compositions and methods described herein include those that contain a combination of CDR-H1, CDR-H2, CDR-H3, CDR-L1, CDR-L2, and CDR-L3 regions set forth in the table below.

TABLE 5

| Ab | | | | |
|---|---|---|---|---|
| No. | Name | CDRH1 | CDRH2 | CDRH3 |
| 1 | 1D8 | SGYSFTGYTM | LINPYNGGTT | CARDYYGSSPDFDYW |
| 2 | 3I21 | SGYSFTDYTM | LINPYNGGTM | CARDNYGSSPDFDYW |
| 3 | 4H10 | SGYSFTGYTM | LINPYNGGTM | CARDNYGSSPYFDYW |
| 4 | 8J23 | SGYSFTGYTM | LINPYNGGTM | CARDNYGSSPYFDYW |
| 5 | 504 | SGYSFTGYTM | LINPYNGGTT | CARDYYGSSPDFDYW |
| 6 | 4H2 | SGFTFSNYAM | SISSGGNTF | CVRYYYGVTYWYFDVW |
| 7 | 5G2 | SGFTFSSYAM | SISSGGSTY | CVRYYYGIRYWYFDVW |
| 8 | 8G8 | SGYSFTAYNI | SIDPYYGDTK | CARRMITMGDWYFDVW |
| 9 | 6M4 | SGYSFTAYSM | SIDPYYGDTK | CARRMITTGDWYFDVW |
| 10 | 2E3 | SGYTFTNFAI | LISSNSGDVS | CARHYGAHNYFDYW |
| 11 | 4E24 | SGYTFTNFAI | LISTSSGDVS | CARHYGANNYFDYW |
| 12 | 4F10 | SGYTFTNFAI | LISSNSGDVS | CARHYGAHNYFDYW |
| 13 | 7J9 | SGYTFTNFAI | LISSNSGDVS | CARHYGAHNYFDYW |
| 14 | 7P9 | SGFNIKDTYM | RIDPANGNTK | CAREENYYGTYYFDYW |
| 15 | 8E24 | SGYSFTSYWM | MIHPSDSETR | CARWGDHDDAMDFW |
| 16 | 6L18 | SGFSLTNYDV | VIWSGGNTD | CARNHGDGYFNWYFDVW |
| 17 | 7H7 | SGFSLTNYDV | VIWSGGNTD | CARNHGDGYYNWYFDVW |
| 18 | 1E7 | SGFTFSNYGM | AINSNGDITY | CARGTAWFTYW |
| 19 | 8J21 | SGYSFTGYTM | LINPYNGGTR | CARDGDDGWDIDVW |
| 20 | 7I11 | SGYIFANYGM | WINTYTGEPT | CARRGTYWHFDVW |
| 21 | 8M9 | SGYNFTNYGM | WINTYTGEPT | CARRGSYWHFDVW |
| 22 | 1P21 | SGYTFTNYGM | WINTYTGEPT | CARRSTLVFDYW |
| 23 | 2H11 | SGYTFTDYYI | WIYPGGGNTR | CARNGYWYFDVW |
| 24 | 3M22 | SGYTFTDYYI | WIYPGGGNTR | CARNGYWYFDVW |
| 25 | 5M6 | SGNTFTNFYL | CIYPGNVKTK | CAKEGDYDGTAYFDYW |
| 26 | 5H8 | SGYTFTNYGM | WINTYTGEPT | CARRRDGNFDYW |
| 27 | 7I19 | SEFTFSNYAM | TISSGGSYTY | CVRHGYFDVW |
| 28 | 1A20 | SGYTFTSYRM | RIDPYDSGTH | CAFYDGAYW |
| 29 | 8E15 | SGFNIKDTYM | RIDPANGNTK | CASYDPDYW |
| 30 | 8C10 | SGYSFTDYTM | LINPYNGGTR | CARDTTATYYFDYW |
| 31 | 3P16 | SGYMFTNHGM | WINTYTGEPT | CARRVATYFDVW |
| 32 | 4F3 | SGYMFTNYGM | WINTYTGEPT | CTRRSHITLDYW |
| 33 | 5M24 | SGYIFTNYGM | WINTYTGEPT | CARRRTTAFDYW |
| 34 | 5024 | SGFNIKDYYI | WIDPENGRTE | CNNGNYVRHYYFDYW |
| 35 | 7B16 | SGYTFINYGM | WINTYTGEPT | CTRRREITFDYW |
| 36 | 1E8 | SGYTFTDYFI | EIYPGSSNTY | CARSGISPFTYW |
| 37 | 2H16 | SGYIFTGYNI | AVYPGNGDTS | CAKYDRFFASW |
| 1 | 1D8 | SQGISNHL | YFTSS | CQQYSNLPYTF |
| 2 | 3121 | SQGIRNYL | YFTSS | CQQYSNLPYTF |
| 3 | 4H10 | SQGISNHL | YFTSS | CQQYSNLPYTF |
| 4 | 8J23 | SQGINNYL | YYTSS | CQQYSKIPYTC |
| 5 | 504 | SQGISNHL | YFTSS | CQQYSNLPYTF |
| 6 | 4H2 | SQSVDHDGDSYM | YAASN | CQQNYEDPTF |
| 7 | 5G2 | SQSVDYDGDSYM | YAASN | CQQSNEDPTF |
| 8 | 8G8 | SQDISNYL | YYTSR | CQQGDALPWTF |
| 9 | 6M4 | SQDISTYL | FYTSR | CQQGNSLPFTF |
| 10 | 2E3 | TSSISSSYL | YGTSN | CQQWSSRPPTF |
| 11 | 4E24 | NSSVSSSYL | YGTSN | CQQYSGYPLTF |
| 12 | 4F10 | TSSISSSYL | YGTSN | CQQYSDYPLTF |
| 13 | 7J9 | TSSISSSYL | YGTSN | CQQRSYFPFTF |
| 14 | 7P9 | SENIYYNL | YNANS | CKQVYDVPFTF |
| 15 | 8E24 | SENIYGYF | YNAKT | CQHHYGTPFTF |
| 16 | 6L18 | SQDINNYI | HYTST | CLQYDNLWTF |
| 17 | 7H7 | SQDINKYI | HYTST | CLQYDNLWTF |
| 18 | 1E7 | SENIYSYL | YNAKT | CQHHYGYPYTF |
| 19 | 8J21 | SQGIRNYL | YHTST | CQQYSNLPLTF |
| 20 | 7I11 | SQDVRTDV | YSASF | CQQHYTSPWTF |
| 21 | 8M9 | SQDVITAV | YSASY | CQQHYSTPWTF |
| 22 | 1P21 | SQSIGTSI | KSASE | CQQSNRWPLTF |
| 23 | 2H11 | SSQSLLNQKNYL | YWAST | CQNDYDYPYTF |
| 24 | 3M22 | SSSVSSSYL | YSTSN | CHQYHRSPLTF |
| 25 | 5M6 | SENIYYNL | YNANS | CQQTFDVPWTF |
| 26 | 5H8 | SQTIGTSI | KNASE | CQQSNSWPLTY |
| 27 | 7I19 | SQSLLYSSDQKNYL | YWAST | CQQYYNYPLTF |
| 28 | 1A20 | NSSVSYM | YDTSK | CQQWSSNPFTF |
| 29 | 8E15 | SENIYYNL | YNANS | CKQAYDVPWTF |
| 30 | 8C10 | SSSLSYM | YDTSN | CQQWSSFPPTF |
| 31 | 3P16 | SQRIGTSM | KSASE | CQQSNSWPLTF |
| 32 | 4F3 | SQSIGTSI | KSASE | CQQSNSWPLTF |
| 33 | 5M24 | SQNIGTSI | KDASE | CQQSDSWPLTF |
| 34 | 5024 | ISSVSYM | YATSN | CQQWSSNPRTF |
| 35 | 7B16 | SQTIATSI | KNASE | CQQSNSWPLTF |
| 36 | 1E8 | SQSLVHSNGNTYL | YKVSN | CWQNTHFPQTF |
| 37 | 2H16 | NESVEYSGTSLM | SAASN | CQQSRQVPLTF |

Antibodies and antigen-binding fragments thereof containing the foregoing CDR sequences of the table above are described, e.g., in US Patent Application Publication No. 2011/0250203, the disclosure of which is incorporated herein by reference as it pertains to anti-CD5 antibodies and antigen binding fragments thereof.

### Anti-CD2 Antibodies

Compositions and methods described herein include, in certain embodiments, an antibody, or fragment thereof, that specifically binds to human CD2. Human CD2 is also referred to as T-cell Surface Antigen T11/Leu-5, T11, CD2 antigen (p50), and Sheep Red Blood Cell Receptor (SRBC). CD2 is expressed on T cells. Two isoforms of human CD2 have been identified. Isoform 1 contains 351 amino acids is described in Seed, B. et al. (1987) 84: 3365-69 (see also Sewell et al. (1986) 83: 8718-22) and below (NCBI Reference Sequence: NP_001758.2):

(SEQ ID NO: 312)
msfpckfvas fllifnvssk gavskeitna letwgalgqd
inldipsfqm sddiddikwe ktsdkkkiaq frkeketfke
kdtyklfkng tlkikhlktd dqdiykvsiy dtkgknvlek
ifdlkigerv skpkiswtci nttltcevmn gtdpelnlyq
dgkhlklsqr vithkwttsl sakfkctagn kvskessvep
vscpekgldi yliigicggg sllmvfvall vfyitkrkkq
rsrrndeele trahrvatee rgrkphqipa stpqnpatsq
hpppppghrs qapshrpppp ghrvqhqpqk rppapsgtqv
hqqkgpplpr prvqpkpphg aaenslspss n

A second isoform of CD2 is 377 amino acids and is identified herein as NCBI Reference Sequence: NP_001315538.1.

T cells and NK cells have been shown to express CD2, which is a cell adhesion molecule and specific marker for such lymphocytes. For instance, CD2 interacts with other adhesion molecules, such as lymphocyte function-associated antigen-3 (LFA-3/CD58), to potentiate T cell activation. Antibodies and antigen-binding fragments thereof capable of binding CD2 may suppress T cell activation and T cell-mediated immune responses against hematopoietic stem cell grafts, for example, by inhibiting the interaction between CD2 and LFA-3. Antibodies and antigen-binding fragments thereof that bind to this cell-surface antigen can be identified using techniques known in the art and described herein, including immunization, computational modeling techniques, and in vitro selection methods, such as the phage display and cell-based display platforms described below.

In certain embodiments, the present invention encompasses an ADC comprising an antibody, or antigen-binding fragment thereof, that specifically binds to a CD2 polypeptide, e.g., a human CD2 polypeptide, and uses thereof.

In one embodiment, an ADC comprises an anti-CD2 antibody comprising a heavy chain variable region comprises one or more complementarity determining regions (CDRs). In one embodiment, the heavy chain variable region comprises a VH CDR1 comprising the amino acid sequence of SEQ ID NO:300. In one embodiment, the heavy chain variable region comprises a VH CDR2 comprising the amino acid sequence of SEQ ID NO:301. In one embodiment, the heavy chain variable region comprises a VH CDR3 comprising the amino acid sequence of SEQ ID NO:302. In one embodiment, the heavy chain variable region comprises one or more VH CDRs selected from the group consisting of SEQ ID NO:300, SEQ ID NO:301, and SEQ ID NO:302. In one embodiment, the heavy chain variable region comprises two or more VH CDRs selected from the group consisting of SEQ ID NO:300, SEQ ID NO:301, and SEQ ID NO:302. In one embodiment, the heavy chain variable region comprises a VH CDR1 comprising SEQ ID NO:300, a VH CDR2 comprising SEQ ID NO:301, and a VH CDR3 comprising SEQ ID NO:302.

In one embodiment, an ADC comprises an anti-CD2 antibody comprising light chain variable region comprises one or more complementarity determining regions (CDRs). In one embodiment, the light chain variable region comprises a VL CDR1 comprising the amino acid sequence of SEQ ID NO:303. In one embodiment, the light chain variable region comprises a VL CDR2 comprising the amino acid sequence of SEQ ID NO:304. In one embodiment, the light chain variable region comprises a VL CDR3 comprising the amino acid sequence of SEQ ID NO:305. In one embodiment, the light chain variable region comprises one or more VL CDRs selected from the group consisting of SEQ ID NO:303, SEQ ID NO:304, and SEQ ID NO:305. In one embodiment, the light chain variable region comprises two or more VL CDRs selected from the group consisting of SEQ ID NO:303, SEQ ID NO:304, and SEQ ID NO:305. In one embodiment, the light chain variable region comprises a VL CDR1 comprising SEQ ID NO:303, a VL CDR2 comprising SEQ ID NO:304, and a VL CDR3 comprising SEQ ID NO:305.

In an exemplary embodiment, the anti-CD2 antibody, or antigen-binding fragment thereof, comprises a heavy chain variable region that comprises a VH CDR1 comprising SEQ ID NO:300, a VH CDR2 comprising SEQ ID NO:301, and a VH CDR3 comprising SEQ ID NO:302, and a light chain variable region that comprises a VL CDR1 comprising SEQ ID NO:303, a VL CDR2 comprising SEQ ID NO:304, and a VL CDR3 comprising SEQ ID NO:305.

In certain embodiments, one or more of the CDRs (i.e., one or more heavy chain CDRs having SEQ ID NOs: 300-302, and/or one or more light chain CDRs having SEQ ID NOs: 303-305) can comprise a conservative amino acid substitution (or 2, 3, 4, or 5 amino acid substitutions) while retaining the CD2 specificity of the antibody (i.e., specificity similar to an antibody, or antigen-binding fragment thereof, comprising heavy chain CDRs of SEQ ID NOs: 300 to 302, and light chain CDRs of SEQ ID NOs:303 to 305).

In one embodiment, the antibody, or antigen-binding fragment thereof, comprises a heavy chain variable region that comprises the amino acid sequence set forth in SEQ ID NO: 306. In another embodiment, the antibody, or antigen-binding fragment thereof, comprises a heavy chain variable region that comprises an amino acid sequence having at least 95% identity to SEQ ID NO: 306, e.g., at least 95%, 96%, 97%, 98%, 99%, or 100% identity to SEQ ID NO: 306. In certain embodiments, an antibody comprises a modified heavy chain (HC) variable region comprising an HC variable domain comprising SEQ ID NO: 306, or a variant of SEQ ID NO: 306, which variant (i) differs from SEQ ID NO: 306 in 1, 2, 3, 4 or 5 amino acids substitutions, additions or deletions; (ii) differs from SEQ ID NO: 306 in at most 5, 4, 3, 2, or 1 amino acids substitutions, additions or deletions; (iii) differs from SEQ ID NO: 306 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO: 306, wherein in any of (i)-(iv), an amino acid substitution may be a conservative amino acid substitution or a non-conservative amino acid substitution; and wherein the modified heavy chain variable region can have an enhanced biological activity relative to the heavy chain variable region of SEQ ID NO: 306, while retaining the CD2 binding specificity of the antibody, i.e. has a binding specificity similar to an antibody, or antigen-binding fragment thereof, comprising SEQ ID NO: 306. In one embodiment, the antibody, or antigen-binding fragment thereof, comprises a heavy chain variable region that differs from the amino acid sequence set forth in SEQ ID NO: 306 at one, two, three or four amino acids. For example, the antibody, or antigen-binding fragment thereof, can comprise a heavy chain variable region that differs from the amino acid sequence set forth in SEQ ID NO: 306 at one, two, three, or four of positions 12, 13, 28, and/or 48. In one embodiment, the heavy chain variable region differs from the amino acid sequence set forth in SEQ ID NO:306 at positions 12, 13, 28, and 48. In one embodiment, the heavy chain variable region comprises one, two, three, or four of the following substitutions with respect to the sequence set forth in SEQ ID NO:306: K12Q; K13R; T28I; and M48V. In one embodiment, the heavy chain variable region comprises the substitutions K12Q; K13R; T28I; and M48V with respect to SEQ ID NO:306.

In one embodiment, the antibody, or antigen-binding fragment thereof, comprises a light chain variable region that comprises the amino acid sequence set forth in SEQ ID NO:307. In another embodiment, the antibody, or antigen-binding fragment thereof, comprises a light chain variable region that comprises an amino acid sequence having at least 95% identity to SEQ ID NO:307, e.g., at least 95%, 96%, 97%, 98%, 99%, or 100% identity to SEQ ID NO:307. In certain embodiments, an antibody comprises a modified light chain (LC) variable region comprising an LC variable domain comprising SEQ ID NO: 307, or a variant of SEQ ID NO: 307, which variant (i) differs from SEQ ID NO: 307 in 1, 2, 3, 4 or 5 amino acids substitutions, additions or deletions; (ii) differs from SEQ ID NO: 307 in at most 5, 4, 3, 2, or 1 amino acids substitutions, additions or deletions; (iii) differs from SEQ ID NO: 307 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO: 307, wherein in any of (i)-(iv), an amino acid substitution may be a conservative amino acid substitution or a non-conservative amino acid substitution; and wherein the modified light chain variable region can have an enhanced biological activity relative to the light chain variable region of SEQ ID NO:307, while retaining the CD2 binding specificity of the antibody, i.e., has a binding specificity similar to an antibody, or antigen-binding fragment thereof, comprising SEQ ID NO:307.

In an exemplary embodiment, the antibody, or antigen-binding fragment thereof, comprises a heavy chain variable region that comprises an amino acid sequence having at least 95% identity to SEQ ID NO: 306, e.g., at least about 95%, about 96%, about 97%, about 98%, about 99%, or 100% identity to SEQ ID NO: 306, and a light chain variable region that comprises an amino acid sequence having at least about 95% identity to SEQ ID NO:307, e.g., at least about 95%, about 96%, about 97%, about 98%, about 99%, or 100% identity to SEQ ID NO:307. In one embodiment, the antibody, or antigen-binding fragment thereof, comprises a heavy chain variable region that comprises SEQ ID NO: 306, and a light chain variable region that comprises SEQ ID NO:307. In one embodiment, the antibody is an Ab1 antibody that comprises a heavy chain variable region comprising SEQ ID NO:306, and a light chain variable region comprising SEQ ID NO:307.

In one embodiment, the antibody, or antigen-binding fragment thereof, comprises a heavy chain variable region that comprises the amino acid sequence set forth in SEQ ID NO:308. In another embodiment, the antibody, or antigen-binding fragment thereof, comprises a heavy chain variable region that comprises an amino acid sequence having at least 95% identity to SEQ ID NO:308, e.g., at least about 95%, about 96%, about 97%, about 98%, about 99%, or 100% identity to SEQ ID NO:308. In an exemplary embodiment, the antibody, or antigen-binding fragment thereof, comprises a heavy chain variable region that comprises an amino acid sequence having at least 95% identity to SEQ ID NO:308, e.g., at least about 95%, about 96%, about 97%, about 98%, about 99%, or 100% identity to SEQ ID NO:308, and a light chain variable region that comprises an amino acid sequence having at least about 95% identity to SEQ ID NO:309, e.g., at least about 95%, about 96%, about 97%, about 98%, about 99%, or 100% identity to SEQ ID NO:309. In one embodiment, the antibody, or antigen-binding fragment thereof, comprises a heavy chain variable region that comprises SEQ ID NO:308, and alight chain variable region that comprises SEQ ID NO:309. In one embodiment, the antibody is an Ab1a antibody that comprises a heavy chain variable region comprising SEQ ID NO:308, and a light chain variable region comprising SEQ ID NO:309.

In one embodiment, the heavy chain variable region comprises one or more complementarity determining regions (CDRs). In one embodiment, the heavy chain variable region comprises a VH CDR1 comprising the amino acid sequence of SEQ ID NO:3131. In one embodiment, the heavy chain variable region comprises a VH CDR2 comprising the amino acid sequence of SEQ ID NO:314. In one embodiment, the heavy chain variable region comprises a VH CDR3 comprising the amino acid sequence of SEQ ID NO:315. In one embodiment, the heavy chain variable region comprises one or more VH CDRs selected from the group consisting of SEQ ID NO:313, SEQ ID NO:314, and SEQ ID NO:315. In one embodiment, the heavy chain variable region comprises two or more VH CDRs selected from the group consisting of SEQ ID NO:313, SEQ ID NO:314, and SEQ ID NO:315. In one embodiment, the heavy chain variable region comprises a VH CDR1 comprising SEQ ID NO:313, a VH CDR2 comprising SEQ ID NO:314, and a VH CDR3 comprising SEQ ID NO:315.

In one embodiment, the heavy chain variable region comprises one or more complementarity determining regions (CDRs). In one embodiment, the heavy chain variable region comprises a VH CDR1 comprising the amino acid sequence of SEQ ID NO:313. In one embodiment, the heavy chain variable region comprises a VH CDR2 comprising the amino acid sequence of SEQ ID NO:314. In one embodiment, the heavy chain variable region comprises a VH CDR3 comprising the amino acid sequence of SEQ ID NO:316. In one embodiment, the heavy chain variable region comprises one or more VH CDRs selected from the group consisting of SEQ ID NO:313, SEQ ID NO:314, and SEQ ID NO:316. In one embodiment, the heavy chain variable region comprises two or more VH CDRs selected from the group consisting of SEQ ID NO:313, SEQ ID NO:314, and SEQ ID NO:316. In one embodiment, the heavy chain variable region comprises a VH CDR1 comprising SEQ ID NO:313, a VH CDR2 comprising SEQ ID NO:314, and a VH CDR3 comprising SEQ ID NO:316.

In one embodiment, the light chain variable region comprises one or more complementarity determining regions (CDRs). In one embodiment, the light chain variable region comprises a VL CDR1 comprising the amino acid sequence of SEQ ID NO:317. In one embodiment, the light chain variable region comprises a VL CDR2 comprising the amino acid sequence of SEQ ID NO:318. In one embodiment, the light chain variable region comprises a VL CDR3 comprising the amino acid sequence of SEQ ID NO:319. In one embodiment, the light chain variable region comprises one or more VL CDRs selected from the group consisting of SEQ ID NO:317, SEQ ID NO:318, and SEQ ID NO:319. In one embodiment, the light chain variable region comprises two or more VL CDRs selected from the group consisting of SEQ ID NO:317, SEQ ID NO:318, and SEQ ID NO:319. In one embodiment, the light chain variable region comprises a VL CDR1 comprising SEQ ID NO:317, a VL CDR2 comprising SEQ ID NO:318, and a VL CDR3 comprising SEQ ID NO:319.

In an exemplary embodiment, the antibody, or antigen-binding fragment thereof, comprises a heavy chain variable region that comprises a VH CDR1 comprising SEQ ID NO:313, a VH CDR2 comprising SEQ ID NO:314, and a VH CDR3 comprising SEQ ID NO:315, and a light chain variable region that comprises a VL CDR1 comprising SEQ ID NO:317, a VL CDR2 comprising SEQ ID NO:318, and a VL CDR3 comprising SEQ ID NO:319.

In an exemplary embodiment, the antibody, or antigen-binding fragment thereof, comprises a heavy chain variable region that comprises a VH CDR1 comprising SEQ ID NO:313, a VH CDR2 comprising SEQ ID NO:314, and a VH CDR3 comprising SEQ ID NO:316, and a light chain variable region that comprises a VL CDR1 comprising SEQ ID NO:317, a VL CDR2 comprising SEQ ID NO:318, and a VL CDR3 comprising SEQ ID NO:319.

In certain embodiments, one or more of the CDRs (i.e., one or more heavy chain CDRs having SEQ ID NOs: 313-316, and/or one or more light chain CDRs having SEQ ID NOs: 317-318) can comprise a conservative amino acid substitution (or 2, 3, 4, or 5 amino acid substitutions) while retaining the CD2 specificity of the antibody (i.e., specificity similar to an antibody, or antigen-binding fragment thereof, comprising heavy chain CDRs of SEQ ID NOs: 313 to 315, and light chain CDRs of SEQ ID NOs:18 to 20; or comprising heavy chain CDRs of SEQ ID NOs: 313, 314, 316, and light chain CDRs of SEQ ID NOs:317 to 319).

In one embodiment, the antibody, or antigen-binding fragment thereof, comprises a heavy chain variable region that comprises the amino acid sequence set forth in SEQ ID NO: 320. In another embodiment, the antibody, or antigen-binding fragment thereof, comprises a heavy chain variable region that comprises an amino acid sequence having at least about 95% identity to SEQ ID NO: 320, e.g., at least about 95%, about 96%, about 97%, about 98%, about 99%, or 100% identity to SEQ ID NO: 320. In certain embodiments, an antibody comprises a modified heavy chain (HC) variable region comprising an HC variable domain comprising SEQ ID NO: 320, or a variant of SEQ ID NO: 320, which variant (i) differs from SEQ ID NO: 320 in 1, 2, 3, 4 or 5 amino acids substitutions, additions or deletions; (ii) differs from SEQ ID NO: 320 in at most 5, 4, 3, 2, or 1 amino acids substitutions, additions or deletions; (iii) differs from SEQ ID NO: 320 in 1-5, 1-3, 1-2, 2-5 or 3-5 amino acids substitutions, additions or deletions and/or (iv) comprises an amino acid sequence that is at least about 75%, about 80%, about 85%, about 90%, about 95%, about 96%, about 97%, about 98% or about 99% identical to SEQ ID NO: 320, wherein in any of (i)-(iv), an amino acid substitution may be a conservative amino acid substitution or a non-conservative amino acid substitution; and wherein the modified heavy chain variable region can have an enhanced biological activity relative to the heavy chain variable region of SEQ ID NO: 320, while retaining the CD2 binding specificity of the antibody, i.e. has a binding specificity similar to an antibody, or antigen-binding fragment thereof, comprising SEQ ID NO: 320.

In one embodiment, the antibody, or antigen-binding fragment thereof, comprises a heavy chain variable region that comprises the amino acid sequence set forth in SEQ ID NO: 321. In another embodiment, the antibody, or antigen-binding fragment thereof, comprises a heavy chain variable region that comprises an amino acid sequence having at least about 95% identity to SEQ ID NO: 321, e.g., at least about 95%, about 96%, about 97%, about 98%, about 99%, or 100% identity to SEQ ID NO: 321. In certain embodiments, an antibody comprises a modified heavy chain (HC) variable region comprising an HC variable domain comprising SEQ ID NO: 320, or a variant of SEQ ID NO: 321, which variant (i) differs from SEQ ID NO: 321 in 1, 2, 3, 4 or 5 amino acids substitutions, additions or deletions; (ii) differs from SEQ ID NO: 321 in at most 5, 4, 3, 2, or 1 amino acids substitutions, additions or deletions; (iii) differs from SEQ ID NO: 321 in 1-5, 1-3, 1-2, 2-5 or 3-5 amino acids substitutions, additions or deletions and/or (iv) comprises an amino acid sequence that is at least about 75%, about 80%, about 85%, about 90%, about 95%, about 96%, about 97%, about 98% or about 99% identical to SEQ ID NO: 321, wherein in any of (i)-(iv), an amino acid substitution may be a conservative amino acid substitution or a non-conservative amino acid substitution; and wherein the modified heavy chain variable region can have an enhanced biological activity relative to the heavy chain variable region of SEQ ID NO: 321, while retaining the CD2 binding specificity of the antibody, i.e. has a binding specificity similar to an antibody, or antigen-binding fragment thereof, comprising SEQ ID NO: 321.

In one embodiment, the antibody, or antigen-binding fragment thereof, comprises a light chain variable region that comprises the amino acid sequence set forth in SEQ ID NO:322. In another embodiment, the antibody, or antigen-binding fragment thereof, comprises a light chain variable region that comprises an amino acid sequence having at least about 95% identity to SEQ ID NO:322, e.g., at least about about 95%, about 96%, about 97%, about 98%, about 99%, or 100% identity to SEQ ID NO:322. In certain embodiments, an antibody comprises a modified light chain (LC) variable region comprising an LC variable domain comprising SEQ ID NO: 322, or a variant of SEQ ID NO: 322, which variant (i) differs from SEQ ID NO: 322 in 1, 2, 3, 4 or 5 amino acids substitutions, additions or deletions; (ii) differs from SEQ ID NO: 322 in at most 5, 4, 3, 2, or 1 amino acids substitutions, additions or deletions; (iii) differs from SEQ ID NO: 322 in 1-5, 1-3, 1-2, 2-5 or 3-5 amino acids substitutions, additions or deletions and/or (iv) comprises an amino acid sequence that is at least about 75%, about 80%, about 85%, about 90%, about 95%, about 96%, about 97%, about 98% or about 99% identical to SEQ ID NO: 322, wherein in any of (i)-(iv), an amino acid substitution may be a conservative amino acid substitution or a non-conservative amino acid substitution; and wherein the modified light chain variable region can have an enhanced biological activity relative to the light chain variable region of SEQ ID NO:322, while retaining the CD2 binding specificity of the antibody, i.e., has a binding specificity similar to an antibody, or antigen-binding fragment thereof, comprising SEQ ID NO:322.

In an exemplary embodiment, the antibody, or antigen-binding fragment thereof, comprises a heavy chain variable region that comprises an amino acid sequence having at least 95% identity to SEQ ID NO: 320, e.g., at least about 95%, about 96%, about 97%, about 98% or about 99%, or 100% identity to SEQ ID NO: 320, and a light chain variable region that comprises an amino acid sequence having at least about 95% identity to SEQ ID NO:322, e.g., at least about 95%, about 96%, about 97%, about 98% or about 99%, or 100% identity to SEQ ID NO:322. In one embodiment, the antibody, or antigen-binding fragment thereof, comprises a heavy chain variable region that comprises SEQ ID NO: 320, and a light chain variable region that comprises SEQ ID NO:322.

In an exemplary embodiment, the antibody, or antigen-binding fragment thereof, comprises a heavy chain variable region that comprises an amino acid sequence having at least about 95% identity to SEQ ID NO: 321, e.g., at least about 95%, about 96%, about 97%, about 98% or about 99%, or 100% identity to SEQ ID NO: 321, and a light chain variable region that comprises an amino acid sequence having at least about 95% identity to SEQ ID NO:322, e.g., at least about 95%, about 96%, about 97%, about 98% or about 99%, or 100% identity to SEQ ID NO:322. In one embodiment, the antibody, or antigen-binding fragment thereof, comprises a heavy chain variable region that comprises SEQ ID NO: 321, and a light chain variable region that comprises SEQ ID NO:322.

Anti-CD2 antibodies that can be used in conjunction with the compositions and methods described herein include those that have one or more, or all, of the following CDRs:

```
a. a CDR-H1 having the amino acid sequence
  (SEQ ID NO: 300)
EYYMY;
b. a CDR-H2 having the amino acid sequence
  (SEQ ID NO: 301)
RIDPEDGSIDYVEKFKK;
c. a CDR-H3 having the amino acid sequence
  (SEQ ID NO: 302)
GKFNYRFAY;
d. a CDR-L1 having the amino acid sequence
  (SEQ ID NO: 303)
RSSQSLLHSSGNTYLN;
e. a CDR-L2 having the amino acid sequence
  (SEQ ID NO: 304)
LVSKLES;
and
f. a CDR-L3 having the amino acid sequence
(SEQ ID NO: 305)
MQFTHYPYT.
```

Antibodies and antigen-binding fragments thereof containing the foregoing CDR sequences are described, e.g., in U.S. Pat. No. 6,849,258, the disclosure of which is incorporated herein by reference as it pertains to anti-CD2 antibodies and antigen-binding fragments thereof.

The antibodies and fragments thereof disclosed in U.S. Pat. Nos. 5,730,979; 5,817,311; 5,951,983; and 7,592,006; such as LO-CD2a, BTI-322, and antibodies produced by the hybridoma cell line deposited as ATCC Deposit No. HB 11423 (e.g., antibodies or antigen-binding fragments thereof containing one or more, or all, of the CDR sequences of antibody LO-CD2a isolated from the hybridoma cell line deposited as ATCC Deposit No. HB 11423) can be used in conjunction with the compositions and methods disclosed herein. Exemplary antibodies that may be used in conjunction with the compositions and methods described herein include humanized antibodies containing one or more, or all, of the CDR sequences of an antibody isolated from the hybridoma cell line deposited as ATCC Deposit No. HB 11423, such as MEDI-507. MEDI-507 is a humanized anti-CD2 monoclonal antibody that contains the CDR-H and CDR-L sequences of (a) through (f) above, and is described in Branco et al., Transplantation 68:1588-1596 (1999). MEDI-507 is additionally described in WO99/03502A1 and WO1994/020619A1; U.S. Pat. Nos. U.S. Pat. Nos. 7,592,006, 6,849,258, 5,951,983, 5,817,311, and 5,730,979; and U.S. Patent Publication Nos. US2011/0280868, US2004/0265315 and 2011/0091453, the disclosures of each of which are incorporated herein by reference as they pertain to anti-CD2 antibodies and antigen-binding fragments thereof, such as the anti-CD2 antibody MEDI-507. In one embodiment, the anti-CD2 antibody is Siplizumab, or an antigen-binding fragment thereof.

Other anti-CD2 antibodies that can be used in conjunction with the compositions and methods described herein include, for instance, anti-CD2 antibodies that are described in U.S. Pat. Nos. 6,541,611 and 7,250,167, the disclosures of each of which are incorporated herein by reference as they pertain to anti-CD2 antibodies and antigen-binding fragments thereof, such as the anti-CD2 antibody LO-CD2b and antibodies produced by the hybridoma cell line deposited as ATCC Deposit No. PTA-802. Exemplary antibodies that may be used in conjunction with the compositions and methods described herein include humanized antibodies containing one or more, or all, of the CDR sequences of an antibody isolated from the hybridoma cell line deposited as ATCC Deposit No. PTA-802.

Other anti-CD2 antibodies that can be used in conjunction with the compositions and methods described herein include, for instance, anti-CD2 antibodies that are described in U.S. Pat. Nos. 5,795,572 and 5,807,734, the disclosures of each of which are incorporated herein by reference as they pertains to anti-CD2 antibodies and antigen-binding fragments thereof, such as the anti-CD2 antibody produced by hybridoma cell line deposited as ATCC Deposit No. HB 69277. For instance, anti-CD2 antibodies and antigen-binding fragments thereof that may be used in conjunction with the compositions and methods described herein include those that contain a hinge region having an amino acid sequence of EPKSSDKTHTSPPSP (SEQ ID NO: 316), such as scFv fragments containing a hinge region having the amino acid sequence of EPKSSDKTHTSPPSP (SEQ ID NO: 316). The incorporation of a hinge region having the amino acid sequence of SEQ ID NO: 316 can be beneficial, as this hinge motif has been mutated relative to wild-type hinge region sequences so as to eliminate potentially reactive cysteine residues that may promote undesirable oxidative dimerization of a single-chain antibody fragment, such as a scFv fragment.

Other anti-CD2 antibodies that can be used in conjunction with the compositions and methods described herein include, for instance, anti-CD2 antibodies that are described in U.S. Pat. No. 6,764,688, such as the anti-CD2 antibody TS2/18 and antibodies produced by hybridoma cell line deposited as ATCC Deposit No. HB-195. The disclosure of U.S. Pat. No. 6,764,688 is incorporated herein by reference as it pertains to anti-CD2 antibodies and antigen-binding fragments thereof.

Other anti-CD2 antibodies that can be used in conjunction with the compositions and methods described herein include, for instance, anti-CD2 antibodies that are described in U.S. Pat. Nos. 6,162,432, 6,558,662, 7,408,039, 7,332,157, 7,638,121, 7,939,062, and 7,115,259, US Patent Application Publication No. 2006/0084107, 2014/0369974, 2002/0051784, and 2013/0183322, and PCT Publication No. WO1992/016563, the disclosures of each of which are incorporated herein by reference as they pertain to anti-CD2 antibodies and antigen binding fragments thereof.

### Anti-Her2 Antibodies

Antibodies specific to Her2 antigen are known to the person skilled in the art, e.g., trastuzumab.

### Anti-PSMA Antibodies

Antibodies specific to prostate-specific membrane antigen (PSMA) comprised in ADCs according to the present invention have been disclosed in WO2020/025564A1, the disclosure of which is herewith incorporated by reference in its entirety.

### Fc Mutations

The antibodies or binding fragments described herein may also include modifications and/or mutations that alter the properties of the antibodies and/or fragments, such as those that increase half-life, increase or decrease ADCC, etc., as is known in the art.

In one embodiment, an antibody, or binding fragment thereof, comprises a variant Fc region, wherein said variant Fc region comprises at least one amino acid modification relative to a wild-type Fc region, such that said molecule has an altered affinity for an FcgammaR. Certain amino acid positions within the Fc region are known through crystallography studies to make a direct contact with FcγR. Specifically amino acids 234-239 (hinge region), amino acids 265-269 (B/C loop), amino acids 297-299 (C′/E loop), and amino acids 327-332 (F/G) loop. (see Sondermann et al., 2000 Nature, 406: 267-273). Thus, the antibodies (e.g., anti-CD117, CD45, CD137, CD2, CD5, CD262, or CD134) described herein may comprise variant Fc regions comprising modification of at least one residue that makes a direct contact with an Fcγ R based on structural and crystallographic analysis. In one embodiment, the Fc region of the antibody (or fragment thereof) comprises an amino acid substitution at amino acid 265 according to the EU index as in Kabat et al., Sequences of Proteins of Immunological Interest, 5th Ed. Public Health Service, NH1, MD (1991), expressly incorporated herein by references. The “EU index as in Kabat” refers to the numbering of the human IgG1 EU antibody. The EU index or EU index as in Kabat or EU numbering scheme refers to the numbering of the EU antibody (Edelman et al., 1969, Proc Natl Acad Sci USA 63:78-85, hereby entirely incorporated by reference.) In one embodiment, the Fc region comprises a D265A mutation. In one embodiment, the Fc region comprises a D265C mutation. In some embodiments, the Fc region of the antibody (or fragment thereof) comprises an amino acid substitution at amino acid 234 according to the EU index as in Kabat. In one embodiment, the Fc region comprises a L234A mutation. In some embodiments, the Fc region of the antibody (or fragment thereof) comprises an amino acid substitution at amino acid 235 according to the EU index as in Kabat. In one embodiment, the Fc region comprises a L235A mutation. In yet another embodiment, the Fc region comprises a L234A and L235A mutation. In a further embodiment, the Fc region of the antibody of an ADC described herein comprises a D265C, L234A, and L235A mutation.

In certain aspects a variant IgG Fc domain comprises one or more amino acid substitutions resulting in decreased or ablated binding affinity for an Fc.gamma.R and/or Clq as compared to the wild type Fc domain not comprising the one or more amino acid substitutions. Fc binding interactions are essential for a variety of effector functions and downstream signaling events including, but not limited to, antibody dependent cell-mediated cytotoxicity (ADCC) and complement dependent cytotoxicity (CDC). Accordingly, in certain aspects, an antibody comprising a modified Fc region (e.g., comprising a L234A, L235A, and a D265C mutation) has substantially reduced or abolished effector functions.

Affinity to an Fc region can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE™. analysis or Octet™ analysis (forteBIO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. One example of a competitive binding assay is a radioimmunoassay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody.

Antibodies may be further engineered to further modulate antibody half-life by introducing additional Fc mutations, such as those described for example in (Dall'Acqua et al. (2006) J Biol Chem 281: 23514-24), (Zalevsky et al. (2010) Nat Biotechnol 28: 157-9), (Hinton et al. (2004) J Biol Chem 279: 6213-6), (Hinton et al. (2006) J Immunol 176: 346-56), (Shields et al. (2001) J Biol Chem 276: 6591-604), (Petkova et al. (2006) Int Immunol 18: 1759-69), (Datta-Mannan et al. (2007) Drug Metab Dispos 35: 86-94), (Vaccaro et al. (2005) Nat Biotechnol 23: 1283-8), (Yeung et al. (2010) Cancer Res 70: 3269-77) and (Kim et al. (1999) Eur J Immunol 29: 2819-25), and include positions 250, 252, 253, 254, 256, 257, 307, 376, 380, 428, 434 and 435. Exemplary mutations that may be made singularly or in combination are T250Q, M252Y, 1253A, S254T, T256E, P2571, T307A, D376V, E380A, M428L, H433K, N434S, N434A, N434H, N434F, H435A and H435R mutations.

Thus, in one embodiment, the Fc region comprises a mutation resulting in a decrease in half life. An antibody having a short half life may be advantageous in certain instances where the antibody is expected to function as a short-lived therapeutic, e.g., the conditioning step described herein where the antibody is administered followed by HSCs. Ideally, the antibody would be substantially cleared prior to delivery of the HSCs, which also generally express an antigen targeted by an ADC described herein, e.g., CD117, but are not the target of the ADC, unlike the endogenous stem cells. In one embodiment, the Fc regions comprise a mutation at position 435 (EU index according to Kabat). In one embodiment, the mutation is an H435A mutation.

In one embodiment, the antibody described herein has a half life of equal to or less than 24 hours, a half life of equal to or less than 22 hours, a half life of equal to or less than 20 hours, a half life of equal to or less than 18 hours, a half life of equal to or less than 16 hours, a half life of equal to or less than 14 hours, equal to or less than 13 hours, equal to or less than 12 hours, or equal to or less than 11 hours. In one embodiment, the half life of the antibody is 11 hours to 24 hours; 12 hours to 22 hours; 10 hours to 20 hours; 8 hours to 18 hours; or 14 hours to 24 hours.

In some aspects, the Fc region comprises two or more mutations that confer reduced half-life and greatly diminish or completely abolish an effector function of the antibody. In some embodiments, the Fc region comprises a mutation resulting in a decrease in half-life and a mutation of at least one residue that can make direct contact with an FcγR (e.g., as based on structural and crystallographic analysis). In one embodiment, the Fc region comprises a H435A mutation, a L234A mutation, and a L235A mutation. In one embodiment, the Fc region comprises a H435A mutation and a D265C mutation. In one embodiment, the Fc region comprises a H435A mutation, a L234A mutation, a L235A mutation, and a D265C mutation.

In some embodiments, the antibody or antigen-binding fragment thereof is conjugated to a cytotoxin (e.g., amatoxin) by way of a cysteine residue in the Fc domain of the antibody or antigen-binding fragment thereof. In some embodiments, the cysteine residue is introduced by way of a mutation in the Fc domain of the antibody or antigen-binding fragment thereof. For instance, the cysteine residue may be selected from the group consisting of Cys118, Cys239, and Cys265. In one embodiment, the Fc region of the anti-CD117 antibody (or fragment thereof) comprises an amino acid substitution at amino acid 265 according to the EU index as in Kabat. In one embodiment, the Fc region comprises a D265C mutation. In one embodiment, the Fc region comprises a D265C and H435A mutation. In one embodiment, the Fc region comprises a D265C, a L234A, and a L235A mutation. In one embodiment, the Fc region comprises a D265C, a L234A, a L235A, and a H435A mutation.

In some embodiments of these aspects, the cysteine residue is naturally occurring in the Fc domain of the antibody or antigen-binding fragment thereof. For instance, the Fc domain may be an IgG Fc domain, such as a human IgG1 Fc domain, and the cysteine residue may be selected from the group consisting of Cys261, Csy321, Cys367, and Cys425.

For example, in one embodiment, the Fc region of Antibody 67 is modified to comprise a D265C mutation (e.g., SEQ ID NO: 111). In another embodiment, the Fc region of Antibody 67 is modified to comprise a D265C, L234A, and L235A mutation (e.g., SEQ ID NO: 112). In yet another embodiment, the Fc region of Antibody 67 is modified to comprise a D265C and H435A mutation (e.g., SEQ ID NO: 113). In a further embodiment, the Fc region of Antibody 67 is modified to comprise a D265C, L234A, L235A, and H435A mutation (e.g., SEQ ID NO: 114).

In regard to Antibody 55, in one embodiment, the Fc region of Antibody 55 is modified to comprise a D265C mutation (e.g., SEQ ID NO: 117). In another embodiment, the Fc region of Antibody 55 is modified to comprise a D265C, L234A, and L235A mutation (e.g., SEQ ID NO: 118). In yet another embodiment, the Fc region of Antibody 55 is modified to comprise a D265C and H435A mutation (e.g., SEQ ID NO: 119). In a further embodiment, the Fc region of Antibody 55 is modified to comprise a D265C, L234A, L235A, and H435A mutation (e.g., SEQ ID NO: 120).

The Fc regions of any one of Antibody 54, Antibody 55, Antibody 56, Antibody 57, Antibody 58, Antibody 61, Antibody 66, Antibody 67, Antibody 68, or Antibody 69 can be modified to comprise a D265C mutation (e.g., as in SEQ ID NO: 123); a D265C, L234A, and L235A mutation (e.g., as in SEQ ID NO: 124); a D265C and H435A mutation (e.g., as in SEQ ID NO: 125); or a D265C, L234A, L235A, and H435A mutation (e.g., as in SEQ ID NO: 126).

The variant Fe domains described herein are defined according to the amino acid modifications that compose them. For all amino acid substitutions discussed herein in regard to the Fc region, numbering is always according to the EU index. Thus, for example, D265C is an Fe variant with the aspartic acid (D) at EU position 265 substituted with cysteine (C) relative to the parent Fc domain. Likewise, e.g., D265C/L234A/L235A defines a variant Fe variant with substitutions at EU positions 265 (D to C), 234 (L to A), and 235 (L to A) relative to the parent Fc domain. A variant can also be designated according to its final amino acid composition in the mutated EU amino acid positions. For example, the L234A/L235A mutant can be referred to as LALA. It is noted that the order in which substitutions are provided is arbitrary.

In one embodiment, the antibody, or antigen binding fragment thereof, comprises variable regions having an amino acid sequence that is at least 95%, 96%, 97% or 99% identical to the SEQ ID Nos disclosed herein. Alternatively, the antibody, or antigen binding fragment thereof, comprises CDRs comprising the SEQ ID Nos disclosed herein with framework regions of the variable regions described herein having an amino acid sequence that is at least 95%, 96%, 97% or 99% identical to the SEQ ID Nos disclosed herein.

In one embodiment, the antibody, or antigen binding fragment thereof, comprises a heavy chain variable region and a heavy chain constant region having an amino acid sequence that is disclosed herein. In another embodiment, the antibody, or antigen binding fragment thereof, comprises a light chain variable region and a light chain constant region having an amino acid sequence that is disclosed herein. In yet another embodiment, the antibody, or antigen binding fragment thereof, comprises a heavy chain variable region, a light chain variable region, a heavy chain constant region and a light chain constant region having an amino acid sequence that is disclosed herein.

### Methods of Identifying Antibodies

Provided herein are novel ADCs that may be used, for example, in conditioning methods for stem cell transplantation. In view of the disclosure herein, other antibodies can be identified that can be used in the ADCs and methods of the invention.

Methods for high throughput screening of antibody, or antibody fragment libraries for molecules capable of binding a cell surface antigen (e.g., CD117, CD45, CD2, CD5, CD134, CD252, CD137) can be used to identify and affinity mature antibodies useful for treating cancers, autoimmune diseases, and conditioning a patient (e.g., a human patient) in need of hematopoietic stem cell therapy as described herein. Such methods include in vitro display techniques known in the art, such as phage display, bacterial display, yeast display, mammalian cell display, ribosome display, mRNA display, and cDNA display, among others. The use of phage display to isolate ligands that bind biologically relevant molecules has been reviewed, for example, in Felici et al., Biotechnol. Annual Rev. 1:149-183, 1995; Katz, Annual Rev. Biophys. Biomol. Struct. 26:27-45, 1997; and Hoogenboom et al., Immunotechnology 4:1-20, 1998, the disclosures of each of which are incorporated herein by reference as they pertain to in vitro display techniques. Randomized combinatorial peptide libraries have been constructed to select for polypeptides that bind cell surface antigens as described in Kay, Perspect. Drug Discovery Des. 2:251-268, 1995 and Kay et al., Mol. Divers. 1:139-140, 1996, the disclosures of each of which are incorporated herein by reference as they pertain to the discovery of antigen-binding molecules. Proteins, such as multimeric proteins, have been successfully phage-displayed as functional molecules (see, for example, EP 0349578; EP 4527839; and EP 0589877, as well as Chiswell and McCafferty, Trends Biotechnol. 10:80-84 1992, the disclosures of each of which are incorporated herein by reference as they pertain to the use of in vitro display techniques for the discovery of antigen-binding molecules). In addition, functional antibody fragments, such as Fab and scFv fragments, have been expressed in in vitro display formats (see, for example, McCafferty et al., Nature 348:552-554, 1990; Barbas et al., Proc. Natl. Acad. Sci. USA 88:7978-7982, 1991; and Clackson et al., Nature 352:624-628, 1991, the disclosures of each of which are incorporated herein by reference as they pertain to in vitro display platforms for the discovery of antigen-binding molecules). These techniques, among others, can be used to identify and improve the affinity of antibodies that bind, e.g., CD117, CD45, CD2, CD5, CD134, CD252, CD137 (e.g., GNNK+CD117) that can in turn be used to deplete endogenous hematopoietic stem cells in a patient (e.g., a human patient) in need of hematopoietic stem cell transplant therapy.

In addition to in vitro display techniques, computational modeling techniques can be used to design and identify antibodies, and antibody fragments, in silico that bind a cell surface antigen (e.g., CD117, CD45, CD2, CD5, CD134, CD252, CD137). For example, using computational modeling techniques, one of skill in the art can screen libraries of antibodies, and antibody fragments, in silico for molecules capable of binding specific epitopes, such as extracellular epitopes of this antigen. The antibodies, and antigen-binding fragments thereof, identified by these computational techniques can be used in conjunction with the therapeutic methods described herein, such as the cancer and autoimmune disease treatment methods described herein and the patient conditioning procedures described herein.

Additional techniques can be used to identify antibodies, and antigen-binding fragments thereof, that bind a cell surface antigen (e.g., CD117) on the surface of a cell (e.g., a cancer cell, autoimmune cell, or hematopoietic stem cell) and that are internalized by the cell, for instance, by receptor-mediated endocytosis. For example, the in vitro display techniques described above can be adapted to screen for antibodies, and antigen-binding fragments thereof, that bind a cell surface antigen (e.g., CD117) on the surface of a cancer cell, autoimmune cell, or hematopoietic stem cell and that are subsequently internalized. Phage display represents one such technique that can be used in conjunction with this screening paradigm. To identify antibodies, and fragments thereof, that bind a cell surface antigen (e.g., CD117) and are subsequently internalized by cancer cells, autoimmune cells, or hematopoietic stem cells, one of skill in the art can adapt the phage display techniques described, for example, in Williams et al., Leukemia 19:1432-1438, 2005, the disclosure of which is incorporated herein by reference in its entirety. For example, using mutagenesis methods known in the art, recombinant phage libraries can be produced that encode antibodies, antibody fragments, such as scFv fragments, Fab fragments, diabodies, triabodies, and ¹⁰Fn3 domains, among others, or ligands that contain randomized amino acid cassettes (e.g., in one or more, or all, of the CDRs or equivalent regions thereof or an antibody or antibody fragment). The framework regions, hinge, Fc domain, and other regions of the antibodies or antibody fragments may be designed such that they are non-immunogenic in humans, for instance, by virtue of having human germline antibody sequences or sequences that exhibit only minor variations relative to human germline antibodies.

Using phage display techniques described herein or known in the art, phage libraries containing randomized antibodies, or antibody fragments, covalently bound to the phage particles can be incubated with a cell surface target antigen (e.g., CD117) antigen, for instance, by first incubating the phage library with blocking agents (such as, for instance, milk protein, bovine serum albumin, and/or IgG so as to remove phage encoding antibodies, or fragments thereof, that exhibit non-specific protein binding and phage that encode antibodies or fragments thereof that bind Fc domains, and then incubating the phage library with a population of hematopoietic stem cells. The phage library can be incubated with the target cells, such as cancer cells, autoimmune cells, or hematopoietic stem cells for a time sufficient to allow cell surface antigen specific antibodies, or antigen-binding fragments thereof, (e.g., CD117-specific antibodies, or antigen-binding fragments thereof) to bind cell-surface antigen (e.g., sell-surface CD117) antigen and to subsequently be internalized by the cancer cells, autoimmune cells, or hematopoietic stem cells (e.g., from 30 minutes to 6 hours at 4° C., such as 1 hour at 4° C.). Phage containing antibodies, or fragments thereof, that do not exhibit sufficient affinity for one or more of these antigens so as to permit binding to, and internalization by, cancer cells, autoimmune cells, or hematopoietic stem cells can subsequently be removed by washing the cells, for instance, with cold (4° C.) 0.1 M glycine buffer at pH 2.8. Phage bound to antibodies, or fragments thereof, that have been internalized by the cancer cells, autoimmune cells, or hematopoietic stem cells can be identified, for instance, by lysing the cells and recovering internalized phage from the cell culture medium. The phage can then be amplified in bacterial cells, for example, by incubating bacterial cells with recovered phage in 2×YT medium using methods known in the art. Phage recovered from this medium can then be characterized, for instance, by determining the nucleic acid sequence of the gene(s) encoding the antibodies, or fragments thereof, inserted within the phage genome. The encoded antibodies, or fragments thereof, can subsequently be prepared de novo by chemical synthesis (for instance, of antibody fragments, such as scFv fragments) or by recombinant expression (for instance, of full-length antibodies).

An exemplary method for in vitro evolution of a cell surface antigen antibody (e.g., anti-CD117) antibodies for use with the compositions and methods described herein is phage display. Phage display libraries can be created by making a designed series of mutations or variations within a coding sequence for the CDRs of an antibody or the analogous regions of an antibody-like scaffold (e.g., the BC, CD, and DE loops of ¹⁰Fn3 domains). The template antibody-encoding sequence into which these mutations are introduced may be, for example, a naive human germline sequence. These mutations can be performed using standard mutagenesis techniques known in the art. Each mutant sequence thus encodes an antibody corresponding to the template save for one or more amino acid variations. Retroviral and phage display vectors can be engineered using standard vector construction techniques known in the art. P3 phage display vectors along with compatible protein expression vectors can be used to generate phage display vectors for antibody diversification.

The mutated DNA provides sequence diversity, and each transformant phage displays one variant of the initial template amino acid sequence encoded by the DNA, leading to a phage population (library) displaying a vast number of different but structurally related amino acid sequences. Due to the well-defined structure of antibody hypervariable regions, the amino acid variations introduced in a phage display screen are expected to alter the binding properties of the binding peptide or domain without significantly altering its overall molecular structure.

In a typical screen, a phage library may be contacted with and allowed to bind one of the foregoing antigens or an epitope thereof. To facilitate separation of binders and non-binders, it is convenient to immobilize the target on a solid support. Phage bearing a cell surface-binding moiety can form a complex with the target on the solid support, whereas non-binding phage remain in solution and can be washed away with excess buffer. Bound phage can then liberated from the target by changing the buffer to an extreme pH (pH 2 or pH 10), changing the ionic strength of the buffer, adding denaturants, or other known means.

The recovered phage can then be amplified through infection of bacterial cells, and the screening process can be repeated with the new pool that is now depleted in non-binding antibodies and enriched for antibodies that bind a target antigen (e.g., CD117). The recovery of even a few binding phage is sufficient to amplify the phage for a subsequent iteration of screening. After a few rounds of selection, the gene sequences encoding the antibodies or antigen-binding fragments thereof derived from selected phage clones in the binding pool are determined by conventional methods, thus revealing the peptide sequence that imparts binding affinity of the phage to the target. During the panning process, the sequence diversity of the population diminishes with each round of selection until desirable peptide-binding antibodies remain. The sequences may converge on a small number of related antibodies or antigen-binding fragments thereof. An increase in the number of phage recovered at each round of selection is an indication that convergence of the library has occurred in a screen.

Another method for identifying antibodies includes using humanizing non-human antibodies that bind a cell surface target antigen (e.g., CD117), for instance, according to the following procedure. Consensus human antibody heavy chain and light chain sequences are known in the art (see e.g., the “VBASE” human germline sequence database; Kabat et al. Sequences of Proteins of Immunological Interest, Fifth Edition, U.S. Department of Health and Human Services, NIH Publication No. 91-3242, 1991; Tomlinson et al., J. Mol. Biol. 227:776-798, 1992; and Cox et al., Eur. J. Immunol. 24:827-836, 1994, the disclosures of each of which are incorporated herein by reference as they pertain to consensus human antibody heavy chain and light chain sequences. Using established procedures, one of skill in the art can identify the variable domain framework residues and CDRs of a consensus antibody sequence (e.g., by sequence alignment). One can substitute one or more CDRs of the heavy chain and/or light chain variable domains of consensus human antibody with one or more corresponding CDRs of a non-human antibody that binds a cell surface antigen (e.g., CD117) as described herein in order to produce a humanized antibody. This CDR exchange can be performed using gene editing techniques described herein or known in the art.

To produce humanized antibodies, one can recombinantly express a polynucleotide encoding the above consensus sequence in which one or more variable region CDRs have been replaced with one or more variable region CDR sequences of a non-human antibody that binds a cell surface target antigen (e.g., CD117). As the affinity of the antibody for the hematopoietic stem cell antigen is determined primarily by the CDR sequences, the resulting humanized antibody is expected to exhibit an affinity for the hematopoietic stem cell antigen that is about the same as that of the non-human antibody from which the humanized antibody was derived. Methods of determining the affinity of an antibody for a target antigen include, for instance, ELISA-based techniques described herein and known in the art, as well as surface plasmon resonance, fluorescence anisotropy, and isothermal titration calorimetry, among others.

The internalizing capacity of an antibody, or fragment thereof, can be assessed, for instance, using radionuclide internalization assays known in the art. For example, antibodies, or fragments thereof, identified using in vitro display techniques described herein or known in the art can be functionalized by incorporation of a radioactive isotope, such as ¹⁸F, ⁷⁵Br, ⁷⁷Br, ¹²²I, ¹²³I, ¹²⁴I, ¹²⁵I, ¹²⁹I, ¹³¹I, ²¹¹At, ⁶⁷Ga, ¹¹¹In, ⁹⁹Tc, ¹⁶⁹Xb, ¹⁸⁶Re, ⁶⁴Cu, ⁶⁷Cu, ¹⁷⁷Lu, ⁷⁷As, ⁷²As, ⁸⁶Y, ⁹⁰Y, ⁸⁹Zr, ²¹²Bi, ²¹³Bi, or ²²⁵Ac. For instance, radioactive halogens, such as ¹⁸F, ⁷⁵Br, ⁷⁷Br, ¹²²I, ¹²³I, ¹²⁴I, ¹²⁵I, ¹²⁹I, ¹³¹I, ²¹¹At, can be incorporated into antibodies, or fragments thereof, using beads, such as polystyrene beads, containing electrophilic halogen reagents (e.g., Iodination Beads, Thermo Fisher Scientific, Inc., Cambridge, MA). Radiolabeled antibodies, or fragments thereof, can be incubated with cancer cells, autoimmune cells, or hematopoietic stem cells for a time sufficient to permit internalization (e.g., from 30 minutes to 6 hours at 4° C., such as 1 hour at 4° C.). The cells can then be washed to remove non-internalized antibodies, or fragments thereof, (e.g., using cold (4° C.) 0.1 M glycine buffer at pH 2.8). Internalized antibodies, or fragments thereof, can be identified by detecting the emitted radiation (e.g., γ-radiation) of the resulting cancer cells, autoimmune cells, or hematopoietic stem cells in comparison with the emitted radiation (e.g., γ-radiation) of the recovered wash buffer.

Antibodies may be produced using recombinant methods and compositions, e.g., as described in U.S. Pat. No. 4,816,567. In one embodiment, isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-CLL-1 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### Pharmaceutical Compositions

According to one aspect of the present invention, the present invention relates to a pharmaceutical composition comprising said conjugate as described.

Said pharmaceutical composition may comprise one or more pharmaceutically acceptable buffers, surfactants, diluents, carriers, excipients, fillers, binders, lubricants, glidants, disintegrants, adsorbents, and/or preservatives.

In aqueous form, said pharmaceutical formulation may be ready for administration, while in lyophilised form said formulation can be transferred into liquid form prior to administration, e.g., by addition of water for injection which may or may not comprise a preservative such as for example, but not limited to, benzyl alcohol, antioxidants like vitamin A, vitamin E, vitamin C, retinyl palmitate, and selenium, the amino acids cysteine and methionine, citric acid and sodium citrate, synthetic preservatives like the parabens methyl paraben and propyl paraben.

Said pharmaceutical formulation may further comprise one or more stabilizer, which may be, e.g., an amino acid, a sugar polyol, a disaccharide and/or a polysaccharide. Said pharmaceutical formulation may further comprise one or more surfactant, one or more isotonizing agents, and/or one or more metal ion chelator, and/or one or more preservative.

The pharmaceutical formulation as described herein can be suitable for at least intravenous, intramuscular or subcutaneous administration. Alternatively, said conjugate according to the present invention may be provided in a depot formulation which allows the sustained release of the biologically active agent over a certain period of time.

In still another aspect of the present invention, a primary packaging, such as a prefilled syringe or pen, a vial, or an infusion bag is provided, which comprises said formulation according to the previous aspect of the invention.

The prefilled syringe or pen may contain the formulation either in lyophilised form (which has then to be solubilised, e.g., with water for injection, prior to administration), or in aqueous form. Said syringe or pen is often a disposable article for single use only, and may have a volume between 0.1 and 20 ml. However, the syringe or pen may also be a multi-use or multi-dose syringe or pen.

Said vial may also contain the formulation in lyophilised form or in aqueous form and may serve as a single or multiple use device. As a multiple use device, said vial can have a bigger volume. Said infusion bag usually contains the formulation in aqueous form and may have a volume between 20 and 5000 ml.

ADCs described herein can be administered to a patient (e.g., a human patient suffering from an immune disease or cancer) in a variety of dosage forms. For instance, ADCs described herein can be administered to a patient suffering from an immune disease or cancer in the form of an aqueous solution, such as an aqueous solution containing one or more pharmaceutically acceptable excipients. Suitable pharmaceutically acceptable excipients for use with the compositions and methods described herein include viscosity-modifying agents. The aqueous solution may be sterilized using techniques known in the art.

Pharmaceutical formulations comprising ADCs as described herein are prepared by mixing such ADC with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

### Methods of Use

ADCs described herein may be administered by a variety of routes, such as orally, transdermally, subcutaneously, intranasally, intravenously, intramuscularly, intraocularly, or parenterally. The most suitable route for administration in any given case will depend on the particular antibody, or antigen-binding fragment, administered, the patient, pharmaceutical formulation methods, administration methods (e.g., administration time and administration route), the patient's age, body weight, sex, severity of the diseases being treated, the patient's diet, and the patient's excretion rate.

The effective dose of an ADC, antibody, or antigen-binding fragment thereof, described herein can range, for example from about 0.001 to about 100 mg/kg of body weight per single (e.g., bolus) administration, multiple administrations, or continuous administration, or to achieve an optimal serum concentration (e.g., a serum concentration of 0.0001-5000 g/mL) of the antibody, antigen-binding fragment thereof. The dose may be administered one or more times (e.g., 2-10 times) per day, week, or month to a subject (e.g., a human) suffering from cancer, an autoimmune disease, or undergoing conditioning therapy in preparation for receipt of a hematopoietic stem cell transplant. In the case of a conditioning procedure prior to hematopoietic stem cell transplantation, the ADC, antibody, or antigen-binding fragment thereof, can be administered to the patient at a time that optimally promotes engraftment of the exogenous hematopoietic stem cells, for instance, from 1 hour to 1 week (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, or about 7 days) or more prior to administration of the exogenous hematopoietic stem cell transplant.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1: Comparative Study of Amanitins

Three different amanitins were tested in parallel to determine their kinetics and tolerability as toxins. FIG. 1A to FIG. 1C provide the structures of three amanitin/linker conjugates. FIG. 1A provides an amanitin/linker conjugate represented by Formula (IV) (also referred to as “Conjugate A” or “ADC A”), where the amanitin has a cleavable linker (BMP-Val-Ala-PAB) linker on AA1 (Asn). The amanitin in Formula (IV) is based on amatoxin having thiotryptophan moiety. FIG. 1B provides an amanitin/linker conjugate represented by Formula (VI) (also referred to as “Conjugate B” or “ADC B”, where the amanitin is conjugated to a non-cleavable linker on indole 6′ oxygen. The amanitin described in Formula (VI) is based on amatoxin having 6-hydroxy-tryptophan sulfoxide moiety. FIG. 1C provides an amanitin/linker conjugate described herein in Formula (IIa) (also referred to as “Conjugate C” or “ADC C”, including a non-cleavable linker on indole 6′ oxygen, where the amanitin is based on amatoxin having 6-hydroxy-thiotryptophan moiety.

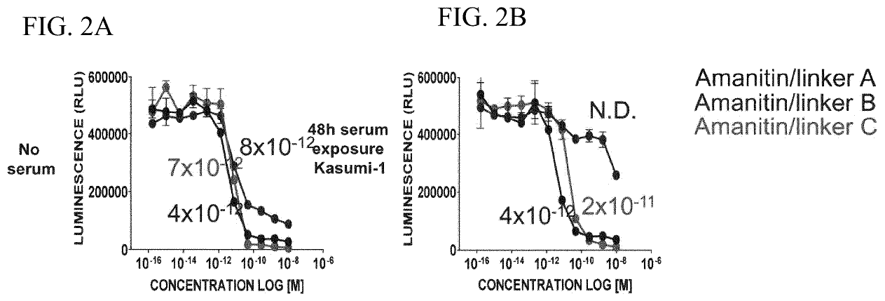

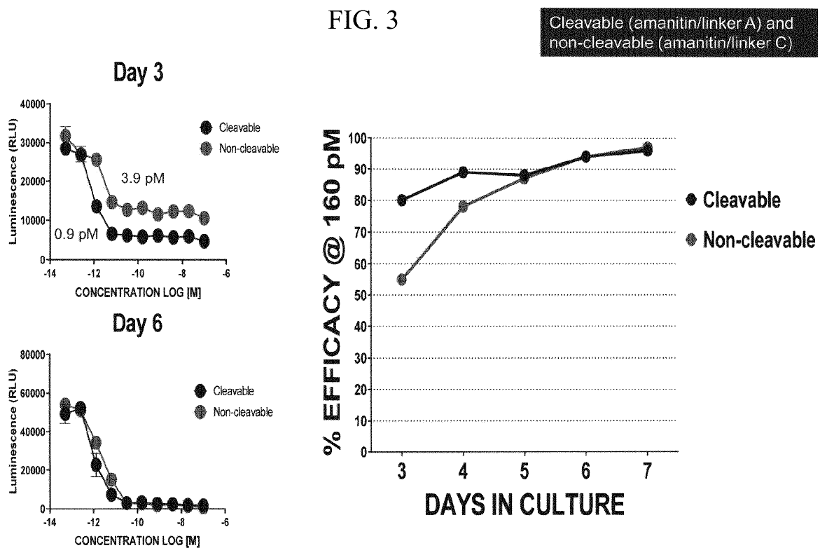

Conjugates A, B and C were tested in parallel for their potency on CD117 expressing cells with or without a pre-incubation in human serum. ADC A, B, and C were either pre-incubated in cell culture media without human serum or 50% human serum at 37C for 48 hours. The titrated ADC was then added to Kasumi-1 cells and incubated for 3 days at 37C and cell killing was determined using CellTiter-Glo. As described in FIG. 2A and FIG. 2B, in the absence of serum incubation, cleavable Conjugate A and non-cleavable Conjugate C reached similar efficacy by day 3, with non-cleavable conjugate B demonstrating reduced maximum killing at higher concentrations. Each ADC conjugate demonstrated picomolar IC50 killing. When ADC was pre-incubated for 48 hours in the presence of human serum, potency was significantly decreased for non-cleavable conjugate B but was maintained for cleavable conjugate A and non-cleavable conjugate C. Thus, cleavable and non-cleavable conjugates A and C were serum stable while non-cleavable conjugate B was inactivated in the presence of human serum as measured by in vitro cytotoxicity.

To determine differences in the kinetics of ADC-mediated cytotoxicity, Kasumi-1 cells were incubated with titrations of cleavable conjugate A or non-cleavable conjugate C for 3-7 days at 37C and cell viability was measured on day 3, 4, 5, 6, and 7 by CellTiter-Glo. As shown in FIG. 3, significant differences were observed in maximum cell killing at high concentrations for cleavable and non-cleavable ADC. When cells were incubated for a total of 6 days at 37C with ADC, both cleavable conjugate A and non-cleavable conjugate C were able to achieve nearly complete cell killing at high concentrations. Killing of greater than 80% of Kasumi-1 cells in culture with 160 pM non-cleavable conjugate C could only be achieved after incubation of Kasumi-1 cells for 5 days or longer, while a similar threshold was achieved for cleavable conjugate A after 3 days or longer incubation.

In sum, the enzyme cleavable amanitin of FIG. 1A (Conjugate A) and the non-cleavable amanitin (conjugate C in FIG. 1C) showed long term stability in serum, while amanitin/linker B showed instability and inactivation in serum. Non-cleavable conjugate C demonstrated extended kinetics of cytotoxicity as measured in an in vitro assay compared to a similar cleavable amanitin conjugate (conjugate A).

### Example 2: Anti-CD117 ADC Demonstrates Potent Killing of AML Cells and Human CD34+ Cells In Vitro, Selectively Depletes Human CD34+ Cells in Humanized Mice, and Demonstrates Anti-AML Activity in Xenograft Models

An anti-CD117 antibody (Ab85 having Fc modifications L234A, L235A, D265C, and H435A) was conjugated to Conjugate C forming anti-CD117 ADC C. Anti-CD117 ADC C was tested both in vitro and in vivo for its ability to target and kill cells expressing CD117 and human HSCs.

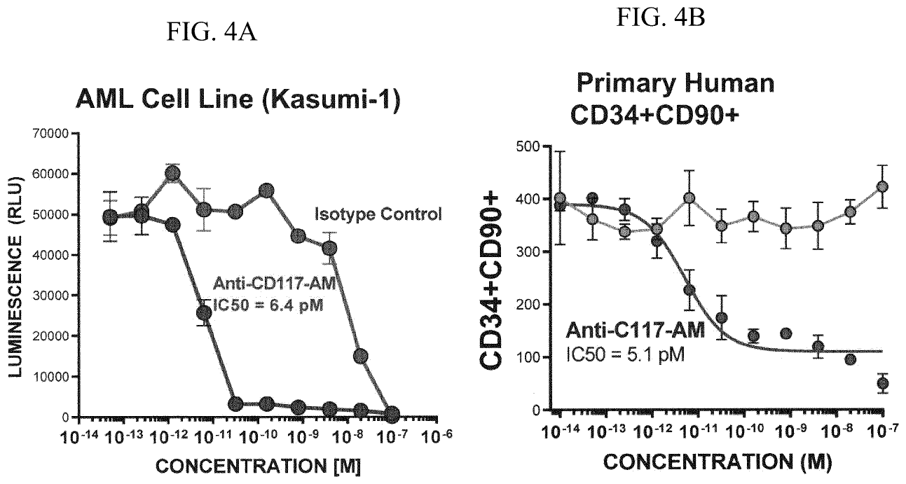

Two in vitro assays were performed using anti-CD117 ADC C. The first tested the ability of the ADC to kill Kasumi-1 cells (an AML cell line), which express human CD117. As described in FIG. 4A, Anti-CD117 ADC C (referred to as “Anti-CD117-AM” in FIG. 4A) was titrated and incubated with Kasumi-1 cells for 6 days at 37C and viability was assessed by CellTiter-Glo. The control was a non-specific isotype matched ADC C. Non-cleavable conjugate C demonstrated potent killing of Kasumi-1 cells with an IC50 of 6.4 pM. In FIG. 4B, a similar cytotoxicity assay using Human CD34+ bone marrow cells collected was performed. Human CD34+ bone marrow cells were incubated with non-cleavable conjugate C or a corresponding isotype control ADC for 6 days. Cell killing was determined by flow cytometry. The non-cleavable conjugate C demonstrated potent killing of primary human CD34+ CD90+ cells with an IC50 of 5.1 pM.

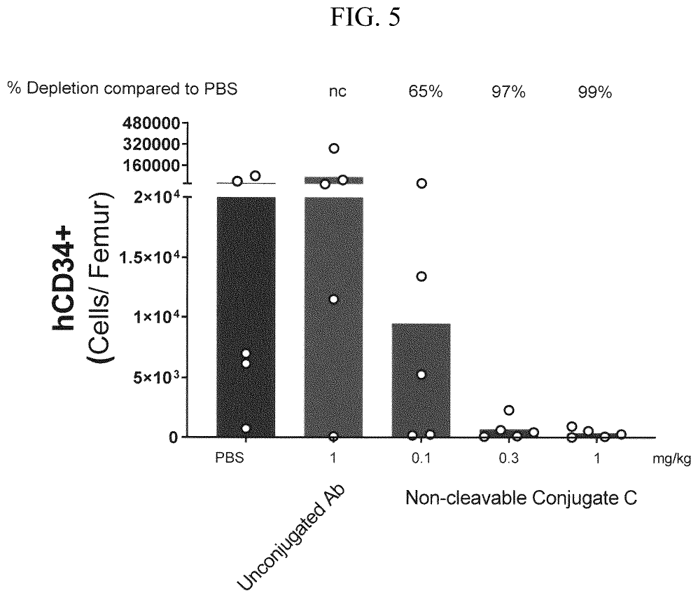

An in vivo assay was also performed to test the ability of anti-CD117 ADC C to selectively deplete human CD34+ cells. A single dose of 0.1, 0.3, or 1.0 mg/kg of the anti-CD117 ADC C was administered to humanized NSG mice. Controls consisted of a 1 mg/kg dose of the unconjugated antibody that comprised part of non-cleavable conjugate C and vehicle (PBS) treatment. At Day 21, the number of human CD34+ cells were determined by flow cytometry analysis of bone marrow collected from the femur of treated humanized NSG mice. As described in FIG. 5, anti-CD117 ADC C was able to potently deplete human CD34+ cells, achieving greater than 95% depletion compared to PBS for both 0.3 and 1 mg/kg doses. The unconjugated antibody demonstrated no depletion as compared to vehicle control.

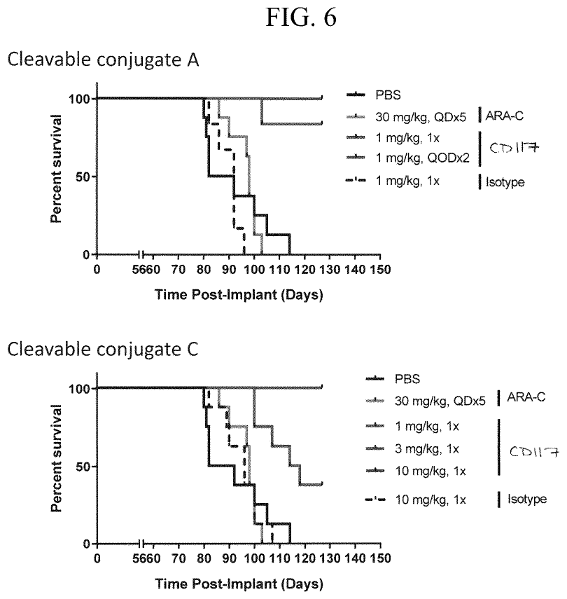

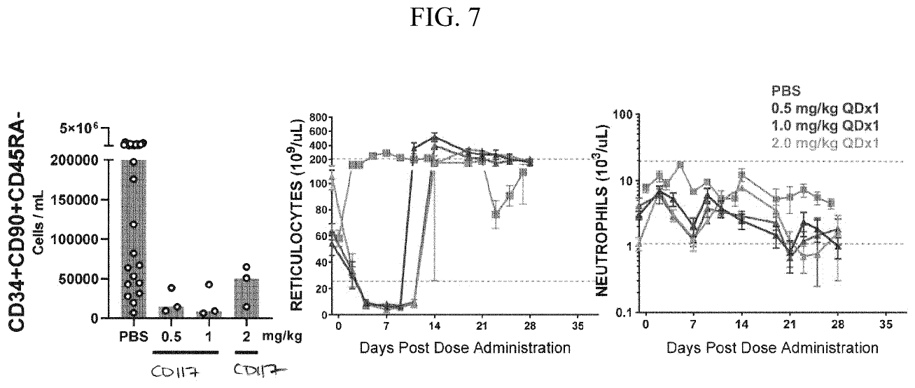

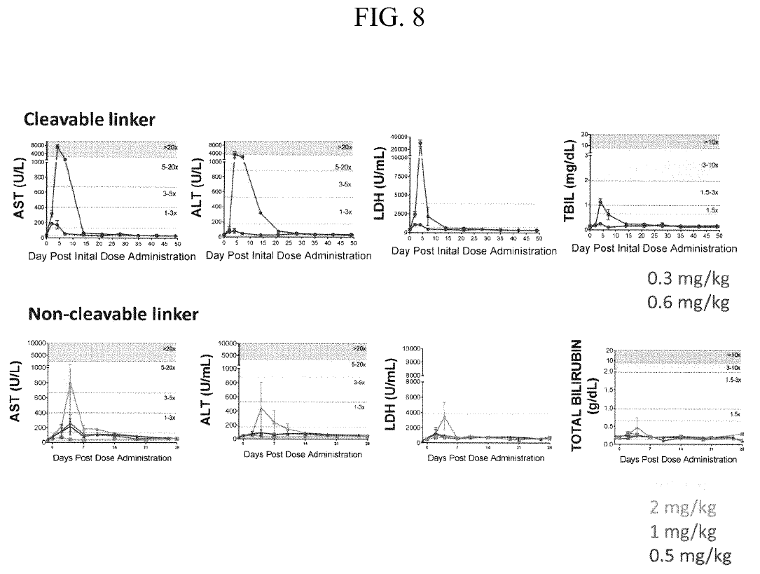

To assess in vivo anti-tumor activity of ADCs, humanized NSG mice were implanted with Kasumi-1 cells and then treated with vehicle, 30 mg/kg QDx5 cytarabine (ARA-C), cleavable conjugate A (1 mg/kg 1×, 1 mg/kg QOD×2) or 10 mg/kg isotype conjugated to cleavable amanitin (FIG. 1A). In addition, Kasumi-1 implanted mice were treated with non-cleavable conjugate C (1 mg/kg 1×, 3 mg/kg 1×, 10 mg/kg 1×) or 10 mg/kg isotype conjugated to non-cleavable amanitin (FIG. 1C). Kaplan-Meier curves demonstrating survival of animals treated as indicated is depicted in FIG. 6. Kasumi-1 implanted mice treated with cleavable conjugate A demonstrate median survival above 50% at day 130 for groups treated with either 1 mg/kg or 1 mg/kg QOD×2 in comparison to isotype, ARA-C, or vehicle treated animals. Mice treated with either 3 mg/kg or 10 mg/kg non-cleavable conjugate C demonstrate full survival at day 130 in comparison to isotype, ARA-C, 1 mg/kg non-cleavable conjugate C, or vehicle treated animals.

In summary, non-cleavable conjugate C demonstrates potent in vitro cell killing of Kasumi-1 cells, an AML cell line, and primary human CD34+ CD90+ bone marrow cells. Conjugate C is capable of depleting more than 95% of human CD34+ cells from the bone marrow of humanized NSG mice. Both cleavable and non-cleavable conjugates demonstrated potent anti-tumor activity in vivo.

### Example 3: Efficacy and Tolerability of ADC C in Non-Human Primates (NHPs)

To determine the efficacy of non-cleavable conjugate C in non-human primates, male cynomologus monkeys were treated with a single dose of non-cleavable conjugate C at 0.5, 1.0, and 2.0 mg/kg. The 0.5 and 1.0 mg/kg doses were with Ab85 with the following Fc modifications: D265C H435A (EU numbering) conjugated to non-cleavable amanitin C (FIG. 1C). The 2.0 mg/kg dose comprised Ab85 with the following Fc modifications: L234A L235A D265C H435A (EU numbering) conjugated to non-cleavable amanitin C (FIG. 1C). Bone marrow asipirates were collected from treated animals on day 7 and efficacy was assessed by quantitiation of CD34+ CD90+ CD45RA− populations by flow cytometry. Significant depletion was observed at 0.5 and 1.0 mg/kg doses. Peripheral blood samples were collected throughout the course of the 28 day study. Reticulocyte counts of animals treated at 0.5, 1.0, and 2.0 mg/kg decreased significantly beginning on day 2 with reticulocyte rebound occurring in a dose-dependent manner. This is expected on-target pharmacology as reticulocytes are short-lived and progenitor populations in this hematopoietic lineage strongly express CD117. Neutrophil depletion was observed beginning on day 18 for all treatment groups. This is also expected on-target pharmacology, as neutrophils are dependent on hematopoietic stem cell differentiation for regeneration.

In addition to efficacy, tolerability of ADCs containing either anti-CD117 ADC A or anti-CD117 ADC C were tested in NHPs. As described in FIG. 8, treatment of cynomologus monkeys with a 0.3 mg/kg dose of cleavable conjugate A conjugated to antibody CK6 (Fc modifications D265C and H435A) resulted in mild transient and reversible elevation of aspartate aminotransferase (AST) levels, with no elevation above normal range for alanine aminotransferase (ALT), lactate dehydrogenase (LDH), or total bilirubin (TBIL) over the course of the study. Treatment of animals with a 0.6 mg/kg dose of conjugate A resulted in more significant elevation of AST, ALT, LDH, and TBIL.

In cynomolgus monkeys treated with 0.5 and 1.0 mg/kg non-cleavable conjugate C, Ab85 with the following Fc modifications: D265C H435A (EU numbering) conjugated to non-cleavable amanitin C (FIG. 1C), demonstrated mild transient and reversible elevation in AST with no elevation above normal range for ALT, LDH, and TBIL throughout the course of the study. In animals treated with 2.0 mg/kg non-cleavable conjugate C, Ab85 with the following Fc modifications: L234A L235A D265C H435A (EU numbering) conjugated to non-cleavable amanitin C (FIG. 1C), more significant elevation was observed for AST and ALT.

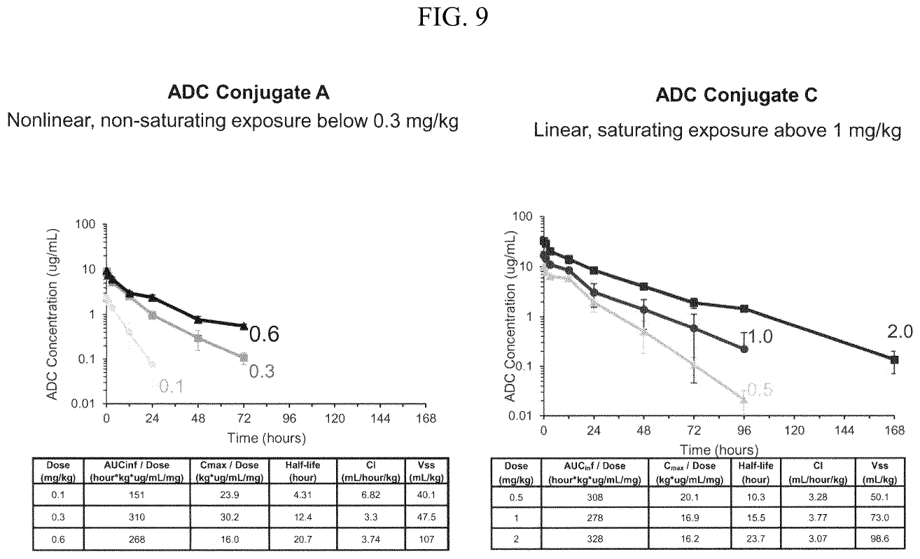

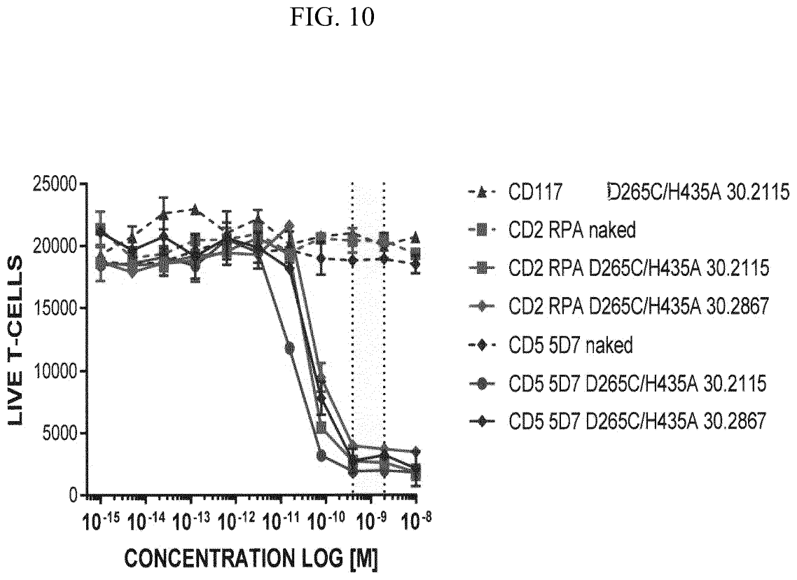

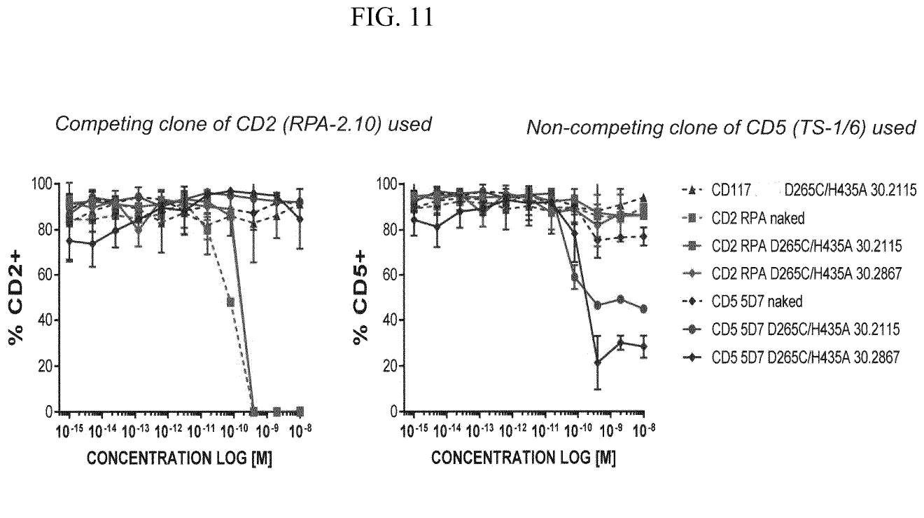

Pharmacokinetic characteristics were also studied in anti-CD117 ADC A vs. C. As described in FIG. 9, well tolerated doses of ADC conjugate C achieved saturating explosure in NHPs compared to ADC Conjugate A. Pharmacokinetic data was also analyzed for ADC conjugate C with various Fc mutations. For both anti-CD117 ADC A and anti-CD117 ADC C, ex vivo cytotoxicity assays revealed that for boths ADCs there was a loss of efficacy at plasma concentrations below 100 ng/ml (data not shown).

In sum, anti-CD117 ADC C demonstrated efficacy at doses as low as 0.5 mg/kg and was tolerated at a single dose of 2.0 mg/kg in cynomologus monkeys while the cleavable conjugate A was efficacious at a dose as low as 0.3 mg/kg but not tolerated at a single dose of 0.6 mg/kg supporting the finding that non-cleavable amanitin (FIG. 1C) is better tolerated.

### Example 4: Primary Human T-Cell Killing with Anti-CD2 ADC C and CD5 ADC C

A study was performed to determine if anti-CD2 and anti-CD5 D265C H435A antibodies used in an ADC C (as described above and in FIG. 1A to FIG. 1C) was as efficacious as Conjugate A for killing primary T cells. Thus, an in vitro cell killing assay was used. The protocol of the study is provided below:

General Protocol T cell killing assay: 384 well format

Coat plate with 0.5 mg/mL anti-CD3 incubate in 37 C. for 2 hours in TC treated
low flange 384 well plate
Wash plate
Thaw primary T cells with AIM-V media
Plate 2500 cells per well + 20 ng recombinant human IL-2
Titrate antibodies and add to cells in duplicate for 30 min on ice
Culture for in 37° C. for 5 days
On day 5, stain cells for CD3, Live/Dead marker for 30 min, using Viaflo aspirate
volume and add PBS
Run cells on Celesta
Readout: viability, CD2 and CD5 expression by flow

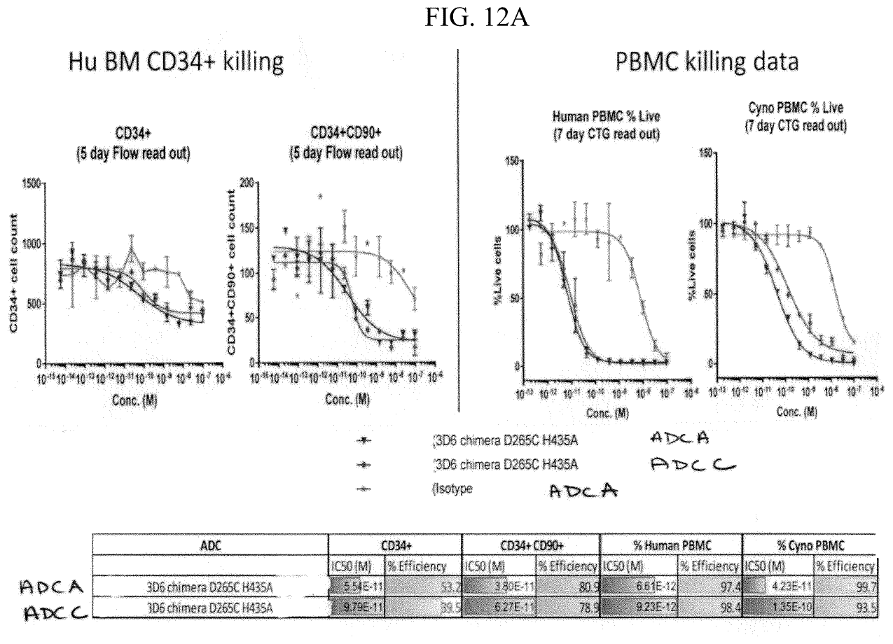

Anti-CD2 ADCs A and C included anti-CD2 antibody RPA with FC modifications D265C/H435A. Naked RPA was used as a control. Anti-CD5 ADCs A and C included anti-CD5 anitbody 5D37 with Fc modifications D265C/H435A. Naked 5D37 was used as a control. As described in FIG. 12A and FIG. 121B, Conjugate C demonstrated T cell depletion comparable to Conjugate A. Efficacy results are also provide in the table below.

| Label | DAR | IC50 (pM) | % Efficacy |
|---|---|---|---|
| Ab85 D265C/H435A ADC A | 1.9 | — | 0.9 |
| CD2 RPA naked | — | — | 0.0 |
| CD2 RPA2.10 D265C/H435A ADC A | 2.1 | 54.0 | 85.7 |
| CD2 RPA2.10 D265C/H435A ADC C | 2.1 | 86.9 | 78.7 |
| CD5 5D7 naked | — | — | 0.0 |
| CD5 5D7 D265C/H435A ADC A | 2 | 22.1 | 89.4 |
| CD5 5D7 D265C/H435A Ab ADC C | 2.0 | 50.8 | 84.9 |

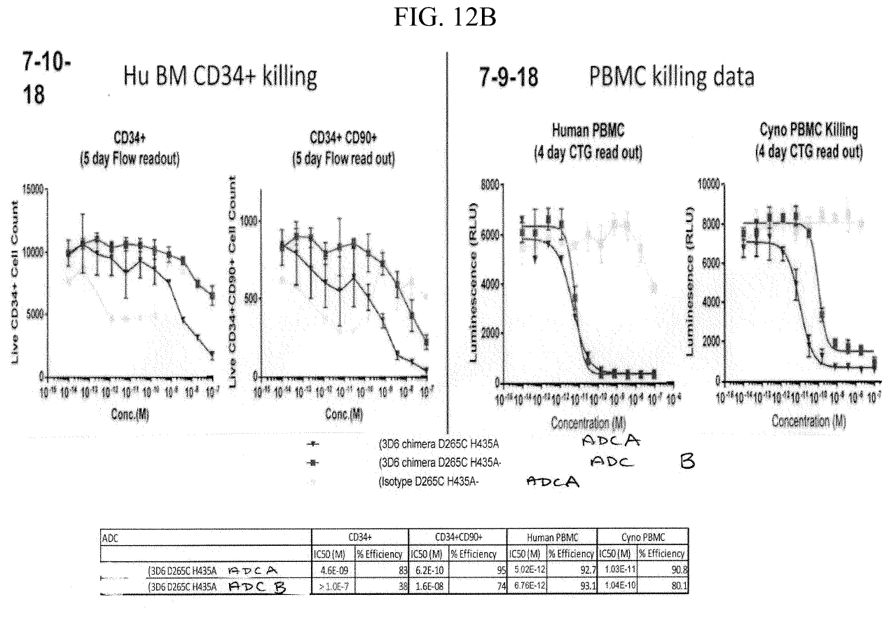

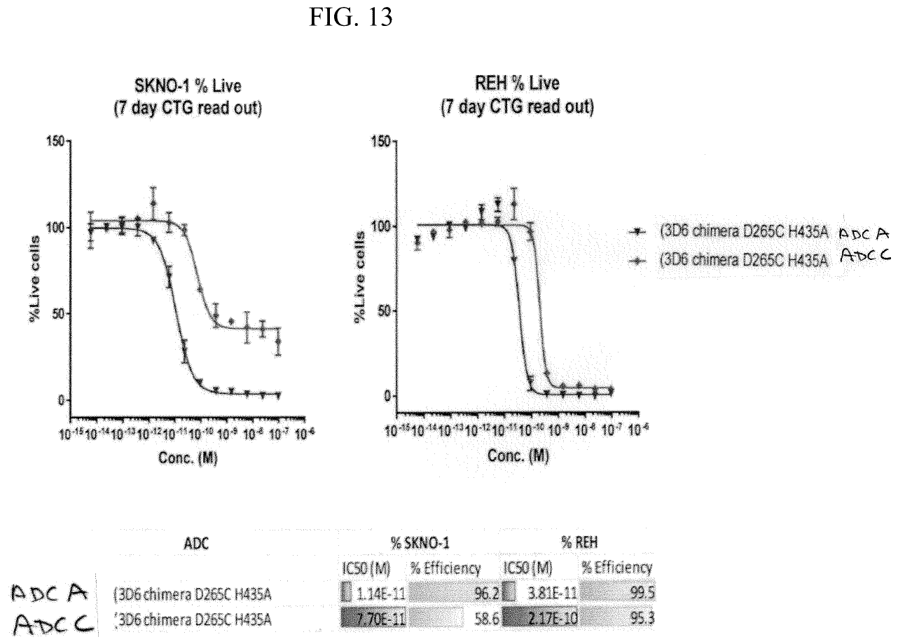

FIG. 13 provides results showing that CD2 is saturated by day 5, whereas some cells still express CD5.

The results above and in FIG. 12A, FIG. 12B and FIG. 13 show that both anti-CD2 and anti-CD5 ADCs C (D265C H435A) were as efficacious as Conjugate A on primary human T-cells in vitro.

### Example 5: Efficacy of Anti-CD45 ADC C

In addition to CD2, CD5, and CD117, Conjugate C was tested in comparison to Conjugate A in the context of anti-CD45 antibodies. A chimeric anti-CD45 antibody 3D6 was used in the below experiments (murine 3D6 variable regions and a human IgG1 framework with Fc modifications D265C and H435A).

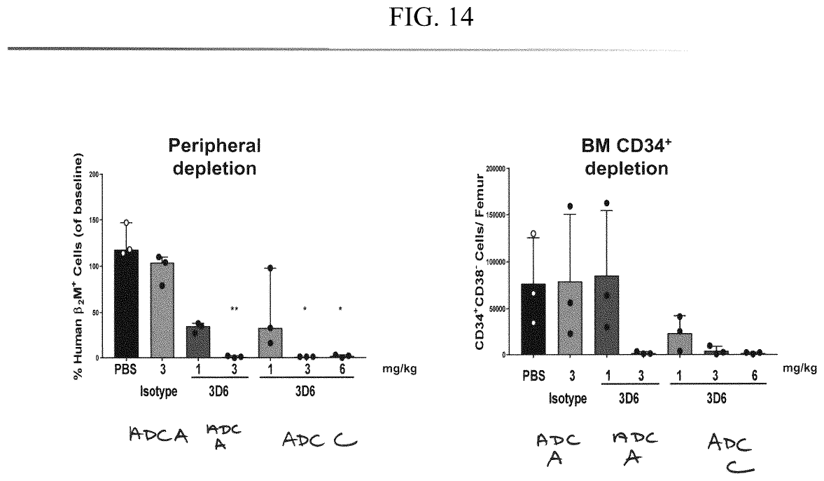

An in vitro cell killing assay was performed comparing anti-CD45 ADC A and anti-CD45 ADC C. As described in FIG. 14A, both ADCs A and C were able to kill human bone marrow CD34+ cells, as well as PBMC. An isotype negative control was also tested. As described in the Table presented in FIG. 14A, both anti-CD45 ADCs A and C were effective at killing CD34+ bone marrow and PBMC cells. This assay was repeated with anti-CD45 ADC A and B, As described in FIG. 14B, anti-CD45 ADC A showed a higher efficiency for killing CD34+ bone marrow cells vs. anti-CD45 ADC B. Both molecules were effective in a similar manner at killing human and cyno PBMCs.

A second in vitro cell killing assay was also performed using anti-CD45 ADCs A and C looking at the ability of each ADC to kill cells expressing either SKNO-1 or REH. The results are provided in FIG. 13 (left panel are the number of live SKNO-1 cells following exposure to anti-CD45 ADC A or C; right panel are the number of live REH cells following exposure to anti-CD45 ADC A or C). The efficiency of each molecule in these assays is described in the table in FIG. 13.

Anti-CD45 ADCs A and C were further tested for the ability of each ADC to deplete CD45+ cells in vivo. Both ADC A (cleavable) and ADC C (non-cleavable) were able to deplete CD45+ cells efficiently in a humanized NSG mouse model. The results of the study are provided in FIG. 14. As described in FIG. 16, both ADCs were able to deplete peripheral CD45+ cells as well as bone marrow (BM) CD34+ cells. The maximum total dose in the mice for anti-CD45 ADC A was 3 mg/kg, whereas a much higher dose of ADC C was tolerated for anti-CD45 ADC C at more than 51 mg/kg. Thus, efficacy was observed for both anti-CD45 ADCs, but the ADC C was tolerated by the mice at higher doses.

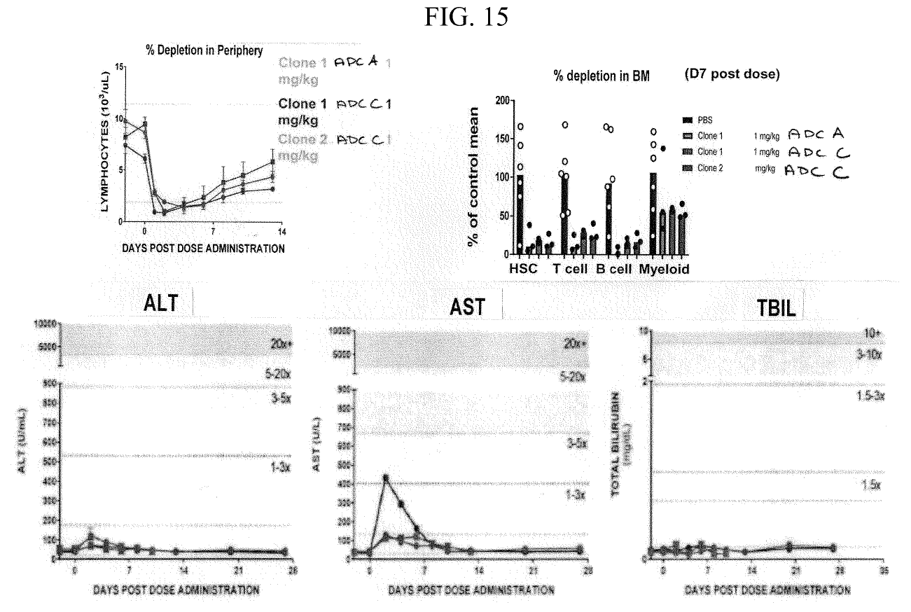

Anti-CD45 ADCs A and C were also tested for their ability to deplete peripheral lymphocytes, bone marrow (BM) HSCs and lymphocytes in humanized NSG mice. Anti-CD45 ADCs A and C were each delivered at a dose of 1 mg/kg to the mice. The results from these studies are described in FIG. 15 and show that anti-CD45 ADCs A and C had comparable depletion of peripheral lymphocytes, HSCs, and BM lymphocytes at day 7 post injection at 1 mg/kg. Anti-CD45 ADC A showed mild, transient reversible liver enzyme elevation at 1 mg/kg, while little to no liver enzyme elevation was observed with anti-CD45 ADC C with the same 1 mg/kg dose.

### Example 6: Anti-CD137 ADC A and C Characterization

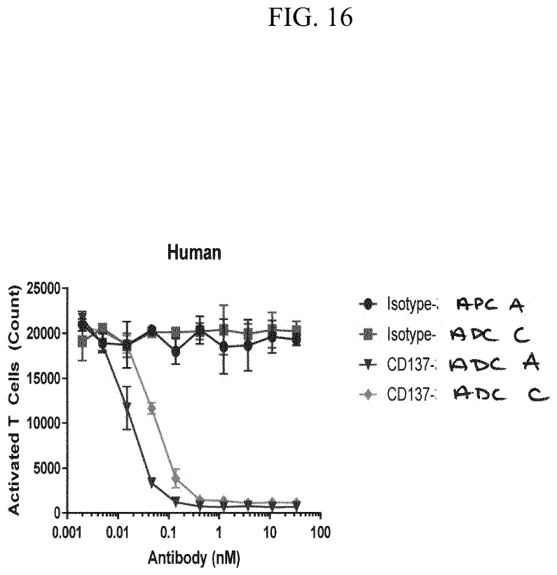

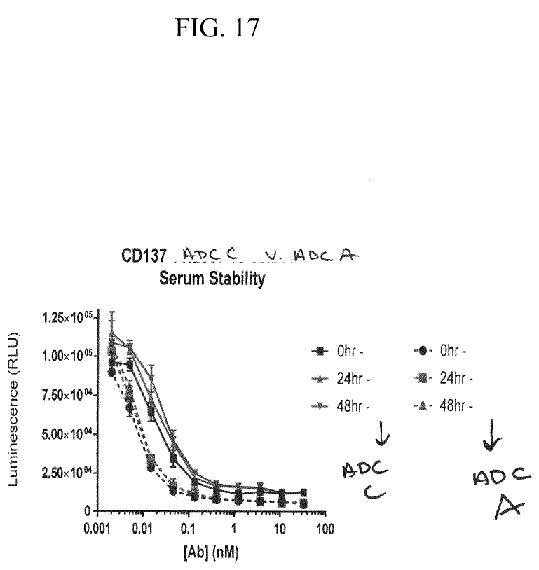

Anti-CD137 ADC A and ADC C were tested for their ability to kill T cells, as well as for serum stability. Anti-CD137 antibody BBK2 was used in this example. An in vitro T cell killin assay was performed, where the results shown in FIG. 16 demonstrated both anti-CD137 ADC A and C were able to kill activated T cells in comparison to the isotype ADC A and C control. In addition, cell line serum stability was tested for both anti-CD137 ADC A and C over 48 hours, as described in FIG. 17.

### Example 7: Synthesis of S-desoxy-5′-hydroxy-amaninamide

The ADCs of formula (IIa) as disclosed herein were prepared from the corresponding linker-amatoxin conjugates according to standard methods known to one of skill in the art. The penultimate amatoxin-linker conjugate to the ADCs of formula (IIa) (compound 14; Scheme 3) may be prepared from amatoxin derivative 11 following the general procedure for O-alkylation of the related amatoxin (α-amanitin) disclosed in U.S. Patent Application Publication No. 2018/0043033, the disclosure of which is incorporated by reference herein in its entirety. Amatoxin derivative 11 may be prepared according to the procedures disclosed in International Patent Application Publication No. WO2019/030173, the disclosure of which is incorporated by reference herein in its entirety. Compound 7 (Scheme 1) may be prepared according to the methods reported in International Patent Application Publication No. WO2014/009025, the disclosure of which is incorporated herein in its entirety.

### Example 8: Amanitin Derivatives

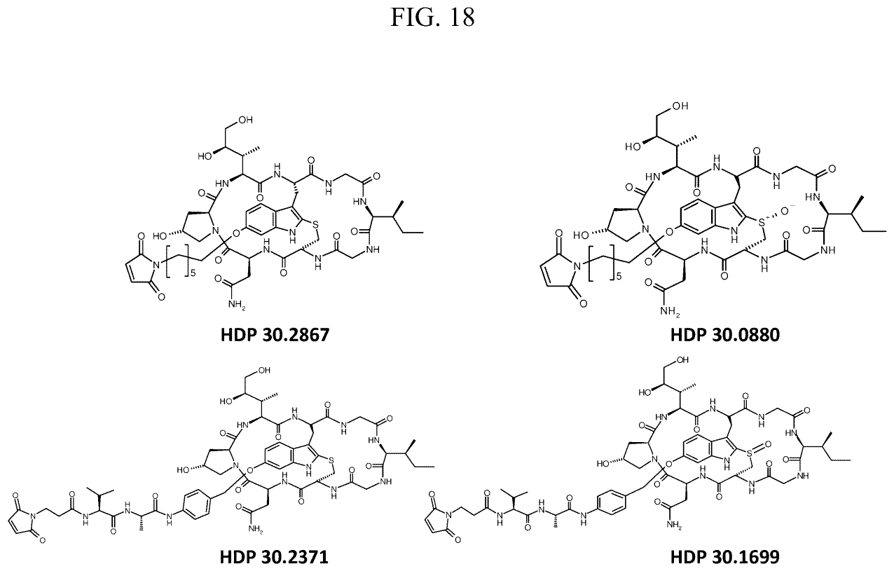

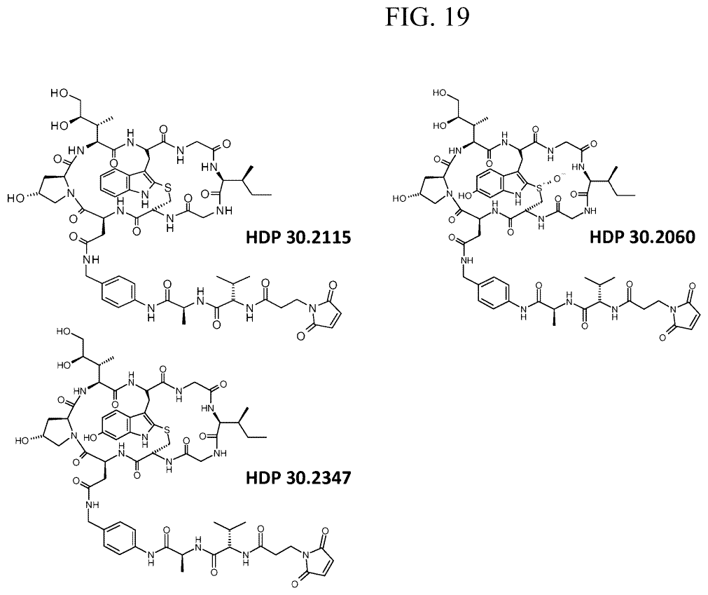

Various amanitin derivatives were used for conjugation with target-specific antibodies (FIG. 18, FIG. 19). They were generated according to the methods as disclosed in WO2018/115466 and WO2019/197654, the disclosures of which are incorporated herein in its entirety.

TABLE 6
Molecular characteristics of amatoxin-linker constructs

| Applicant's | Tryptophan | Sulfur | | |
|---|---|---|---|---|
| identifier | (Trp) | bridge | Linker at | Linker |
| 30.2867 | 6-Hydroxy-Trp | thioether | aa4 | stable C6-linker |
| 30.0880 | 6-Hydroxy-Trp | sulfoxide | aa4 | stable C6-linker |
| 30.1699 | 6-Hydroxy-Trp | sulfoxide | aa4 | cleavable Val-Ala |
| 30.2371 | 6-Hydroxy-Trp | thioether | aa4 | cleavable Val-Ala |
| 30.2060 | 6-Hydroxy-Trp | sulfoxide | aa1 | cleavable Val-Ala |
| 30.2115 | Trp | thioether | aa1 | cleavable Val-Ala |
| 30.2347 | 6-Hydroxy-Trp | thioether | aa1 | cleavable Val-Ala |

### Example 9: Method of Conjugation

Antibodies directed to Her2 or prostate-specific membrane antigen (PSMA) were conjugated to the amatoxin linker conjugates by means of the so-called Thiomab technology. In this approach, the conjugation takes place by conjugation of the maleimide residue of the toxin linker construct to the free SH group of an engineered cysteine residue in the antibody, as shown in the following reaction scheme:

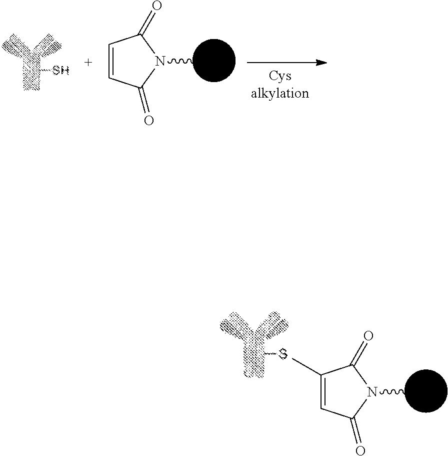

The principles of this conjugation method, are disclosed in Junutula et al (2008), the content of which is incorporated herein by reference.

The antibodies directed to Her2 (Trastuzumab) or prostate-specific membrane antigen (PSMA) used in the present experiments comprise a D265C substitution in both Fc domains, in order to provide a cysteine residue that has such free SH group. The respective technology is disclosed in WO2016142049 A1 assigned to the present applicant, the content of which is incorporated herein by reference, and delivers a homogenous product with a fixed drug to antibody ratio (“DAR”) of 2 and a site specific conjugation.

### Example 10: Cytotoxicity Assay with Anti-Her2-ADCs In Vitro

Cytotoxic activity of anti-Her2 amatoxin conjugates was evaluated in vitro with target-positive tumor cell lines and the chemiluminescent BrdU incorporation assay (Roche Diagnostics). Cell viability was determined after 96 h incubation with different concentrations of conjugates at 37° C. and 5% CO₂ by measurement of fixed and permeabilized cells with an anti-BrdU-HRP antibody in a BMG Labtech Optima microplate reader. EC₅₀ value of dose-response curve was calculated by Graphpad Prism 4.0 software.

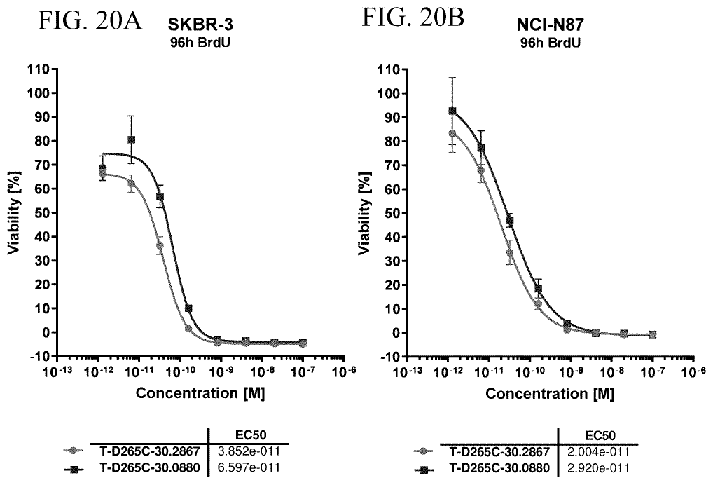

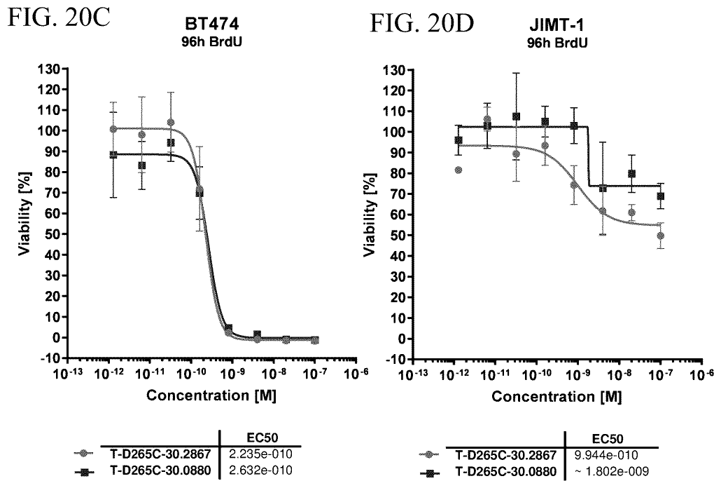

The cytotoxic activity of Trastuzumab carrying a D265C mutation (T-D265C) conjugated to compound 30.2867 (formula (A)), and to compound 30.0880 (formula (B)), respectively, via its D265C residue was tested on SKBR-3, NCI-N87, BT474 and JIMT-1 cell lines (FIG. 20A to FIG. 20D). T-D265C-30.2867 slightly outperformed T-D265C-30.0880 on all tested cell lines.

### Example 11: Stability of Anti-Her2-ADCs in Human, Mouse, and Cynomolgus Plasma

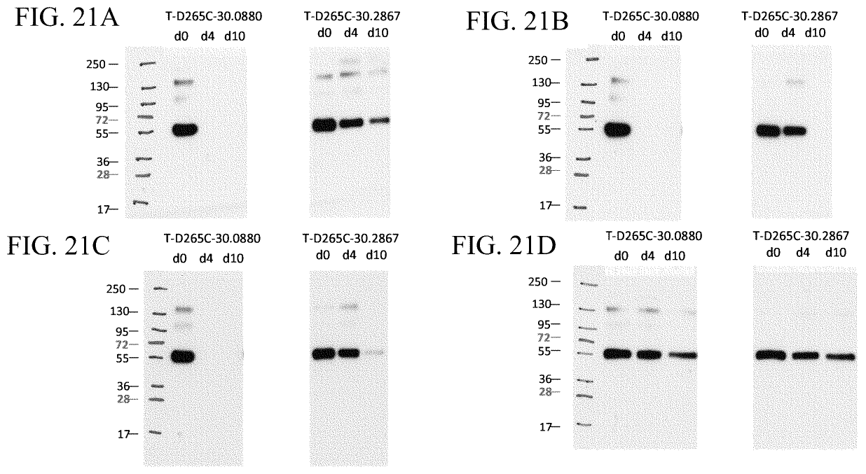

ADC conjugates T-D265C-30.2867 and T-D265C-30.0880, respectively, directed to Her2 antigen, were incubated with human, mouse, and cynomolgus plasma, and phosphate buffered saline (PBS, control), respectively, for 0, 4, and 10 days. Samples were subjected to sodium dodecyl sulphate (SDS) polyacryl amide (PAA) gel electrophoresis SDS-PAGE), and antibody heavy chain molecules were detected with an amanitin-specific polyclonal antiserum by Western blotting (FIG. 21A to FIG. 21D).

The ADC conjugate T-D265C-30.2867 showed higher plasma stability over all tested species compared to the ADC conjugate T-D265C-30.0880. T-D265C-30.2867 showed lower stability in MP than in HP and CP, respectively.

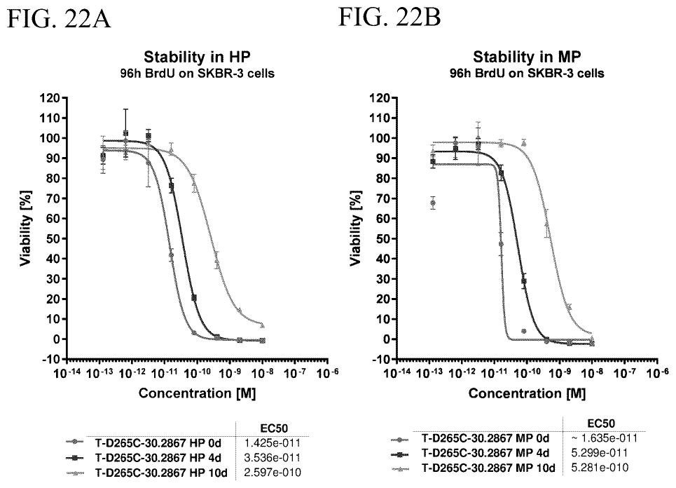

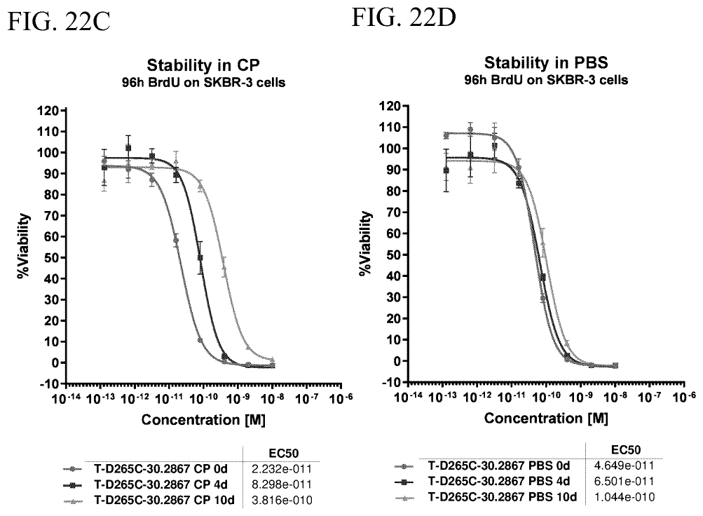

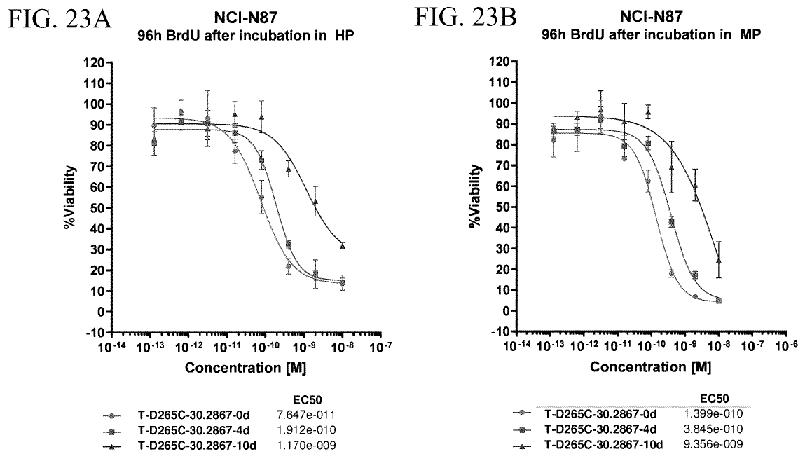

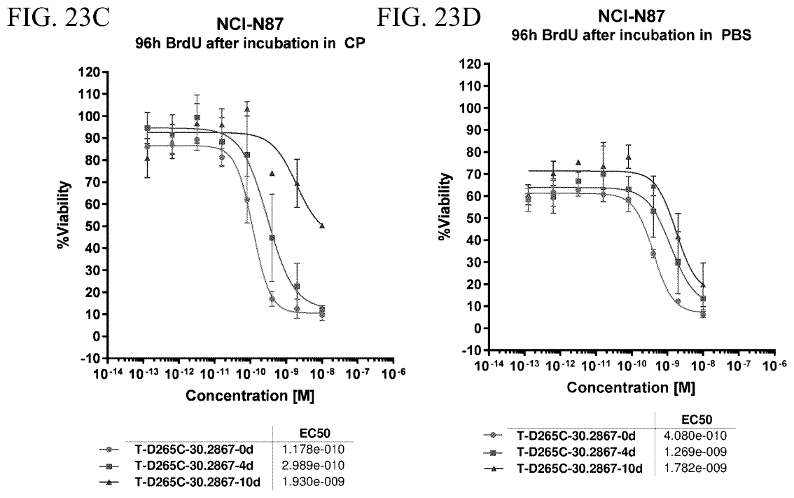

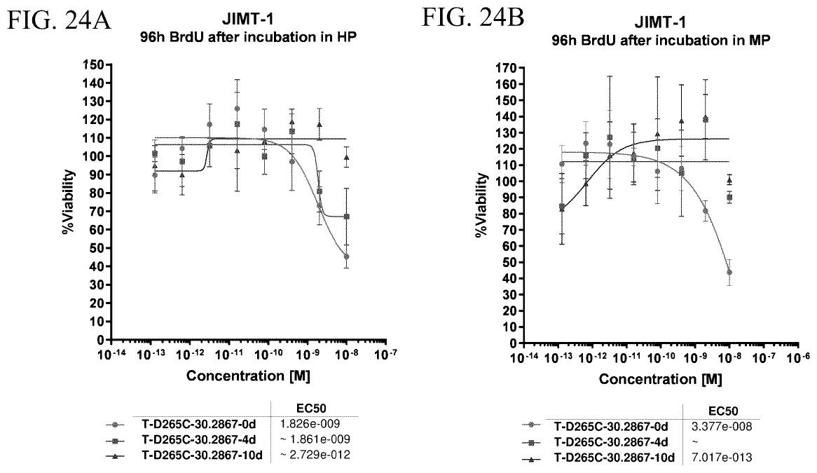

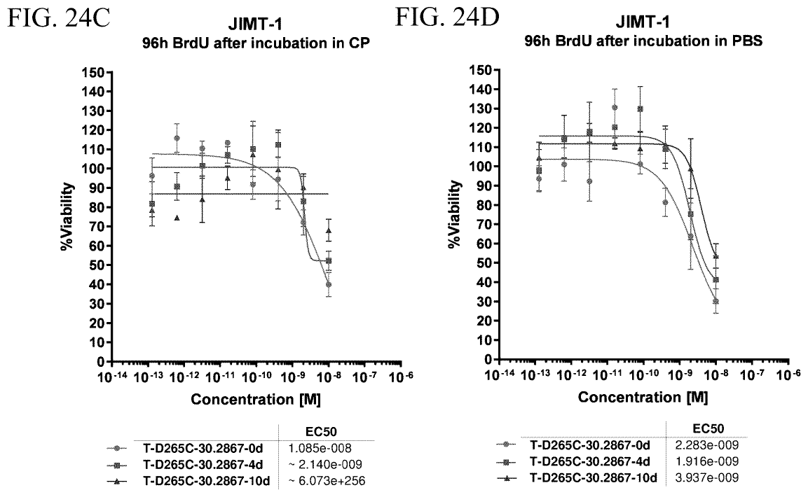

For further assessment of stability, the cytotoxic activity in vitro of ADC compound T-D265C-30.2867 after incubation for 0, 4 and 10 days, respectively, in human plasma, mouse plasma, cynomolgus plasma, and PBS (phosphate buffered saline, control), respectively, was analyzed on SKBR-3 cells (FIG. 22A to FIG. 22D), on NCI-N87 cells (FIG. 23A to FIG. 23D), and on JIMT-1 cells (FIG. 24A to FIG. 24D).

After 4-day incubation in human plasma, mouse plasma, and cynomolgus plasma, respectively, the cytotoxic potential of T-D265C-30.2867 on SKBR-3 cells was only slightly reduced. After 10-day incubation in human plasma and cynomolgus plasma, the cytotoxic potential was reduced by factor 20, in mouse plasma, however, a reduction by factor 33 was observed. Incubation in PBS at 37° C. had almost no effect on cytotoxic potential of T-D265C-30.2867 on SKBR-3 cells (FIG. 22A to FIG. 22D, Table 7).

TABLE 7
Stability and cytotoxic potential on SKBR-3 cells
of T-D265C-30.2867 in human, mouse and cynomolgus
plasma; values are EC50 values [nM]

| | | | | Reduction of cytotoxic |
|---|---|---|---|---|
| Matrix | 0 days | 4 days | 10 days | potential compared to day 0 |
| Human plasma | 14 | 35 | 260 | 18.6× |
| Mouse plasma | 16 | 53 | 528 | 33.0× |
| Cynomolgus | 22 | 83 | 382 | 17.4× |
| plasma | | | | |
| PBS | 46 | 65 | 104 | 2.3× |

After 4-day incubation in human plasma, mouse plasma and cynomolgus plasma, respectively, the cytotoxic potential of T-D265C-30.2867 on NCI-N87 cells was only slightly reduced. After 10-day incubation in human plasma and cynomolgus plasma, the cytotoxic potential was reduced by factor 15, however, app. 50% residual cell viability was observed after 10-day incubation in cynomolgus plasma. After 10-day incubation in mouse plasma, a reduction by factor 67 was observed. Incubation in PBS at 37° C. had a low effect on cytotoxic potential of T-D265C-30.2867 on NCI-N87 cells (FIG. 23A to FIG. 23D).

After 4-day incubation in human plasma and cynomolgus plasma, the cytotoxic potential of T-D265C-30.2867 on JIMT-1 cells was only slightly reduced. After 4-day incubation in mouse plasma, T-D265C-30.2867 showed no cytotoxic potential at all on JIMT-1 cells. After 10-day incubation in human plasma, mouse plasma and cynomolgus plasma, no cytotoxic potential at all was observed on JIMT-1 cells. Incubation in PBS at 37° C. had almost no effect on cytotoxic potential of T-D265C-30.2867 on JIMT-1 cells (FIG. 24A to FIG. 24D).

### Example 12: Efficacy of Anti-Her2-ADCs in Mouse Xenograft Tumor Models In Vivo

In the JIMT-1 mouse xenograft model, female NMRI nude mice were inoculated with 5×10⁶ JIMT-1 breast cancer cells per mouse subcutaneously in the right flank. At a mean tumor vol. of ˜120 mm³, animals were allocated to three groups on day 0. On the same day, the animals received a single intravenous dose of amanitin-based anti-Her2 antibody drug conjugates (ADCs). The tumor volume and body weight were determined twice per week.

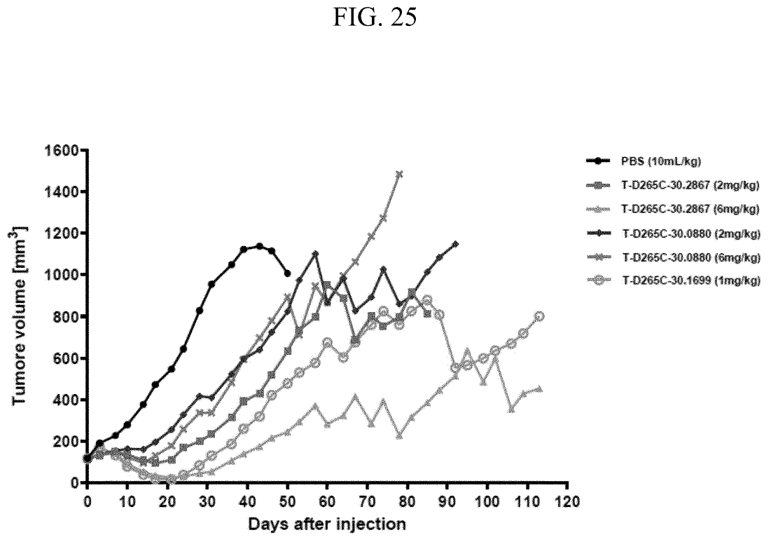

As shown in FIG. 25, the efficacy of T-D265C-30.2867 in vivo was better than that of T-D265C-30.0880. At 113 days after treatment, 0/10 animals which had received a dose of 2 mg/kg, and 4/10 animals which had received a dose of 6 mg/kg T-D265C-30.2867 were alive; in the latter group, 1/4 animals were tumor free. In contrast, at 113 days after treatment, 0/10 animals which had received a dose of 2 mg/kg, and 0/10 animals which had received a dose of 6 mg/kg T-D265C-30.0880 were alive. At 113 days after treatment, 4/10 animals which had received a dose of 1 mg/kg T-D265C-30.1699 were alive, and 1/4 animals was tumor free.

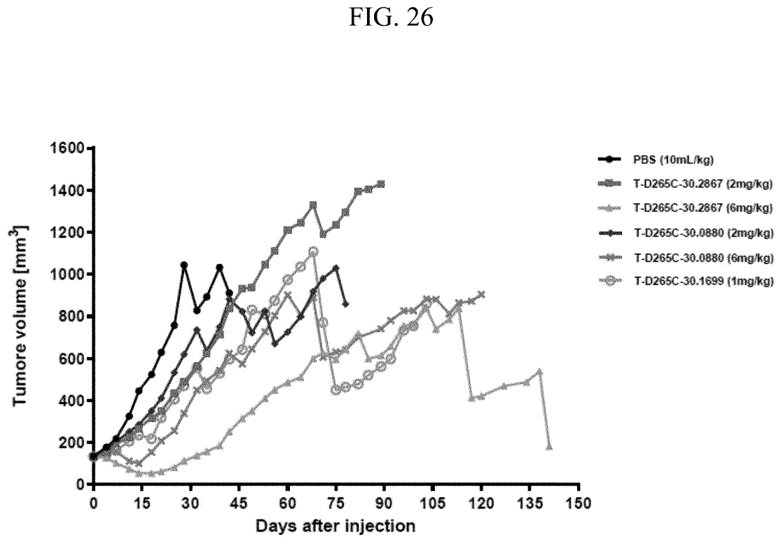

With a respective NCI-N87 mouse tumor xenograft model, as shown in FIG. 26, again, the cytotoxic efficacy of T-D265C-30.2867 in vivo was better than that of T-D265C-30.0880. At 141 days after treatment, 0/10 animals which had received a dose of 2 mg/kg, and 3/10 animals which had received a dose of 6 mg/kg T-D265C-30.2867 were alive; in the latter group, however, 0/3 animals were tumor free. In contrast, at 141 days after treatment, 0/10 animals which had received a dose of 2 mg/kg, and 0/10 animals which had received a dose of 6 mg/kg T-D265C-30.0880 were alive. At 141 days after treatment, 0/10 animals which had received a dose of 1 mg/kg T-D265C-30.1699 was alive either.

### Example 13: Cytotoxicity Assay with Anti-PSMA-ADCs In Vitro

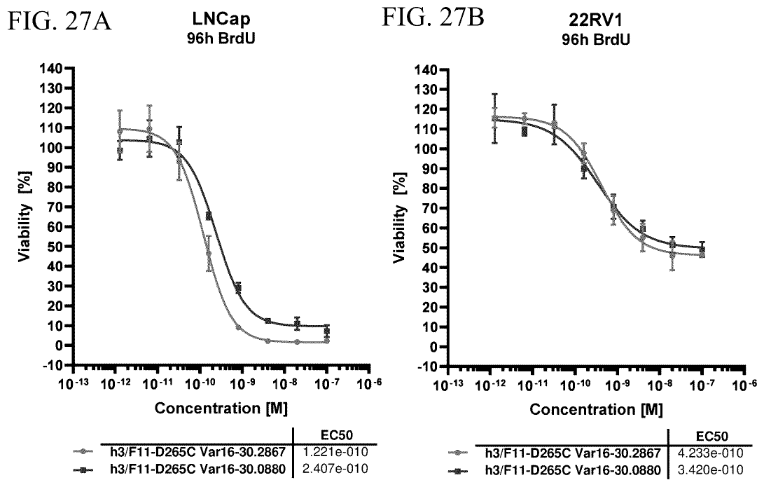

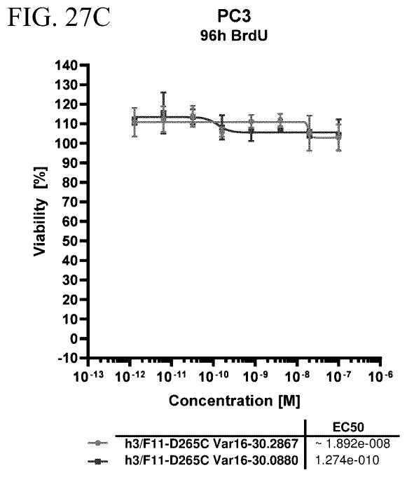

The cytotoxic activity in vitro of an anti-PSMA antibody carrying a D265C mutation (h3/F11-D265C-Var16) conjugated to compound 30.2867 (formula (A)), and to compound 30.0880 (formula (B)), respectively, via its D265C residue was tested on LNCap-, 22RV1-(both PSMA-positive), and PC3 (PSMA-negative) cell lines (FIG. 27A to FIG. 27C). The compound h3/F11-D265C-Var16-30.2867 slightly outperformed h3/F11-D265C-Var16-30.0880 on one out of two tested cell lines. No cytotoxic potential was observed on the PSMA-negative cell line PC3.

### Example 14: Stability of Anti-PSMA-ADCs in Human, Mouse, and Cynomolgus Plasma

ADC conjugates h3/F11-D265C-Var16-30.2867 and h3/F11-D265C-Var16-30.0880, respectively, directed to PSMA antigen, were incubated in human, mouse, and cynomolgus plasma, and phosphate buffered saline (PBS, control), respectively, for 0, 4, and 10 days. Samples were subjected to sodium dodecyl sulphate (SDS) polyacryl amide (PAA) gel electrophoresis SDS-PAGE), and antibody heavy chain molecules were detected with an amanitin-specific polyclonal antiserum by Western blotting.

h3/F11-D265C-Var16-30.2867 showed higher plasma stability over all tested plasma species compared to h3/F11-D265C-Var16-30.0880. h3/F11-D265C-Var16-30.2867 showed lower stability in mouse plasma than in human plasma and cynomolgus plasma, respectively.

### Example 15: Efficacy of Anti-PSMA-ADCs in Mouse Xenograft Tumor Model In Vivo

In the LNCap xenograft tumor mouse model, male CB17 SCID mice were inoculated with 2.5×10⁶ LNCap prostate carcinoma cells per mouse subcutaneously in the right flank. At a mean tumor vol. of ˜150 mm³, animals were allocated to groups on day 0. On the same day, the animals received a single intravenous dose of amanitin-based anti-PSMA antibody drug conjugates (ADCs). Tumor volume and body weight were determined twice per week.

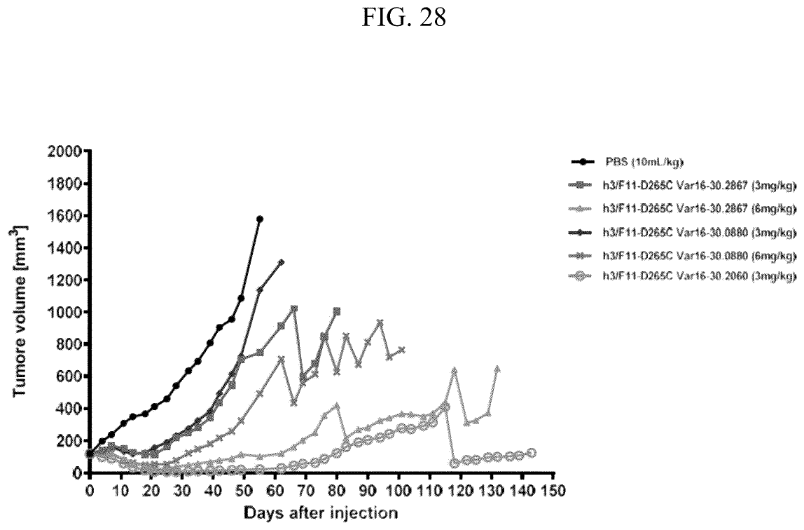

As shown in FIG. 28, the efficacy of h3/F11-D265C-Var16-30.2867 in vivo was better than that of h3/F11-D265C-Var16-30.0880.

### Example 16: Tolerability Study in Monkeys

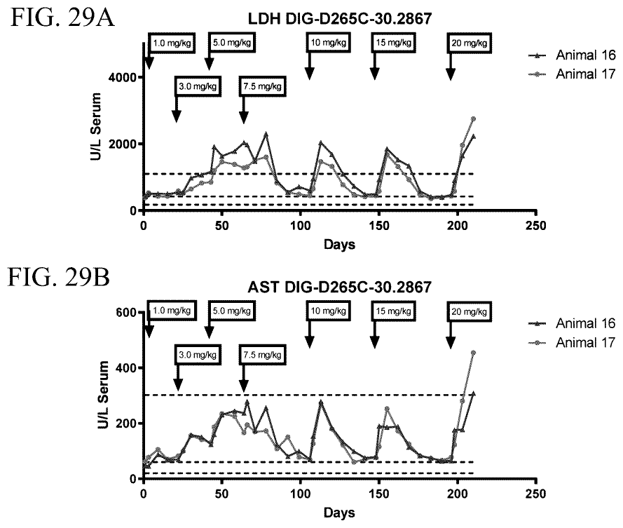

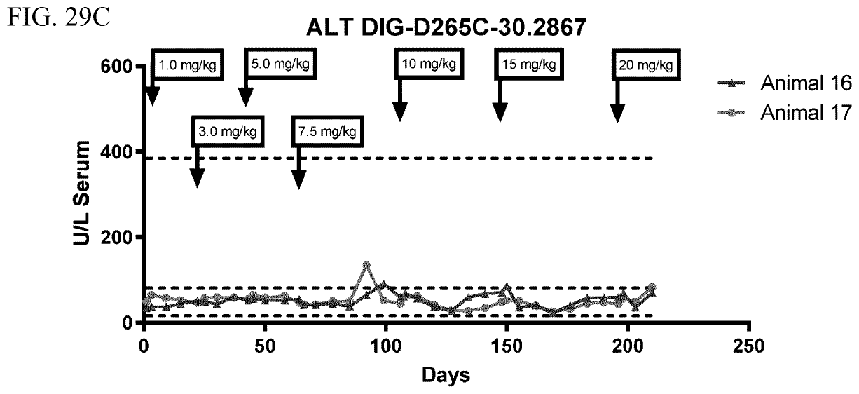

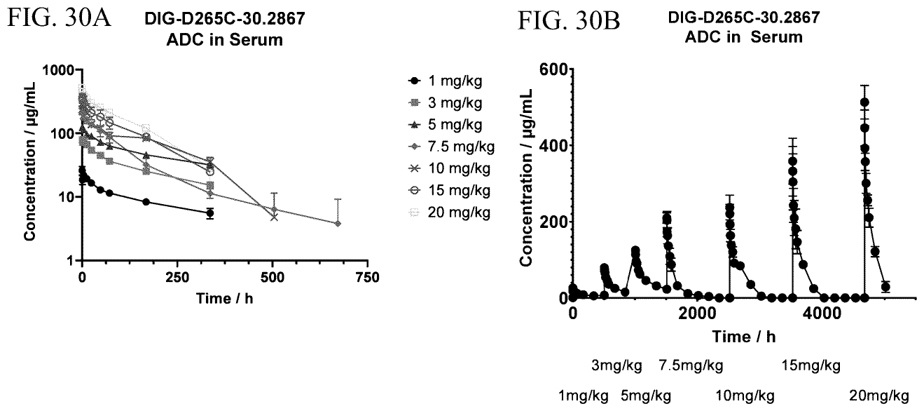

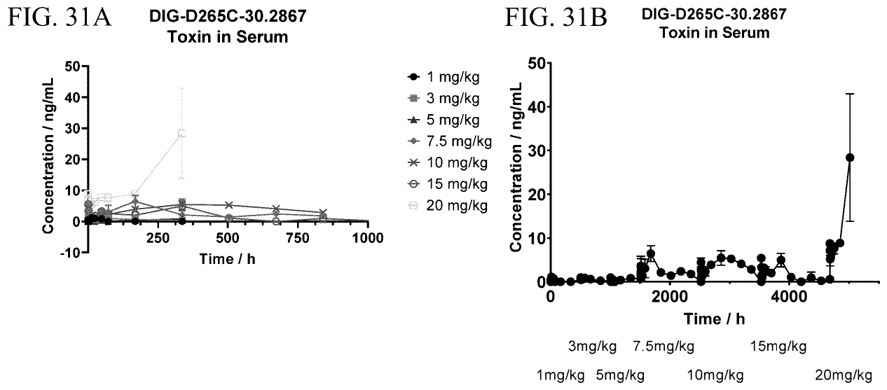

A tolerability study in monkeys with a non-binding Anti-Digoxigenin ADC (DIG-D265C-30.2867) using escalating doses from 1 mg/kg to 20 mg/kg was performed. Results for assessment of LDH, AST, ALT parameters indicated a good tolerability (FIG. 29A to FIG. 29C). Pharmacokinetic data for the ADC and the amatoxin in serum are shown in FIG. 30A and FIG. 30B and FIG. 31A and FIG. 31B, respectively. Both animals died at 15 days and 19 days, respectively, after the last dosing (20 mg/kg). The maximum tolerated dose (MTD) was assessed to be 15 mg/kg<MTD<20 mg/kg.

### Example 17: Cytotoxicity Assay In Vitro Using Structurally Different Amanitin Derivatives

The cytotoxic activity in vitro of ADCs listed in Table 8, which are comprising an anti-Her2 antibody carrying a D265C mutation (T-D265C, anti-Her2-D265C) conjugated to structurally different amanitin derivatives via its D265C residue was tested on JIMT-1 cells, NCI-N87 cells and SKBR-3 cells.

TABLE 8
Her2-specific ADCs comprising structurally
different amanitin derivatives

| ADC Compound | DAR | Specification |
|---|---|---|
| anti-Her2-D265C-30.2060 | 1.92 | SO; 6-OH-Trp; |
| | | cleavable VA linker at aa1 |
| anti-Her2-D265C-30.2115 | 2.0 | S; Trp; cleavable VA linker at aa1 |
| anti-Her2-D265C-30.2347 | 1.90 | S; 6-OH-Trp; |
| | | cleavable VA linker at aa1 |
| anti-Her2-D265C-30.1699 | 2.0 | SO; 6-OH-Trp; |
| | | cleavable VA linker at aa4 |
| anti-Her2-D265C-30.2371 | 2.0 | S; 6-OH-Trp; cleavable |
| | | VA linker at aa4 |
| anti-Her2-D265C-30.0880 | 2.0 | SO; 6-OH-Trp; C6 linker at aa4 |
| anti-Her2-D265C-30.2867 | 2.0 | S; 6-OH-Trp; C6 linker at aa4 |

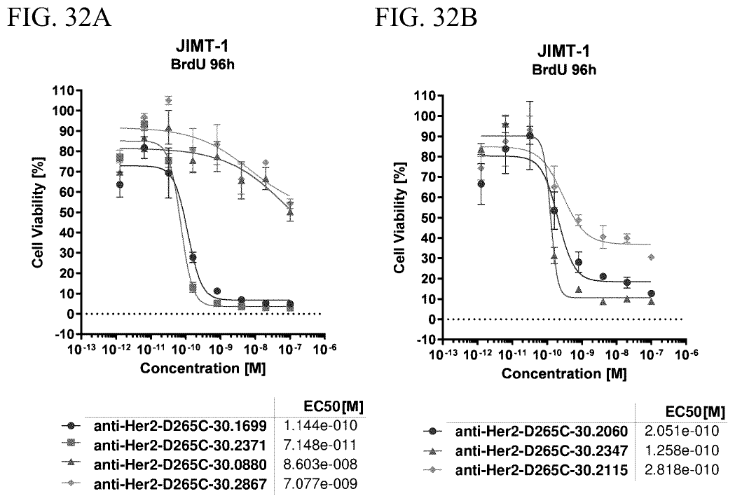

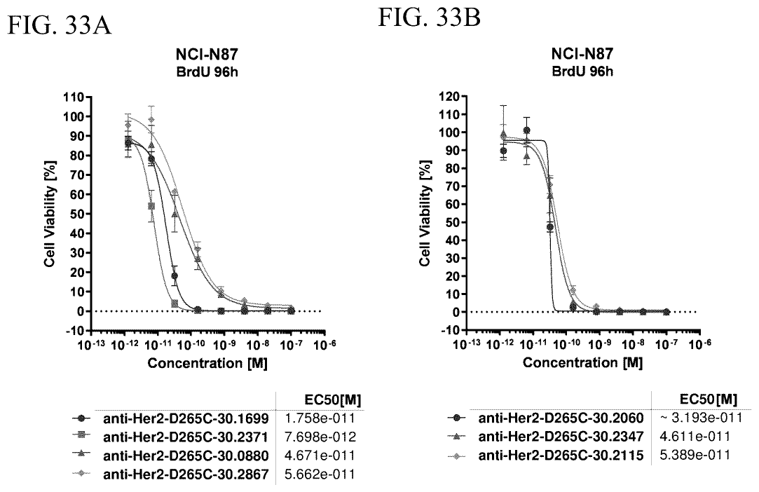

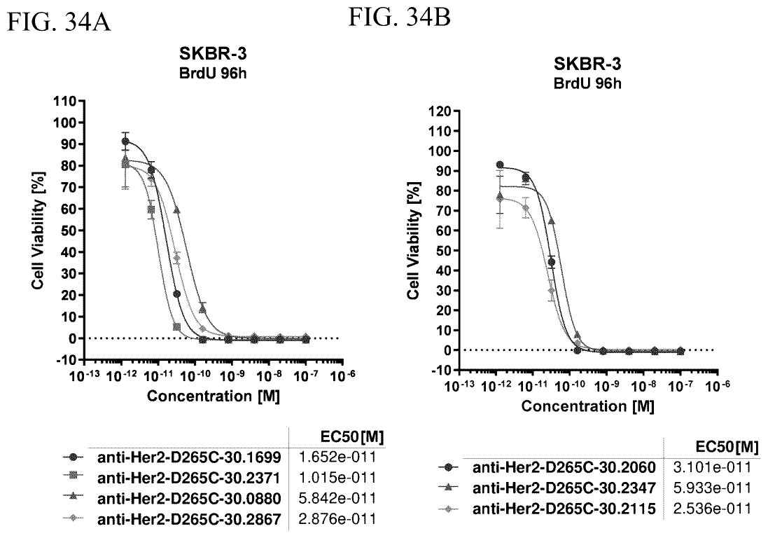

Cytotoxic efficacies of respective ADCs on JIMT-1 cells (FIG. 32A and FIG. 32B), NCI-N87 cells (FIG. 33A and FIG. 33B) and SKBR-3 cells (FIG. 34A and FIG. 34B), respectively, are shown in Table 9.

TABLE 9
Cytotoxic activities of Her2-specific ADCs comprising
structurally different amanitin derivatives

| | EC50 | EC50 | EC50 | |
|---|---|---|---|---|
| ADC | [pM] | [pM] | [pM] | MTD |
| Compound | on JIMT-1 | on NCI-N87 | on SKBR-3 | [mg/kg] |
| anti-Her2- | 205 (nfb: 20%) | 32 | 31 | ≥30 |
| D265C- | | | | |
| 30.2060 | | | | |
| anti-Her2- | 282 (nfb: 40%) | 54 | 25 | ≥25 |
| D265C- | | | | |
| 30.2115 | | | | |
| anti-Her2- | 126 (nfb: 10%) | 46 | 59 | ≥10 (<25) |
| D265C- | | | | |
| 30.2347 | | | | |
| anti-Her2- | 114 | 18 | 17 | 15 |
| D265C- | | | | |
| 30.1699 | | | | |
| anti-Her2- | 71 | 8 | 10 | ≥10 (<25) |
| D265C- | | | | |
| 30.2371 | | | | |
| anti-Her2- | 86000 | 47 | 58 | ≥60 |
| D265C- | (nfb: 50%) | | | |
| 30.0880 | | | | |
| anti-Her2- | 7077 (nfb: | 57 | 29 | ≥80 |
| D265C- | 50%) | | | |
| 30.2867 | | | | |

The cytotoxic activity in vitro of ADCs listed in Table 10, which are comprising an anti-PSMA antibody carrying a D265C mutation (h3/F11-D265C-Var16, anti-PSMA-D265C) conjugated to structurally different amanitin derivatives via its D265C residue was tested on LNCap- and 22RV1 cell lines (both PSMA-positive).

TABLE 10
PSMA-specific ADCs comprising structurally different amanitin derivatives

| ADC Compound | DAR | Specification |
|---|---|---|
| anti-PSMA-D265C-30.2060 | 1.89 | SO; 6-OH-Trp; cleavable VA linker at |
| | | aa1 |
| anti- PSMA-D265C-30.2115 | 2.0 | S; Trp; cleavable VA linker at aa1 |
| anti- PSMA-D265C-30.2347 | 1.91 | S; 6-OH-Trp; cleavable VA linker at aa1 |
| anti- PSMA-D265C-30.1699 | 2.0 | SO; 6-OH-Trp; cleavable VA linker at |
| | | aa4 |
| anti- PSMA-D265C-30.2371 | 2.0 | S; 6-OH-Trp; cleavable VA linker at aa4 |
| anti- PSMA-D265C-30.0880 | 2.0 | SO; 6-OH-Trp; C6 linker at aa4 |
| anti- PSMA-D265C-30.2867 | 2.0 | S; 6-OH-Trp; C6 linker at aa4 |

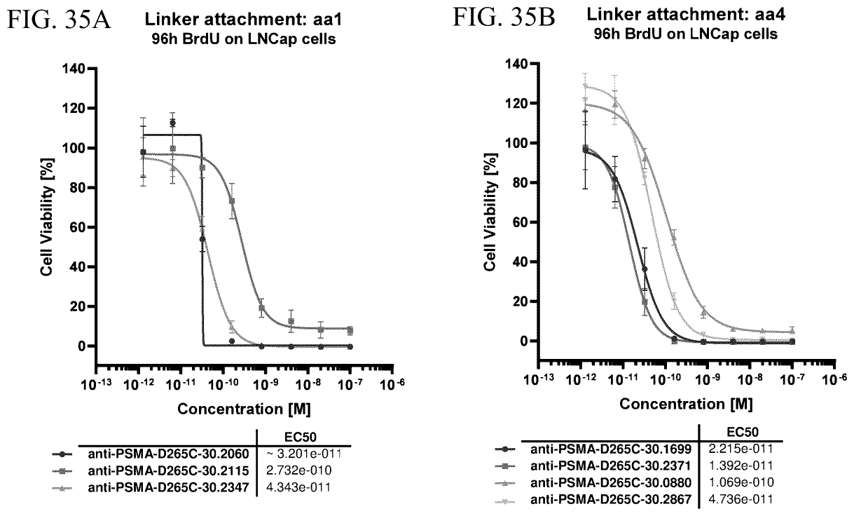

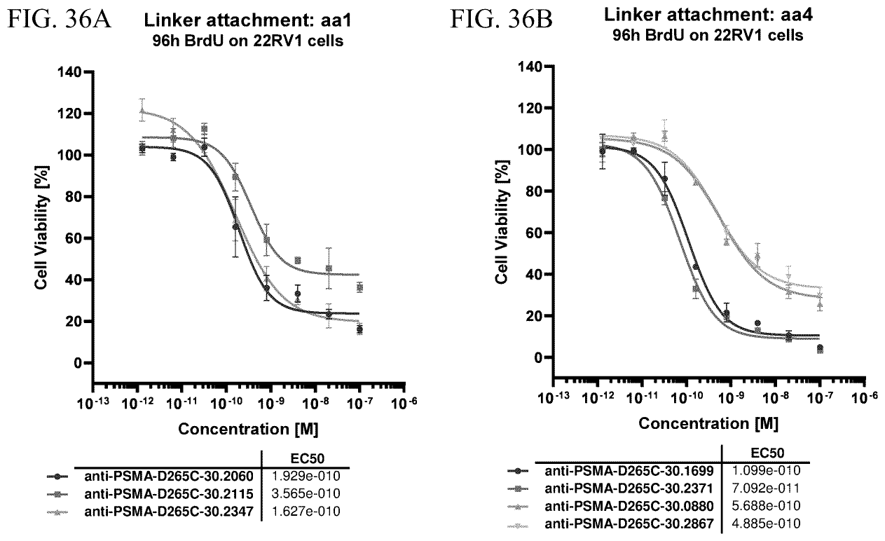

Cytotoxic efficacies of respective ADCs on LNCap cells (FIG. 35A and FIG. 35B) and 22RV1 cells (FIG. 36A and FIG. 36B), respectively, are shown in Table 11.

TABLE 11
Cytotoxic activities of PSMA-specific ADCs comprising
structurally different amanitin derivatives

| | EC50 | EC50 | |
|---|---|---|---|
| | [pM] | [pM] | MTD |
| ADC Compound | on LNCap | on 22RV1 | [mg/kg] |
| anti- | 32 | 193 (20%) | 50 < MTD < 80 |
| PSMA-D265C-30.2060 | | | |
| anti- | 273 | 357 (40%) | 30 |
| PSMA-D265C-30.2115 | | | |
| anti- | 43 | 163 (20%) | 15 |
| PSMA-D265C-30.2347 | | | |
| anti- | 22 | 110 (10%) | 10 |
| PSMA-D265C-30.1699 | | | |
| anti- | 14 | 71 (10%) | 8 |
| PSMA-D265C-30.2371 | | | |
| anti- | 107 | 569 (30%) | 30 |
| PSMA-D265C-30.0880 | | | |
| anti- | 47 | 486 (30%) | >80 |
| PSMA-D265C-30.2867 | | | |

### Example 18: Efficacy of Anti-PSMA-ADCs Comprising Structurally Different Amanitin Derivatives in Mouse Xenograft Tumor Model In Vivo

In the LNCap xenograft tumor mouse model, male CB17 SCID mice were inoculated with 2.5×10⁶ LNCap prostate carcinoma cells per mouse subcutaneously in the right flank. At a mean tumor vol. of ˜150 mm³, animals were allocated to groups on day 0. On the same day, the animals received a single intravenous dose of one of the structurally different amanitin-based anti-PSMA antibody drug conjugates (ADCs) listed in Table 6. Tumor volume and body weight were determined twice per week.

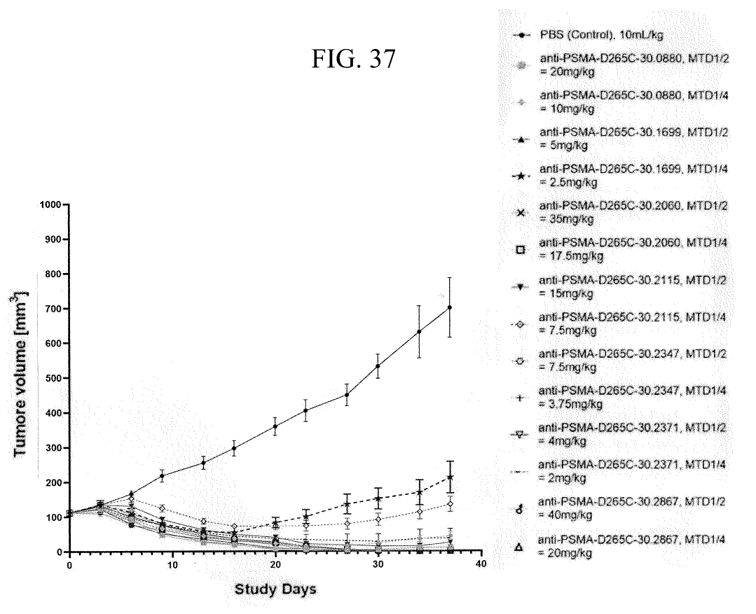

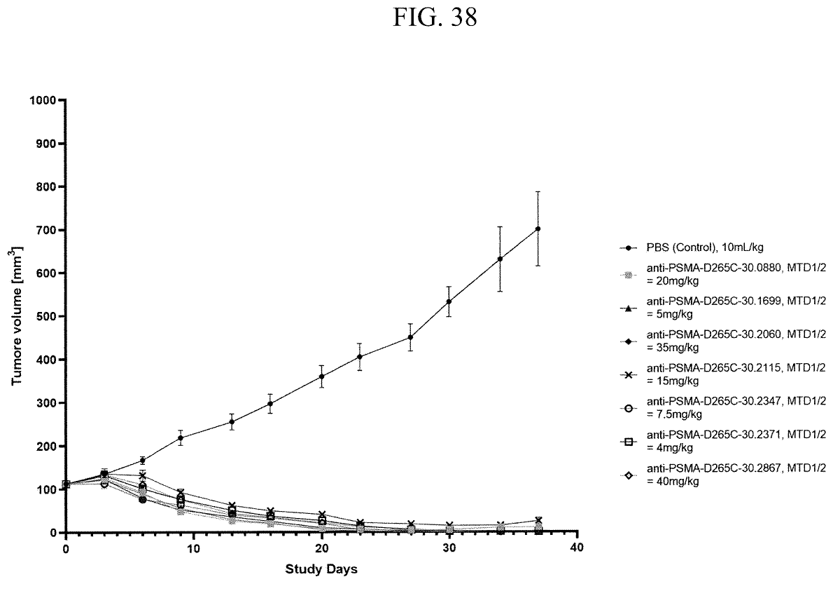

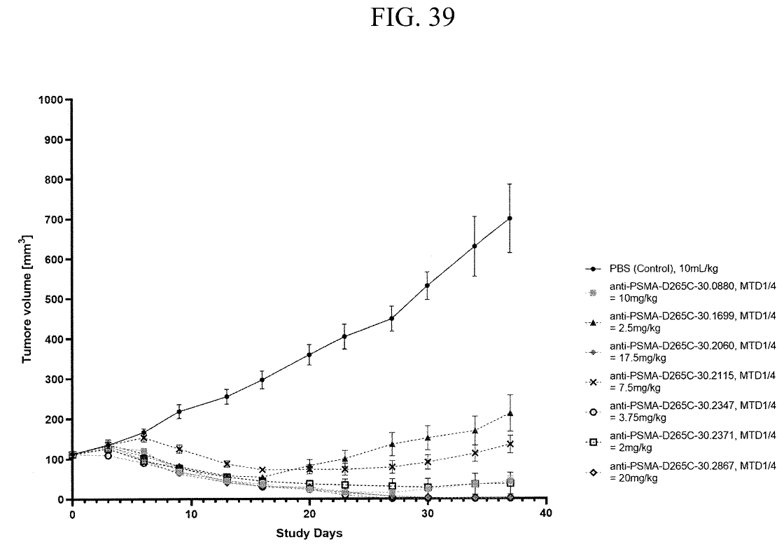

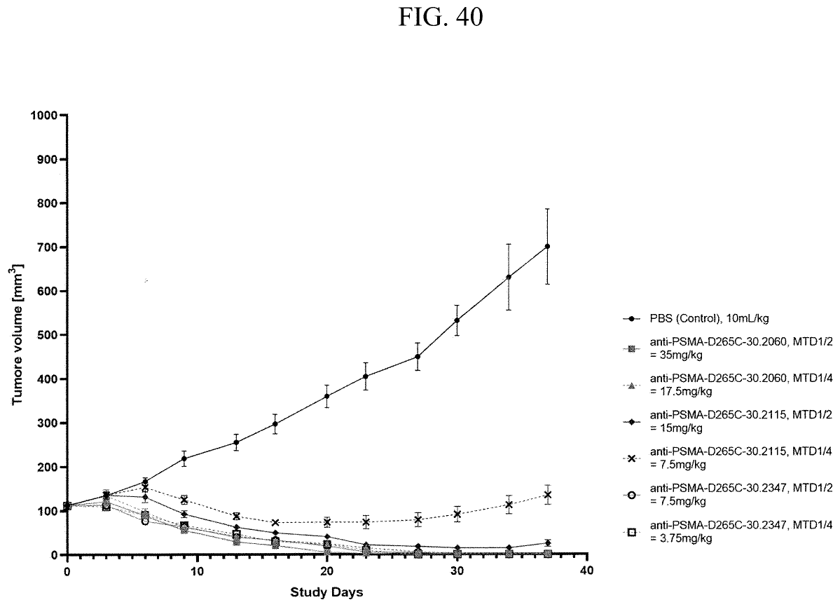

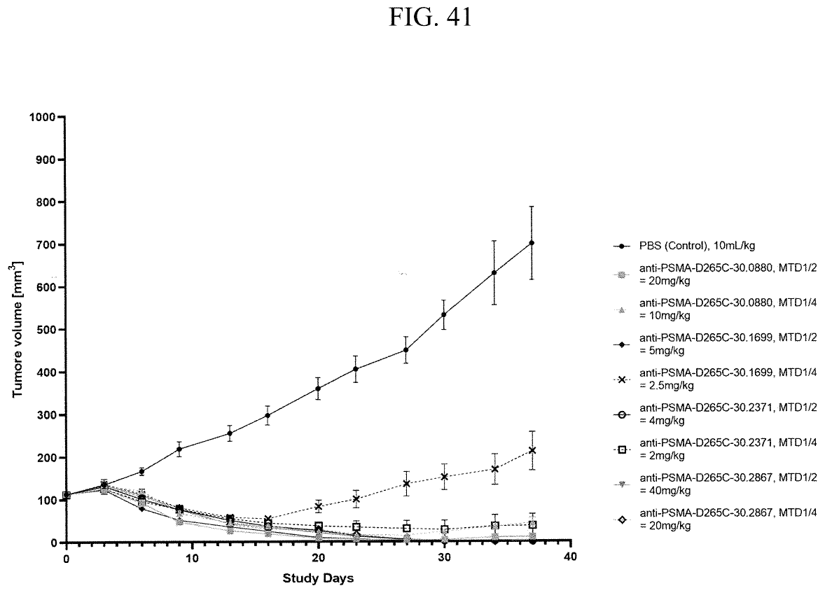

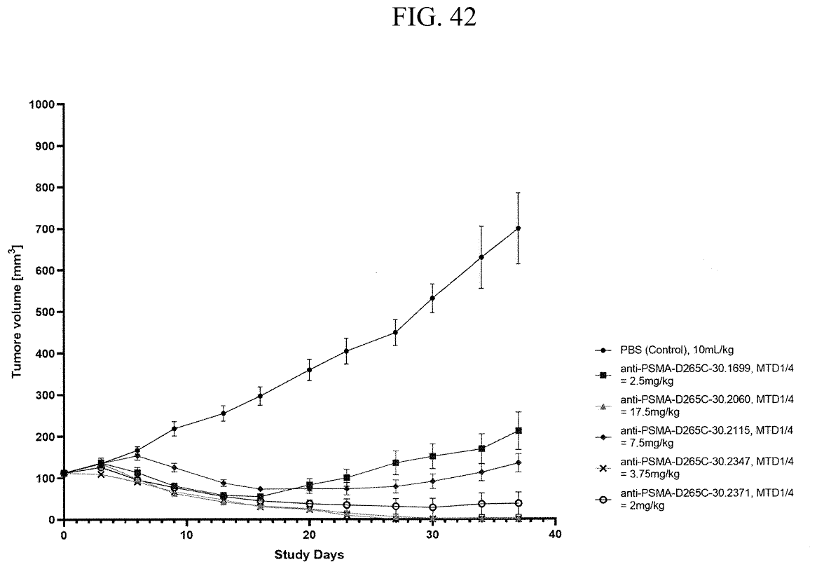

Results for the ADCs comprising differently structured amanitin derivatives are shown in FIG. 37 to FIG. 42 and Table 12. ADCs comprising non-cleavable linkers conjugated to the 6′-hydroxy group of tryptophan (amino acid 4) of the amatoxin, in particular h3/F11-D265C-Var16-30.2867 with a thioether bridge between Trp and Cys, were shown to have a high cytotoxic efficacy in vivo, as compared to ADCs comprising cleavable linkers conjugated to either amino acid 4 or amino acid 1 of the amatoxin.

TABLE 12
Survival rate and tumor load

| | | Alive | |
|---|---|---|---|
| Treatment Group | Dosing | Animals | Tumor-free Animals |
| Vehicle control | | 9/10 | 0/9 |
| anti-PSMA-D265C-30.0880 | MTD 1/2 | 10/10 | 5/10 |
| anti-PSMA-D265C-30.0880 | MTD 1/4 | 10/10 | 3/10 |
| anti-PSMA-D265C-30.1699 | MTD 1/2 | 10/10 | 10/10 |
| anti-PSMA-D265C-30.1699 | MTD 1/4 | 10/10 | 2/10 |
| anti-PSMA-D265C-30.2060 | MTD 1/2 | 4/10 | 4/4 |
| anti-PSMA-D265C-30.2060 | MTD 1/4 | 10/10 | 9/10 |
| anti-PSMA-D265C-30.2115 | MTD 1/2 | 10/10 | 3/10 |
| anti-PSMA-D265C-30.2115 | MTD 1/4 | 10/10 | 0/10 |
| anti-PSMA-D265C-30.2347 | MTD 1/2 | 10/10 | 10/10 |
| anti-PSMA-D265C-30.2347 | MTD 1/4 | 10/10 | 10/10 |
| anti-PSMA-D265C-30.2371 | MTD 1/2 | 10/10 | 10/10 |
| anti-PSMA-D265C-30.2371 | MTD 1/4 | 9/10 | 7/9 |
| anti-PSMA-D265C-30.2867 | MTD 1/2 | 10/10 | 9/10 |
| anti-PSMA-D265C-30.2867 | MTD 1/4 | 10/10 | 10/10 |

### Example 19: Efficacy of ADCs Comprising Antibodies with LALA and/or D265C Mutation

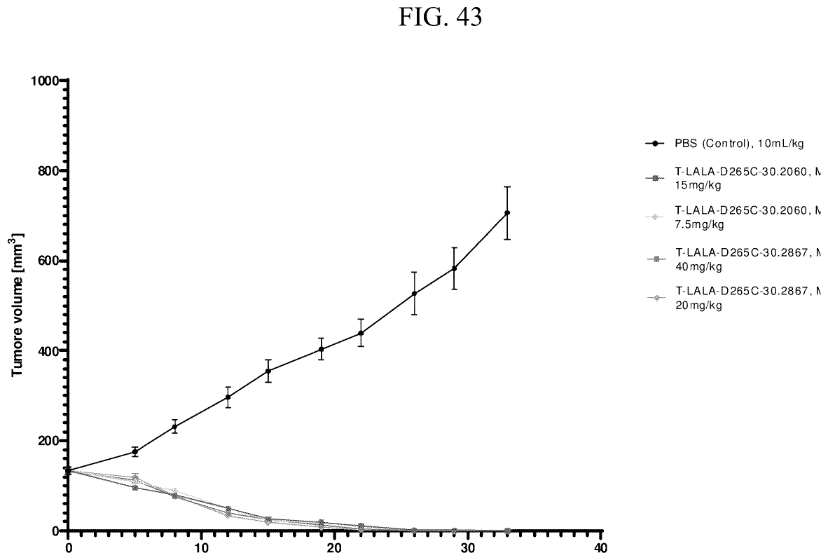

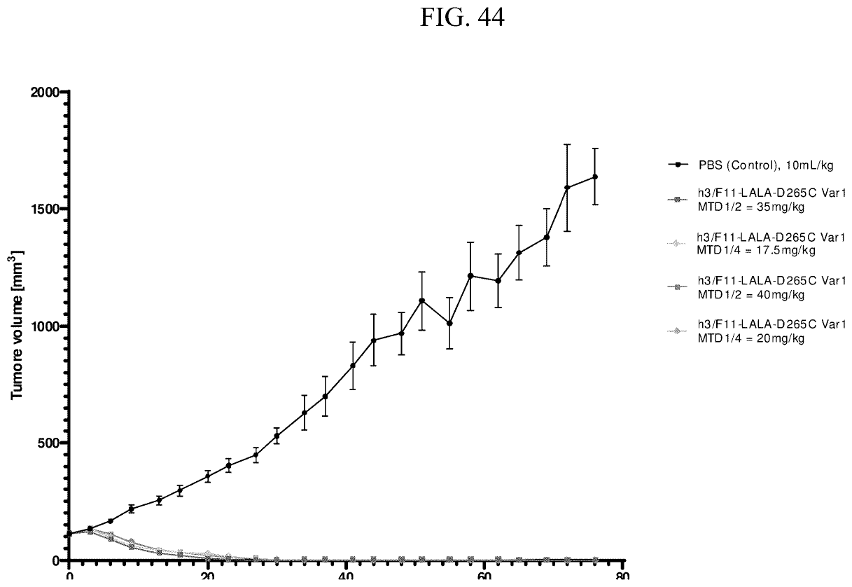

The efficacy of ADCs comprising antibodies having both L234A and L235A mutations and D265C mutation were tested in cell line-derived tumor xenograft (CDX) mouse models. Anti-Her2 ADCs comprising the triple L234A/L235A/D265C mutations and the amatoxin-linker construct HDP30.2060 and HDP30.2867, respectively (T-LALA-D265C-30.2060 and T-LALA-D265C-30.2867, respectively) were tested on Her2-positive NCI-N87 cells. Results are shown in FIG. 43. Anti-PSMA ADCs comprising the triple L234A/L235A/D265C mutations and the amatoxin-linker construct HDP30.2060 and HDP30.2867, respectively (h3/F11-LALA-D265C Var16-30.2060 and h3/F11-LALA-D265C Var16-30.2867, respectively) were tested on PSMA-positive LNCap cells. Results are shown in FIG. 44. In both studies, the ADCs carrying the triple mutation were shown to be efficient in reducing tumor volumes in the respective CDX mouse models.

As compared to ADCs without the L234A/L235A/D265C mutations, with ADCs comprising antibodies having L234A/L235A mutations and the D265C mutation, the same range of production yields were obtained. Furthermore, the mutations did not have an impact on the conjugation yields obtained with respective antibodies, as compared to antibodies without the mutations.

### Example 20: Cytotoxicity Assay In Vitro on Target-Versus Non-Target Cells

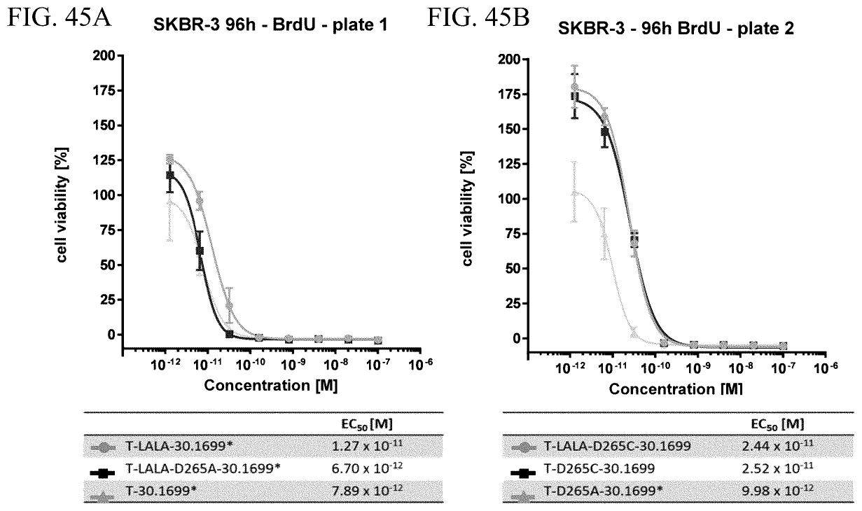

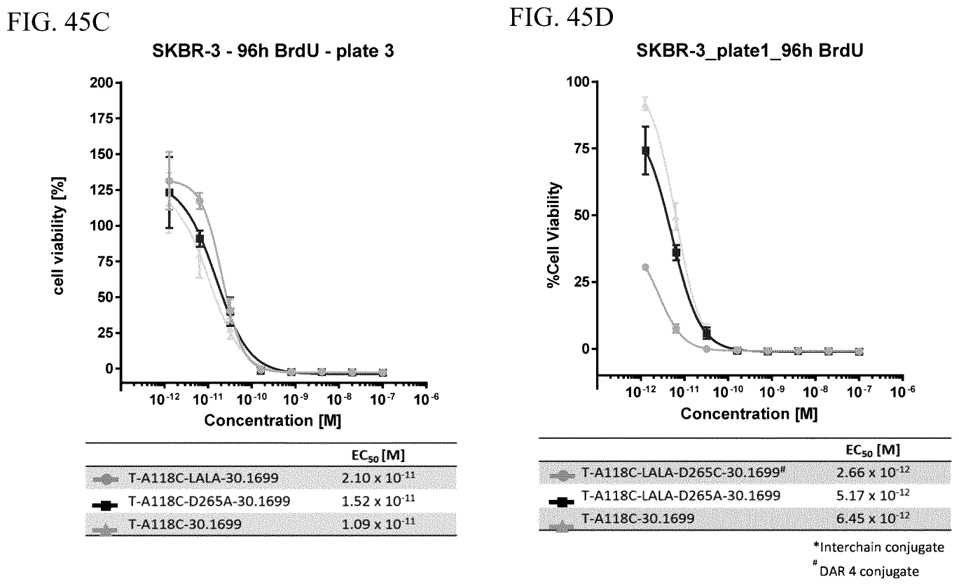

As shown in FIG. 45A to FIG. 45D, the cytotoxic potential in vitro of Her2-specific ADCs comprising an antibody carrying L234A/L235A mutations and/or the D265C mutation, as compared to ADCs without mutation and to control ADCs comprising the same antibody carrying other mutations (A118C, D265A), assessed in a 96-hour BrdU assay on Her2-positive SKBR-3 cells was comparable; full cytotoxicity was observed for all mutants and control ADCs.

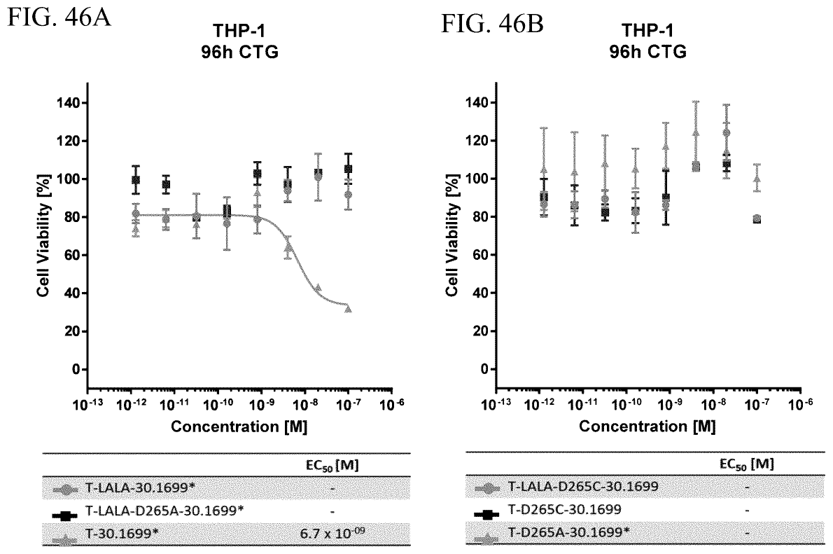

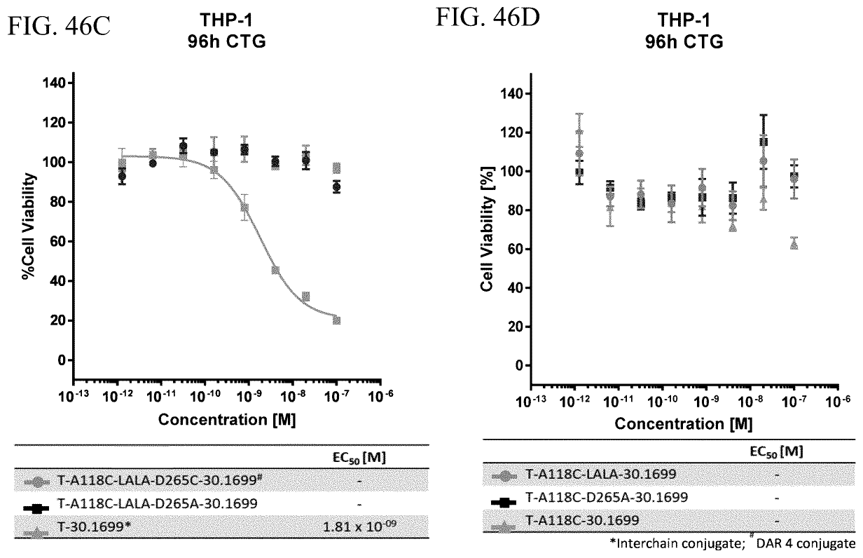

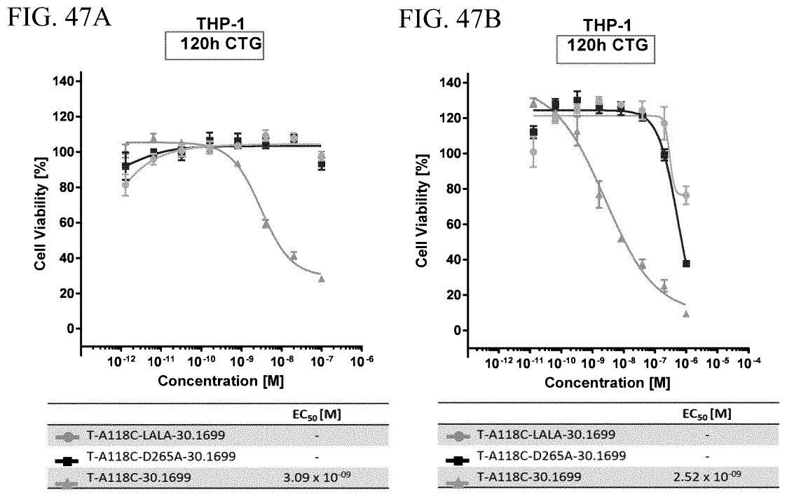

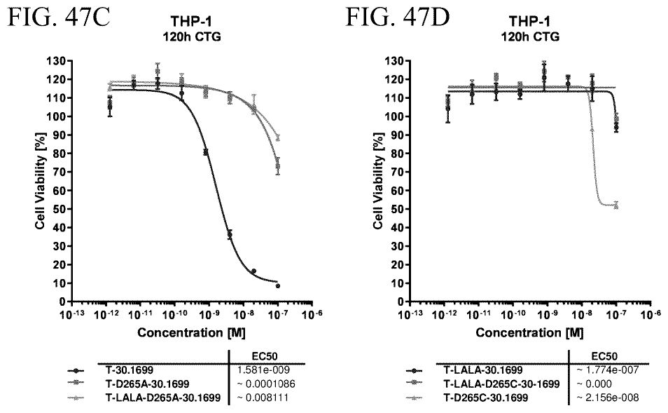

In contrast, when the cytotoxic potential in vitro of Her2-specific ADCs comprising an antibody carrying L234A/L235A mutations and/or the D265C mutation was assessed in a 96-hour CTG assay (FIG. 46A to FIG. 46D) and in a 120-hour CTG assay (FIG. 47A to FIG. 47D) on the Her2-negative macrophage cell line THP-1 in comparison to the respective ADC without mutations in the same antibody backbone, unspecific toxicity was observed with the ADC without mutation in the antibody (T-30.1699, FIG. 46A, FIG. 46C; FIG. 47C) at ADC concentrations higher than 10⁻¹⁰ to 10⁻⁹ M. With ADC comprising an antibody carrying L234A/L235A (“LALA”) mutations (T-LALA-30.1699), or the D265C mutation (T-D265C), or all three mutations (T-LALA-D265C-30.1699), no such unspecific toxicity on non-target THP-1 cells was observed in the 96-hour CTG assay (FIG. 46A, FIG. 46B) even at 10- to 100-fold higher ADC concentrations. With ADC comprising an antibody carrying a D265A mutation (T-D265A-30.1699), no unspecific toxicity on non-target THP-1 cells was observed either. In the 120-hour CTG assay, these data were essentially confirmed; the unspecific toxicity of ADC without mutation in the antibody was drastically higher than ADCs comprising an antibody carrying a single or triple mutation (FIG. 47C). The combination of LALA and D265C (or D265A) mutations appear to have an additive or even synergistic effect in terms of reducing unspecific toxicity on non-target cells (FIG. 47C, FIG. 47D). Unspecific cytotoxicity on THP-1 cells was shown to be Fc7 receptor mediated which confirms that those ATACs, which do not show cytotoxicity on THP-1 cells (LALA, D265C and D265A variants), have reduced Fcγ receptor interaction and in turn a reduced FcγR mediated uptake.

In summary, L234A/L235A and D265A/C mutation eliminate unspecific toxicity of ADCs on THP-1 cells under tested conditions.

### Example 21: Impact of LALA and/or D265C Mutations in ADCs on Fc□R Binding

Fc□R interaction of ADCs were tested by biolayer interferometry (BLI). Results of these binding studies are shown in Table 13.

TABLE 13
Fc□R binding of ADCs

| ADC/ | Fc□RI | | Fc□RIIa_{H167/R167} | | Fc□RIIIa_{V176/F176} | |
|---|---|---|---|---|---|---|
| Antibody | K_{D} [nM] | K_{D}/K_{D·wt} | K_{D} [μM] | K_{D}/K_{D·wt} | K_{D} [μM] | K_{D}/K_{D·wt} |
| T-wt | 0.37 | 1 | 1.30/0.68 | 1 | 0.44/1.30 | 1 |
| T-30.1699 | 0.31 | 0.84 | 1.44/0.50 | 1.1/1.36 | 0.65/1.65 | 1.48/1.26 |
| T-D265C- | 1.53 | 4.1 | (≈143)/(≈10) | (≈110)/(≈8) | >>10 | >>10 |
| 30.1699 | | | | | | |
| T-D265A- | 27.9 | 75 | >>10 | >1000 | >>10 | >>10 |
| 30.1699 | | | | | | |
| T-LALA- | 218 | 590 | >>10/>>10 | >>10/>>10 | 3.7/>>10 | 8.4/>>10 |
| 30.1699 | | | | | | |
| T-LALA- | 2950 | ~8000 | >>10/>>10 | >>10/>>10 | >>10/>>10 | >>10/>>10 |
| D265C- | | | | | | |
| 30.1699 | | | | | | |
| T-LALA- | >>5000 | >>5000 | >>10 | >>10 | >>10 | >>10 |
| D265A- | | | | | | |
| 30.1699 | | | | | | |

As shown in Table 13, introduction of LALA, D265A or D265C mutations into the antibody comprised in the ADC significantly reduced the affinity for Fc□RI, and also reduced binding to Fc□RIIa and Fc□RIIIa. The combination with D265 mutation (D265C, D265A) further drastically decreased Fc□RI binding.

### Example 22: Impact of LALA and/or D265C Mutations in ADCs on Tolerability In Vivo

Tolerability of ADCs comprising a Her2- or PSMA-specific antibody, respectively, carrying mutations was assessed in mice. The results are shown in Table 14. All antibodies used had the same backbone and were not cross-reactive in mice.

TABLE 14
Tolerability in mice of ADCs comprising
antibodies carrying LALA, D265C mutations

| | MTD, | MTD, |
|---|---|---|
| | Antibody | Antibody with |
| ADC Compound | with D265C | LALA-D265C |
| T(Anti-Her2)-30.2060 | 10 mg/kg | 30 mg/kg |
| h3/F11-Var16 | 40 mg/kg | 70 mg/kg |
| (Anti-PSMA)-30.2060 | | |
| T(Anti-Her2)-30.2867 | ≥80 mg/kg | >80 mg/kg |
| h3/F11-Var16 | ≥80 mg/kg | ≥80 mg/kg |
| (Anti-PSMA)-30.2867 | | |

MTD, maximum tolerated dose

As shown in Tab. 14, with ADCs comprising the amatoxin/linker compound HDP 30.2060 and antibodies carrying LALA and D265C mutations the maximum tolerated dose was significantly higher than with ADCs comprising said amatoxin/linker compound and antibodies carrying the D265C mutation only. With ADCs comprising the amatoxin/linker compound HDP 30.2867 and antibodies carrying LALA and D265C mutations the tolerability was even higher.

Tolerability of ADCs comprising antibodies carrying respective mutations was also assessed in non-human primates. All antibodies used had the same backbone and are non-binders in non-human primates. The anti-PSMA ADCs h3/F11-LALA-D265C Var16-30.2060 (carrying LALA and D265C mutations), h3/F11-D265C Var16-30.2060 (carrying D265C mutation only) and the Anti-Digoxigenin ADCs DIG-D265C-30.2060 and DIG-D265C-30.2867 (carrying the D265C mutation only), were assessed for a dose-escalating tolerability study in cynomolgus monkeys.

Groups of 2 female animals were injected with h3/F11-LALA-D265C Var16-30.2060 at days 1 (1 mg/kg), 23 (3 mg/kg), 44 (5 mg/kg), 65 (6 mg/kg), 86 (7.5 mg/kg), 128 (10 mg/kg), with h3/F11-D265C Var16-30.2060 at days 1 (1 mg/kg), 23 (3 mg/kg), and 44 (5 mg/kg), with DIG-D265C-30.2060 at days 1 (1 mg/kg), 22 (3 mg/kg), 43 (5 mg/kg), 64 (7.5 mg/kg), or with DIG-D265C-30.2867 at days 1 (1 mg/kg), 22 (3 mg/kg), 43 (5 mg/kg), 64 (7.5 mg/kg), 106 (10 mg/kg), 148 (15 mg/kg), and 196 (20 mg/kg). Animals were monitored over time for biochemical and haematological blood parameters, body weight, food consumption, clinical signs and mortality. In addition, blood samples were collected for pharmacokinetic studies. At the end of the experiments tissue samples were used for histopathological examinations.

Up to 7.5 mg/kg h3/F11-LALA-D265C Var16-30.2060 (carrying LALA and D265C mutations), 3 mg/kg h3/F11-D265C Var16-30.2060 (carrying D265C mutation only) 5 mg/kg DIG-D265C-30.2060 and 15 mg/kg DIG-D265C-30.2867 (carrying the D265C mutation only) doses were well tolerated with no signs of kidney damage by serum parameters and unaffected body weight and food consumption. Doses of 10 mg/kg h3/F11-LALA-D265C Var16-30.2060, 5 mg/kg h3/F11-D265C Var16-30.2060, 7.5 mg/kg DIG-D265C-30.2060 and 20 mg/kg DIG-D265C-30.2867 resulted in dead animals. The results are shown in Table 15.

TABLE 15
Tolerability in non-human primates of ADCs comprising
antibodies carrying LALA, D265C mutations

Amatoxin

| | MTD, Anti- | MTD, | MTD, Anti- |
|---|---|---|---|
| | Digoxigenin | Anti-PSMA | PSMA |
| Linker | Antibody with | Antibody with | Antibody |
| Compound | D265C | LALA-D265C | with D265C |
| HDP30.2060 | 5 mg/kg | 7.5 mg/kg | 3 mg/kg |
| HDP30.2867 | 15 mg/kg | n.a. | n.a. |

MTD, maximum tolerated dose

The data in non-human primates confirmed that with ADCs comprising antibodies carrying LALA and D265C mutations the maximum tolerated dose was significantly higher than with ADCs comprising antibodies carrying the D265C mutation only. With ADCs comprising the amatoxin/linker compound HDP 30.2867 and antibodies carrying LALA and D265C mutations the tolerability was particularly high.

SEQUENCE TABLE

| Sequence | | |
|---|---|---|
| Identifier | Description | Sequence |
| SEQ ID NO: 1 | CK6 CDR-H1 | SYWIG |
| SEQ ID NO: 2 | CK6 CDR-H2 | IIYPGDSDTRYSPSFQG |
| SEQ ID NO: 3 | CK6 CDR-H3 | HGRGYNGYEGAFDI |
| SEQ ID NO: 4 | CK6 CDR-L1 | RASQGISSALA |
| SEQ ID NO: 5 | CK6 CDR-L2 | DASSLES |
| SEQ ID NO: 6 | CK6 CDR-L3 | CQQFNSYPLT |
| SEQ ID NO: 7 | Consensus human | EVQLVESGGGLVQPGGSLRLSCAASGFTFS |
| | Ab | DYAMSWVRQAPGKGLEWVAVISENGSDT |
| | Heavy chain variable | YYADSVKGRFTISRDDSKNTLYLQMNSLR |
| | domain | AEDTAVYYCARDRGGAVSYFDVWGQGT |
| | | LVTVSS |
| SEQ ID NO: 8 | Consensus human | DIQMTQSPSSLSASVGDRVTITCRASQDVS |
| | Ab | SYLAWYQQKPGKAPKLLIYAASSLESGVP |
| | Light chain variable | SRFSGSGSGTDFTLTISSLQPEDFATYYCQQ |
| | domain | YNSLPYTFGQGTKVEIKRT |
| SEQ ID NO: 9 | Ab67 Heavy chain | EVQLVESGGGLVQPGGSLRLSCAASGFTFS |
| | variable region (e.g., | DADMDWVRQAPGKGLEWVGRTRNKAGS |
| | as found in HC-67) | YTTEYAASVKGRFTISRDDSKNSLYLQMN |
| | (CDRs in bold) | SLKTEDTAVYYCAREPKYWIDFDLWGRG |
| | | TLVTVSS |
| SEQ ID NO: | Ab67 Light chain | DIQMTQSPSSLSASVGDRVTITCRASQSISS |
| 10 | variable region (e.g., | YLNWYQQKPGKAPKLLIYAASSLQSGVPS |
| | as found in LC-67) | RFSGSGSGTDFTLTISSLQPEDFATYYCQQS |
| | (CDRs in bold) | YIAPYTFGGGTKVEIK |
| SEQ ID NO: | Ab67 CDR-H1 | FTFSDADMD |
| 11 | | |
| SEQ ID NO: | Ab67 CDR-H2 | RTRNKAGSYTTEYAASVKG |
| 12 | | |
| SEQ ID NO: | Ab67 CDR-H3 | AREPKYWIDFDL |
| 13 | | |
| SEQ ID NO: | Ab67 CDR-L1 | RASQSISSYLN |
| 14 | | |
| SEQ ID NO: | Ab67 CDR-L2 | AASSLQS |
| 15 | | |
| SEQ ID NO: | Ab67 CDR-L3 | QQSYIAPYT |
| 16 | | |
| SEQ ID NO: | Ab67 Heavy chain | GAGGTGCAGCTGGTGGAGTCTGGGGGAG |
| 17 | variable region | GCTTGGTCCAGCCTGGAGGGTCCCTGAG |
| | (nucl) | ACTCTCCTGTGCAGCCTCTGGATTCACCT |
| | | TCAGTGACGCCGACATGGACTGGGTCCG |
| | | CCAGGCTCCAGGGAAGGGGCTGGAGTGG |
| | | GTTGGCCGTACTAGAAACAAAGCAGGAA |
| | | GTTACACCACAGAATACGCCGCGTCTGT |
| | | GAAAGGCAGATTCACCATCTCAAGAGAT |
| | | GATTCAAAGAACTCACTGTATCTGCAAAT |
| | | GAACAGCCTGAAAACCGAGGACACGGCG |
| | | GTGTACTACTGCGCCAGAGAGCCTAAAT |
| | | ACTGGATCGACTTCGACCTATGGGGGAG |
| | | AGGTACCTTGGTCACCGTCTCCTCA |
| SEQ ID NO: | Ab67 Light chain | GACATCCAGATGACCCAGTCTCCATCCTC |
| 18 | variable region | CCTGTCTGCATCTGTAGGAGACAGAGTC |
| | (nucl) | ACCATCACTTGCCGGGCAAGTCAGAGCA |
| | | TTAGCAGCTATTTAAATTGGTATCAGCAG |
| | | AAACCAGGGAAAGCCCCTAAGCTCCTGA |
| | | TCTATGCTGCATCCAGTTTGCAAAGTGGG |
| | | GTCCCATCAAGGTTCAGTGGCAGTGGAT |
| | | CTGGGACAGATTTCACTCTCACCATCAGC |
| | | AGTCTGCAACCTGAAGATTTTGCAACTTA |
| | | CTACTGTCAGCAAAGCTACATCGCCCCTT |
| | | ACACTTTTGGCGGAGGGACCAAGGTTGA |
| | | GATCAAA |
| SEQ ID NO: | Ab55 Heavy chain | QVQLVQSGAEVKKPGSSVKVSCKASGGTF |
| 19 | variable region (e.g., | RIYAISWVRQAPGQGLEWMGGIIPDFGVA |
| | as found in HC-55) | NYAQKFQGRVTITADESTSTAYMELSSLRS |
| | (CDRs in bold) | EDTAVYYCARGGLDTDEFDLWGRGTLVT |
| | | VSS |
| SEQ ID NO: | Ab55 Light chain | DIQMTQSPSSLSASVGDRVTITCRASQSINS |
| 20 | variable region (e.g., | YLNWYQQKPGKAPKLLIYAASSLQSGVPS |
| | as found in LC-55) | RFSGSGSGTDFTLTISSLQPEDFATYYCQQ |
| | (CDRs in bold) | GVSDITFGGGTKVEIK |
| SEQ ID NO: | Ab55 CDR-H1 | GTFRIYAIS |
| 21 | | |
| SEQ ID NO: | Ab55 CDR-H2 | GIIPDFGVANYAQKFQG |
| 22 | | |
| SEQ ID NO: | Ab55 CDR-H3 | ARGGLDTDEFDL |
| 23 | | |
| SEQ ID NO: | Ab55 CDR-L1 | RASQSINSYLN |
| 24 | | |
| SEQ ID NO: | Ab55 CDR-L2 | AASSLQS |
| 25 | | |
| SEQ ID NO: | Ab55 CDR-L3 | QQGVSDIT |
| 26 | | |
| SEQ ID NO: | Ab55 Heavy chain | CAGGTGCAGCTGGTGCAGTCTGGGGCTG |
| 27 | variable region | AGGTGAAGAAGCCTGGGTCCTCGGTGAA |
| | (nucl) | GGTCTCCTGCAAGGCTTCTGGAGGCACCT |
| | | TCCGAATCTATGCTATCAGCTGGGTGCGA |
| | | CAGGCCCCTGGACAAGGGCTTGAGTGGA |
| | | TGGGAGGGATCATCCCTGACTTCGGTGTA |
| | | GCAAACTACGCACAGAAGTTCCAGGGCA |
| | | GAGTCACGATTACCGCGGACGAATCCAC |
| | | GAGCACAGCCTACATGGAGCTGAGCAGC |
| | | CTGAGATCTGAGGACACGGCGGTGTACT |
| | | ACTGCGCCAGAGGTGGATTGGACACAGA |
| | | CGAGTTCGACCTATGGGGGAGAGGTACC |
| | | TTGGTCACCGTCTCCTCA |
| SEQ ID NO: | Ab55 Light chain | GACATCCAGATGACCCAGTCTCCATCCTC |
| 28 | variable region | CCTGTCTGCATCTGTAGGAGACAGAGTC |
| | (nucl) | ACCATCACTTGCCGGGCAAGTCAGAGCA |
| | | TTAACAGCTATTTAAATTGGTATCAGCAG |
| | | AAACCAGGGAAAGCCCCTAAGCTCCTGA |
| | | TCTATGCTGCATCCAGTTTGCAAAGTGGG |
| | | GTCCCATCAAGGTTCAGTGGCAGTGGAT |
| | | CTGGGACAGATTTCACTCTCACCATCAGC |
| | | AGTCTGCAACCTGAAGATTTTGCAACTTA |
| | | CTACTGTCAGCAAGGAGTCAGTGACATC |
| | | ACTTTTGGCGGAGGGACCAAGGTTGAGA |
| | | TCAAA |
| SEQ ID NO: | Ab54 Heavy chain | QVQLVQSGAEVKKPGSSVKVSCKASGGTF |
| 29 | variable region (e.g., | SSYAISWVRQAPGQGLEWMGGIIPIFGTA |
| | as found in HC-54) | NYAQKFQGRVTITADESTSTAYMELSSLRS |
| | hIgG1 backbone | EDTAVYYCARGGLDTDEFDLWGRGTLVT |
| | (CDRs in bold) | VSS |
| SEQ ID NO: | Ab54 Light chain | DIQMTQSPSSLSASVGDRVTITCRASQSINS |
| 30 | variable region (e.g., | YLNWYQQKPGKAPKLLIYAASSLQSGVPS |
| | as found in LC-54) | RFSGSGSGTDFTLTISSLQPEDFATYYCQQ |
| | (CDRs in bold) | GVSDITFGGGTKVEIK |
| SEQ ID NO: | Ab54 CDR-H1 | GTFSSYAIS |
| 31 | | |
| SEQ ID NO: | Ab54 CDR-H2 | GIIPIFGTANYAQKFQG |
| 32 | | |
| SEQ ID NO: | Ab54 CDR-H3 | ARGGLDTDEFDL |
| 33 | | |
| SEQ ID NO: | Ab54 CDR-L1 | RASQSINSYLN |
| 34 | | |
| SEQ ID NO: | Ab54 CDR-L2 | AASSLQS |
| 35 | | |
| SEQ ID NO: | Ab54 CDR-L3 | QQGVSDIT |
| 36 | | |
| SEQ ID NO: | Ab54 Heavy chain | CAGGTGCAGCTGGTGCAGTCTGGGGCTG |
| 37 | variable region | AGGTGAAGAAGCCTGGGTCCTCGGTGAA |
| | (nucl) | GGTCTCCTGCAAGGCTTCTGGAGGCACCT |
| | | TCAGCAGCTATGCTATCAGCTGGGTGCG |
| | | ACAGGCCCCTGGACAAGGGCTTGAGTGG |
| | | ATGGGAGGGATCATCCCTATCTTTGGTAC |
| | | AGCAAACTACGCACAGAAGTTCCAGGGC |
| | | AGAGTCACGATTACCGCGGACGAATCCA |
| | | CGAGCACAGCCTACATGGAGCTGAGCAG |
| | | CCTGAGATCTGAGGACACGGCGGTGTAC |
| | | TACTGCGCCAGAGGTGGATTGGACACAG |
| | | ACGAGTTCGACCTATGGGGGAGAGGTAC |
| | | CTTGGTCACCGTCTCCTCA |
| SEQ ID NO: | Ab54 Light chain | GACATCCAGATGACCCAGTCTCCATCCTC |
| 38 | variable region | CCTGTCTGCATCTGTAGGAGACAGAGTC |
| | (nucl) | ACCATCACTTGCCGGGCAAGTCAGAGCA |
| | | TTAACAGCTATTTAAATTGGTATCAGCAG |
| | | AAACCAGGGAAAGCCCCTAAGCTCCTGA |
| | | TCTATGCTGCATCCAGTTTGCAAAGTGGG |
| | | GTCCCATCAAGGTTCAGTGGCAGTGGAT |
| | | CTGGGACAGATTTCACTCTCACCATCAGC |
| | | AGTCTGCAACCTGAAGATTTTGCAACTTA |
| | | CTACTGTCAGCAAGGAGTCAGTGACATC |
| | | ACTTTTGGCGGAGGGACCAAGGTTGAGA |
| | | TCAAA |
| SEQ ID NO: | Ab56 Heavy chain | QVQLVQSGAEVKKPGSSVKVSCKASGGTF |
| 39 | variable region | SLYAISWVRQAPGQGLEWMGGIIPAFGTA |
| | (e.g., as found in | NYAQKFQGRVTITADESTSTAYMELSSLRS |
| | HC-56) | EDTAVYYCARGGLDTDEFDLWGRGTLVT |
| | (CDRs in bold) | VSS |
| SEQ ID NO: | Ab56 Light chain | DIQMTQSPSSLSASVGDRVTITCRASQSINS |
| 40 | variable region (e.g., | YLNWYQQKPGKAPKLLIYAASSLQSGVPS |
| | as found in LC-56) | RFSGSGSGTDFTLTISSLQPEDFATYYCQQ |
| | (CDRs in bold) | GVSDITFGGGTKVEIK |
| SEQ ID NO: | Ab56 CDR-H1 | GTFSLYAIS |
| 41 | | |
| SEQ ID NO: | Ab56 CDR-H2 | GIIPAFGTANYAQKFQG |
| 42 | | |
| SEQ ID NO: | Ab56 CDR-H3 | ARGGLDTDEFDL |
| 43 | | |
| SEQ ID NO: | Ab56 CDR-L1 | RASQSINSYLN |
| 44 | | |
| SEQ ID NO: | Ab56 CDR-L2 | AASSLQS |
| 45 | | |
| SEQ ID NO: | Ab56 CDR-L3 | QQGVSDIT |
| 46 | | |
| SEQ ID NO: | Ab56 Heavy chain | CAGGTGCAGCTGGTGCAGTCTGGGGCTG |
| 47 | variable region | AGGTGAAGAAGCCTGGGTCCTCGGTGAA |
| | (nucl) | GGTCTCCTGCAAGGCTTCTGGAGGCACCT |
| | | TCAGCCTCTATGCTATCTCCTGGGTGCGA |
| | | CAGGCCCCTGGACAAGGGCTTGAGTGGA |
| | | TGGGAGGGATCATCCCTGCCTTCGGTACC |
| | | GCAAACTACGCACAGAAGTTCCAGGGCA |
| | | GAGTCACGATTACCGCGGACGAATCCAC |
| | | GAGCACAGCCTACATGGAGCTGAGCAGC |
| | | CTGAGATCTGAGGACACGGCGGTGTACT |
| | | ACTGCGCCAGAGGTGGATTGGACACAGA |
| | | CGAGTTCGACCTATGGGGGAGAGGTACC |
| | | TTGGTCACCGTCTCCTCA |
| SEQ ID NO: | Ab56 Light chain | GACATCCAGATGACCCAGTCTCCATCCTC |
| 48 | variable region | CCTGTCTGCATCTGTAGGAGACAGAGTC |
| | (nucl) | ACCATCACTTGCCGGGCAAGTCAGAGCA |
| | | TTAACAGCTATTTAAATTGGTATCAGCAG |
| | | AAACCAGGGAAAGCCCCTAAGCTCCTGA |
| | | TCTATGCTGCATCCAGTTTGCAAAGTGGG |
| | | GTCCCATCAAGGTTCAGTGGCAGTGGAT |
| | | CTGGGACAGATTTCACTCTCACCATCAGC |
| | | AGTCTGCAACCTGAAGATTTTGCAACTTA |
| | | CTACTGTCAGCAAGGAGTCAGTGACATC |
| | | ACTTTTGGCGGAGGGACCAAGGTTGAGA |
| | | TCAAA |
| SEQ ID NO: | Ab57 Heavy chain | QVQLVQSGAEVKKPGSSVKVSCKASGGTF |
| 49 | variable region (e.g., | SLYAISWVRQAPGQGLEWMGGIIPHFGLA |
| | as found in HC-57) | NYAQKFQGRVTITADESTSTAYMELSSLRS |
| | hIgG1 backbone | EDTAVYYCARGGLDTDEFDLWGRGTLVT |
| | (CDRs in bold) | VSS |
| SEQ ID NO: | Ab57 Light chain | DIQMTQSPSSLSASVGDRVTITCRASQSINS |
| 50 | variable region (e.g., | YLNWYQQKPGKAPKLLIYAASSLQSGVPS |
| | as found in LC-57) | RFSGSGSGTDFTLTISSLQPEDFATYYCQQ |
| | hKappa backbone | GVSDITFGGGTKVEIK |
| | (CDRs in bold) | |
| SEQ ID NO: | Ab57 CDR-H1 | GTFSLYAIS |
| 51 | | |
| SEQ ID NO: | Ab57 CDR-H2 | GIIPHFGLANYAQKFQG |
| 52 | | |
| SEQ ID NO: | Ab57 CDR-H3 | ARGGLDTDEFDL |
| 53 | | |
| SEQ ID NO: | Ab57 CDR-L1 | RASQSINSYLN |
| 54 | | |
| SEQ ID NO: | Ab57 CDR-L2 | AASSLQS |
| 55 | | |
| SEQ ID NO: | Ab57 CDR-L3 | QQGVSDIT |
| 56 | | |
| SEQ ID NO: | Ab57 Heavy chain | CAGGTGCAGCTGGTGCAGTCTGGGGCTG |
| 57 | variable region | AGGTGAAGAAGCCTGGGTCCTCGGTGAA |
| | (nucl) | GGTCTCCTGCAAGGCTTCTGGAGGCACCT |
| | | TCTCCCTCTATGCTATCAGCTGGGTGCGA |
| | | CAGGCCCCTGGACAAGGGCTTGAGTGGA |
| | | TGGGAGGGATCATCCCTCACTTCGGTCTC |
| | | GCAAACTACGCACAGAAGTTCCAGGGCA |
| | | GAGTCACGATTACCGCGGACGAATCCAC |
| | | GAGCACAGCCTACATGGAGCTGAGCAGC |
| | | CTGAGATCTGAGGACACGGCGGTGTACT |
| | | ACTGCGCCAGAGGTGGATTGGACACAGA |
| | | CGAGTTCGACCTATGGGGGAGAGGTACC |
| | | TTGGTCACCGTCTCCTCA |
| SEQ ID NO: | Ab57 Light chain | GACATCCAGATGACCCAGTCTCCATCCTC |
| 58 | variable region | CCTGTCTGCATCTGTAGGAGACAGAGTC |
| | (nucl) | ACCATCACTTGCCGGGCAAGTCAGAGCA |
| | | TTAACAGCTATTTAAATTGGTATCAGCAG |
| | | AAACCAGGGAAAGCCCCTAAGCTCCTGA |
| | | TCTATGCTGCATCCAGTTTGCAAAGTGGG |
| | | GTCCCATCAAGGTTCAGTGGCAGTGGAT |
| | | CTGGGACAGATTTCACTCTCACCATCAGC |
| | | AGTCTGCAACCTGAAGATTTTGCAACTTA |
| | | CTACTGTCAGCAAGGAGTCAGTGACATC |
| | | ACTTTTGGCGGAGGGACCAAGGTTGAGA |
| | | TCAAA |
| SEQ ID NO: | Ab58 Heavy chain | EVQLLESGGGLVQPGGSLRLSCAASGFTFS |
| 59 | variable region (e.g., | NYAMSWVRQAPGKGLEWVSAISGSGGST |
| | as found in HC-58) | YYADSVKGRFTISRDNSKNTLYLQMNSLR |
| | (CDRs in bold) | AEDTAVYYCAKGPPTYHTNYYYMDVWG |
| | | KGTTVTVSS |
| SEQ ID NO: | Ab58 Light chain | DIQMTQSPSSVSASVGDRVTITCRASQGISS |
| 60 | variable region (e.g., | WLAWYQQKPGKAPKLLIYAASSLQSGVPS |
| | as found in LC-58) | RFSGSGSGTDFTLTISSLQPEDFATYYCQQT |
| | (CDRs in bold) | NSFPYTFGGGTKVEIK |
| SEQ ID NO: | Ab58 CDR-H1 | FTFSNYAMS |
| 61 | | |
| SEQ ID NO: | Ab58 CDR-H2 | AISGSGGSTYYADSVKG |
| 62 | | |
| SEQ ID NO: | Ab58 CDR-H3 | AKGPPTYHTNYYYMDV |
| 63 | | |
| SEQ ID NO: | Ab58 CDR-L1 | RASQGISSWLA |
| 64 | | |
| SEQ ID NO: | Ab58 CDR-L2 | AASSLQS |
| 65 | | |
| SEQ ID NO: | Ab58 CDR-L3 | QQTNSFPYT |
| 66 | | |
| SEQ ID NO: | Ab58 Heavy chain | GAGGTGCAGCTGTTGGAGTCTGGGGGAG |
| 67 | variable region | GCTTGGTACAGCCTGGGGGGTCCCTGAG |
| | (nucl) | ACTCTCCTGTGCAGCCTCTGGATTCACCT |
| | | TTAGCAATTATGCCATGAGCTGGGTCCGC |
| | | CAGGCTCCAGGGAAGGGGCTGGAGTGGG |
| | | TCTCAGCTATTAGTGGTAGTGGTGGTAGC |
| | | ACATACTACGCAGACTCCGTGAAGGGCC |
| | | GGTTCACCATCTCCAGAGACAATTCCAA |
| | | GAACACGCTGTATCTGCAAATGAACAGC |
| | | CTGAGAGCCGAGGACACGGCGGTGTACT |
| | | ACTGCGCCAAGGGCCCTCCTACATACCA |
| | | CACAAACTACTACTACATGGACGTATGG |
| | | GGCAAGGGTACAACTGTCACCGTCTCCTC |
| | | A |
| SEQ ID NO: | Ab58 Light chain | GACATCCAGATGACCCAGTCTCCATCTTC |
| 68 | variable region | CGTGTCTGCATCTGTAGGAGACAGAGTC |
| | (nucl) | ACCATCACTTGTCGGGCGAGTCAGGGTA |
| | | TTAGCAGCTGGTTAGCCTGGTATCAGCAG |
| | | AAACCAGGGAAAGCCCCTAAGCTCCTGA |
| | | TCTATGCTGCATCCAGTTTGCAAAGTGGG |
| | | GTCCCATCAAGGTTCAGCGGCAGTGGAT |
| | | CTGGGACAGATTTCACTCTCACCATCAGC |
| | | AGCCTGCAGCCTGAAGATTTTGCAACTTA |
| | | TTACTGTCAGCAAACAAATAGTTTCCCTT |
| | | ACACTTTTGGCGGAGGGACCAAGGTTGA |
| | | GATCAAA |
| SEQ ID NO: | Ab61 Heavy chain | EVQLLESGGGLVQPGGSLRLSCAASGFTFS |
| 69 | variable region (e.g., | SYVMIWVRQAPGKGLEWVSSISGDSVTTY |
| | as found in HC-61) | YADSVKGRFTISRDNSKNTLYLQMNSLRA |
| | hIgG1 backbone | EDTAVYYCAKGPPTYHTNYYYMDVWGK |
| | (CDRs in bold) | GTTVTVSS |
| SEQ ID NO: | Ab61 Light chain | DIQMTQSPSSVSASVGDRVTITCRASQGISS |
| 70 | variable region (e.g., | WLAWYQQKPGKAPKLLIYAASSLQSGVPS |
| | as found in LC-61) | RFSGSGSGTDFTLTISSLQPEDFATYYCQQT |
| | (CDRs in bold) | NSFPYTFGGGTKVEIK |
| SEQ ID NO: | Ab61 CDR-H1 | FTFSSYVMI |
| 71 | | |
| SEQ ID NO: | Ab61 CDR-H2 | SISGDSVTTYYADSVKG |
| 72 | | |
| SEQ ID NO: | Ab61 CDR-H3 | AKGPPTYHTNYYYMDV |
| 73 | | |
| SEQ ID NO: | Ab61 CDR-L1 | RASQGISSWLA |
| 74 | | |
| SEQ ID NO: | Ab61 CDR-L2 | AASSLQS |
| 75 | | |
| SEQ ID NO: | Ab61 CDR-L3 | QQTNSFPYT |
| 76 | | |
| SEQ ID NO: | Ab61 Heavy chain | GAGGTGCAGCTGTTGGAGTCTGGGGGAG |
| 77 | variable region | GCTTGGTACAGCCTGGGGGGTCCCTGAG |
| | (nucl) | ACTCTCCTGTGCAGCCTCTGGATTCACCT |
| | | TTAGCAGCTATGTCATGATCTGGGTCCGC |
| | | CAGGCTCCAGGGAAGGGGCTGGAGTGGG |
| | | TCTCAAGCATTAGTGGTGACAGCGTAAC |
| | | AACATACTACGCAGACTCCGTGAAGGGC |
| | | CGGTTCACCATCTCCAGAGACAATTCCAA |
| | | GAACACGCTGTATCTGCAAATGAACAGC |
| | | CTGAGAGCCGAGGACACGGCGGTGTACT |
| | | ACTGCGCCAAGGGCCCTCCTACATACCA |
| | | CACAAACTACTACTACATGGACGTATGG |
| | | GGCAAGGGTACAACTGTCACCGTCTCCTC |
| | | A |
| SEQ ID NO: | Ab61 Light chain | GACATCCAGATGACCCAGTCTCCATCTTC |
| 78 | variable region | CGTGTCTGCATCTGTAGGAGACAGAGTC |
| | (nucl) | ACCATCACTTGTCGGGCGAGTCAGGGTA |
| | | TTAGCAGCTGGTTAGCCTGGTATCAGCAG |
| | | AAACCAGGGAAAGCCCCTAAGCTCCTGA |
| | | TCTATGCTGCATCCAGTTTGCAAAGTGGG |
| | | GTCCCATCAAGGTTCAGCGGCAGTGGAT |
| | | CTGGGACAGATTTCACTCTCACCATCAGC |
| | | AGCCTGCAGCCTGAAGATTTTGCAACTTA |
| | | TTACTGTCAGCAAACAAATAGTTTCCCTT |
| | | ACACTTTTGGCGGAGGGACCAAGGTTGA |
| | | GATCAAA |
| SEQ ID NO: | Ab66 Heavy chain | EVQLVESGGGLVQPGGSLRLSCAASGFTFS |
| | variable region (e.g., | DHYMDWVRQAPGKGLEWVGRTRNKASS |
| 79 | as found in HC-66) | YTTEYAASVKGRFTISRDDSKNSLYLQMN |
| | (CDRs in bold) | SLKTEDTAVYYCAREPKYWIDFDLWGRG |
| | | TLVTVSS |
| SEQ ID NO: | Ab66 Light chain | DIQMTQSPSSLSASVGDRVTITCRASQSISS |
| 80 | variable region (e.g., | YLNWYQQKPGKAPKLLIYAASSLQSGVPS |
| | as found in LC-66) | RFSGSGSGTDFTLTISSLQPEDFATYYCQQS |
| | hKappa backbone | YIAPYTFGGGTKVEIK |
| | (CDRs in bold) | |
| SEQ ID NO: | Ab66 CDR-H1 | FTFSDHYMD |
| 81 | | |
| SEQ ID NO: | Ab66 CDR-H2 | RTRNKASSYTTEYAASVKG |
| 82 | | |
| SEQ ID NO: | Ab66 CDR-H3 | AREPKYWIDFDL |
| 83 | | |
| SEQ ID NO: | Ab66 CDR-L1 | RASQSISSYLN |
| 84 | | |
| SEQ ID NO: | Ab66 CDR-L2 | AASSLQS |
| 85 | | |
| SEQ ID NO: | Ab66 CDR-L3 | QQSYIAPYT |
| 86 | | |
| SEQ ID NO: | Ab66 Heavy chain | GAGGTGCAGCTGGTGGAGTCTGGGGGAG |
| 87 | variable region | GCTTGGTCCAGCCTGGAGGGTCCCTGAG |
| | (nucl) | ACTCTCCTGTGCAGCCTCTGGATTCACCT |
| | | TCAGTGACCACTACATGGACTGGGTCCG |
| | | CCAGGCTCCAGGGAAGGGGCTGGAGTGG |
| | | GTTGGCCGTACTAGAAACAAAGCTAGTA |
| | | GTTACACCACAGAATACGCCGCGTCTGT |
| | | GAAAGGCAGATTCACCATCTCAAGAGAT |
| | | GATTCAAAGAACTCACTGTATCTGCAAAT |
| | | GAACAGCCTGAAAACCGAGGACACGGCG |
| | | GTGTACTACTGCGCCAGAGAGCCTAAAT |
| | | ACTGGATCGACTTCGACCTATGGGGGAG |
| | | AGGTACCTTGGTCACCGTCTCCTCA |
| SEQ ID NO: | Ab66 Light chain | GACATCCAGATGACCCAGTCTCCATCCTC |
| 88 | variable region | CCTGTCTGCATCTGTAGGAGACAGAGTC |
| | (nucl) | ACCATCACTTGCCGGGCAAGTCAGAGCA |
| | | TTAGCAGCTATTTAAATTGGTATCAGCAG |
| | | AAACCAGGGAAAGCCCCTAAGCTCCTGA |
| | | TCTATGCTGCATCCAGTTTGCAAAGTGGG |
| | | GTCCCATCAAGGTTCAGTGGCAGTGGAT |
| | | CTGGGACAGATTTCACTCTCACCATCAGC |
| | | AGTCTGCAACCTGAAGATTTTGCAACTTA |
| | | CTACTGTCAGCAAAGCTACATCGCCCCTT |
| | | ACACTTTTGGCGGAGGGACCAAGGTTGA |
| | | GATCAAA |
| SEQ ID NO: | Ab68 Heavy chain | EVQLVESGGGLVQPGRSLRLSCTASGFTFS |
| 89 | variable region (e.g., | DHDMNWVRQAPGKGLEWVGRTRNAAGS |
| | as found in HC-68) | YTTEYAASVKGRFTISRDDSKNSLYLQMN |
| | (CDRs in bold) | SLKTEDTAVYYCAREPKYWIDFDLWGRG |
| | | TLVTVSS |
| SEQ ID NO: | Ab68 Light chain | DIQMTQSPSSLSASVGDRVTITCRASQSISS |
| 90 | variable region (e.g., | YLNWYQQKPGKAPKLLIYAASSLQSGVPS |
| | as found in LC-68) | RFSGSGSGTDFTLTISSLQPEDFATYYCQQS |
| | (CDRs in bold) | YIAPYTFGGGTKVEIK |
| SEQ ID NO: | Ab68 CDR-H1 | FTFSDHDMN |
| 91 | | |
| SEQ ID NO: | Ab68 CDR-H2 | RTRNAAGSYTTEYAASVKG |
| 92 | | |
| SEQ ID NO: | Ab68 CDR-H3 | AREPKYWIDFDL |
| 93 | | |
| SEQ ID NO: | Ab68 CDR-L1 | RASQSISSYLN |
| 94 | | |
| SEQ ID NO: | Ab68 CDR-L2 | AASSLQS |
| 95 | | |
| SEQ ID NO: | Ab68 CDR-L3 | QQSYIAPYT |
| 96 | | |
| SEQ ID NO: | Ab68 Heavy chain | GAGGTGCAGCTGGTGGAGTCTGGGGGAG |
| 97 | variable region | GCTTGGTACAGCCAGGGCGGTCCCTGAG |
| | (nucl) | ACTCTCCTGTACAGCTTCTGGATTCACCT |
| | | TCAGTGACCACGACATGAACTGGGTCCG |
| | | CCAGGCTCCAGGGAAGGGGCTGGAGTGG |
| | | GTTGGCCGTACTAGAAACGCCGCTGGAA |
| | | GTTACACCACAGAATACGCCGCGTCTGT |
| | | GAAAGGCAGATTCACCATCTCAAGAGAT |
| | | GATTCAAAGAACTCACTGTATCTGCAAAT |
| | | GAACAGCCTGAAAACCGAGGACACGGCG |
| | | GTGTACTACTGCGCCAGAGAGCCTAAAT |
| | | ACTGGATCGACTTCGACCTATGGGGGAG |
| | | AGGTACCTTGGTCACCGTCTCCTCA |
| SEQ ID NO: | Ab68 Light chain | GACATCCAGATGACCCAGTCTCCATCCTC |
| 98 | variable region | CCTGTCTGCATCTGTAGGAGACAGAGTC |
| | (nucl) | ACCATCACTTGCCGGGCAAGTCAGAGCA |
| | | TTAGCAGCTATTTAAATTGGTATCAGCAG |
| | | AAACCAGGGAAAGCCCCTAAGCTCCTGA |
| | | TCTATGCTGCATCCAGTTTGCAAAGTGGG |
| | | GTCCCATCAAGGTTCAGTGGCAGTGGAT |
| | | CTGGGACAGATTTCACTCTCACCATCAGC |
| | | AGTCTGCAACCTGAAGATTTTGCAACTTA |
| | | CTACTGTCAGCAAAGCTACATCGCCCCTT |
| | | ACACTTTTGGCGGAGGGACCAAGGTTGA |
| | | GATCAAA |
| SEQ ID NO: | Ab69 Heavy chain | EVQLVESGGGLVQPGGSLRLSCAASGFTF |
| 99 | variable region (e.g., | VDHDMDWVRQAPGKGLEWVGRTRNKL |
| | as found in HC-69) | GSYTTEYAASVKGRFTISRDDSKNSLYLQ |
| | (CDRs in bold) | MNSLKTEDTAVYYCAREPKYWIDFDLWG |
| | | RGTLVTVSS |
| SEQ ID NO: | Ab69 Light chain | DIQMTQSPSSLSASVGDRVTITCRASQSISS |
| 100 | variable region (e.g., | YLNWYQQKPGKAPKLLIYAASSLQSGVPS |
| | as found in LC-69) | RFSGSGSGTDFTLTISSLQPEDFATYYCQQS |
| | (CDRs in bold) | YIAPYTFGGGTKVEIK |
| SEQ ID NO: | Ab69 CDR-H1 | FTFVDHDMD |
| 101 | | |
| SEQ ID NO: | Ab69 CDR-H2 | RTRNKLGSYTTEYAASVKG |
| 102 | | |
| SEQ ID NO: | Ab69 CDR-H3 | AREPKYWIDFDL |
| 103 | | |
| SEQ ID NO: | Ab69 CDR-L1 | RASQSISSYLN |
| 104 | | |
| SEQ ID NO: | Ab69 CDR-L2 | AASSLQS |
| 105 | | |
| SEQ ID NO: | Ab69 CDR-L3 | QQSYIAPYT |
| 106 | | |
| SEQ ID NO: | Ab69 Heavy chain | GAGGTGCAGCTGGTGGAGTCTGGGGGAG |
| 107 | variable region | GCTTGGTCCAGCCTGGAGGGTCCCTGAG |
| | (nucl) | ACTCTCCTGTGCAGCCTCTGGATTCACCT |
| | | TCGTAGACCACGACATGGACTGGGTCCG |
| | | CCAGGCTCCAGGGAAGGGGCTGGAGTGG |
| | | GTTGGCCGTACTAGAAACAAACTAGGAA |
| | | GTTACACCACAGAATACGCCGCGTCTGT |
| | | GAAAGGCAGATTCACCATCTCAAGAGAT |
| | | GATTCAAAGAACTCACTGTATCTGCAAAT |
| | | GAACAGCCTGAAAACCGAGGACACGGCG |
| | | GTGTACTACTGCGCCAGAGAGCCTAAAT |
| | | ACTGGATCGACTTCGACCTATGGGGGAG |
| | | AGGTACCTTGGTCACCGTCTCCTCA |
| SEQ ID NO: | Ab69 Light chain | GACATCCAGATGACCCAGTCTCCATCCTC |
| 108 | variable region | CCTGTCTGCATCTGTAGGAGACAGAGTC |
| | (nucl) | ACCATCACTTGCCGGGCAAGTCAGAGCA |
| | | TTAGCAGCTATTTAAATTGGTATCAGCAG |
| | | AAACCAGGGAAAGCCCCTAAGCTCCTGA |
| | | TCTATGCTGCATCCAGTTTGCAAAGTGGG |
| | | GTCCCATCAAGGTTCAGTGGCAGTGGAT |
| | | CTGGGACAGATTTCACTCTCACCATCAGC |
| | | AGTCTGCAACCTGAAGATTTTGCAACTTA |
| | | CTACTGTCAGCAAAGCTACATCGCCCCTT |
| | | ACACTTTTGGCGGAGGGACCAAGGTTGA |
| | | GATCAAA |
| SEQ ID NO: | Ab67 Light chain | DIQMTQSPSSLSASVGDRVTITCRASQS |
| 109 | LC constant region | ISSYLNWYQQKPGKAPKLLIYAASSLQ |
| | underlined | SGVPSRFSGSGSGTDFTLTISSLQPEDFA |
| | | TYYCQQSYIAPYTFGGGTKVEIKRTVA |
| | | APSVFIFPPSDEQLKSGTASVVCLLNNF |
| | | YPREAKVQWKVDNALQSGNSQESVTE |
| | | QDSKDSTYSLSSTLTLSKADYEKHKVY |
| | | ACEVTHQGLSSPVTKSFNRGEC |
| SEQ ID NO: | Ab67 Heavy chain | EVQLVESGGGLVQPGGSLRLSCAASGF |
| 110 | HC constant region | TFSDADMDWVRQAPGKGLEWVGRTR |
| | underlined | NKAGSYTTEYAASVKGRFTISRDDSKN |
| | | SLYLQMNSLKTEDTAVYYCAREPKYW |
| | | IDFDLWGRGTLVTVSSASTKGPSVFPL |
| | | APSSKSTSGGTAALGCLVKDYFPEPVT |
| | | VSWNSGALTSGVHTFPAVLQSSGLYSL |
| | | SSVVTVPSSSLGTQTYICNVNHKPSNTK |
| | | VDKKVEPKSCDKTHTCPPCPAPELLGG |
| | | PSVFLFPPKPKDTLMISRTPEVTCVVVD |
| | | VSHEDPEVKFNWYVDGVEVHNAKTKP |
| | | REEQYNSTYRVVSVLTVLHQDWLNGK |
| | | EYKCKVSNKALPAPIEKTISKAKGQPRE |
| | | PQVYTLPPSRDELTKNQVSLTCLVKGF |
| | | YPSDIAVEWESNGQPENNYKTTPPVLD |
| | | SDGSFFLYSKLTVDKSRWQQGNVFSCS |
| | | VMHEALHNHYTQKSLSLSPGK |
| SEQ ID NO: | Ab67 Heavy chain | EVQLVESGGGLVQPGGSLRLSCAASGF |
| 111 | (D265C)* | TFSDADMDWVRQAPGKGLEWVGRTR |
| | HC constant region | NKAGSYTTEYAASVKGRFTISRDDSKN |
| | underlined | SLYLQMNSLKTEDTAVYYCAREPKYW |
| | | IDFDLWGRGTLVTVSSASTKGPSVFPL |
| | | APSSKSTSGGTAALGCLVKDYFPEPVT |
| | | VSWNSGALTSGVHTFPAVLQSSGLYSL |
| | | SSVVTVPSSSLGTQTYICNVNHKPSNTK |
| | | VDKKVEPKSCDKTHTCPPCPAPELLGG |
| | | PSVFLFPPKPKDTLMISRTPEVTCVVVC |
| | | VSHEDPEVKFNWYVDGVEVHNAKTKP |
| | | REEQYNSTYRVVSVLTVLHQDWLNGK |
| | | EYKCKVSNKALPAPIEKTISKAKGQPRE |
| | | PQVYTLPPSRDELTKNQVSLTCLVKGF |
| | | YPSDIAVEWESNGQPENNYKTTPPVLD |
| | | SDGSFFLYSKLTVDKSRWQQGNVFSCS |
| | | VMHEALHNHYTQKSLSLSPGK |
| SEQ ID NO: | Ab67 Heavy chain | EVQLVESGGGLVQPGGSLRLSCAASGF |
| 112 | (L234A/L235A/ | TFSDADMDWVRQAPGKGLEWVGRTR |
| | D265C)* | NKAGSYTTEYAASVKGRFTISRDDSKN |
| | HC constant region | SLYLQMNSLKTEDTAVYYCAREPKYW |
| | underlined | IDFDLWGRGTLVTVSSASTKGPSVFPL |
| | | APSSKSTSGGTAALGCLVKDYFPEPVT |
| | | VSWNSGALTSGVHTFPAVLQSSGLYSL |
| | | SSVVTVPSSSLGTQTYICNVNHKPSNTK |
| | | VDKKVEPKSCDKTHTCPPCPAPEAAGG |
| | | PSVFLFPPKPKDTLMISRTPEVTCVVVC |
| | | VSHEDPEVKFNWYVDGVEVHNAKTKP |
| | | REEQYNSTYRVVSVLTVLHQDWLNGK |
| | | EYKCKVSNKALPAPIEKTISKAKGQPRE |
| | | PQVYTLPPSRDELTKNQVSLTCLVKGF |
| | | YPSDIAVEWESNGQPENNYKTTPPVLD |
| | | SDGSFFLYSKLTVDKSRWQQGNVFSCS |
| | | VMHEALHNHYTQKSLSLSPGK |
| SEQ ID NO: | Ab67 Heavy chain | EVQLVESGGGLVQPGGSLRLSCAASGF |
| 113 | (D265C/H435A)* | TFSDADMDWVRQAPGKGLEWVGRTR |
| | HC constant region | NKAGSYTTEYAASVKGRFTISRDDSKN |
| | underlined | SLYLQMNSLKTEDTAVYYCAREPKYW |
| | | IDFDLWGRGTLVTVSSASTKGPSVFPL |
| | | APSSKSTSGGTAALGCLVKDYFPEPVT |
| | | VSWNSGALTSGVHTFPAVLQSSGLYSL |
| | | SSVVTVPSSSLGTQTYICNVNHKPSNTK |
| | | VDKKVEPKSCDKTHTCPPCPAPELLGG |
| | | PSVFLFPPKPKDTLMISRTPEVTCVVVC |
| | | VSHEDPEVKENWYVDGVEVHNAKTKP |
| | | REEQYNSTYRVVSVLTVLHQDWLNGK |
| | | EYKCKVSNKALPAPIEKTISKAKGQPRE |
| | | PQVYTLPPSRDELTKNQVSLTCLVKGF |
| | | YPSDIAVEWESNGQPENNYKTTPPVLD |
| | | SDGSFFLYSKLTVDKSRWQQGNVFSCS |
| | | VMHEALHNAYTQKSLSLSPGK |
| SEQ ID NO: | Ab67 Heavy chain | EVQLVESGGGLVQPGGSLRLSCAASGF |
| 114 | (L234A/L235A/ | TFSDADMDWVRQAPGKGLEWVGRTR |
| | D265C/H435A)* | NKAGSYTTEYAASVKGRFTISRDDSKN |
| | HC constant region | SLYLQMNSLKTEDTAVYYCAREPKYW |
| | underlined | IDFDLWGRGTLVTVSSASTKGPSVFPL |
| | | APSSKSTSGGTAALGCLVKDYFPEPVT |
| | | VSWNSGALTSGVHTFPAVLQSSGLYSL |
| | | SSVVTVPSSSLGTQTYICNVNHKPSNTK |
| | | VDKKVEPKSCDKTHTCPPCPAPEAAGG |
| | | PSVFLFPPKPKDTLMISRTPEVTCVVVC |
| | | VSHEDPEVKFNWYVDGVEVHNAKTKP |
| | | REEQYNSTYRVVSVLTVLHQDWLNGK |
| | | EYKCKVSNKALPAPIEKTISKAKGQPRE |
| | | PQVYTLPPSRDELTKNQVSLTCLVKGF |
| | | YPSDIAVEWESNGQPENNYKTTPPVLD |
| | | SDGSFFLYSKLTVDKSRWQQGNVFSCS |
| | | VMHEALHNAYTQKSLSLSPGK |
| SEQ ID NO: | Ab55 Light chain | DIQMTQSPSSLSASVGDRVTITCRASQSINS |
| 115 | LC constant region | YLNWYQQKPGKAPKLLIYAASSLQSGVPS |
| | underlined | RFSGSGSGTDFTLTISSLQPEDFATYYCQQG |
| | | VSDITFGGGTKVEIKRTVAAPSVFIFPPSD |
| | | EQLKSGTASVVCLLNNFYPREAKVQW |
| | | KVDNALQSGNSQESVTEQDSKDSTYSL |
| | | SSTLTLSKADYEKHKVYACEVTHQGLS |
| | | SPVTKSFNRGEC |
| SEQ ID NO: | Ab55 Heavy chain | QVQLVQSGAEVKKPGSSVKVSCKASGGTF |
| 116 | HC constant region | RIYAISWVRQAPGQGLEWMGGIIPDFGVAN |
| | underlined | YAQKFQGRVTITADESTSTAYMELSSLRSE |
| | | DTAVYYCARGGLDTDEFDLWGRGTLVTV |
| | | SSASTKGPSVFPLAPSSKSTSGGTAALG |
| | | CLVKDYFPEPVTVSWNSGALTSGVHTF |
| | | PAVLQSSGLYSLSSVVTVPSSSLGTQTY |
| | | ICNVNHKPSNTKVDKKVEPKSCDKTHT |
| | | CPPCPAPELLGGPSVFLFPPKPKDTLMI |
| | | SRTPEVTCVVVDVSHEDPEVKFNWYV |
| | | DGVEVHNAKTKPREEQYNSTYRVVSV |
| | | LTVLHQDWLNGKEYKCKVSNKALPAP |
| | | IEKTISKAKGQPREPQVYTLPPSRDELT |
| | | KNQVSLTCLVKGFYPSDIAVEWESNGQ |
| | | PENNYKTTPPVLDSDGSFFLYSKLTVD |
| | | KSRWQQGNVFSCSVMHEALHNHYTQK |
| | | SLSLSPGK |
| SEQ ID NO: | Ab55 Heavy chain | QVQLVQSGAEVKKPGSSVKVSCKASGGTF |
| 117 | (D265C)* | RIYAISWVRQAPGQGLEWMGGIIPDFGVAN |
| | HC constant region | YAQKFQGRVTITADESTSTAYMELSSLRSE |
| | underlined | DTAVYYCARGGLDTDEFDLWGRGTLVTV |
| | | SSASTKGPSVFPLAPSSKSTSGGTAALG |
| | | CLVKDYFPEPVTVSWNSGALTSGVHTF |
| | | PAVLQSSGLYSLSSVVTVPSSSLGTQTY |
| | | ICNVNHKPSNTKVDKKVEPKSCDKTHT |
| | | CPPCPAPELLGGPSVFLFPPKPKDTLMI |
| | | SRTPEVTCVVVCVSHEDPEVKFNWYV |
| | | DGVEVHNAKTKPREEQYNSTYRVVSV |
| | | LTVLHQDWLNGKEYKCKVSNKALPAP |
| | | IEKTISKAKGQPREPQVYTLPPSRDELT |
| | | KNQVSLTCLVKGFYPSDIAVEWESNGQ |
| | | PENNYKTTPPVLDSDGSFFLYSKLTVD |
| | | KSRWQQGNVFSCSVMHEALHNHYTQK |
| | | SLSLSPGK |
| SEQ ID NO: | Ab55 Heavy chain | QVQLVQSGAEVKKPGSSVKVSCKASGGTF |
| 118 | (L234A/L235A/ | RIYAISWVRQAPGQGLEWMGGIIPDFGVAN |
| | D265C)* | YAQKFQGRVTITADESTSTAYMELSSLRSE |
| | HC constant region | DTAVYYCARGGLDTDEFDLWGRGTLVTV |
| | underlined | SSASTKGPSVFPLAPSSKSTSGGTAALG |
| | | CLVKDYFPEPVTVSWNSGALTSGVHTF |
| | | PAVLQSSGLYSLSSVVTVPSSSLGTQTY |
| | | ICNVNHKPSNTKVDKKVEPKSCDKTHT |
| | | CPPCPAPEAAGGPSVFLFPPKPKDTLMI |
| | | SRTPEVTCVVVCVSHEDPEVKFNWYV |
| | | DGVEVHNAKTKPREEQYNSTYRVVSV |
| | | LTVLHQDWLNGKEYKCKVSNKALPAP |
| | | IEKTISKAKGQPREPQVYTLPPSRDELT |
| | | KNQVSLTCLVKGFYPSDIAVEWESNGQ |
| | | PENNYKTTPPVLDSDGSFFLYSKLTVD |
| | | KSRWQQGNVFSCSVMHEALHNHYTQK |
| | | SLSLSPGK |
| SEQ ID NO: | Ab55 Heavy chain | QVQLVQSGAEVKKPGSSVKVSCKASGGTF |
| 119 | (D265C/H435A)* | RIYAISWVRQAPGQGLEWMGGIIPDFGVAN |
| | HC constant region | YAQKFQGRVTITADESTSTAYMELSSLRSE |
| | underlined | DTAVYYCARGGLDTDEFDLWGRGTLVTV |
| | | SSASTKGPSVFPLAPSSKSTSGGTAALG |
| | | CLVKDYFPEPVTVSWNSGALTSGVHTF |
| | | PAVLQSSGLYSLSSVVTVPSSSLGTQTY |
| | | ICNVNHKPSNTKVDKKVEPKSCDKTHT |
| | | CPPCPAPELLGGPSVFLFPPKPKDTLMI |
| | | SRTPEVTCVVVCVSHEDPEVKFNWYV |
| | | DGVEVHNAKTKPREEQYNSTYRVVSV |
| | | LTVLHQDWLNGKEYKCKVSNKALPAP |
| | | IEKTISKAKGQPREPQVYTLPPSRDELT |
| | | KNQVSLTCLVKGFYPSDIAVEWESNGQ |
| | | PENNYKTTPPVLDSDGSFFLYSKLTVD |
| | | KSRWQQGNVFSCSVMHEALHNAYTQK |
| | | SLSLSPGK |
| SEQ ID NO: | Ab55 Heavy chain | QVQLVQSGAEVKKPGSSVKVSCKASGGTF |
| 120 | (L234A/L235A/ | RIYAISWVRQAPGQGLEWMGGIIPDFGVAN |
| | D265C/H435A)* | YAQKFQGRVTITADESTSTAYMELSSLRSE |
| | HC constant region | DTAVYYCARGGLDTDEFDLWGRGTLVTV |
| | underlined | SSASTKGPSVFPLAPSSKSTSGGTAALG |
| | | CLVKDYFPEPVTVSWNSGALTSGVHTF |
| | | PAVLQSSGLYSLSSVVTVPSSSLGTQTY |
| | | ICNVNHKPSNTKVDKKVEPKSCDKTHT |
| | | CPPCPAPEAAGGPSVFLFPPKPKDTLMI |
| | | SRTPEVTCVVVCVSHEDPEVKFNWYV |
| | | DGVEVHNAKTKPREEQYNSTYRVVSV |
| | | LTVLHQDWLNGKEYKCKVSNKALPAP |
| | | IEKTISKAKGQPREPQVYTLPPSRDELT |
| | | KNQVSLTCLVKGFYPSDIAVEWESNGQ |
| | | PENNYKTTPPVLDSDGSFFLYSKLTVD |
| | | KSRWQQGNVFSCSVMHEALHNAYTQK |
| | | SLSLSPGK |
| SEQ ID NO: | Light chain constant | RTVAAPSVFIFPPSDEQLKSGTASVVCL |
| 121 | region of LC-54, | LNNFYPREAKVQWKVDNALQSGNSQE |
| | LC-55, LC-56, LC- | SVTEQDSKDSTYSLSSTLTLSKADYEK |
| | 57, LC-58, LC-61, | HKVYACEVTHQGLSSPVTKSFNRGEC |
| | LC-66, LC-67, LC- | |
| | 68, LC-69 | |
| SEQ ID NO: | Heavy chain | ASTKGPSVFPLAPSSKSTSGGTAALGCL |
| 122 | constant region of | VKDYFPEPVTVSWNSGALTSGVHTFPA |
| | WT | VLQSSGLYSLSSVVTVPSSSLGTQTYIC |
| | | NVNHKPSNTKVDKKVEPKSCDKTHTC |
| | | PPCPAPELLGGPSVFLFPPKPKDTLMIS |
| | | RTPEVTCVVVDVSHEDPEVKFNWYVD |
| | | GVEVHNAKTKPREEQYNSTYRVVSVL |
| | | TVLHQDWLNGKEYKCKVSNKALPAPI |
| | | EKTISKAKGQPREPQVYTLPPSRDELTK |
| | | NQVSLTCLVKGFYPSDIAVEWESNGQP |
| | | ENNYKTTPPVLDSDGSFFLYSKLTVDK |
| | | SRWQQGNVFSCSVMHEALHNHYTQKS |
| | | LSLSPGK |
| SEQ ID NO: | Heavy chain | ASTKGPSVFPLAPSSKSTSGGTAALGCL |
| 123 | constant region | VKDYFPEPVTVSWNSGALTSGVHTFPA |
| | (D265C)* | VLQSSGLYSLSSVVTVPSSSLGTQTYIC |
| | | NVNHKPSNTKVDKKVEPKSCDKTHTC |
| | | PPCPAPELLGGPSVFLFPPKPKDTLMIS |
| | | RTPEVTCVVVCVSHEDPEVKFNWYVD |
| | | GVEVHNAKTKPREEQYNSTYRVVSVL |
| | | TVLHQDWLNGKEYKCKVSNKALPAPI |
| | | EKTISKAKGQPREPQVYTLPPSRDELTK |
| | | NQVSLTCLVKGFYPSDIAVEWESNGQP |
| | | ENNYKTTPPVLDSDGSFFLYSKLTVDK |
| | | SRWQQGNVFSCSVMHEALHNHYTQKS |
| | | LSLSPGK |
| SEQ ID NO: | Heavy chain | ASTKGPSVFPLAPSSKSTSGGTAALGCL |
| 124 | constant region | VKDYFPEPVTVSWNSGALTSGVHTFPA |
| | (L234A / L235A / | VLQSSGLYSLSSVVTVPSSSLGTQTYIC |
| | D265C)* | NVNHKPSNTKVDKKVEPKSCDKTHTC |
| | | PPCPAPEAAGGPSVFLFPPKPKDTLMIS |
| | | RTPEVTCVVVCVSHEDPEVKFNWYVD |
| | | GVEVHNAKTKPREEQYNSTYRVVSVL |
| | | TVLHQDWLNGKEYKCKVSNKALPAPI |
| | | EKTISKAKGQPREPQVYTLPPSRDELTK |
| | | NQVSLTCLVKGFYPSDIAVEWESNGQP |
| | | ENNYKTTPPVLDSDGSFFLYSKLTVDK |
| | | SRWQQGNVFSCSVMHEALHNHYTQKS |
| | | LSLSPGK |
| SEQ ID NO: | Heavy chain | ASTKGPSVFPLAPSSKSTSGGTAALGCL |
| 125 | constant region | VKDYFPEPVTVSWNSGALTSGVHTFPA |
| | (H435A/D265C)* | VLQSSGLYSLSSVVTVPSSSLGTQTYIC |
| | | NVNHKPSNTKVDKKVEPKSCDKTHTC |
| | | PPCPAPELLGGPSVFLFPPKPKDTLMIS |
| | | RTPEVTCVVVCVSHEDPEVKFNWYVD |
| | | GVEVHNAKTKPREEQYNSTYRVVSVL |
| | | TVLHQDWLNGKEYKCKVSNKALPAPI |
| | | EKTISKAKGQPREPQVYTLPPSRDELTK |
| | | NQVSLTCLVKGFYPSDIAVEWESNGQP |
| | | ENNYKTTPPVLDSDGSFFLYSKLTVDK |
| | | SRWQQGNVFSCSVMHEALHNAYTQKS |
| | | LSLSPGK |
| SEQ ID NO: | Heavy chain | ASTKGPSVFPLAPSSKSTSGGTAALGCL |
| 126 | constant region | VKDYFPEPVTVSWNSGALTSGVHTFPA |
| | (L234A/L235A/ | VLQSSGLYSLSSVVTVPSSSLGTQTYIC |
| | H435A/D265C)* | NVNHKPSNTKVDKKVEPKSCDKTHTC |
| | | PPCPAPEAAGGPSVFLFPPKPKDTLMIS |
| | | RTPEVTCVVVCVSHEDPEVKFNWYVD |
| | | GVEVHNAKTKPREEQYNSTYRVVSVL |
| | | TVLHQDWLNGKEYKCKVSNKALPAPI |
| | | EKTISKAKGQPREPQVYTLPPSRDELTK |
| | | NQVSLTCLVKGFYPSDIAVEWESNGQP |
| | | ENNYKTTPPVLDSDGSFFLYSKLTVDK |
| | | SRWQQGNVFSCSVMHEALHNAYTQKS |
| | | LSLSPGK |
| SEQ ID NO: | Consensus sequence | GTF(S/R)(S/I/L)YAIS |
| 127 | of variable heavy | |
| | chain CDR1 (Abs | |
| | 54-57) | |
| SEQ ID NO: | Consensus sequence | GIIP(I/D/A/H)FG(T/V/L)ANYAQKFQG |
| 128 | of variable heavy | |
| | chain CDR2 (Abs | |
| | 54-57) | |
| SEQ ID NO: | Variable heavy chain | ARGGLDTDEFDL |
| 129 | CDR3 (Abs 54-57) | |
| SEQ ID NO: | Variable light chain | RASQSINSYLN |
| 130 | CDR1 (Abs 54-57) | |
| SEQ ID NO: | Variable light chain | AASSLQS |
| 131 | CDR2 (Abs 54-57) | |
| SEQ ID NO: | Variable light chain | QQGVSDIT |
| 132 | CDR3 (Abs 54-57) | |
| SEQ ID NO: | Consensus sequence | FTFS(N/S)Y(A/V)M(S/I) |
| 133 | of variable heavy | |
| | chain CDR1 (Abs | |
| | 58, 61) | |
| SEQ ID NO: | Consensus sequence | (A/S)ISG(S/D)(G/S)(G/V)(S/T)TYYADSVKG |
| 134 | of variable heavy | |
| | chain CDR2 (Abs | |
| | 58, 61) | |
| SEQ ID NO: | Variable heavy chain | AKGPPTYHTNYYYMDV |
| 135 | CDR3 (Abs 58, 61) | |
| SEQ ID NO: | Variable light chain | RASQGISSWLA |
| 136 | CDR1 (Abs 58, 61) | |
| SEQ ID NO: | Variable light chain | AASSLQS |
| 137 | CDR2 (Abs 58, 61) | |
| SEQ ID NO: | Variable light chain | QQTNSFPYT |
| 138 | CDR3 (Abs 58, 61) | |
| SEQ ID NO: | Consensus sequence | FTF(S/V)D(H/A)(Y/D)M(D/N) |
| 139 | of variable heavy | |
| | chain CDR1 (Abs | |
| | 66-69) | |
| SEQ ID NO: | Consensus sequence | RTRN(K/A)(A/L)(S/G)SYTTEYAASVKG |
| 140 | of variable heavy | |
| | chain CDR2 (Abs | |
| | 66-69) | |
| SEQ ID NO: | Variable heavy chain | AREPKYWIDFDL |
| 141 | CDR3 (Abs 66-69) | |
| SEQ ID NO: | Variable light chain | RASQSISSYLN |
| 142 | CDR1 (Abs 66-69) | |
| SEQ ID NO: | Variable light chain | AASSLQS |
| 143 | CDR2 (Abs 66-69) | |
| SEQ ID NO: | Variable light chain | QQSYIAPYT |
| 144 | CDR3 (Abs 66-69) | |
| SEQ ID NO: | Human CD117 | MRGARGAWDFLCVLLLLLRVQTGSSQ |
| 145 | (mast/stem cell | PSVSPGEPSPPSIHPGKSDLIVRVGDEIR |
| | growth factor | LLCTDPGFVKWTFEILDETNENKQNEW |
| | receptor Kit isoform | ITEKAEATNTGKYTCTNKHGLSNSIYV |
| | 1 precursor) | FVRDPAKLFLVDRSLYGKEDNDTLVRC |
| | Protein NCBI | PLTDPEVTNYSLKGCQGKPLPKDLRFIP |
| | Reference Sequence: | DPKAGIMIKSVKRAYHRLCLHCSVDQE |
| | NP_000213.1 | GKSVLSEKFILKVRPAFKAVPVVSVSK |
| | | ASYLLREGEEFTVTCTIKDVSSSVYSTW |
| | | KRENSQTKLQEKYNSWHHGDFNYERQ |
| | | ATLTISSARVNDSGVFMCYANNTFGSA |
| | | NVTTTLEVVDKGFINIFPMINTTVFVND |
| | | GENVDLIVEYEAFPKPEHQQWIYMNRT |
| | | FTDKWEDYPKSENESNIRYVSELHLTR |
| | | LKGTEGGTYTFLVSNSDVNAAIAFNVY |
| | | VNTKPEILTYDRLVNGMLQCVAAGFPE |
| | | PTIDWYFCPGTEQRCSASVLPVDVQTL |
| | | NSSGPPFGKLVVQSSIDSSAFKHNGTVE |
| | | CKAYNDVGKTSAYFNFAFKGNNKEQI |
| | | HPHTLFTPLLIGFVIVAGMMCIIVMILT |
| | | YKYLQKPMYEVQWKVVEEINGNNYV |
| | | YIDPTQLPYDHKWEFPRNRLSFGKTLG |
| | | AGAFGKVVEATAYGLIKSDAAMTVAV |
| | | KMLKPSAHLTEREALMSELKVLSYLGN |
| | | HMNIVNLLGACTIGGPTLVITEYCCYG |
| | | DLLNFLRRKRDSFICSKQEDHAEAALY |
| | | KNLLHSKESSCSDSTNEYMDMKPGVS |
| | | YVVPTKADKRRSVRIGSYIERDVTPAI |
| | | MEDDELALDLEDLLSFSYQVAKGMAF |
| | | LASKNCIHRDLAARNILLTHGRITKICD |
| | | FGLARDIKNDSNYVVKGNARLPVKWM |
| | | APESIFNCVYTFESDVWSYGIFLWELFS |
| | | LGSSPYPGMPVDSKFYKMIKEGFRMLS |
| | | PEHAPAEMYDIMKTCWDADPLKRPTF |
| | | KQIVQLIEKQISESTNHIYSNLANCSPNR |
| | | QKPVVDHSVRINSVGSTASSSQPLLVH |
| | | DDV |
| SEQ ID NO: | Human CD117 | MRGARGAWDFLCVLLLLLRVQTGSSQ |
| 146 | (mast/stem cell | PSVSPGEPSPPSIHPGKSDLIVRVGDEIR |
| | growth factor | LLCTDPGFVKWTFEILDETNENKQNEW |
| | receptor Kit isoform | ITEKAEATNTGKYTCTNKHGLSNSIYV |
| | 2 precursor) | FVRDPAKLFLVDRSLYGKEDNDTLVRC |
| | Protein NCBI | PLTDPEVTNYSLKGCQGKPLPKDLRFIP |
| | Reference Sequence: | DPKAGIMIKSVKRAYHRLCLHCSVDQE |
| | NP_001087241.1 | GKSVLSEKFILKVRPAFKAVPVVSVSK |
| | | ASYLLREGEEFTVTCTIKDVSSSVYSTW |
| | | KRENSQTKLQEKYNSWHHGDFNYERQ |
| | | ATLTISSARVNDSGVFMCYANNTFGSA |
| | | NVTTTLEVVDKGFINIFPMINTTVFVND |
| | | GENVDLIVEYEAFPKPEHQQWIYMNRT |
| | | FTDKWEDYPKSENESNIRYVSELHLTR |
| | | LKGTEGGTYTFLVSNSDVNAAIAFNVY |
| | | VNTKPEILTYDRLVNGMLQCVAAGFPE |
| | | PTIDWYFCPGTEQRCSASVLPVDVQTL |
| | | NSSGPPFGKLVVQSSIDSSAFKHNGTVE |
| | | CKAYNDVGKTSAYFNFAFKEQIHPHTL |
| | | FTPLLIGFVIVAGMMCIIVMILTYKYLQ |
| | | KPMYEVQWKVVEEINGNNYVYIDPTQ |
| | | LPYDHKWEFPRNRLSFGKTLGAGAFG |
| | | KVVEATAYGLIKSDAAMTVAVKMLKP |
| | | SAHLTEREALMSELKVLSYLGNHMNIV |
| | | NLLGACTIGGPTLVITEYCCYGDLLNFL |
| | | RRKRDSFICSKQEDHAEAALYKNLLHS |
| | | KESSCSDSTNEYMDMKPGVSYVVPTK |
| | | ADKRRSVRIGSYIERDVTPAIMEDDELA |
| | | LDLEDLLSFSYQVAKGMAFLASKNCIH |
| | | RDLAARNILLTHGRITKICDFGLARDIK |
| | | NDSNYVVKGNARLPVKWMAPESIFNC |
| | | VYTFESDVWSYGIFLWELFSLGSSPYPG |
| | | MPVDSKFYKMIKEGFRMLSPEHAPAE |
| | | MYDIMKTCWDADPLKRPTFKQIVQLIE |
| | | KQISESTNHIYSNLANCSPNRQKPVVD |
| | | HSVRINSVGSTASSSQPLLVHDDV |
| SEQ ID NO: | Heavy chain variable | QVQLVQSGAAVKKPGESLKISCKGSGYRFT |
| 147 | region of HC-1 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | AIQLTQSPSSLSASVGDRVTITCRASQGVSS |
| 148 | region of LC-1 | ALAWYQQKPGKAPKLLIYDASSLESGVPSR |
| | | FSGSGSGTDFTLTISSLQPEDFATYYCQQFN |
| | | SYPLTFGGGTKVEIK |
| SEQ ID NO: | Heavy chain variable | QVQLVQSGAAVKKPGESLKISCKGSGYRFT |
| 147 | region of HC-2 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | DIQLTQSPSSLSASVGDRVTITCRASQGIRT |
| 149 | region of LC-2 | DLGWYQQKPGKAPKLLIYDASSLESGVPSR |
| | | FSGSGSGTDFTLTISSLQPEDFATYYCQQFN |
| | | SYPLTFGGGTKVEIK |
| SEQ ID NO: | Heavy chain variable | QVQLVQSGAAVKKPGESLKISCKGSGYRFT |
| 147 | region of HC-3 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | AIRMTQSPSSLSASVGDRVTITCRASQGIRN |
| 150 | region of LC-3 | DLAWYQQKPGKTPKLLIYDASSLESGVPSR |
| | | FSGSGSGTDFTLTISSLQPEDFATYYCQQFN |
| | | SYPLTFGGGTKVEIK |
| SEQ ID NO: | Heavy chain variable | QVQLVQSGAAVKKPGESLKISCKGSGYRFT |
| 147 | region of HC-4 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | AIQMTQSPSSLSASVGDRVTITCRASQGIRN |
| 151 | region of LC-4 | DLGWYQQKPGKAPKLLIYDASSLESGVPSR |
| | | FSGSGSGTDFTLTISSLQPEDFATYYCQQFN |
| | | SYPLTFGGGTKVDIK |
| SEQ ID NO: | Heavy chain variable | QVQLVQSGAAVKKPGESLKISCKGSGYRFT |
| 147 | region of HC-5 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | NIQMTQSPSSLSASVGDRVTITCRASQAISD |
| 152 | region of LC-5 | YLAWFQQKPGKAPKLLIYDASNLETGVPS |
| | | RFSGSGSGTDFTLTISSLQPEDFATYYCQQL |
| | | NSYPLTFGGGTKVEIK |
| SEQ ID NO: | Heavy chain variable | QVQLVQSGAAVKKPGESLKISCKGSGYRFT |
| 147 | region of HC-6 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | AIRMTQSPSSLSASVGDRVIIACRASQGIGG |
| 153 | region of LC-6 | ALAWYQQKPGNAPKVLVYDASTLESGVPS |
| | | RFSGGGSGTDFTLTISSLQPEDFATYYCQQF |
| | | NSYPLTFGGGTKLEIK |
| SEQ ID NO: | Heavy chain variable | QVQLVQSGAAVKKPGESLKISCKGSGYRFT |
| 147 | region of HC-7 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | DIAMTQSPPSLSAFVGDRVTITCRASQGIISS |
| 154 | region of LC-7 | LAWYQQKPGKAPKLLIYDASSLESGVPSRF |
| | | SGSGSGTDFTLTIRSLQPEDFATYYCQQFNS |
| | | YPLTFGGGTKLEIK |
| SEQ ID NO: | Heavy chain variable | QVQLVQSGAAVKKPGESLKISCKGSGYRFT |
| 147 | region of HC-8 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | DIQMTQSPSSLSASVGDRVTITCRASQGISS |
| 155 | region of LC-8 | ALAWYQQKAGKAPKVLISDASSLESGVPS |
| | | RFSGSGSGTDFTLSISSLQPEDFATYYCQQF |
| | | NGYPLTFGGGTKVDIK |
| SEQ ID NO: | Heavy chain variable | QVQLVQSGAAVKKPGESLKISCKGSGYRFT |
| 147 | region of HC-9 | TYWIGWVRQMPGKGLEWMGIIYPGDSDTR |
| | amino acid sequence | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | AIRMTQSPSSLSASVGDRVTITCQASQGIRN |
| 156 | region of LC-9 | DLGWYQQKPGKAPKLLIYDASNLETGVPS |
| | | RFSGSGSGTDFTFTISSLQPEDIATYYCQQF |
| | | NSYPLTFGGGTKLEIK |
| SEQ ID NO: | Heavy chain variable | QVQLVQSGAAVKKPGESLKISCKGSGYRFT |
| 147 | region of HC-10 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | NIQMTQSPSSLSTSVGDRVTITCRASQGIGT |
| 157 | region of LC-10 | SLAWYQQKPGKPPKLLIYDASSLESGVPSR |
| | | LSGSGSGTDFTLTISSLQPEDFATYYCQQSN |
| | | SYPITFGQGTRLEIK |
| SEQ ID NO: | Heavy chain variable | QVQLVQSGAAVKKPGESLKISCKGSGYRFT |
| 147 | region of HC-11 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | AIQLTQSPSSLSASVGDRVTITCRASQSIGD |
| 158 | region of LC-11 | YLTWYQQKPGKAPKVLIYGASSLQSGVPP |
| | | RFSGSGSGTDFTLTVSSLQPEDFATYYCQQ |
| | | LNSYPLTFGGGTKLEIK |
| SEQ ID NO: | Heavy chain variable | QVQLVQSGAAVKKPGESLKISCKGSGYRFT |
| 147 | region of HC-12 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | DIQLTQSPSSLSASVGDRVTITCRASQGVRS |
| 159 | region of LC-12 | TLAWYQQKPGKAPKLLIYDASILESGVPSR |
| | | FSGSGSGTDFTLTISSLQPEDFATYYCQQFN |
| | | GYPLTFGQGTRLEIK |
| SEQ ID NO: | Heavy chain variable | QVQLVQSGAAVKKPGESLKISCKGSGYRFT |
| 147 | region of HC-13 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | DIVMTQSPSSLSASVGDRVTITCRASQGIRN |
| 160 | region of LC-13 | DLGWYQQKPGKAPKLLIYDASSLESGVPSR |
| | | FSGSGSGTDFTLTISSLQPEDFATYYCQQFN |
| | | SYPLTFGGGTKLEIK |
| SEQ ID NO: | Heavy chain variable | QVQLVQSGAAVKKPGESLKISCKGSGYRFT |
| 147 | region of HC-14 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | DIQLTQSPSSLSASVGDRVTITCRASQGISSF |
| 161 | region of LC-14 | LAWYQQKPGKAPKLLIYDASTLQSGVPSRF |
| | | SGSASGTDFTLTISSLQPEDFATYYCQQLNG |
| | | YPLTFGGGTKVEIK |
| SEQ ID NO: | Heavy chain variable | QVQLVQSGAAVKKPGESLKISCKGSGYRFT |
| 147 | region of HC-15 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | AIQLTQSPSSLSASVGDRVTITCRASQGIGS |
| 162 | region of LC-15 | ALAWYQQKPGIGPKLLIYDASTLESGVPAR |
| | | FSGSGSRTDFTLTITSLQPEDFATYYCQQFN |
| | | GYPLTFGGGTKLEIK |
| SEQ ID NO: | Heavy chain variable | QVQLVQSGAAVKKPGESLKISCKGSGYRFT |
| 147 | region of HC-16 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | AIQLTQSPSSLSASVGDRVTITCRASQGITS |
| 163 | region of LC-16 | ALAWYQEKPGKAPNLLIYDASSLESGVPSR |
| | | FSGSGYGTDFTLTISSLQPEDFATYYCQQLN |
| | | SYPLTFGGGTKVDIK |
| SEQ ID NO: | Heavy chain variable | QIQLVQSGPELRKPGESVKISCKASGYTFTD |
| 164 | region of HC-17 | YAMYWVKQAPGKGLKWMGWINTYTGKP |
| | | TYADDFKGRFVFSLEASANTANLQISNLKN |
| | | EDTATYFCARARGLVDDYVMDAWGQGTS |
| | | VTVSS |
| SEQ ID NO: | Light chain variable | SYELIQPPSASVTLGNTVSLTCVGDELSKRY |
| 165 | region of LC-17 | AQWYQQKPDKTIVSVIYKDSERPSGISDRF |
| | | SGSSSGTTATLTIHGTLAEDEADYYCLSTYS |
| | | DDNLPVFGGGTKLTVL |
| SEQ ID NO: | Heavy chain variable | EVQLQQYGAELGKPGTSVRLSCKVSGYNI |
| 166 | region of HC-18 | RNTYIHWVNQRPGEGLEWIGRIDPTNGNTI |
| | | SAEKFKTKATLTADTSSHTAYLQFSQLKSD |
| | | DTAIYFCALNYEGYADYWGQGVMVTGSS |
| SEQ ID NO: | Light chain variable | DIQMTQSPSFLSASVGDRVTINCKASQNIN |
| 167 | region of LC-18 | KYLNWYQQKVGEAPKRLIFKTNSLQTGIPS |
| | | RFSGSGSGTDYTLTISSLQTEDVATYFCFQY |
| | | NIGYTFGAGTKVELK |
| SEQ ID NO: | Heavy chain variable | EVQLQESGPGLVKPSQSLSLTCSVTGYSISS |
| 168 | region of HC-19 | NYRWNWIRKFPGNKVEWMGYINSAGSTN |
| | | YNPSLKSRISMTRDTSKNQFFLQVNSVTTE |
| | | DTATYYCARSLRGYITDYSGFFDYWGQGV |
| | | MVTVSS |
| SEQ ID NO: | Light chain variable | DIRMTQSPASLSASLGETVNIECLASEDIFS |
| 169 | region of LC-19 | DLAWYQQKPGKSPQLLIYNANSLQNGVPS |
| | | RFSGSGSGTRYSLKINSLQSEDVATYFCQQ |
| | | YKNYPLTFGSGTKLEIK |
| SEQ ID NO: | Heavy chain variable | EVQLQQYGAELGKPGTSVRLSCKLSGYKIR |
| 170 | region of HC-20 | NTYIHWVNQRPGKGLEWIGRIDPANGNTIY |
| | | AEKFKSKVTLTADTSSNTAYMQLSQLKSD |
| | | DTALYFCAMNYEGYEDYWGQGVMVTVSS |
| SEQ ID NO: | Light chain variable | DIQMTQSPSFLSASVGDSVTINCKASQNINK |
| 171 | region of LC-20 | YLNWYQQKLGEAPKRLIHKTDSLQTGIPSR |
| | | FSGSGSGTDYTLTISSLQPEDVATYFCFQYK |
| | | SGFMFGAGTKLELK |
| SEQ ID NO: | Heavy chain variable | QIQLVQSGPELKKPGESVKISCKASGYTFTD |
| 172 | region of HC-21 | YAVYWVIQAPGKGLKWMGWINTYTGKPT |
| | | YADDFKGRFVFSLETSASTANLQISNLKNE |
| | | DTATYFCARGAGMTKDYVMDAWGRGVL |
| | | VTVS |
| SEQ ID NO: | Light chain variable | SYELIQPPSASVTLGNTVSLTCVGDELSKRY |
| 173 | region of LC-21 | AQWYQQKPDKTIVSVIYKDSERPSDISDRF |
| | | SGSSSGTTATLTIHGTLAEDEADYYCLSTYS |
| | | DDNLPVFGGGTKLTVL |
| SEQ ID NO: | Heavy chain variable | QVQLKESGPGLVQPSQTLSLTCTVSGFSLTS |
| 174 | region of HC-22 | YLVHWVRQPPGKTLEWVGLMWNDGDTS |
| | | YNSALKSRLSISRDTSKSQVFLKMHSLQAE |
| | | DTATYYCARESNLGFTYWGHGTLVTVSS |
| SEQ ID NO: | Light chain variable | DIQMTQSPASLSASLEEIVTITCKASQGIDD |
| 175 | region of LC-22 | DLSWYQQKPGKSPQLLIYDVTRLADGVPS |
| | | RFSGSRSGTQYSLKISRPQVADSGIYYCLQS |
| | | YSTPYTFGAGTKLELK |
| SEQ ID NO: | Heavy chain variable | EVQLQQYGAELGKPGTSVRLSCKVSGYNI |
| 176 | region of HC-23 | RNTYIHWVHQRPGEGLEWIGRIDPTNGNTI |
| | | SAEKFKSKATLTADTSSNTAYMQFSQLKSD |
| | | DTAIYFCAMNYEGYADYWGQGVMVTVSS |
| SEQ ID NO: | Light chain variable | DIQMTQSPSFLSASVGDRLTINCKASQNINK |
| 177 | region of LC-23 | YLNWYQQKLGEAPKRLIFKTNSLQTGIPSR |
| | | FSGSGSGTDYTLTISSLQPEDVATYFCFQYN |
| | | IGFTFGAGTKLELK |
| SEQ ID NO: | Heavy chain variable | EVQLVESGGGLVQSGRSLKLSCAASGFTVS |
| 178 | region of HC-24 | DYYMAWVRQAPTKGLEWVATINYDGSTT |
| | | YHRDSVKGRFTISRDNAKSTLYLQMDSLRS |
| | | EDTATYYCARHGDYGYHYGAYYFDYWG |
| | | QGVMVTVSS |
| SEQ ID NO: | Light chain variable | DIVLTQSPALAVSLGQRATISCRASQTVSLS |
| 179 | region of LC-24 | GYNLIHWYQQRTGQQPKLLIYRASNLAPGI |
| | | PARFSGSGSGTDFTLTISPVQSDDIATYYCQ |
| | | QSRESWTFGGGTNLEMK |
| SEQ ID NO: | Heavy chain variable | QIQLVQSGPELKKPGESVKISCKASGYTFTD |
| 180 | region of HC-25 | YAIHWVKQAPGQGLRWMAWINTETGKPT |
| | | YADDFKGRFVFSLEASASTAHLQISNLKNE |
| | | DTATFFCAGGSHWFAYWGQGTLVTVSS |
| SEQ ID NO: | Light chain variable | SYELIQPPSASVTLENTVSITCSGDELSNKY |
| 181 | region of LC-25 | AHWYQQKPDKTILEVIYNDSERPSGISDRFS |
| | | GSSSGTTAILTIRDAQAEDEADYYCLSTFSD |
| | | DDLPIFGGGTKLTVL |
| SEQ ID NO: | Heavy chain variable | QIQLVQSGPELKKPGESVKISCKASGYTFTD |
| 172 | region of HC-26 | YAVYWVIQAPGKGLKWMGWINTYTGKPT |
| | | YADDFKGRFVFSLETSASTANLQISNLKNE |
| | | DTATYFCARGAGMTKDYVMDAWGRGVL |
| | | VTVS |
| SEQ ID NO: | Light chain variable | SYELIQPPSTSVTLGNTVSLTCVGNELPKRY |
| 182 | region of LC-26 | AYWFQQKPDQSIVRLIYDDDRRPSGISDRF |
| | | SGSSSGTTATLTIRDAQAEDEAYYYCHSTY |
| | | TDDKVPIFGGGTKLTVL |
| SEQ ID NO: | Heavy chain variable | EVQLVESGGGLVQPGRSMKLSCKASGFTF |
| 183 | region of HC-27 | SNYDMAWVRQAPTRGLEWVASISYDGITA |
| | | YYRDSVKGRFTISRENAKSTLYLQLVSLRS |
| | | EDTATYYCTTEGGYVYSGPHYFDYWGQG |
| | | VMVTVSS |
| SEQ ID NO: | Light chain variable | DIQMTQSPSSMSVSLGDTVTITCRASQDVGI |
| 184 | region of LC-27 | FVNWFQQKPGRSPRRMIYRATNLADGVPS |
| | | RFSGSRSGSDYSLTISSLESEDVADYHCLQY |
| | | DEFPRTFGGGTKLELK |
| SEQ ID NO: | Heavy chain variable | EVQLQQYGAELGKPGTSVRLSCKVSGYKI |
| 185 | region of HC-28 | RNTYIHWVNQRPGKGLEWIGRIDPANGNTI |
| | | YAEKFKSKVTLTADTSSNTAYMQLSQLKS |
| | | DDTALYFCAMNYEGYEDYWGQGVMVTV |
| | | SS |
| SEQ ID NO: | Light chain variable | DIQMTQSPSFLSASVGDSVTINCKASQNINK |
| 186 | region of LC-28 | YLNWYQQKLGEAPKRLIHKTNSLQPGFPSR |
| | | FSGSGSGTDYTLTISSLQPEDVAAYFCFQY |
| | | NSGFTFGAGTKLELK |
| SEQ ID NO: | Heavy chain variable | QVQLVQSGAEVKKPGASVKVSCKASGYTF |
| 187 | region of HC-29 | TDYYIHWVRQAPGQGLEWMGWMNPHSG |
| | | DTGYAQKFQGRVTMTRDTSTSTVYMELSS |
| | | LRSEDTAVYYCARHGRGYNGYEGAFDIW |
| | | GQGTLVTVSSAS |
| SEQ ID NO: | Light chain variable | DIQMTQSPSSLSASVGDRVTITCRASQGIGN |
| 188 | region of LC-29 | ELGWYQQKPGKAPKLLIYAASNLQSGVPS |
| | | RFSGSGSGTDFTLTISSLQPEDFATYYCQQY |
| | | DNLPLTFGQGTKVEIK |
| SEQ ID NO: | Heavy chain variable | QVQLVQSGAEVKKPGASVKVSCKASGYTF |
| 189 | region of HC-30 | TGYYLHWVRQAPGQGLEWMGWINPNSGD |
| | | TNYAQNFQGRVTMTRDTSTSTVYMELSSL |
| | | RSEDTAVYYCARHGRGYNGYEGAFDIWG |
| | | QGTLVTVSSAS |
| SEQ ID NO: | Light chain variable | DIQMTQSPSSLSASVGDRVTITCRASQGIRN |
| 190 | region of LC-30 | DLGWYQQKPGKAPKLLIYDASSLESGVPSR |
| | | FSGSGSGTDFTLTISSLQPEDFATYYCQQLN |
| | | GYPLTFGGGTKVEIK |
| SEQ ID NO: | Heavy chain variable | QVQLVQSGAEVKKPGASVKVSCKASGYTF |
| 191 | region of HC-31 | TGYYLHWVRQAPGQGLEWMGWINPNSGG |
| | | TNYAQKFQGRVTMTRDTSTSTVYMELSSL |
| | | RSEDTAVYYCARHGRGYEGYEGAFDIWG |
| | | QGTLVTVSSAS |
| SEQ ID NO: | Light chain variable | DIQMTQSPSSLSASVGDRVTITCRASQGIRN |
| 192 | region of LC-31 | DLGWYQQKPGKAPKLLIYDASELETGVPS |
| | | RFSGSGSGTDFTLTISSLQPEDFATYYCQQL |
| | | NGYPITFGQGTKVEIK |
| SEQ ID NO: | Heavy chain variable | QVQLVQSGAEVKKPGASVKVSCKASGYTF |
| 193 | region of HC-32 | TSYYIHWVRQAPGQGLEWMGWLNPSGGG |
| | | TSYAQKFQGRVTMTRDTSTSTVYMELSSL |
| | | RSEDTAVYYCARHGRGYDGYEGAFDIWG |
| | | QGTLVTVSSAS |
| SEQ ID NO: | Light chain variable | DIQMTQSPSSLSASVGDRVTITCRASQGIRN |
| 194 | region of LC-32 | DLGWYQQKPGKAPKLLIYDASNLETGVPS |
| | | RFSGSGSGTDFTLTISSLQPEDFATYYCQQL |
| | | NGYPLTFGGGTKVEIK |
| SEQ ID NO: | Heavy chain variable | QVQLVQSGAEVKKPGASVKVSCKASGYTF |
| 195 | region of HC-33 | STYYMHWVRQAPGQGLEWMGIINPSGGST |
| | | SYAQKFQGRVTMTRDTSTSTVYMKLSSLR |
| | | SEDTAVYYCARHGRGYEGYEGAFDIWGQ |
| | | GTLVTVSSAS |
| SEQ ID NO: | Light chain variable | DIQMTQSPSSLSASVGDRVTITCRASQGIRD |
| 196 | region of LC-33 | DLGWYQQKPGKAPKLLIYDASNLETGVPS |
| | | RFSGSGSGTDFTLTISSLQPEDFATYYCQQA |
| | | NGFPLTFGGGTKVEIK |
| SEQ ID NO: | Heavy chain variable | QVQLVQSGAEVKKPGASVKVSCKASGYTF |
| 197 | region of HC-34 | TGYYIHWVRQAPGQGLEWMGIINPSGGNT |
| | | NYAQNFQGRVTMTRDTSTSTVYMELSSLR |
| | | SEDTAVYYCARHGRGYNAYEGAFDIWGQ |
| | | GTLVTVSSAS |
| SEQ ID NO: | Light chain variable | DIQMTQSPSSLSASVGDRVTITCRASQGIRN |
| 198 | region of LC-34 | DLGWYQQKPGKAPKLLIYDASNLETGVPS |
| | | RFSGSGSGTDFTLTISSLQPEDFATYYCQQV |
| | | NGYPLTFGGGTKVEIK |
| SEQ ID NO: | Heavy chain variable | QVQLVQSGAEVKKPGASVKVSCKASGGTF |
| 199 | region of HC-35 | SSYAISWVRQAPGQGLEWMGVINPTVGGA |
| | | NYAQKFQGRVTMTRDTSTSTVYMELSSLR |
| | | SEDTAVYYCARHGRGYNEYEGAFDIWGQ |
| | | GTLVTVSSAS |
| SEQ ID NO: | Light chain variable | DIQMTQSPSSLSASVGDRVTITCQASQDISD |
| 200 | region of LC-35 | YLNWYQQKPGKAPKLLIYDASNLETGVPS |
| | | RFSGSGSGTDFTLTISSLQPEDFATYYCQQG |
| | | NSFPLTFGGGTKLEIK |
| SEQ ID NO: | Heavy chain variable | QVQLVQSGAEVKKLGASVKVSCKASGYTF |
| 201 | region of HC-36 | SSYYMHWVRQAPGQGLEWMGVINPNGAG |
| | | TNFAQKFQGRVTMTRDTSTSTVYMELSSL |
| | | RSEDTAVYYCARHGRGYEGYEGAFDIWG |
| | | QGTLVTVSSAS |
| SEQ ID NO: | Light chain variable | DIQMTQSPSSLSASVGDRVTITCRASQGIRN |
| 190 | region of LC-36 | DLGWYQQKPGKAPKLLIYDASSLESGVPSR |
| | | FSGSGSGTDFTLTISSLQPEDFATYYCQQLN |
| | | GYPLTFGGGTKVEIK |
| SEQ ID NO: | Heavy chain variable | QVQLVQSGAEVKKPGASVKVSCKASGYTF |
| 202 | region of HC-37 | TTYYMHWVRQAPGQGLEWMGWINPTGG |
| | | GTNYAQNFQGRVTMTRDTSTSTVYMELSS |
| | | LRSEDTAVYYCARHGRGYEGYEGAFDIWG |
| | | QGTLVTVSSAS |
| SEQ ID NO: | Light chain variable | DIQMTQSPSSLSASVGDRVTITCRASQGIRN |
| 203 | region of LC-37 | DVSWYQQKPGKAPKLLIYDASNLETGVPS |
| | | RFSGSGSGTDFTLTISSLQPEDFATYYCQQL |
| | | SGYPITFGQGTKLEIK |
| SEQ ID NO: | Heavy chain variable | QVQLVQSGAEVKKPGASVKVSCKASGYTF |
| 204 | region of HC-38 | TSYYIHWVRQAPGQGLEWMGMINPSGGST |
| | | NYAQKFQGRVTMTRDTSTSTVYMELSSLR |
| | | SEDTAVYYCARHGRGYNDYEGAFDIWGQ |
| | | GTLVTVSSAS |
| SEQ ID NO: | Light chain variable | DIQMTQSPSSLSASVGDRVTITCRASQSISD |
| 205 | region of LC-38 | WLAWYQQKPGKAPKLLIYEASNLEGGVPS |
| | | RFSGSGSGTDFTLTISSLQPEDFATYYCQQA |
| | | NSFPYTFGQGTKVEIK |
| SEQ ID NO: | Heavy chain variable | QVQLVQSGAEVKKPGASVKVSCKASGYIF |
| 206 | region of HC-39 | SAYYIHWVRQAPGQGLEWMGIINPSGGST |
| | | RYAQKFQGRVTMTRDTSTSTVYMELSSLR |
| | | SEDTAVYYCARHGRGYGGYEGAFDIWDQ |
| | | GTLVTVSSAS |
| SEQ ID NO: | Light chain variable | DIQMTQSPSSLSASVGDRVTITCRASQGIGD |
| 207 | region of LC-39 | YVAWYQQKPGKAPKLLIYDASNLETGVPS |
| | | RFSGSGSGTDFTLTISSLQPEDFATYYCQQL |
| | | NGYPITFGQGTRLEIK |
| SEQ ID NO: | Heavy chain variable | EVQLVQSGAEVKKPGESLKISCKGSGYRFT |
| 208 | region of HC-40 | SYWIGWVRQMPGKGLEWMGIIYPDDSDTR |
| | | YSPSFQGQVTISVDKSNSTAYLQWSSLKAS |
| | | DTAMYYCARHGRGYNGYEGAFDIWGQGT |
| | | LVTVSSAS |
| SEQ ID NO: | Light chain variable | DIQMTQSPSSLSASVGDRVTITCRASQGISS |
| 209 | region of LC-40 | YLAWYQQKPGKAPKLLIYDASNLETGVPS |
| | | RFSGSGSGTYFTLTISSLQPEDFATYYCQQG |
| | | ASFPITFGQGTKVEIK |
| SEQ ID NO: | Heavy chain variable | EVQLVQSGAEVKKPGESLKISCKGSGSSFP |
| 210 | region of HC-41 | NSWIAWVRQMPGKGLEWMGIIYPSDSDTR |
| | | YSPSFQGQVTISADKSISTAYLQWSSLEASD |
| | | TAMYYCARHGRGYNGYEGAFDIWGQGTL |
| | | VTVSSAS |
| SEQ ID NO: | Light chain variable | DIQMTQSPSSLSASVGDRVTITCRASQGIRN |
| 211 | region of LC-41 | YLAWYQQKPGKAPKLLIYDASSLQSGVPS |
| | | RFSGSGSGTDFTLTISSLQPEDFATYYCQQL |
| | | NSYPLTFGGGTKVEIK |
| SEQ ID NO: | Heavy chain variable | EVQLVQSGAEVKKPGESLKISCKGSGYSFD |
| 212 | region of HC-42 | SYWIGWVRQMPGKGLEWMGIMYPGDSDT |
| | | RYSPSFQGQVTISADKSISTAYLQWSSLKAS |
| | | DTAMYYCARHGRGYNAYEGAFDIWGQGT |
| | | LVTVSSAS |
| SEQ ID NO: | Light chain variable | DIQMTQSPSSLSASVGDRVTITCRASQSINN |
| 213 | region of LC-42 | WLAWYQQKPGKAPKLLIYDAFILQSGVPS |
| | | RFSGSGSGTDFTLTISSLQPEDFATYYCLQL |
| | | NSYPLTFGPGTKVDIK |
| SEQ ID NO: | Heavy chain variable | EVQLVQSGAEVKKPGESLKISCKGSGYSFT |
| 214 | region of HC-43 | NWIAWVRQMPGKGLEWMGIIYPGDSETRY |
| | | SPSFQGQVTISADKSISTAYLQWSSLKASDT |
| | | AMYYCARHGRGYYGYEGAFDIWGQGTLV |
| | | TVSSAS |
| SEQ ID NO: | Light chain variable | DIQMTQSPSSLSASVGDRVTITCRASQGISD |
| 215 | region of LC-43 | NLNWYQQKPGKAPKLLIYDASNLETGVPS |
| | | RFSGSGSGTDFTLTISSLQPEDFATYYCQQA |
| | | ISFPLTFGQGTKVEIK |
| SEQ ID NO: | Heavy chain variable | EVQLVQSGAEVKKPGESLKISCKGSGYNFT |
| 216 | region of HC-44 | SYWIGWVRQMPGKGLEWMGVIYPDDSET |
| | | RYSPSFQGQVTISADKSISTAYLQWSSLKAS |
| | | DTAMYYCARHGRGYNGYEGAFDIWGQGT |
| | | LVTVSSAS |
| SEQ ID NO: | Light chain variable | DIQMTQSPSSLSASVGDRVTITCRASRDIRD |
| 217 | region of LC-44 | DLGWYQQKPGKAPKLLIYDASNLETGVPS |
| | | RFSGSGSGTDFTLTISSLQPEDFATYYCQQA |
| | | NSFPLTFGGGTKVEIK |
| SEQ ID NO: | Heavy chain variable | EVQLVQSGAEVKKPGESLKISCKGSGYTFN |
| 218 | region of HC-45 | TYIGWVRQMPGKGLEWMGIIYPGDSGTRY |
| | | SPSFQGQVTISADKAISTAYLQWSSLKASD |
| | | TAMYYCARHSRGYNGYEGAFDIWGQGTL |
| | | VTVSSAS |
| SEQ ID NO: | Light chain variable | DIQMTQSPSSLSASVGDRVTITCRASQGISN |
| 219 | region of LC-45 | YLAWYQQKPGKAPKLLIYDASNLETGVPS |
| | | RFSGSGSGTDFTLTISSLQPEDFATYYCQQA |
| | | NSFPVTFGQGTKVEIK |
| SEQ ID NO: | Heavy chain variable | EVQLVQSGAEVKKPGESLKISCKGSGYNFT |
| 220 | region of HC-46 | TYWIGWVRQMPGKGLEWMGIIHPADSDTR |
| | | YNPSFQGQVTISADKSISTAYLQWSSLKAS |
| | | DTAMYYCARHGRGYNGYEGAFDIWGQGT |
| | | LVTVSSAS |
| SEQ ID NO: | Light chain variable | DIQMTQSPSSLSASVGDRVTITCRVSQGISS |
| 221 | region of LC-46 | YLAWYQQKPGKAPKLLIYDASNLETGVPS |
| | | RFSGSGSGTDFTLTISSLQPEDFATYYCQQA |
| | | NSFPLTFGGGTKVEIK |
| SEQ ID NO: | Heavy chain variable | EVQLVQSGAEVKKPGESLKISCKGSGYRFS |
| 222 | region of HC-47 | NYWIAWVRQMPGKGLEWMGIIYPDNSDT |
| | | RYSPSFQGQVTISADKSISTAYLQWSSLKAS |
| | | DTAMYYCARHGRGYDGYEGAFDIWGQGT |
| | | LVTVSSAS |
| SEQ ID NO: | Light chain variable | DIQMTQSPSSLSASVGDRVTITCRASQGIRS |
| 223 | region of LC-47 | DLAWYQQKPGKAPKLLIYGASSLQSGVPS |
| | | RFSGSGSGTDFTLTISSLQPEDFATYYCQQA |
| | | NSFPLSFGQGTKVEIK |
| SEQ ID NO: | Heavy chain variable | EVQLVQSGAEVKKPGESLKISCKGSGYRFA |
| 224 | region of HC-48 | SYWIGWVRQMPGKGLEWMGITYPGDSET |
| | | RYNPSQGQVTISADKSISTAYLQWSSLKAS |
| | | DTAMYYCARHGRGYGGYEGAFDIWGQGT |
| | | LVTVSSAS |
| SEQ ID NO: | Light chain variable | DIQMTQSPSSLSASVGDRVTITCRASQGIRN |
| 225 | region of LC-48 | DLGWYQQKPGKAPKLLIYDASNLETGVPS |
| | | RFSGSGSGTDFTLTISSLQPEDFATYYCQQA |
| | | NSFPLTFGGGTKVEIK |
| SEQ ID NO: | Heavy chain variable | EVQLVQSGAEVKKPGESLKISCKGSGYSFT |
| 226 | region of HC-49 | SYWIGWVRQMPGKGLEWMGIIYPGDSDTR |
| | | YSPSFQGQVTISADKSISTAYLQWSSLKAS |
| | | DTAMYYCARHGRGYNGYEGAFDIWGQGT |
| | | LVTVSSAS |
| SEQ ID NO: | Light chain variable | DIQMTQSPSSLSASVGDRVTITCRASQSISN |
| 227 | region of LC-49 | WLAWYQQKPGKAPKLLIYDASNLETGVPS |
| | | RFSGSGSGTDFTLTISSLQPEDFATYYCQQT |
| | | NSFPLTFGQGTRLEIK |
| SEQ ID NO: | Heavy chain variable | QVQLVQSGAAVKKPGESLKISCKGSGYRFT |
| 147 | region of HC-74 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | DIQLTQSPSSLSASVGDRVTITCRASQGVIS |
| 228 | region of LC-74 | ALAWYQQKPGKAPKLLIYDASSLESGVPSR |
| | | FSGSGSGTDFTLTISSLQPEDFATYYCQQFN |
| | | SYPLTFGGGTKVEIK |
| SEQ ID NO: | Heavy chain variable | QVQLVQSGAAVKKPGESLKISCKGSGYRFT |
| 147 | region of HC-75 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | DIQLTQSPSSLSASVGDRVTITCRASQGIRS |
| 229 | region of LC-75 | ALAWYQQKPGKAPKLLIYDASSLESGVPSR |
| | | FSGSGSGTDFTLTISSLQPEDFATYYCQQFN |
| | | SYPLTFGGGTKVEIK |
| SEQ ID NO: | Heavy chain variable | QVQLVQSGAAVKKPGESLKISCKGSGYRFT |
| 147 | region of HC-76 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | DIQLTQSPSSLSASVGDRVTITCRASQGVGS |
| 230 | region of LC-76 | ALAWYQQKPGKAPKLLIYDASSLESGVPSR |
| | | FSGSGSGTDFTLTISSLQPEDFATYYCQQFN |
| | | SYPLTFGGGTKVEIK |
| SEQ ID NO: | Heavy chain variable | QVQLVQSGAAVKKPGESLKISCKGSGYRFT |
| 147 | region of HC-77 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | DIQLTQSPSSLSASVGDRVTITCRASQGVIS |
| 231 | region of LC-77 | ALAWYQQKPGKAPKLLIYDASILESGVPSR |
| | | FSGSGSGTDFTLTISSLQPEDFATYYCQQFN |
| | | SYPLTFGGGTKVEIK |
| SEQ ID NO: | Heavy chain variable | QVQLVQSGAAVKKPGESLKISCKGSGYRFT |
| 147 | region of HC-78 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | DIQLTQSPSSLSASVGDRVTITCRASQGIRS |
| 232 | region of LC-78 | ALAWYQQKPGKAPKLLIYDASILESGVPSR |
| | | FSGSGSGTDFTLTISSLQPEDFATYYCQQFN |
| | | SYPLTFGGGTKVEIK |
| SEQ ID NO: | Heavy chain variable | QVQLVQSGAAVKKPGESLKISCKGSGYRFT |
| 147 | region of HC-79 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | DIQLTQSPSSLSASVGDRVTITCRASQGVGS |
| 233 | region of LC-79 | ALAWYQQKPGKAPKLLIYDASILESGVPSR |
| | | FSGSGSGTDFTLTISSLQPEDFATYYCQQFN |
| | | SYPLTFGGGTKVEIK |
| SEQ ID NO: | Heavy chain variable | QVQLVQSGAAVKKPGESLKISCKGSGYRFT |
| 147 | region of HC-80 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | DIQLTQSPSSLSASVGDRVTITCRASQGISS |
| 234 | region of LC-80 | ALAWYQQKPGKAPKLLIYDASILESGVPSR |
| | | FSGSGSGTDFTLTISSLQPEDFATYYCQQFN |
| | | SYPLTFGGGTKVEIK |
| SEQ ID NO: | Heavy chain variable | QVQLVQSGAAVKKPGESLKISCKGSGYRFT |
| 147 | region of HC-81 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | DIQLTQSPSSLSASVGDRVTITCRASQGVIS |
| 235 | region of LC-81 | ALAWYQQKPGKAPKLLIYDASTLESGVPS |
| | | RFSGSGSGTDFTLTISSLQPEDFATYYCQQF |
| | | NSYPLTFGGGTKVEIK |
| SEQ ID NO: | Heavy chain variable | QVQLVQSGAAVKKPGESLKISCKGSGYRFT |
| 147 | region of HC-82 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | DIQLTQSPSSLSASVGDRVTITCRASQGIRS |
| 236 | region of LC-82 | ALAWYQQKPGKAPKLLIYDASTLESGVPS |
| | | RFSGSGSGTDFTLTISSLQPEDFATYYCQQF |
| | | NSYPLTFGGGTKVEIK |
| SEQ ID NO: | Heavy chain variable | QVQLVQSGAAVKKPGESLKISCKGSGYRFT |
| 147 | region of HC-83 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | DIQLTQSPSSLSASVGDRVTITCRASQGVGS |
| 237 | region of LC-83 | ALAWYQQKPGKAPKLLIYDASTLESGVPS |
| | | RFSGSGSGTDFTLTISSLQPEDFATYYCQQF |
| | | NSYPLTFGGGTKVEIK |
| SEQ ID NO: | Heavy chain variable | QVQLVQSGAAVKKPGESLKISCKGSGYRFT |
| 147 | region of HC-84 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | DIQLTQSPSSLSASVGDRVTITCRASQGVGS |
| 237 | region of LC-84 | ALAWYQQKPGKAPKLLIYDASTLESGVPS |
| | | RFSGSGSGTDFTLTISSLQPEDFATYYCQQF |
| | | NSYPLTFGGGTKVEIK |
| SEQ ID NO: | Heavy chain variable | EVQLVQSGAEVKKPGESLKISCKGSGYRFT |
| 238 | region of HC-245 | TSWIGWVRQMPGKGLEWMGIIYPGDSDTR |
| | | YSPSFQGQVTISADKSISTAYLQWSSLKAS |
| | | DTAMYYCARHGLGYNGYEGAFDIWGQGT |
| | | LVTVSS |
| SEQ ID NO: | Light chain variable | DIQMTQSPSSLSASVGDRVTITCRASQGIGS |
| 239 | region of LC-245 | ALAWYQQKPGKAPKLLIYDASTLESGVPS |
| | | RFSGSGSGTDFTLTISSLQPEDFATYYCQQF |
| | | NGYPLTFGQGTRLEIK |
| SEQ ID NO: | Heavy chain variable | QVQLVQSGAAVKKPGESLKISCKGSGYRFT |
| 147 | region of HC-246 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | DIQMTQSPSSLSASVGDRVTITCRASQGIGS |
| 239 | region of LC-246 | ALAWYQQKPGKAPKLLIYDASTLESGVPS |
| | | RFSGSGSGTDFTLTISSLQPEDFATYYCQQF |
| | | NGYPLTFGQGTRLEIK |
| SEQ ID NO: | Heavy chain variable | QVQLVQSGAAVKKPGESLKISCKGSGYRFT |
| 147 | region of HC-247 | TYWIGWVRQMPGKGLEWMGIIYPGDSDTR |
| | | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | DIQMTQSPSSLSASVGDRVTITCRASRGISD |
| 240 | region of LC-247 | YLAWYQQKPGKAPKLLIYDASNLETGVPS |
| | | RFSGSGSGTDFTLTISSLQPEDFATYYCQQA |
| | | NSFPITFGQGTRLEIK |
| SEQ ID NO: | Heavy chain variable | EVQLVQSGAEVKKPGESLKISCKGSGYRFT |
| 238 | region of HC-248 | TSWIGWVRQMPGKGLEWMGIIYPGDSDTR |
| | | YSPSFQGQVTISADKSISTAYLQWSSLKAS |
| | | DTAMYYCARHGLGYNGYEGAFDIWGQGT |
| | | LVTVSS |
| SEQ ID NO: | Light chain variable | DIQMTQSPSSLSASVGDRVTITCRASQGIGS |
| 241 | region of LC-248 | ALAWYQQKPGKAPKLLIYDASTLESGVPS |
| | | RFSGSGSGTDFTLTISSLQPEDFATYYCQQL |
| | | NGYPLTFGQGTRLEIK |
| SEQ ID NO: | Heavy chain variable | EVQLVQSGAEVKKPGESLKISCKGSGYRFT |
| 238 | region of HC-249 | TSWIGWVRQMPGKGLEWMGIIYPGDSDTR |
| | | YSPSFQGQVTISADKSISTAYLQWSSLKAS |
| | | DTAMYYCARHGLGYNGYEGAFDIWGQGT |
| | | LVTVSS |
| SEQ ID NO: | Light chain variable | DIQMTQSPSSLSASVGDRVTITCRASQGIGS |
| 242 | region of LC-249 | ALAWYQQKPGKAPKLLIYDASNLETGVPS |
| | | RFSGSGSGTDFTLTISSLQPEDFATYYCQQL |
| | | NGYPLTFGQGTRLEIK |
| SEQ ID NO: | Heavy chain variable | EVQLVQSGAEVKKPGESLKISCKGSGYSFT |
| 243 | region of Ab 85 | NYWIGWVRQMPGKGLEWMAIINPRDSDT |
| | | RYRPSFQGQVTISADKSISTAYLQWSSLKA |
| | | SDTAMYYCARHGRGYEGYEGAFDIWGQG |
| | | TLVTVSS |
| SEQ ID NO: | Light chain variable | DIQMTQSPSSLSASVGDRVTITCRSSQGIRS |
| 244 | region of Ab 85 | DLGWYQQKPGKAPKLLIYDASNLETGVPS |
| | | RFSGSGSGTDFTLTISSLQPEDFATYYCQQA |
| | | NGFPLTFGGGTKVEIK |
| SEQ ID NO: | Ab85 CDR-H1 | NYWIG |
| 245 | | |
| SEQ ID NO: | Ab85 CDR-H2 | IINPRDSDTRYRPSFQG |
| 246 | | |
| SEQ ID NO: | Ab85 CDR-H3 | HGRGYEGYEGAFDI |
| 247 | | |
| SEQ ID NO: | Ab85 CDR-L1 | RSSQGIRSDLG |
| 248 | | |
| SEQ ID NO: | Ab85 CDR-L2 | DASNLET |
| 249 | Ab249 CDR-L2 | |
| SEQ ID NO: | Ab85 CDR-L3 | QQANGFPLT |
| 250 | | |
| SEQ ID NO: | Heavy chain variable | EVQLVQSGAEVKKPGESLKISCKGSGYSFT |
| 251 | region of Ab 86 | NYWIGWVRQMPGKGLEWMGIIYPGDSDIR |
| | | YSPSLQGQVTISVDTSTSTAYLQWNSLKPS |
| | | DTAMYYCARHGRGYNGYEGAFDIWGQGT |
| | | LVTVSS |
| SEQ ID NO: | Light chain variable | DIQMTQSPSSLSASVGDRVTITCRASQGIGD |
| 252 | region of Ab 86 | SLAWYQQKPGKAPKLLIYDASNLETGVPS |
| | | RFSGSGSGTDFTLTISSLQPEDFATYYCQQL |
| | | NGYPITFGQGTKVEIK |
| SEQ ID NO: | Ab86 CDR-H1 | NYWIG |
| 245 | | |
| SEQ ID NO: | Ab86 CDR-H2 | IIYPGDSDIRYSPSLQG |
| 253 | | |
| SEQ ID NO: 3 | Ab86 CDR-H3 | HGRGYNGYEGAFDI |
| SEQ ID NO: | Ab86 CDR-L1 | RASQGIGDSLA |
| 254 | | |
| SEQ ID NO: | Ab86 CDR-L2 | DASNLET |
| 249 | | |
| SEQ ID NO: | Ab86 CDR-L3 | QQLNGYPIT |
| 255 | | |
| SEQ ID NO: | Heavy chain variable | EVQLVQSGAEVKKPGESLKISCKGSGYSFT |
| 243 | region of Ab 87 | NYWIGWVRQMPGKGLEWMAIINPRDSDT |
| | | RYRPSFQGQVTISADKSISTAYLQWSSLKA |
| | | SDTAMYYCARHGRGYEGYEGAFDIWGQG |
| | | TLVTVSS |
| SEQ ID NO: | Light chain variable | DIQMTQSPSSLSASVGDRVTITCRASQGIRN |
| 256 | region of Ab 87 | DLGWYQQKPGKAPKLLIYDASSLESGVPSR |
| | | FSGSGSGTDFTLTISSLQPEDFATYYCQQLN |
| | | GYPITFGQGTKVEIK |
| SEQ ID NO: | Ab87 CDR-H1 | NYWIG |
| 245 | | |
| SEQ ID NO: | Ab87 CDR-H2 | IINPRDSDTRYRPSFQG |
| 246 | | |
| SEQ ID NO: | Ab87 CDR-H3 | HGRGYEGYEGAFDI |
| 247 | | |
| SEQ ID NO: | Ab87 CDR-L1 | RASQGIRNDLG |
| 257 | | |
| SEQ ID NO: 5 | Ab87 CDR-L2 | DASSLES |
| SEQ ID NO: | Ab87 CDR-L3 | QQLNGYPIT |
| 255 | | |
| SEQ ID NO: | Heavy chain variable | EVQLVQSGAEVKKPGESLKISCKGSGYSFT |
| 258 | region of Ab 88 | NYWIGWVRQMPGKGLEWMGIIYPGDSLTR |
| | | YSPSFQGQVTISADKSISTAYLQWSSLKAS |
| | | DTAMYYCARHGRGYNGYEGAFDIWGQGT |
| | | LVTVSS |
| SEQ ID NO: | Light chain variable | DIQMTQSPSSLSASVGDRVTITCRASQGIRN |
| 256 | region of Ab 88 | DLGWYQQKPGKAPKLLIYDASSLESGVPSR |
| | | FSGSGSGTDFTLTISSLQPEDFATYYCQQLN |
| | | GYPITFGQGTKVEIK |
| SEQ ID NO: | Ab88 CDR-H1 | NYWIG |
| 245 | | |
| SEQ ID NO: | Ab88 CDR-H2 | IIYPGDSLTRYSPSFQG |
| 259 | | |
| SEQ ID NO: 3 | Ab88 CDR-H3 | HGRGYNGYEGAFDI |
| SEQ ID NO: | Ab88 CDR-L1 | RASQGIRNDLG |
| 257 | | |
| SEQ ID NO:5 | Ab88 CDR-L2 | DASSLES |
| SEQ ID NO: | Ab88 CDR-L3 | QQLNGYPIT |
| 255 | | |
| SEQ ID NO: | Heavy chain variable | EVQLVQSGAEVKKPGESLKISCKGSGYSFT |
| 260 | region of Ab89 | NYWIGWVRQMPGKGLEWMGIIYPGDSDT |
| | | RYSPSFQGQVTISADKSISTAYLQWSSLKAS |
| | | DTAMYYCARHGRGYNGYEGAFDIWGQGT |
| | | LVTVSS |
| SEQ ID NO: | Light chain variable | DIQMTQSPSSLSASVGDRVTITCRASQGIGD |
| 252 | region of Ab89 | SLAWYQQKPGKAPKLLIYDASNLETGVPS |
| | | RFSGSGSGTDFTLTISSLQPEDFATYYCQQL |
| | | NGYPITFGQGTKVEIK |
| SEQ ID NO: | Ab89 CDR-H1 | NYWIG |
| 245 | | |
| SEQ ID NO: 2 | Ab89 CDR-H2 | IIYPGDSDTRYSPSFQG |
| SEQ ID NO: 3 | Ab89 CDR-H3 | HGRGYNGYEGAFDI |
| SEQ ID NO: | Ab89 CDR-L1 | RASQGIGDSLA |
| 254 | | |
| SEQ ID NO: | Ab89 CDR-L2 | DASNLET |
| 249 | | |
| SEQ ID NO: | Ab89 CDR-L3 | QQLNGYPIT |
| 255 | | |
| SEQ ID NO: | Heavy chain variable | QVQLVQSGAAVKKPGESLKISCKGSGYRFT |
| 261 | region amino acid | SYWIGWVRQMPGKGLEWMGIIYPGDSDTR |
| | sequence of CK6 | YSPSFQGQVTISAGKSISTAYLQWSSLKAS |
| | | DTAMYYCARHGRGYNGYEGAFDIWGQGT |
| | | MVTVSS |
| SEQ ID NO: | Light chain variable | AIQLTQSPSSLSASVGDRVTITCRASQGISS |
| 262 | region amino acid | ALAWYQQKPGKAPKLLIYDASSLESGVPSR |
| | sequence of CK6 | FSGSGSGTDFTLTISSLQPEDFATYYCQQFN |
| | | SYPLTFGGGTKVEIK |
| SEQ ID NO: | Ab77 CDR-H1 | TYWIG |
| 263 | | |
| SEQ ID NO: 2 | Ab77 CDR-H2 | IIYPGDSDTRYSPSFQG |
| SEQ ID NO: 3 | Ab77 CDR-H3 | HGRGYNGYEGAFDI |
| SEQ ID NO: | Ab77 CDR-L1 | RASQGVISALA |
| 264 | | |
| SEQ ID NO: | Ab77 CDR-L2 | DASILES |
| 265 | | |
| SEQ ID NO: | Ab77 CDR-L3 | QQFNSYPLT |
| 266 | | |
| SEQ ID NO: | Ab79 CDR-H1 | TYWIG |
| 263 | | |
| SEQ ID NO: 2 | Ab79 CDR-H2 | IIYPGDSDTRYSPSFQG |
| SEQ ID NO: 3 | Ab79 CDR-H3 | HGRGYNGYEGAFDI |
| SEQ ID NO: | Ab79 CDR-L1 | RASQGVGSALA |
| 267 | | |
| SEQ ID NO: | Ab79 CDR-L2 | DASILES |
| 265 | | |
| SEQ ID NO: | Ab79 CDR-L3 | QQFNSYPLT |
| 266 | | |
| SEQ ID NO: | Ab81 CDR-H1 | TYWIG |
| 263 | | |
| SEQ ID NO: 2 | Ab81 CDR-H2 | IIYPGDSDTRYSPSFQG |
| SEQ ID NO: 3 | Ab81 CDR-H3 | HGRGYNGYEGAFDI |
| SEQ ID NO: | Ab81 CDR-L1 | RASQGVISALA |
| 264 | | |
| SEQ ID NO: | Ab81 CDR-L2 | DASTLES |
| 268 | | |
| SEQ ID NO: | Ab81 CDR-L3 | QQFNSYPLT |
| 266 | | |
| SEQ ID NO: | Heavy chain | ASTKGPSVFPLAPSSKSTSGGTAALGCLVK |
| 269 | constant region | DYFPEPVTVSWNSGALTSGVHTFPAVLQSS |
| | (Wild type (WT)) | GLYSLSSVVTVPSSSLGTQTYICNVNHKPS |
| | | NTKVDKKVEPKSCDKTHTCPPCPAPELLG |
| | | GPSVFLFPPKPKDTLMISRTPEVTCVVVDV |
| | | SHEDPEVKFNWYVDGVEVHNAKTKPREE |
| | | QYNSTYRVVSVLTVLHQDWLNGKEYKCK |
| | | VSNKALPAPIEKTISKAKGQPREPQVYTLPP |
| | | SRDELTKNQVSLTCLVKGFYPSDIAVEWES |
| | | NGQPENNYKTTPPVLDSDGSFFLYSKLTVD |
| | | KSRWQQGNVFSCSVMHEALHNHYTQKSL |
| | | SLSPGK |
| SEQ ID NO: | Heavy chain | ASTKGPSVFPLAPSSKSTSGGTAALGCLVK |
| 270 | constant region with | DYFPEPVTVSWNSGALTSGVHTFPAVLQSS |
| | L234A, L235A | GLYSLSSVVTVPSSSLGTQTYICNVNHKPS |
| | (LALA) mutations | NTKVDKKVEPKSCDKTHTCPPCPAPEAAG |
| | (mutations in bold)* | GPSVFLFPPKPKDTLMISRTPEVTCVVVDV |
| | | SHEDPEVKFNWYVDGVEVHNAKTKPREE |
| | | QYNSTYRVVSVLTVLHQDWLNGKEYKCK |
| | | VSNKALPAPIEKTISKAKGQPREPQVYTLPP |
| | | SRDELTKNQVSLTCLVKGFYPSDIAVEWES |
| | | NGQPENNYKTTPPVLDSDGSFFLYSKLTVD |
| | | KSRWQQGNVFSCSVMHEALHNHYTQKSL |
| | | SLSPGK |
| SEQ ID NO: | Heavy chain constant | ASTKGPSVFPLAPSSKSTSGGTAALGCLVK |
| 271 | region with D265C | DYFPEPVTVSWNSGALTSGVHTFPAVLQSS |
| | mutation | GLYSLSSVVTVPSSSLGTQTYICNVNHKPS |
| | (mutation in bold)* | NTKVDKKVEPKSCDKTHTCPPCPAPELLGG |
| | | PSVFLFPPKPKDTLMISRTPEVTCVVVCVS |
| | | HEDPEVKFNWYVDGVEVHNAKTKPREEQ |
| | | YNSTYRVVSVLTVLHQDWLNGKEYKCKV |
| | | SNKALPAPIEKTISKAKGQPREPQVYTLPPS |
| | | RDELTKNQVSLTCLVKGFYPSDIAVEWESN |
| | | GQPENNYKTTPPVLDSDGSFFLYSKLTVDK |
| | | SRWQQGNVFSCSVMHEALHNHYTQKSLSL |
| | | SPGK |
| SEQ ID NO: | Heavy chain constant | ASTKGPSVFPLAPSSKSTSGGTAALGCLVK |
| 272 | region with H435A | DYFPEPVTVSWNSGALTSGVHTFPAVLQSS |
| | mutation | GLYSLSSVVTVPSSSLGTQTYICNVNHKPS |
| | (mutation in bold)* | NTKVDKKVEPKSCDKTHTCPPCPAPELLGG |
| | | PSVFLFPPKPKDTLMISRTPEVTCVVVDVS |
| | | HEDPEVKFNWYVDGVEVHNAKTKPREEQ |
| | | YNSTYRVVSVLTVLHQDWLNGKEYKCKV |
| | | SNKALPAPIEKTISKAKGQPREPQVYTLPPS |
| | | RDELTKNQVSLTCLVKGFYPSDIAVEWESN |
| | | GQPENNYKTTPPVLDSDGSFFLYSKLTVDK |
| | | SRWQQGNVFSCSVMHEALHNAYTQKSLSL |
| | | SPGK |
| SEQ ID NO: | Heavy chain | ASTKGPSVFPLAPSSKSTSGGTAALGCLVK |
| 273 | constant region: | DYFPEPVTVSWNSGALTSGVHTFPAVLQSS |
| | modified Fc region | GLYSLSSVVTVPSSSLGTQTYICNVNHKPS |
| | with L234A, L235A, | NTKVDKKVEPKSCDKTHTCPPCPAPEAAG |
| | D265C mutations | GPSVFLFPPKPKDTLMISRTPEVTCVVVCV |
| | (mutations in bold)* | SHEDPEVKFNWYVDGVEVHNAKTKPREE |
| | | QYNSTYRVVSVLTVLHQDWLNGKEYKCK |
| | | VSNKALPAPIEKTISKAKGQPREPQVYTLPP |
| | | SRDELTKNQVSLTCLVKGFYPSDIAVEWES |
| | | NGQPENNYKTTPPVLDSDGSFFLYSKLTVD |
| | | KSRWQQGNVFSCSVMHEALHNHYTQKSL |
| | | SLSPGK |
| SEQ ID NO: | Heavy chain | ASTKGPSVFPLAPSSKSTSGGTAALGCLVK |
| 274 | constant region: | DYFPEPVTVSWNSGALTSGVHTFPAVLQSS |
| | modified Fc region | GLYSLSSVVTVPSSSLGTQTYICNVNHKPS |
| | with L234A, L235A, | NTKVDKKVEPKSCDKTHTCPPCPAPEAAG |
| | D265C, H435A | GPSVFLFPPKPKDTLMISRTPEVTCVVVCV |
| | mutations (mutations | SHEDPEVKFNWYVDGVEVHNAKTKPREE |
| | in bold)* | QYNSTYRVVSVLTVLHQDWLNGKEYKCK |
| | | VSNKALPAPIEKTISKAKGQPREPQVYTLPP |
| | | SRDELTKNQVSLTCLVKGFYPSDIAVEWES |
| | | NGQPENNYKTTPPVLDSDGSFFLYSKLTVD |
| | | KSRWQQGNVFSCSVMHEALHNAYTQKSL |
| | | SLSPGK |
| SEQ ID NO: | Ab85 full length | EVQLVQSGAEVKKPGESLKISCKGSGYSFT |
| 275 | heavy chain | NYWIGWVRQMPGKGLEWMAIINPRDSDT |
| | sequence; constant | RYRPSFQGQVTISADKSISTAYLQWSSLKA |
| | region underlined | SDTAMYYCARHGRGYEGYEGAFDIWGQG |
| | | TLVTVSSASTKGPSVFPLAPSSKSTSGGTAA |
| | | LGCLVKDYFPEPVTVSWNSGALTSGVHTFP |
| | | AVLQSSGLYSLSSVVTVPSSSLGTQTYICNV |
| | | NHKPSNTKVDKKVEPKSCDKTHTCPPCPA |
| | | PELLGGPSVFLFPPKPKDTLMISRTPEVTCV |
| | | VVDVSHEDPEVKFNWYVDGVEVHNAKTK |
| | | PREEQYNSTYRVVSVLTVLHQDWLNGKEY |
| | | KCKVSNKALPAPIEKTISKAKGQPREPQVY |
| | | TLPPSRDELTKNQVSLTCLVKGFYPSDIAVE |
| | | WESNGQPENNYKTTPPVLDSDGSFFLYSKL |
| | | TVDKSRWQQGNVFSCSVMHEALHNHYTQ |
| | | KSLSLSPGK |
| SEQ ID NO: | Ab85 full length | EVQLVQSGAEVKKPGESLKISCKGSGYSFT |
| 276 | heavy chain | NYWIGWVRQMPGKGLEWMAIINPRDSDT |
| | sequence; constant | RYRPSFQGQVTISADKSISTAYLQWSSLKA |
| | region underlined; | SDTAMYYCARHGRGYEGYEGAFDIWGQG |
| | modified Fc region | TLVTVSSASTKGPSVFPLAPSSKSTSGGTAA |
| | with L234A, L235A | LGCLVKDYFPEPVTVSWNSGALTSGVHTFP |
| | mutations (mutations | AVLQSSGLYSLSSVVTVPSSSLGTQTYICNV |
| | in bold)* | NHKPSNTKVDKKVEPKSCDKTHTCPPCPA |
| | | PEAAGGPSVFLFPPKPKDTLMISRTPEVTCV |
| | | VVDVSHEDPEVKFNWYVDGVEVHNAKTK |
| | | PREEQYNSTYRVVSVLTVLHQDWLNGKEY |
| | | KCKVSNKALPAPIEKTISKAKGQPREPQVY |
| | | TLPPSRDELTKNQVSLTCLVKGFYPSDIAVE |
| | | WESNGQPENNYKTTPPVLDSDGSFFLYSKL |
| | | TVDKSRWQQGNVFSCSVMHEALHNHYTQ |
| | | KSLSLSPGK |
| SEQ ID NO: | Ab85 full length | EVQLVQSGAEVKKPGESLKISCKGSGYSFT |
| 277 | heavy chain | NYWIGWVRQMPGKGLEWMAIINPRDSDT |
| | sequence: constant | RYRPSFQGQVTISADKSISTAYLQWSSLKA |
| | region underlined; | SDTAMYYCARHGRGYEGYEGAFDIWGQG |
| | modified Fc region | TLVTVSSASTKGPSVFPLAPSSKSTSGGTAA |
| | with L234A, L235A, | LGCLVKDYFPEPVTVSWNSGALTSGVHTFP |
| | D265C mutations | AVLQSSGLYSLSSVVTVPSSSLGTQTYICNV |
| | (mutations in bold)* | NHKPSNTKVDKKVEPKSCDKTHTCPPCPA |
| | | PEAAGGPSVFLFPPKPKDTLMISRTPEVTCV |
| | | VVCVSHEDPEVKFNWYVDGVEVHNAKTK |
| | | PREEQYNSTYRVVSVLTVLHQDWLNGKEY |
| | | KCKVSNKALPAPIEKTISKAKGQPREPQVY |
| | | TLPPSRDELTKNQVSLTCLVKGFYPSDIAVE |
| | | WESNGQPENNYKTTPPVLDSDGSFFLYSKL |
| | | TVDKSRWQQGNVFSCSVMHEALHNHYTQ |
| | | KSLSLSPGK |
| SEQ ID NO: | Ab85 full length | EVQLVQSGAEVKKPGESLKISCKGSGYSFT |
| 278 | heavy chain | NYWIGWVRQMPGKGLEWMAIINPRDSDT |
| | sequence (LALA- | RYRPSFQGQVTISADKSISTAYLQWSSLKA |
| | D265C-H435A | SDTAMYYCARHGRGYEGYEGAFDIWGQG |
| | mutant); constant | TLVTVSSASTKGPSVFPLAPSSKSTSGGTAA |
| | region underlined | LGCLVKDYFPEPVTVSWNSGALTSGVHTFP |
| | | AVLQSSGLYSLSSVVTVPSSSLGTQTYICNV |
| | | NHKPSNTKVDKKVEPKSCDKTHTCPPCPA |
| | | PEAAGGPSVFLFPPKPKDTLMISRTPEVTCV |
| | | VVCVSHEDPEVKFNWYVDGVEVHNAKTK |
| | | PREEQYNSTYRVVSVLTVLHQDWLNGKEY |
| | | KCKVSNKALPAPIEKTISKAKGQPREPQVY |
| | | TLPPSRDELTKNQVSLTCLVKGFYPSDIAVE |
| | | WESNGQPENNYKTTPPVLDSDGSFFLYSKL |
| | | TVDKSRWQQGNVFSCSVMHEALHNAYTQ |
| | | KSLSLSPGK |
| SEQ ID NO: | Ab249 full length | EVQLVQSGAEVKKPGESLKISCKGSGYRFT |
| 279 | heavy chain | TSWIGWVRQMPGKGLEWMGIIYPGDSDTR |
| | sequence; constant | YSPSFQGQVTISADKSISTAYLQWSSLKAS |
| | region underlined | DTAMYYCARHGLGYNGYEGAFDIWGQGT |
| | | LVTVSSASTKGPSVFPLAPSSKSTSGGTAAL |
| | | GCLVKDYFPEPVTVSWNSGALTSGVHTFP |
| | | AVLQSSGLYSLSSVVTVPSSSLGTQTYICNV |
| | | NHKPSNTKVDKKVEPKSCDKTHTCPPCPA |
| | | PELLGGPSVFLFPPKPKDTLMISRTPEVTCV |
| | | VVDVSHEDPEVKFNWYVDGVEVHNAKTK |
| | | PREEQYNSTYRVVSVLTVLHQDWLNGKEY |
| | | KCKVSNKALPAPIEKTISKAKGQPREPQVY |
| | | TLPPSRDELTKNQVSLTCLVKGFYPSDIAVE |
| | | WESNGQPENNYKTTPPVLDSDGSFFLYSKL |
| | | TVDKSRWQQGNVFSCSVMHEALHNHYTQ |
| | | KSLSLSPGK |
| SEQ ID NO: | Ab249 full length | EVQLVQSGAEVKKPGESLKISCKGSGYRFT |
| 280 | heavy chain | TSWIGWVRQMPGKGLEWMGIIYPGDSDTR |
| | sequence; constant | YSPSFQGQVTISADKSISTAYLQWSSLKAS |
| | region underlined | DTAMYYCARHGLGYNGYEGAFDIWGQGT |
| | (LALA mutations)* | LVTVSSASTKGPSVFPLAPSSKSTSGGTAAL |
| | | GCLVKDYFPEPVTVSWNSGALTSGVHTFP |
| | | AVLQSSGLYSLSSVVTVPSSSLGTQTYICNV |
| | | NHKPSNTKVDKKVEPKSCDKTHTCPPCPA |
| | | PEAAGGPSVFLFPPKPKDTLMISRTPEVTCV |
| | | VVDVSHEDPEVKFNWYVDGVEVHNAKTK |
| | | PREEQYNSTYRVVSVLTVLHQDWLNGKEY |
| | | KCKVSNKALPAPIEKTISKAKGQPREPQVY |
| | | TLPPSRDELTKNQVSLTCLVKGFYPSDIAVE |
| | | WESNGQPENNYKTTPPVLDSDGSFFLYSKL |
| | | TVDKSRWQQGNVFSCSVMHEALHNHYTQ |
| | | KSLSLSPGK |
| SEQ ID NO: | Ab249 full length | EVQLVQSGAEVKKPGESLKISCKGSGYRFT |
| 281 | heavy chain | TSWIGWVRQMPGKGLEWMGIIYPGDSDTR |
| | sequence; constant | YSPSFQGQVTISADKSISTAYLQWSSLKAS |
| | region underlined | DTAMYYCARHGLGYNGYEGAFDIWGQGT |
| | (LALA-D265C | LVTVSSASTKGPSVFPLAPSSKSTSGGTAAL |
| | mutations)* | GCLVKDYFPEPVTVSWNSGALTSGVHTFP |
| | | AVLQSSGLYSLSSVVTVPSSSLGTQTYICNV |
| | | NHKPSNTKVDKKVEPKSCDKTHTCPPCPA |
| | | PEAAGGPSVFLFPPKPKDTLMISRTPEVTCV |
| | | VVCVSHEDPEVKFNWYVDGVEVHNAKTK |
| | | PREEQYNSTYRVVSVLTVLHQDWLNGKEY |
| | | KCKVSNKALPAPIEKTISKAKGQPREPQVY |
| | | TLPPSRDELTKNQVSLTCLVKGFYPSDIAVE |
| | | WESNGQPENNYKTTPPVLDSDGSFFLYSKL |
| | | TVDKSRWQQGNVFSCSVMHEALHNHYTQ |
| | | KSLSLSPGK |
| SEQ ID NO: | Ab249 full length | EVQLVQSGAEVKKPGESLKISCKGSGYRFT |
| 282 | heavy chain | TSWIGWVRQMPGKGLEWMGIIYPGDSDTR |
| | sequence; constant | YSPSFQGQVTISADKSISTAYLQWSSLKAS |
| | region underlined; | DTAMYYCARHGLGYNGYEGAFDIWGQGT |
| | (LALA-D265C- | LVTVSSASTKGPSVFPLAPSSKSTSGGTAAL |
| | H435A mutations)* | GCLVKDYFPEPVTVSWNSGALTSGVHTFP |
| | | AVLQSSGLYSLSSVVTVPSSSLGTQTYICNV |
| | | NHKPSNTKVDKKVEPKSCDKTHTCPPCPA |
| | | PEAAGGPSVFLFPPKPKDTLMISRTPEVTCV |
| | | VVCVSHEDPEVKFNWYVDGVEVHNAKTK |
| | | PREEQYNSTYRVVSVLTVLHQDWLNGKEY |
| | | KCKVSNKALPAPIEKTISKAKGQPREPQVY |
| | | TLPPSRDELTKNQVSLTCLVKGFYPSDIAVE |
| | | WESNGQPENNYKTTPPVLDSDGSFFLYSKL |
| | | TVDKSRWQQGNVFSCSVMHEALHNAYTQ |
| | | KSLSLSPGK |
| SEQ ID NO: | Light chain constant | RTVAAPSVFIFPPSDEQLKSGTASVVCLLN |
| 283 | region | NFYPREAKVQWKVDNALQSGNSQESVTEQ |
| | | DSKDSTYSLSSTLTLSKADYEKHKVYACEV |
| | | THQGLSSPVTKSFNRGEC |
| SEQ ID NO: | Ab85 full length | DIQMTQSPSSLSASVGDRVTITCRSSQGIRS |
| 284 | light chain; constant | DLGWYQQKPGKAPKLLIYDASNLETGVPS |
| | region underlined | RFSGSGSGTDFTLTISSLQPEDFATYYCQQA |
| | | NGFPLTFGGGTKVEIKRTVAAPSVFIFPPSD |
| | | EQLKSGTASVVCLLNNFYPREAKVQWKVD |
| | | NALQSGNSQESVTEQDSKDSTYSLSSTLTL |
| | | SKADYEKHKVYACEVTHQGLSSPVTKSFN |
| | | RGEC |
| SEQ ID NO: | Ab249 light chain; | DIQMTQSPSSLSASVGDRVTITCRASQGIGS |
| 285 | constant region | ALAWYQQKPGKAPKLLIYDASNLETGVPS |
| | underlined | RFSGSGSGTDFTLTISSLQPEDFATYYCQQL |
| | | NGYPLTFGQGTRLEIKRTVAAPSVFIFPPSD |
| | | EQLKSGTASVVCLLNNFYPREAKVQWKVD |
| | | NALQSGNSQESVTEQDSKDSTYSLSSTLTL |
| | | SKADYEKHKVYACEVTHQGLSSPVTKSFN |
| | | RGEC |
| SEQ ID NO: | Ab249 HC-CDR1 | TSWIG |
| 286 | | |
| SEQ ID NO: | Ab249 HC-CDR3 | HGLGYNGYEGAFDI |
| 287 | | |
| SEQ ID NO: | Ab249 LC-CDR1 | RASQGIGSALA |
| 288 | | |
| SEQ ID NO: | Ab249 LC-CDR3 | CQQLNGYPLT |
| 289 | | |
| SEQ ID NO: | ch-BBK2 Heavy | QVQLQQPGAELVRPGASVKLSCKASGYTFTSY |
| 290 | Chain (Variable | WINWVKQRPGQGLEWIGNIYPSDSYTNYNQK |
| | region italicized) | FKDKATLTVDKSSNTVYMQLNSPTSEDSAVYY |
| | | CTRNGVEGYPHYYAMEYWGQGTSVTVSSAST |
| | | KGPSVFPLAPSSKSTSGGTAALGCLVKDYF |
| | | PEPVTVSWNSGALTSGVHTFPAVLQSSGLY |
| | | SLSSVVTVPSSSLGTQTYICNVNHKPSNTK |
| | | VDKKVEPKSCDKTHTCPPCPAPELLGGPSV |
| | | FLFPPKPKDTLMISRTPEVTCVVVDVSHED |
| | | PEVKFNWYVDGVEVHNAKTKPREEQYNS |
| | | TYRVVSVLTVLHQDWLNGKEYKCKVSNK |
| | | ALPAPIEKTISKAKGQPREPQVYTLPPSRDE |
| | | LTKNQVSLTCLVKGFYPSDIAVEWESNGQP |
| | | ENNYKTTPPVLDSDGSFFLYSKLTVDKSRW |
| | | QQGNVFSCSVMHEALHNHYTQKSLSLSPG |
| | | K |
| SEQ ID NO: | ch-BBK2 Light | DIQMTQTTSALSASLGDRVTIGCRASQDLSNH |
| 291 | Chain (Variable | LYWYQQKPDGTVKLLIYYTSRLHSGVPSRFSG |
| | region italicized) | SGSGTDYSLTIRNLEQEDVATYFCQQGYTLPY |
| | | TFGGGTKLEIKRTVAAPSVFIFPPSDEQLKS |
| | | GTASVVCLLNNFYPREAKVQWKVDNALQ |
| | | SGNSQESVTEQDSKDSTYSLSSTLTLSKAD |
| | | YEKHKVYACEVTHQGLSSPVTKSFNRGEC |
| SEQ ID NO: | ch-BBK2 VH CDR1 | SYWIN |
| 292 | | |
| SEQ ID NO: | ch-BBK2 VH CDR2 | NIYPSDSYTNYNQKFKD |
| 293 | | |
| SEQ ID NO: | ch-BBK2 VH CDR3 | NGVEGYPHYYAMEY |
| 294 | | |
| SEQ ID NO: | ch-BBK2 VL CDR1 | RASQDLSNHLY |
| 295 | | |
| SEQ ID NO: | ch-BBK2 VL CDR2 | YTSRLHS |
| 296 | | |
| SEQ ID NO: | ch-BBK2 VL CDR3 | QQGYTLPYT |
| 297 | | |
| SEQ ID NO: | ch-BBK2 Light | QVQLQQPGAELVRPGASVKLSCKASGYTF |
| 298 | Chain Variable | TSYWINWVKQRPGQGLEWIGNIYPSDSYT |
| | Region | NYNQKFKDKATLTVDKSSNTVYMQLNSPT |
| | | SEDSAVYYCTRNGVEGYPHYYAMEYWGQ |
| | | GTSVTVSS |
| SEQ ID NO: | ch-BBK2 Heavy | DIQMTQTTSALSASLGDRVTIGCRASQDLS |
| 299 | Chain Variable | NHLYWYQQKPDGTVKLLIYYTSRLHSGVP |
| | Region | SRFSGSGSGTDYSLTIRNLEQEDVATYFCQ |
| | | QGYTLPYTFGGGTKLEIK. |
| SEQ ID NO: | Anti CD2 Ab1 CDR- | EYYMY |
| 300 | H1 | |
| SEQ ID NO: | Ab1 CDR-H2 | RIDPEDGSIDYVEKFKK |
| 301 | | |
| SEQ ID NO: | Ab1 CDR-H3 | GKFNYRFAY |
| 302 | | |
| SEQ ID NO: | Ab1 CDR-L1 | RSSQSLLHSSGNTYLN |
| 303 | | |
| SEQ ID NO: | Ab1 CDR-L2 | LVSKLES |
| 304 | | |
| SEQ ID NO: | Ab1 CDR-L3 | MQFTHYPYT |
| 305 | | |
| SEQ ID NO: | Ab1 Heavy chain | QVQLVQSGAEVKKPGASVKVSCKASGYTF |
| 306 | variable region | TEYYMYWVRQAPGQGLELMGRIDPEDGSI |
| | | DYVEKFKKKVTLTADTSSSTAYMELSSLTS |
| | | DDTAVYYCARGKFNYRFAYWGQGTLVTV |
| | | SS |
| SEQ ID NO: | Ab1 Light chain | DVVMTQSPPSLLVTLGQPASISCRSSQSLLH |
| 307 | variable region | SSGNTYLNWLLQRPGQSPQPLIYLVSKLES |
| | | GVPDRFSGSGSGTDFTLKISGVEAEDVGVY |
| | | YCMQFTHYPYTFGQGTKLEIK |
| SEQ ID NO: | Abla Heavy chain | QVQLVQSGAEVQRPGASVKVSCKASGYIF |
| 308 | variable region | TEYYMYWVRQAPGQGLELVGRIDPEDGSI |
| | | DYVEKFKKKVTLTADTSSSTAYMELSSLTS |
| | | DDTAVYYCARGKFNYRFAYWGQGTLVTV |
| | | SS |
| SEQ ID NO: | Abla Light chain | DVVMTQSPPSLLVTLGQPASISCRSSQSLLH |
| 309 | variable region | SSGNTYLNWLLQRPGQSPQPLIYLVSKLES |
| | | GVPDRFSGSGSGTDFTLKISGVEAEDVGVY |
| | | YCMQFTHYPYTFGQGTKLEIK |
| SEQ ID NO: | Consensus human Ab | EVQLVESGGGLVQPGGSLRLSCAASGFTFS |
| 310 | Heavy chain variable | DYAMSWVRQAPGKGLEWVAVISENGSDT |
| | domain | YYADSVKGRFTISRDDSKNTLYLQMNSLR |
| | | AEDTAVYYCARDRGGAVSYFDVWGQGT |
| | | LVTVSS |
| SEQ ID NO: | Consensus human Ab | DIQMTQSPSSLSASVGDRVTITCRASQDVS |
| 311 | Light chain variable | SYLAWYQQKPGKAPKLLIYAASSLESGVP |
| | domain | SRFSGSGSGTDFTLTISSLQPEDFATYYCQQ |
| | | YNSLPYTFGQGTKVEIKRT |
| SEQ ID NO: | Human CD2 | MSFPCKFVASFLLIFNVSSKGAVSKEITNAL |
| 312 | sequence | ETWGALGQDINLDIPSFQMSDDIDDIKWEK |
| | | TSDKKKIAQFRKEKETFKEKDTYKLFKNGT |
| | | LKIKHLKTDDQDIYKVSIYDTKGKNVLEKI |
| | | FDLKIQERVSKPKISWTCINTTLTCEVMNG |
| | | TDPELNLYQDGKHLKLSQRVITHKWTTSLS |
| | | AKFKCTAGNKVSKESSVEPVSCPEKGLDIY |
| | | LIIGICGGGSLLMVFVALLVFYITKRKKQRS |
| | | RRNDEELETRAHRVATEERGRKPHQIPAST |
| | | PQNPATSQHPPPPPGHRSQAPSHRPPPPGHR |
| | | VQHQPQKRPPAPSGTQVHQQKGPPLPRPR |
| | | VQPKPPHGAAENSLSPSSN |
| SEQ ID NO: | RPA-2.10 CDR-H1 | GFTFSSY |
| 313 | | |
| SEQ ID NO: | Anti-CD2 RPA-2.10 | SGGGF |
| 314 | CDR-H2 | |
| SEQ ID NO: | RPA-2.10 CDR-H3 | SSYGEIMDY |
| 315 | Variant 1 | |
| SEQ ID NO: | RPA-2.10 CDR-H3 | SSYGELMDY |
| 316 | Variant 2 | |
| SEQ ID NO: | RPA-2.10 CDR-L1 | RASQRIGTSIH |
| 317 | | |
| SEQ ID NO: | RPA-2.10 CDR-L2 | YASESIS |
| 318 | | |
| SEQ ID NO: | RPA-2.10 CDR-L3 | QQSHGWPFTF |
| 319 | | |
| SEQ ID NO: | RPA-2.10 Heavy | EVKLVESGGGLVKPGGSLKLSCAASGFTFS |
| 320 | chain variable region | SYDMSWVRQTPEKRLEWVASISGGGFLYY |
| | Variant 1 | LDSVKGRFTISRDNARNILYLHMTSLRSED |
| | | TAMYYCARSSYGEIMDYWGQGTSVTVSS |
| SEQ ID NO: | RPA-2.10 Heavy | EVKLVESGGGLVKPGGSLKLSCAASGFTFS |
| 321 | chain variable region | SYDMSWVRQTPEKRLEWVASISGGGFLYY |
| | Variant 2 | LDSVKGRFTISRDNARNILYLHMTSLRSED |
| | | TAMYYCARSSYGELMDYWGQGTSVTVSS |
| SEQ ID NO: | RPA-2.10 Light | DILLTQSPAILSVSPGERVSFSCRASQRIGTS |
| 322 | chain variable region | IHWYQQRTTGSPRLLIKYASESISGIPSRFSG |
| | | SGSGTDFTLSINSVESEDVADYYCQQSHGW |
| | | PFTFGGGTKLEIE |
| SEQ ID NO: | RPA-2.10 Heavy | AKTTPPSVYPLAPGSAAQTNSMVTLGCLV |
| 323 | chain constant region | KGYFPEPVTVTWNSGSLSSGVHTFPAVLQS |
| | | DLYTLSSSVTVPSSTWPSETVTCNVAHPAS |
| | | STKVDKKIVPRDCGCKPCICTVPEVSSVFIF |
| | | PPKPKDVLTITLTPKVTCVVVDISKDDPEV |
| | | QFSWFVDDVEVHTAQTQPREEQFNSTFRS |
| | | VSELPIMHQDWLNGKEFKCRVNSAAFPAPI |
| | | EKTISKTKGRPKAPQVYTIPPPKEQMAKDK |
| | | VSLTCMITDFFPEDITVEWQWNGQPAENY |
| | | KNTQPIMDTDGSYFVYSKLNVQKSNWEAG |
| | | NTFTCSVLHEGLHNHHTEKSLSHSPGK |
| SEQ ID NO: | RPA-2.10 Light | RADAAPTVSIFPPSSEQLTSGGASVVCFLNN |
| 324 | chain constant region | FYPKDINVKWKIDGSERQNGVLNSWTDQD |
| | | SKDSTYSMSSTLTLTKDEYERHNSYTCEAT |
| | | HKTSTSPIVKSFNRNEC |
| SEQ ID NO: | Anti-CD5 antibody | DIQMTQSPSSMSASLGDRVTITCRASQDINS |
| 325 | Light chain variable | YLSWFQQKPGKSPKTLIYRANRLVDGVPSR |
| | region | FSGSGSGTDYTLTISSLQYEDFGIYYCQQYD |
| | | ESPWTFGGGTKLEIK |
| SEQ ID NO: | Heavy chain variable | QIQLVQSGPGLKKPGGSVRISCAASGYTFT |
| 326 | region | NYGMNWVKQAPGKGLRWMGWINTHTGE |
| | | PTYADDFKGRFTFSLDTSKSTAYLQINSLR |
| | | AEDTATYFCTRRGYDWY |
| | | FDVWGQGTTVTVSS |
| SEQ ID NO: | CDR-H1 | GYTFTNY |
| 327 | | |
| SEQ ID NO: | CDR-H2 | NTHTGE |
| 328 | | |
| SEQ ID NO: | CDR-H3 | RGYDWYFDV |
| 329 | | |
| SEQ ID NO: | CDR-L1 | RASQDINSYLS |
| 330 | | |
| SEQ ID NO: | CDR-L2 | RANRLVD |
| 331 | | |
| SEQ ID NO: | CDR-L3 | QQYDESPWT |
| 332 | | |
| SEQ ID NO: | Light chain variable | DIQMTQSPSSLSASVGDRVTITCRASQDINS |
| 333 | region | YLSWFQQKPGKAPKTLIYRANRLESGVPSR |
| | | FSGSGSGTDYTLTISSLQYEDFGIYYCQQYD |
| | | ESPWTFGGGTKLEIK |
| SEQ ID NO: | Heavy chain variable | EIQLVQSGGGLVKPGGSVRISCAASGYTFT |
| 334 | region | NYGMNWVRQAPGKGLEWMGWINTHYGE |
| | | PTYADSFKGTRTFSLDDSKNTAYLQINSLR |
| | | AEDTAVYFCTRRGYDWYFDVWGQGGTTV |
| | | TVSS |
| SEQ ID NO: | CDR-H1 | GYTFTNY |
| 335 | | |
| SEQ ID NO: | CDR-H2 | NTHYGE |
| 336 | | |
| SEQ ID NO: | CDR-H3 | RRGYDWYFDV |
| 337 | | |
| SEQ ID NO: | CDR-L1 | RASQDINSYLS |
| 338 | | |
| SEQ ID NO: | CDR-L2 | RANRLES |
| 339 | | |
| SEQ ID NO: | CDR-L3 | QQYDESPWT |
| 340 | | |
| SEQ ID NO: | CDR-H1 | GYSITSGYY |
| 341 | | |
| SEQ ID NO: | CDR-H2 | ISYSGFT |
| 342 | | |
| SEQ ID NO: | CDR-H3 | AGDRTGSWFAY |
| 343 | | |
| SEQ ID NO: | CDR-L1 | QDISNY |
| 344 | | |
| SEQ ID NO: | CDR-L2 | ATS |
| 345 | | |
| SEQ ID NO: | CDR-L3 | LQYASYPFT |
| 346 | | |
| SEQ ID NO: | CDR-H1 | GYIFTNYG |
| 347 | | |
| SEQ ID NO: | CDR-H2 | INTYNGEP |
| 348 | | |
| SEQ ID NO: | CDR-H3 | ARGDYYGYEDY |
| 349 | | |
| SEQ ID NO: | CDR-L1 | QGISNY |
| 350 | | |
| SEQ ID NO: | CDR-L2 | YTS |
| 351 | | |
| SEQ ID NO: | CDR-L3 | QQYSKLPWT |
| 352 | | |
| SEQ ID NO: | 5D7 CDR-H1 | FSLSTSGMG |
| 353 | | |
| SEQ ID NO: | 5D7 CDR-H2 | WWDDD |
| 354 | | |
| SEQ ID NO: | 5D7 CDR-H3 | RRATGTGFDY |
| 355 | | |
| SEQ ID NO: | 5D7 CDR-L1 | QDVGTA |
| 356 | | |
| SEQ ID NO: | 5D7 CDR-L2 | WTSTRHT |
| 357 | | |
| SEQ ID NO: | 5D7 CDR-L3 | YNSYNT |
| 358 | | |
| SEQ ID NO: | Humanized 5D7 | QVTLKESGPVLVKPTETLTLTCTFSGFSLST |
| 359 | Heavy chain variable | SGMGVGWIRQAPGKGLEWVAHIWWDDD |
| | region (CDRs in | VYYNPSLKSRLTITKDASKDQVSLKLSSVT |
| | bold) | AADTAVYYCVRRRATGTGFDYWGQGTL |
| | | VTVSS |
| SEQ ID NO: | Humanized 5D7 | NIVMTQSPSSLSASVGDRVTITCQASQDVG |
| 360 | Light chain variable | TAVAWYQQKPDQSPKLLIYWTSTRHTGV |
| | region | PDRFTGSGSGTDFTLTISSLQPEDIATYFCH |
| | (CDRs in bold) | QYNSYNTFGSGTKLEIK |
| SEQ ID NO: | Consensus human | EVQLVESGGGLVQPGGSLRLSCAASGFTFS |
| 361 | Heavy chain variable | DYAMSWVRQAPGKGLEWVAVISENGSDT |
| | domain (CDRs in | YYADSVKGRFTISRDDSKNTLYLQMNSLR |
| | bold) | AEDTAVYYCARDRGGAVSYFDVWGQGT |
| | | LVTVSS |
| SEQ ID NO: | Consensus human | DIQMTQSPSSLSASVGDRVTITCRASQDVS |
| 362 | Light chain variable | SYLAWYQQKPGKAPKLLIYAASSLESGVP |
| | domain (CDRs in | SRFSGSGSGTDFTLTISSLQPEDFATYYCQQ |
| | bold) | YNSLPYTFGQGTKVEIKRT |
| SEQ ID NO: | Human CD5 amino | MVCSQSWGRS SKQWEDPSQA |
| 363 | acid sequence | SKVCQRLNCG VPLSLGPFLV TYTPQSSIIC |
| | | YGQLGSFSNCSHSRNDMCHS |
| | | LGLTCLEPQK TTPPTTRPPP TTTPEPTAPP |
| | | RLQLVAQSGG |
| | | QHCAGVVEFYSGSLGGTISY |
| | | EAQDKTQDLE NFLCNNLQCG |
| | | SFLKHLPETE AGRAQDPGEP |
| | | REHQPLPIQWKIQNSSCTSL EHCFRKIKPQ |
| | | KSGRVLALLC SGFQPKVQSR |
| | | LVGGSSICEG |
| | | TVEVRQGAQWAALCDSSSAR |
| | | SSLRWEEVCR EQQCGSVNSY |
| | | RVLDAGDPTS RGLFCPHQKL |
| | | SQCHELWERNSYCKKVFVTC |
| | | QDPNPAGLAA GTVASIILAL |
| | | VLLVVLLVVC GPLAYKKLVK |
| | | KFRQKKQRQWIGPTGMNQNM |
| | | SFHRNHTATV RSHAENPTAS |
| | | HVDNEYSQPP RNSHLSAYPA |
| | | LEGALHRSSMQPDNSSDSDY DLHGAQRL |
| SEQ ID NO: | Trastuzumab HC | EVQLVESGGGLVQPGGSLRLSCAASGFNIK |
| 364 | | DTYIHWVRQAPGKGLEWVARIYPTNGYTR |
| | | YADSVKGRFTISADTSKNTAYLQMNSLRA |
| | | EDTAVYYCSRWGGDGFYAMDYWGQGTL |
| | | VTVSSASTKGPSVFPLAPSSKSTSGGTAAL |
| | | GCLVKDYFPEPVTVSWNSGALTSGVHTFP |
| | | AVLQSSGLYSLSSVVTVPSSSLGTQTYICNV |
| | | NHKPSNTKVDKKVEPKSCDKTHTCPPCPA |
| | | PELLGGPSVFLFPPKPKDTLMISRTPEVTCV |
| | | VVDVSHEDPEVKFNWYVDGVEVHNAKTK |
| | | PREEQYNSTYRVVSVLTVLHQDWLNGKEY |
| | | KCKVSNKALPAPIEKTISKAKGQPREPQVY |
| | | TLPPSREEMTKNQVSLTCLVKGFYPSDIAV |
| | | EWESNGQPENNYKTTPPVLDSDGSFFLYSK |
| | | LTVDKSRWQQGNVFSCSVMHEALHNHYT |
| | | QKSLSLSPGK |
| SEQ ID NO: | Trastuzumab D265C | EVQLVESGGGLVQPGGSLRLSCAASGFNIK |
| 365 | HC | DTYIHWVRQAPGKGLEWVARIYPTNGYTR |
| | | YADSVKGRFTISADTSKNTAYLQMNSLRA |
| | | EDTAVYYCSRWGGDGFYAMDYWGQGTL |
| | | VTVSSASTKGPSVFPLAPSSKSTSGGTAAL |
| | | GCLVKDYFPEPVTVSWNSGALTSGVHTFP |
| | | AVLQSSGLYSLSSVVTVPSSSLGTQTYICNV |
| | | NHKPSNTKVDKKVEPKSCDKTHTCPPCPA |
| | | PELLGGPSVFLFPPKPKDTLMISRTPEVTCV |
| | | VVCVSHEDPEVKFNWYVDGVEVHNAKTK |
| | | PREEQYNSTYRVVSVLTVLHQDWLNGKEY |
| | | KCKVSNKALPAPIEKTISKAKGQPREPQVY |
| | | TLPPSREEMTKNQVSLTCLVKGFYPSDIAV |
| | | EWESNGQPENNYKTTPPVLDSDGSFFLYSK |
| | | LTVDKSRWQQGNVFSCSVMHEALHNHYT |
| | | QKSLSLSPGK |
| SEQ ID NO: | Trastuzumab LALA | EVQLVESGGGLVQPGGSLRLSCAASGFNIK |
| 366 | HC | DTYIHWVRQAPGKGLEWVARIYPTNGYTR |
| | | YADSVKGRFTISADTSKNTAYLQMNSLRA |
| | | EDTAVYYCSRWGGDGFYAMDYWGQGTL |
| | | VTVSSASTKGPSVFPLAPSSKSTSGGTAAL |
| | | GCLVKDYFPEPVTVSWNSGALTSGVHTFP |
| | | AVLQSSGLYSLSSVVTVPSSSLGTQTYICNV |
| | | NHKPSNTKVDKKVEPKSCDKTHTCPPCPA |
| | | PEAAGGPSVFLFPPKPKDTLMISRTPEVTCV |
| | | VVDVSHEDPEVKFNWYVDGVEVHNAKTK |
| | | PREEQYNSTYRVVSVLTVLHQDWLNGKEY |
| | | KCKVSNKALPAPIEKTISKAKGQPREPQVY |
| | | TLPPSREEMTKNQVSLTCLVKGFYPSDIAV |
| | | EWESNGQPENNYKTTPPVLDSDGSFFLYSK |
| | | LTVDKSRWQQGNVFSCSVMHEALHNHYT |
| | | QKSLSLSPGK |
| SEQ ID NO: | Trastuzumab LALA | EVQLVESGGGLVQPGGSLRLSCAASGFNIK |
| 367 | D265C HC | DTYIHWVRQAPGKGLEWVARIYPTNGYTR |
| | | YADSVKGRFTISADTSKNTAYLQMNSLRA |
| | | EDTAVYYCSRWGGDGFYAMDYWGQGTL |
| | | VTVSSASTKGPSVFPLAPSSKSTSGGTAAL |
| | | GCLVKDYFPEPVTVSWNSGALTSGVHTFP |
| | | AVLQSSGLYSLSSVVTVPSSSLGTQTYICNV |
| | | NHKPSNTKVDKKVEPKSCDKTHTCPPCPA |
| | | PEAAGGPSVFLFPPKPKDTLMISRTPEVTCV |
| | | VVCVSHEDPEVKFNWYVDGVEVHNAKTK |
| | | PREEQYNSTYRVVSVLTVLHQDWLNGKEY |
| | | KCKVSNKALPAPIEKTISKAKGQPREPQVY |
| | | TLPPSREEMTKNQVSLTCLVKGFYPSDIAV |
| | | EWESNGQPENNYKTTPPVLDSDGSFFLYSK |
| | | LTVDKSRWQQGNVFSCSVMHEALHNHYT |
| | | QKSLSLSPGK |
| SEQ ID NO: | Trastuzumab HC | EVQLVESGGGLVQPGGSLRLSCAASGFNIK |
| 368 | Variable Region | DTYIHWVRQAPGKGLEWVARIYPTNGYTR |
| | | YADSVKGRFTISADTSKNTAYLQMNSLRA |
| | | EDTAVYYCSRWGGDGFYAMDYWGQGTL |
| | | VTVSS |
| SEQ ID NO: | Trastuzumab LC | DIQMTQSPSSLSASVGDRVTITCRASQDVN |
| 369 | | TAVAWYQQKPGKAPKLLIYSASFLYSGVPS |
| | | RFSGSRSGTDFTLTISSLQPEDFATYYCQQH |
| | | YTTPPTFGQGTKVEIKRTVAAPSVFIFPPSD |
| | | EQLKSGTASVVCLLNNFYPREAKVQWKVD |
| | | NALQSGNSQESVTEQDSKDSTYSLSSTLTL |
| | | SKADYEKHKVYACEVTHQGLSSPVTKSFN |
| | | RGEC |
| SEQ ID NO: | Trastuzumab LC | DIQMTQSPSSLSASVGDRVTITCRASQDVN |
| 370 | Variable Region | TAVAWYQQKPGKAPKLLIYSASFLYSGVPS |
| | | RFSGSRSGTDFTLTISSLQPEDFATYYCQQH |
| | | YTTPPTFGQGTKVEIK |
| SEQ ID NO: | Anti-PSMA | QVQLVQSGAEVKKPGASVKVSCKASGYTF |
| 371 | Antibody h3/F11- | TYFDINWLRQAPGQGLEWMGGISPGDSNV |
| | Var16 HC | NYAQKFQGRVTLTIDTSTSTAYMELSSLRS |
| | | EDTAVYYCARDGNFPYYAMDSWGQGTLV |
| | | TVSSASTKGPSVFPLAPSSKSTSGGTAALGC |
| | | LVKDYFPEPVTVSWNSGALTSGVHTFPAV |
| | | LQSSGLYSLSSVVTVPSSSLGTQTYICNVNH |
| | | KPSNTKVDKKVEPKSCDKTHTCPPCPAPEL |
| | | LGGPSVFLFPPKPKDTLMISRTPEVTCVVV |
| | | DVSHEDPEVKFNWYVDGVEVHNAKTKPR |
| | | EEQYNSTYRVVSVLTVLHQDWLNGKEYK |
| | | CKVSNKALPAPIEKTISKAKGQPREPQVYT |
| | | LPPSREEMTKNQVSLTCLVKGFYPSDIAVE |
| | | WESNGQPENNYKTTPPVLDSDGSFFLYSKL |
| | | TVDKSRWQQGNVFSCSVMHEALHNHYTQ |
| | | KSLSLSPGK |
| SEQ ID NO: | Anti-PSMA | QVQLVQSGAEVKKPGASVKVSCKASGYTF |
| 372 | Antibody h3/F11- | TYFDINWLRQAPGQGLEWMGGISPGDSNV |
| | Var16 D265C HC | NYAQKFQGRVTLTIDTSTSTAYMELSSLRS |
| | | EDTAVYYCARDGNFPYYAMDSWGQGTLV |
| | | TVSSASTKGPSVFPLAPSSKSTSGGTAALGC |
| | | LVKDYFPEPVTVSWNSGALTSGVHTFPAV |
| | | LQSSGLYSLSSVVTVPSSSLGTQTYICNVNH |
| | | KPSNTKVDKKVEPKSCDKTHTCPPCPAPEL |
| | | LGGPSVFLFPPKPKDTLMISRTPEVTCVVV |
| | | CVSHEDPEVKFNWYVDGVEVHNAKTKPR |
| | | EEQYNSTYRVVSVLTVLHQDWLNGKEYK |
| | | CKVSNKALPAPIEKTISKAKGQPREPQVYT |
| | | LPPSREEMTKNQVSLTCLVKGFYPSDIAVE |
| | | WESNGQPENNYKTTPPVLDSDGSFFLYSKL |
| | | TVDKSRWQQGNVFSCSVMHEALHNHYTQ |
| | | KSLSLSPGK |
| SEQ ID NO: | Anti-PSMA | QVQLVQSGAEVKKPGASVKVSCKASGYTF |
| 373 | Antibody h3/F11- | TYFDINWLRQAPGQGLEWMGGISPGDSNV |
| | Var16 LALA HC | NYAQKFQGRVTLTIDTSTSTAYMELSSLRS |
| | | EDTAVYYCARDGNFPYYAMDSWGQGTLV |
| | | TVSSASTKGPSVFPLAPSSKSTSGGTAALGC |
| | | LVKDYFPEPVTVSWNSGALTSGVHTFPAV |
| | | LQSSGLYSLSSVVTVPSSSLGTQTYICNVNH |
| | | KPSNTKVDKKVEPKSCDKTHTCPPCPAPEA |
| | | AGGPSVFLFPPKPKDTLMISRTPEVTCVVV |
| | | DVSHEDPEVKFNWYVDGVEVHNAKTKPR |
| | | EEQYNSTYRVVSVLTVLHQDWLNGKEYK |
| | | CKVSNKALPAPIEKTISKAKGQPREPQVYT |
| | | LPPSREEMTKNQVSLTCLVKGFYPSDIAVE |
| | | WESNGQPENNYKTTPPVLDSDGSFFLYSKL |
| | | TVDKSRWQQGNVFSCSVMHEALHNHYTQ |
| | | KSLSLSPGK |
| SEQ ID NO: | Anti-PSMA | QVQLVQSGAEVKKPGASVKVSCKASGYTF |
| 374 | Antibody h3/F11- | TYFDINWLRQAPGQGLEWMGGISPGDSNV |
| | Var16 LALA D265C | NYAQKFQGRVTLTIDTSTSTAYMELSSLRS |
| | HC | EDTAVYYCARDGNFPYYAMDSWGQGTLV |
| | | TVSSASTKGPSVFPLAPSSKSTSGGTAALGC |
| | | LVKDYFPEPVTVSWNSGALTSGVHTFPAV |
| | | LQSSGLYSLSSVVTVPSSSLGTQTYICNVNH |
| | | KPSNTKVDKKVEPKSCDKTHTCPPCPAPEA |
| | | AGGPSVFLFPPKPKDTLMISRTPEVTCVVV |
| | | CVSHEDPEVKFNWYVDGVEVHNAKTKPR |
| | | EEQYNSTYRVVSVLTVLHQDWLNGKEYK |
| | | CKVSNKALPAPIEKTISKAKGQPREPQVYT |
| | | LPPSREEMTKNQVSLTCLVKGFYPSDIAVE |
| | | WESNGQPENNYKTTPPVLDSDGSFFLYSKL |
| | | TVDKSRWQQGNVFSCSVMHEALHNHYTQ |
| | | KSLSLSPGK |
| SEQ ID NO: | Anti-PSMA | QVQLVQSGAEVKKPGASVKVSCKASGYTF |
| 375 | Antibody h3/F11- | TYFDINWLRQAPGQGLEWMGGISPGDSNV |
| | Var16 HC Variable | NYAQKFQGRVTLTIDTSTSTAYMELSSLRS |
| | Region | EDTAVYYCARDGNFPYYAMDSWGQGTLV |
| | | TVSS |
| SEQ ID NO: | Anti-PSMA | DIVMTQSPLSLPVTPGEPASISCRSSQSLVH |
| 376 | Antibody h3/F11- | SSGQTYLHWYQQKPGQSPQLLIYTVSNRAS |
| | Var16 LC | GVPDRFSGSGSGTDFTLKISRVEAEDVGTY |
| | | YCSQSTHVPTFGGGTKVEIKRTVAAPSVFIF |
| | | PPSDEQLKSGTASVVCLLNNFYPREAKVQ |
| | | WKVDNALQSGNSQESVTEQDSKDSTYSLS |
| | | STLTLSKADYEKHKVYACEVTHQGLSSPV |
| | | TKSFNRGEC |
| SEQ ID NO: | Anti-PSMA | DIVMTQSPLSLPVTPGEPASISCRSSQSLVH |
| 377 | Antibody h3/F11- | SSGQTYLHWYQQKPGQSPQLLIYTVSNRAS |
| | Var16 LC Variable | GVPDRFSGSGSGTDFTLKISRVEAEDVGTY |
| | Region | YCSQSTHVPTFGGGTKVEIK |
| SEQ ID NO: | Anti-PSMA | GYTFTYF |
| 378 | Antibody h3/F11- | |
| | Var16 CDRH1 | |
| SEQ ID NO: | Anti-PSMA | GISPGDSNVNYAQKFQG |
| 379 | Antibody h3/F11- | |
| | Var16 CDRH2 | |
| SEQ ID NO: | Anti-PSMA | DGNFPYYAMDS |
| 380 | Antibody h3/F11- | |
| | Var16 CDRH3 | |
| SEQ ID NO: | Anti-PSMA | RSSQSLVHSSGQTYLH |
| 381 | Antibody h3/F11- | |
| | Var16 CDRL1 | |
| SEQ ID NO: | Anti-PSMA | TVSNRAS |
| 382 | Antibody h3/F11- | |
| | Var16 CDRL2 | |
| SEQ ID NO: | Anti-PSMA | SQSTHVPT |
| 383 | Antibody h3/F11- | |
| | Var16 CDRL3 | |

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the embodiments.

1. An antibody-drug conjugate (ADC), comprising:
an antibody, or an antigen-binding fragment thereof, conjugated to an amatoxin, or a derivative or analogue thereof, via a linker, wherein the antibody, or the antigen binding fragment thereof, comprises an Fc region comprising at least two mutations consisting of L234A and L235A (according to EU index),
wherein said Fc region further comprises a mutation consisting of D265C (according to EU index), and
wherein said antibody, or antigen binding fragment thereof, specifically binds to an antigen expressed on the cell surface of a cancer cell, and
wherein said antibody, or antigen binding fragment thereof, specifically binds to prostate-specific membrane antigen (PSMA), Her2 antigen, CD37, or CD123.

2. The antibody-drug conjugate (ADC) according to claim 1, wherein said antibody, or antigen binding fragment thereof, specifically binds to PSMA and comprises a CDRH1 according to SEQ ID NO. 378, a CDRH2 according to SEQ ID NO. 379, a CDRH3 according to SEQ ID NO. 380, a CDRL1 according to SEQ ID NO. 381, a CDRL2 according to SEQ ID NO. 382, and a CDRL3 according to SEQ ID NO. 383.

3. The antibody-drug conjugate (ADC) according to claim 2, wherein said antibody, or antigen binding fragment thereof, comprises a heavy chain variable region according to SEQ ID NO. 375 and a light chain variable region according to SEQ ID NO. 377.

4. The antibody-drug conjugate (ADC) according to claim 3, wherein said antibody comprises a heavy chain according to SEQ ID NO. 372, SEQ ID NO. 373, or SEQ ID NO. 374, and a light chain according to SEQ ID NO. 376, or an antigen binding fragment thereof.

5. The antibody-drug conjugate (ADC) according to claim, wherein said antibody, or antigen binding fragment thereof, is conjugated to any compound which is selected from the group consisting of HDP30.2060, HDP30.2115, HDP30.2347, HDP30.1699, HDP30.2371, HDP30.0880:

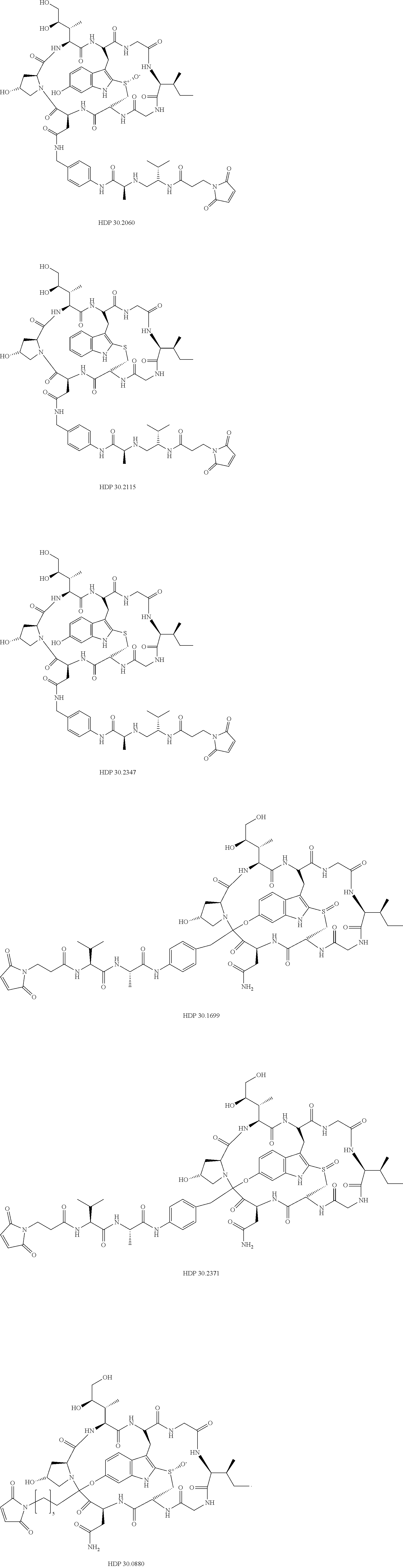

6. An antibody-drug conjugate (ADC) according to claim 5, wherein the drug antibody ratio (DAR) is about 1, 2, 3, or 4, preferably wherein the DAR is 2.

7. A method, comprising:
treating cancer in a patient with the antibody-drug conjugate (ADC) of claim 6.

8. A pharmaceutical composition, comprising:
The antibody-drug conjugate (ADC) according to claim 1, and
at least one pharmaceutically acceptable carrier.

9. A method, comprising:
treating of B lymphocyte-associated malignancies, or B cell-mediated autoimmune diseases selected from the group consisting of non-Hodgkin's lymphoma, follicular lymphoma, diffuse large B cell non-Hodgkin's lymphoma, chronic lymphocytic leukemia, rheumatoid arthritis, granulomatosis with polyangiitis and microscopic polyangiitis and pemphigus vulgaris with the pharmaceutical composition according to claim 8.


SEQUENCE LISTING (564 sequences)

SEQ ID NO: 1 - protein, 5 aa, synthetic construct
  REGION 1..5: note = Synthetic
SYWIG

SEQ ID NO: 2 - protein, 17 aa, synthetic construct
  REGION 1..17: note = Synthetic
IIYPGDSDTRYSPSFQG

SEQ ID NO: 3 - protein, 14 aa, synthetic construct
  REGION 1..14: note = Synthetic
HGRGYNGYEGAFDI

SEQ ID NO: 4 - protein, 11 aa, synthetic construct
  REGION 1..11: note = Synthetic
RASQGISSALA

SEQ ID NO: 5 - protein, 7 aa, synthetic construct
  REGION 1..7: note = Synthetic
DASSLES

SEQ ID NO: 6 - protein, 10 aa, synthetic construct
  REGION 1..10: note = Synthetic
CQQFNSYPLT

SEQ ID NO: 7 - protein, 120 aa, synthetic construct
  REGION 1..120: note = Synthetic
EVQLVESGGGLVQPGGSLRLSCAASGFTFSDYAMSWVRQAPGKGLEWVAVISENGSDTYYADSVKGRFTISRDDSKNTLYLQMNSLRAEDTAVYYCARDRGGAVSYFDVWGQGTLVTVSS

SEQ ID NO: 8 - protein, 109 aa, synthetic construct
  REGION 1..109: note = Synthetic
DIQMTQSPSSLSASVGDRVTITCRASQDVSSYLAWYQQKPGKAPKLLIYAASSLESGVPSRFSGSGSGTDFTLTISSLQPEDFATYYCQQYNSLPYTFGQGTKVEIKRT

SEQ ID NO: 9 - protein, 121 aa, synthetic construct
  REGION 1..121: note = Synthetic
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSS

SEQ ID NO: 10 - protein, 107 aa, synthetic construct
  REGION 1..107: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYIAPYTFGGGTKVEIK

SEQ ID NO: 11 - protein, 9 aa, synthetic construct
  REGION 1..9: note = Synthetic
FTFSDADMD

SEQ ID NO: 12 - protein, 19 aa, synthetic construct
  REGION 1..19: note = Synthetic
RTRNKAGSYTTEYAASVKG

SEQ ID NO: 13 - protein, 12 aa, synthetic construct
  REGION 1..12: note = Synthetic
AREPKYWIDFDL

SEQ ID NO: 14 - protein, 11 aa, synthetic construct
  REGION 1..11: note = Synthetic
RASQSISSYLN

SEQ ID NO: 15 - protein, 7 aa, synthetic construct
  REGION 1..7: note = Synthetic
AASSLQS

SEQ ID NO: 16 - protein, 9 aa, synthetic construct
  REGION 1..9: note = Synthetic
QQSYIAPYT

SEQ ID NO: 17 - DNA, 363 bp, synthetic construct
  misc_feature 1..363: note = Synthetic
  source 1..363: mol_type = other DNA
gaggtgcagctggtggagtctgggggaggcttggtccagcctggagggtccctgagactctcctgtgcagcctctggattcaccttcagtgacgccgacatggactgggtccgccaggctccagggaaggggctggagtgggttggccgtactagaaacaaagcaggaagttacaccacagaatacgccgcgtctgtgaaaggcagattcaccatctcaagagatgattcaaagaactcactgtatctgcaaatgaacagcctgaaaaccgaggacacggcggtgtactactgcgccagagagcctaaatactggatcgacttcgacctatgggggagaggtaccttggtcaccgtctcctca

SEQ ID NO: 18 - DNA, 321 bp, synthetic construct
  misc_feature 1..321: note = Synthetic
  source 1..321: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaagctacatcgccccttacacttttggcggagggaccaaggttgagatcaaa

SEQ ID NO: 19 - protein, 119 aa, synthetic construct
  REGION 1..119: note = Synthetic
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSS

SEQ ID NO: 20 - protein, 106 aa, synthetic construct
  REGION 1..106: note = Synthetic
DIQMTQSPSSLSASVGDRVTITCRASQSINSYLNWYQQKPGKAPKLLIYAASSLQSGVPSRFSGSGSGTDFTLTISSLQPEDFATYYCQQGVSDITFGGGTKVEIK

SEQ ID NO: 21 - protein, 9 aa, synthetic construct
  REGION 1..9: note = Synthetic
GTFRIYAIS

SEQ ID NO: 22 - protein, 17 aa, synthetic construct
  REGION 1..17: note = Synthetic
GIIPDFGVANYAQKFQG

SEQ ID NO: 23 - protein, 12 aa, synthetic construct
  REGION 1..12: note = Synthetic
ARGGLDTDEFDL

SEQ ID NO: 24 - protein, 11 aa, synthetic construct
  REGION 1..11: note = Synthetic
RASQSINSYLN

SEQ ID NO: 25 - protein, 7 aa, synthetic construct
  REGION 1..7: note = Synthetic
AASSLQS

SEQ ID NO: 26 - protein, 8 aa, synthetic construct
  REGION 1..8: note = Synthetic
QQGVSDIT

SEQ ID NO: 27 - DNA, 357 bp, synthetic construct
  misc_feature 1..357: note = Synthetic
  source 1..357: mol_type = other DNA
caggtgcagctggtgcagtctggggctgaggtgaagaagcctgggtcctcggtgaaggtctcctgcaaggcttctggaggcaccttccgaatctatgctatcagctgggtgcgacaggcccctggacaagggcttgagtggatgggagggatcatccctgacttcggtgtagcaaactacgcacagaagttccagggcagagtcacgattaccgcggacgaatccacgagcacagcctacatggagctgagcagcctgagatctgaggacacggcggtgtactactgcgccagaggtggattggacacagacgagttcgacctatgggggagaggtaccttggtcaccgtctcctca

SEQ ID NO: 28 - DNA, 318 bp, synthetic construct
  misc_feature 1..318: note = Synthetic
  source 1..318: mol_type = other DNA
gacatccagatgacccagtctccatcctccctgtctgcatctgtaggagacagagtcaccatcacttgccgggcaagtcagagcattaacagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaggagtcagtgacatcacttttggcggagggaccaaggttgagatcaaa

SEQ ID NO: 29 - protein, 119 aa, synthetic construct
  REGION 1..119: note = Synthetic
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGGLDTDEFDLWGRGTLVTVSS

SEQ ID NO: 30 - protein, 106 aa, synthetic construct; identical to SEQ ID NO: 20
  REGION 1..106: note = Synthetic

SEQ ID NO: 31 - protein, 9 aa, synthetic construct
  REGION 1..9: note = Synthetic
GTFSSYAIS

SEQ ID NO: 32 - protein, 17 aa, synthetic construct
  REGION 1..17: note = Synthetic
GIIPIFGTANYAQKFQG

SEQ ID NO: 33 - protein, 12 aa, synthetic construct
  REGION 1..12: note = Synthetic
ARGGLDTDEFDL

SEQ ID NO: 34 - protein, 11 aa, synthetic construct
  REGION 1..11: note = Synthetic
RASQSINSYLN

SEQ ID NO: 35 - protein, 7 aa, synthetic construct
  REGION 1..7: note = Synthetic
AASSLQS

SEQ ID NO: 36 - protein, 8 aa, synthetic construct
  REGION 1..8: note = Synthetic
QQGVSDIT

SEQ ID NO: 37 - DNA, 357 bp, synthetic construct
  misc_feature 1..357: note = Synthetic
  source 1..357: mol_type = other DNA
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggcggtgtactactgcgccagaggtggattggacacagacgagttcgacctatgggggagaggtaccttggtcaccgtctcctca

SEQ ID NO: 38 - DNA, 318 bp, synthetic construct; identical to SEQ ID NO: 28
  misc_feature 1..318: note = Synthetic
  source 1..318: mol_type = other DNA

SEQ ID NO: 39 - protein, 119 aa, synthetic construct
  REGION 1..119: note = Synthetic
QVQLVQSGAEVKKPGSSVKVSCKASGGTFSLYAISWVRQAPGQGLEWMGGIIPAFGTANYAQKFQGRVTITADESTSTAYMELSSLRSEDTAVYYCARGGLDTDEFDLWGRGTLVTVSS

SEQ ID NO: 40 - protein, 106 aa, synthetic construct; identical to SEQ ID NO: 20
  REGION 1..106: note = Synthetic

SEQ ID NO: 41 - protein, 9 aa, synthetic construct
  REGION 1..9: note = Synthetic
GTFSLYAIS

SEQ ID NO: 42 - protein, 17 aa, synthetic construct
  REGION 1..17: note = Synthetic
GIIPAFGTANYAQKFQG

SEQ ID NO: 43 - protein, 12 aa, synthetic construct
  REGION 1..12: note = Synthetic
ARGGLDTDEFDL

SEQ ID NO: 44 - protein, 11 aa, synthetic construct
  REGION 1..11: note = Synthetic
RASQSINSYLN

SEQ ID NO: 45 - protein, 7 aa, synthetic construct
  REGION 1..7: note = Synthetic
AASSLQS

SEQ ID NO: 46 - protein, 8 aa, synthetic construct
  REGION 1..8: note = Synthetic
QQGVSDIT

SEQ ID NO: 47 - DNA, 357 bp, synthetic construct
  misc_feature 1..357: note = Synthetic
  source 1..357: mol_type = other DNA
caggtgcagctggtgcagtctggggctgaggtgaagaagcctgggtcctcggtgaaggtctcctgcaaggcttctggaggcaccttcagcctctatgctatctcctgggtgcgacaggcccctggacaagggcttgagtggatgggagggatcatccctgccttcggtaccgcaaactacgcacagaagttccagggcagagtcacgattaccgcggacgaatccacgagcacagcctacatggagctgagcagcctgagatctgaggacacggcggtgtactactgcgccagaggtggattggacacagacgagttcgacctatgggggagaggtaccttggtcaccgtctcctca

SEQ ID NO: 48 - DNA, 318 bp, synthetic construct; identical to SEQ ID NO: 28
  misc_feature 1..318: note = Synthetic
  source 1..318: mol_type = other DNA

SEQ ID NO: 49 - protein, 119 aa, synthetic construct
  REGION 1..119: note = Synthetic
QVQLVQSGAEVKKPGSSVKVSCKASGGTFSLYAISWVRQAPGQGLEWMGGIIPHFGLANYAQKFQGRVTITADESTSTAYMELSSLRSEDTAVYYCARGGLDTDEFDLWGRGTLVTVSS

SEQ ID NO: 50 - protein, 106 aa, synthetic construct; identical to SEQ ID NO: 20
  REGION 1..106: note = Synthetic

SEQ ID NO: 51 - protein, 9 aa, synthetic construct
  REGION 1..9: note = Synthetic
GTFSLYAIS

SEQ ID NO: 52 - protein, 17 aa, synthetic construct
  REGION 1..17: note = Synthetic
GIIPHFGLANYAQKFQG

SEQ ID NO: 53 - protein, 12 aa, synthetic construct
  REGION 1..12: note = Synthetic
ARGGLDTDEFDL

SEQ ID NO: 54 - protein, 11 aa, synthetic construct
  REGION 1..11: note = Synthetic
RASQSINSYLN

SEQ ID NO: 55 - protein, 7 aa, synthetic construct
  REGION 1..7: note = Synthetic
AASSLQS

SEQ ID NO: 56 - protein, 8 aa, synthetic construct
  REGION 1..8: note = Synthetic
QQGVSDIT

SEQ ID NO: 57 - DNA, 357 bp, synthetic construct
  misc_feature 1..357: note = Synthetic
  source 1..357: mol_type = other DNA
caggtgcagctggtgcagtctggggctgaggtgaagaagcctgggtcctcggtgaaggtctcctgcaaggcttctggaggcaccttctccctctatgctatcagctgggtgcgacaggcccctggacaagggcttgagtggatgggagggatcatccctcacttcggtctcgcaaactacgcacagaagttccagggcagagtcacgattaccgcggacgaatccacgagcacagcctacatggagctgagcagcctgagatctgaggacacggcggtgtactactgcgccagaggtggattggacacagacgagttcgacctatgggggagaggtaccttggtcaccgtctcctca

SEQ ID NO: 58 - DNA, 318 bp, synthetic construct; identical to SEQ ID NO: 28
  misc_feature 1..318: note = Synthetic
  source 1..318: mol_type = other DNA

SEQ ID NO: 59 - protein, 123 aa, synthetic construct
  REGION 1..123: note = Synthetic
EVQLLESGGGLVQPGGSLRLSCAASGFTFSNYAMSWVRQAPGKGLEWVSAISGSGGSTYYADSVKGRFTISRDNSKNTLYLQMNSLRAEDTAVYYCAKGPPTYHTNYYYMDVWGKGTTVTVSS

SEQ ID NO: 60 - protein, 107 aa, synthetic construct
  REGION 1..107: note = Synthetic
DIQMTQSPSSVSASVGDRVTITCRASQGISSWLAWYQQKPGKAPKLLIYAASSLQSGVPSRFSGSGSGTDFTLTISSLQPEDFATYYCQQTNSFPYTFGGGTKVEIK

SEQ ID NO: 61 - protein, 9 aa, synthetic construct
  REGION 1..9: note = Synthetic
FTFSNYAMS

SEQ ID NO: 62 - protein, 17 aa, synthetic construct
  REGION 1..17: note = Synthetic
AISGSGGSTYYADSVKG

SEQ ID NO: 63 - protein, 16 aa, synthetic construct
  REGION 1..16: note = Synthetic
AKGPPTYHTNYYYMDV

SEQ ID NO: 64 - protein, 11 aa, synthetic construct
  REGION 1..11: note = Synthetic
RASQGISSWLA

SEQ ID NO: 65 - protein, 7 aa, synthetic construct
  REGION 1..7: note = Synthetic
AASSLQS

SEQ ID NO: 66 - protein, 9 aa, synthetic construct
  REGION 1..9: note = Synthetic
QQTNSFPYT

SEQ ID NO: 67 - DNA, 369 bp, synthetic construct
  misc_feature 1..369: note = Synthetic
  source 1..369: mol_type = other DNA
gaggtgcagctgttggagtctgggggaggcttggtacagcctggggggtccctgagactctcctgtgcagcctctggattcacctttagcaattatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggcggtgtactactgcgccaagggccctcctacataccacacaaactactactacatggacgtatggggcaagggtacaactgtcaccgtctcctca

SEQ ID NO: 68 - DNA, 321 bp, synthetic construct
  misc_feature 1..321: note = Synthetic
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttattactgtcagcaaacaaatagtttcccttacacttttggcggagggaccaaggttgagatcaaa

SEQ ID NO: 69 - protein, 123 aa, synthetic construct
  REGION 1..123: note = Synthetic
EVQLLESGGGLVQPGGSLRLSCAASGFTFSSYVMIWVRQAPGKGLEWVSSISGDSVTTYYADSVKGRFTISRDNSKNTLYLQMNSLRAEDTAVYYCAKGPPTYHTNYYYMDVWGKGTTVTVSS

SEQ ID NO: 70 - protein, 107 aa, synthetic construct; identical to SEQ ID NO: 60
  REGION 1..107: note = Synthetic

SEQ ID NO: 71 - protein, 9 aa, synthetic construct
  REGION 1..9: note = Synthetic
FTFSSYVMI

SEQ ID NO: 72 - protein, 17 aa, synthetic construct
  REGION 1..17: note = Synthetic
SISGDSVTTYYADSVKG

SEQ ID NO: 73 - protein, 16 aa, synthetic construct
  REGION 1..16: note = Synthetic
AKGPPTYHTNYYYMDV

SEQ ID NO: 74 - protein, 11 aa, synthetic construct
  REGION 1..11: note = Synthetic
RASQGISSWLA

SEQ ID NO: 75 - protein, 7 aa, synthetic construct
  REGION 1..7: note = Synthetic
AASSLQS

SEQ ID NO: 76 - protein, 9 aa, synthetic construct
  REGION 1..9: note = Synthetic
QQTNSFPYT

SEQ ID NO: 77 - DNA, 369 bp, synthetic construct
  misc_feature 1..369: note = Synthetic
  source 1..369: mol_type = other DNA
gaggtgcagctgttggagtctgggggaggcttggtacagcctggggggtccctgagactctcctgtgcagcctctggattcacctttagcagctatgtcatgatctgggtccgccaggctccagggaaggggctggagtgggtctcaagcattagtggtgacagcgtaacaacatactacgcagactccgtgaagggccggttcaccatctccagagacaattccaagaacacgctgtatctgcaaatgaacagcctgagagccgaggacacggcggtgtactactgcgccaagggccctcctacataccacacaaactactactacatggacgtatggggcaagggtacaactgtcaccgtctcctca

SEQ ID NO: 78 - DNA, 321 bp, synthetic construct; identical to SEQ ID NO: 68
  misc_feature 1..321: note = Synthetic
  source 1..321: mol_type = other DNA

SEQ ID NO: 79 - protein, 121 aa, synthetic construct
  REGION 1..121: note = Synthetic
EVQLVESGGGLVQPGGSLRLSCAASGFTFSDHYMDWVRQAPGKGLEWVGRTRNKASSYTTEYAASVKGRFTISRDDSKNSLYLQMNSLKTEDTAVYYCAREPKYWIDFDLWGRGTLVTVSS

SEQ ID NO: 80 - protein, 107 aa, synthetic construct; identical to SEQ ID NO: 10
  REGION 1..107: note = Synthetic

SEQ ID NO: 81 - protein, 9 aa, synthetic construct
  REGION 1..9: note = Synthetic
FTFSDHYMD

SEQ ID NO: 82 - protein, 19 aa, synthetic construct
  REGION 1..19: note = Synthetic
RTRNKASSYTTEYAASVKG

SEQ ID NO: 83 - protein, 12 aa, synthetic construct
  REGION 1..12: note = Synthetic
AREPKYWIDFDL

SEQ ID NO: 84 - protein, 11 aa, synthetic construct
  REGION 1..11: note = Synthetic
RASQSISSYLN

SEQ ID NO: 85 - protein, 7 aa, synthetic construct
  REGION 1..7: note = Synthetic
AASSLQS

SEQ ID NO: 86 - protein, 9 aa, synthetic construct
  REGION 1..9: note = Synthetic
QQSYIAPYT

SEQ ID NO: 87 - DNA, 363 bp, synthetic construct
  misc_feature 1..363: note = Synthetic
  source 1..363: mol_type = other DNA
gaggtgcagctggtggagtctgggggaggcttggtccagcctggagggtccctgagactctcctgtgcagcctctggattcaccttcagtgaccactacatggactgggtccgccaggctccagggaaggggctggagtgggttggccgtactagaaacaaagctagtagttacaccacagaatacgccgcgtctgtgaaaggcagattcaccatctcaagagatgattcaaagaactcactgtatctgcaaatgaacagcctgaaaaccgaggacacggcggtgtactactgcgccagagagcctaaatactggatcgacttcgacctatgggggagaggtaccttggtcaccgtctcctca

SEQ ID NO: 88 - DNA, 321 bp, synthetic construct; identical to SEQ ID NO: 18
  misc_feature 1..321: note = Synthetic
  source 1..321: mol_type = other DNA

SEQ ID NO: 89 - protein, 121 aa, synthetic construct
  REGION 1..121: note = Synthetic
EVQLVESGGGLVQPGRSLRLSCTASGFTFSDHDMNWVRQAPGKGLEWVGRTRNAAGSYTTEYAASVKGRFTISRDDSKNSLYLQMNSLKTEDTAVYYCAREPKYWIDFDLWGRGTLVTVSS

SEQ ID NO: 90 - protein, 107 aa, synthetic construct; identical to SEQ ID NO: 10
  REGION 1..107: note = Synthetic

SEQ ID NO: 91 - protein, 9 aa, synthetic construct
  REGION 1..9: note = Synthetic
FTFSDHDMN

SEQ ID NO: 92 - protein, 19 aa, synthetic construct
  REGION 1..19: note = Synthetic
RTRNAAGSYTTEYAASVKG

SEQ ID NO: 93 - protein, 12 aa, synthetic construct
  REGION 1..12: note = Synthetic
AREPKYWIDFDL

SEQ ID NO: 94 - protein, 11 aa, synthetic construct
  REGION 1..11: note = Synthetic
RASQSISSYLN

SEQ ID NO: 95 - protein, 7 aa, synthetic construct
  REGION 1..7: note = Synthetic
AASSLQS

SEQ ID NO: 96 - protein, 9 aa, synthetic construct
  REGION 1..9: note = Synthetic
QQSYIAPYT

SEQ ID NO: 97 - DNA, 363 bp, synthetic construct
  misc_feature 1..363: note = Synthetic
  source 1..363: mol_type = other DNA
gaggtgcagctggtggagtctgggggaggcttggtacagccagggcggtccctgagactctcctgtacagcttctggattcaccttcagtgaccacgacatgaactgggtccgccaggctccagggaaggggctggagtgggttggccgtactagaaacgccgctggaagttacaccacagaatacgccgcgtctgtgaaaggcagattcaccatctcaagagatgattcaaagaactcactgtatctgcaaatgaacagcctgaaaaccgaggacacggcggtgtactactgcgccagagagcctaaatactggatcgacttcgacctatgggggagaggtaccttggtcaccgtctcctca

SEQ ID NO: 98 - DNA, 321 bp, synthetic construct; identical to SEQ ID NO: 18
  misc_feature 1..321: note = Synthetic
  source 1..321: mol_type = other DNA

SEQ ID NO: 99 - protein, 121 aa, synthetic construct
  REGION 1..121: note = Synthetic
EVQLVESGGGLVQPGGSLRLSCAASGFTFVDHDMDWVRQAPGKGLEWVGRTRNKLGSYTTEYAASVKGRFTISRDDSKNSLYLQMNSLKTEDTAVYYCAREPKYWIDFDLWGRGTLVTVSS

SEQ ID NO: 100 - protein, 107 aa, synthetic construct; identical to SEQ ID NO: 10
  REGION 1..107: note = Synthetic

SEQ ID NO: 101 - protein, 9 aa, synthetic construct
  REGION 1..9: note = Synthetic
FTFVDHDMD

SEQ ID NO: 102 - protein, 19 aa, synthetic construct
  REGION 1..19: note = Synthetic
RTRNKLGSYTTEYAASVKG

SEQ ID NO: 103 - protein, 12 aa, synthetic construct
  REGION 1..12: note = Synthetic
AREPKYWIDFDL

SEQ ID NO: 104 - protein, 11 aa, synthetic construct
  REGION 1..11: note = Synthetic
RASQSISSYLN

SEQ ID NO: 105 - protein, 7 aa, synthetic construct
  REGION 1..7: note = Synthetic
AASSLQS

SEQ ID NO: 106 - protein, 9 aa, synthetic construct
  REGION 1..9: note = Synthetic
QQSYIAPYT

SEQ ID NO: 107 - DNA, 363 bp, synthetic construct
  misc_feature 1..363: note = Synthetic
  source 1..363: mol_type = other DNA
gaggtgcagctggtggagtctgggggaggcttggtccagcctggagggtccctgagactctcctgtgcagcctctggattcaccttcgtagaccacgacatggactgggtccgccaggctccagggaaggggctggagtgggttggccgtactagaaacaaactaggaagttacaccacagaatacgccgcgtctgtgaaaggcagattcaccatctcaagagatgattcaaagaactcactgtatctgcaaatgaacagcctgaaaaccgaggacacggcggtgtactactgcgccagagagcctaaatactggatcgacttcgacctatgggggagaggtaccttggtcaccgtctcctca

SEQ ID NO: 108 - DNA, 321 bp, synthetic construct; identical to SEQ ID NO: 18
  misc_feature 1..321: note = Synthetic
  source 1..321: mol_type = other DNA

SEQ ID NO: 109 - protein, 214 aa, synthetic construct
  REGION 1..214: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYIAPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 451 aa, synthetic construct
  REGION 1..451: note = Synthetic
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 451 aa, synthetic construct
  REGION 1..451: note = Synthetic
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 451 aa, synthetic construct
  REGION 1..451: note = Synthetic
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 451 aa, synthetic construct
  REGION 1..451: note = Synthetic
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 114 - protein, 451 aa, synthetic construct
  REGION 1..451: note = Synthetic
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 115 - protein, 213 aa, synthetic construct
  REGION 1..213: note = Synthetic
DIQMTQSPSSLSASVGDRVTITCRASQSINSYLNWYQQKPGKAPKLLIYAASSLQSGVPSRFSGSGSGTDFTLTISSLQPEDFATYYCQQGVSDITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 449 aa, synthetic construct
  REGION 1..449: note = Synthetic
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 449 aa, synthetic construct
  REGION 1..449: note = Synthetic
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 449 aa, synthetic construct
  REGION 1..449: note = Synthetic
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 449 aa, synthetic construct
  REGION 1..449: note = Synthetic
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 120 - protein, 449 aa, synthetic construct
  REGION 1..449: note = Synthetic
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 121 - protein, 107 aa, synthetic construct [sequence omitted]

SEQ ID NO: 122 - protein, 330 aa, Homo sapiens [sequence omitted]

SEQ ID NO: 123 - protein, 330 aa, synthetic construct
  REGION 1..330: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 330 aa, synthetic construct
  REGION 1..330: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 330 aa, synthetic construct
  REGION 1..330: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 126 - protein, 330 aa, synthetic construct
  REGION 1..330: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 127 - protein, 9 aa, synthetic construct
  REGION 1..9: note = Synthetic
  SITE 1..9: note = /note="Variant residues given in the sequence have no preference with respect to those in the annotations for variant positions"
  VARIANT 4: note = /replace="Arg"
  VARIANT 5: note = /replace="Ile" or "Leu"
GTFSSYAIS

SEQ ID NO: 128 - protein, 17 aa, synthetic construct
  REGION 1..17: note = Synthetic
  SITE 1..17: note = /note="Variant residues given in the sequence have no preference with respect to those in the annotations for variant positions"
  VARIANT 5: note = /replace="Asp" or "Ala" or "His"
  VARIANT 8: note = /replace="Val" or "Leu"
GIIPIFGTANYAQKFQG

SEQ ID NO: 129 - protein, 12 aa, synthetic construct
  REGION 1..12: note = Synthetic
ARGGLDTDEFDL

SEQ ID NO: 130 - protein, 11 aa, synthetic construct
  REGION 1..11: note = Synthetic
RASQSINSYLN

SEQ ID NO: 131 - protein, 7 aa, synthetic construct
  REGION 1..7: note = Synthetic
AASSLQS

SEQ ID NO: 132 - protein, 8 aa, synthetic construct
  REGION 1..8: note = Synthetic
QQGVSDIT

SEQ ID NO: 133 - protein, 9 aa, synthetic construct
  REGION 1..9: note = Synthetic
  SITE 1..9: note = /note="Variant residues given in the sequence have no preference with respect to those in the annotations for variant positions"
  VARIANT 5: note = /replace="Ser"
  VARIANT 7: note = /replace="Val"
  VARIANT 9: note = /replace="Ile"
FTFSNYAMS

SEQ ID NO: 134 - protein, 17 aa, synthetic construct
  REGION 1..17: note = Synthetic
  VARIANT 1: note = /replace="Ser"
  SITE 1..17: note = /note="Variant residues given in the sequence have no preference with respect to those in the annotations for variant positions"
  VARIANT 5: note = /replace="Asp"
  VARIANT 6: note = /replace="Ser"
  VARIANT 7: note = /replace="Val"
  VARIANT 8: note = /replace="Thr"
AISGSGGSTYYADSVKG

SEQ ID NO: 135 - protein, 16 aa, synthetic construct
  REGION 1..16: note = Synthetic
AKGPPTYHTNYYYMDV

SEQ ID NO: 136 - protein, 11 aa, synthetic construct
  REGION 1..11: note = Synthetic
RASQGISSWLA

SEQ ID NO: 137 - protein, 7 aa, synthetic construct
  REGION 1..7: note = Synthetic
AASSLQS

SEQ ID NO: 138 - protein, 9 aa, synthetic construct
  REGION 1..9: note = Synthetic
QQTNSFPYT

SEQ ID NO: 139 - protein, 9 aa, synthetic construct
  REGION 1..9: note = Synthetic
  SITE 1..9: note = /note="Variant residues given in the sequence have no preference with respect to those in the annotations for variant positions"
  VARIANT 4: note = /replace="Val"
  VARIANT 6: note = /replace="Ala"
  VARIANT 7: note = /replace="Asp"
  VARIANT 9: note = /replace="Asn"
FTFSDHYMD

SEQ ID NO: 140 - protein, 19 aa, synthetic construct
  REGION 1..19: note = Synthetic
  SITE 1..19: note = /note="Variant residues given in the sequence have no preference with respect to those in the annotations for variant positions"
  VARIANT 5: note = /replace="Ala"
  VARIANT 6: note = /replace="Leu"
  VARIANT 7: note = /replace="Gly"
RTRNKASSYTTEYAASVKG

SEQ ID NO: 141 - protein, 12 aa, synthetic construct
  REGION 1..12: note = Synthetic
AREPKYWIDFDL

SEQ ID NO: 142 - protein, 11 aa, synthetic construct
  REGION 1..11: note = Synthetic
RASQSISSYLN

SEQ ID NO: 143 - protein, 7 aa, synthetic construct
  REGION 1..7: note = Synthetic
AASSLQS

SEQ ID NO: 144 - protein, 9 aa, synthetic construct
  REGION 1..9: note = Synthetic
QQSYIAPYT

SEQ ID NO: 145 - protein, 976 aa, Homo sapiens [sequence omitted]

SEQ ID NO: 146 - protein, 972 aa, Homo sapiens
MRGARGAWDFLCVLLLLLRVQTGSSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 147 - protein, 123 aa, synthetic construct
  REGION 1..123: note = Synthetic
QVQLVQSGAAVKKPGESLKISCKGSGYRFTTYWIGWVRQMPGKGLEWMGIIYPGDSDTRYSPSFQGQVTISAGKSISTAYLQWSSLKASDTAMYYCARHGRGYNGYEGAFDIWGQGTMVTVSS

SEQ ID NO: 148 - protein, 107 aa, synthetic construct
  REGION 1..107: note = Synthetic
AIQLTQSPSSLSASVGDRVTITCRASQGVSSALAWYQQKPGKAPKLLIYDASSLESGVPSRFSGSGSGTDFTLTISSLQPEDFATYYCQQFNSYPLTFGGGTKVEIK

SEQ ID NO: 149 - protein, 107 aa, synthetic construct
  REGION 1..107: note = Synthetic
DIQLTQSPSSLSASVGDRVTITCRASQGIRTDLGWYQQKPGKAPKLLIYDASSLESGVPSRFSGSGSGTDFTLTISSLQPEDFATYYCQQFNSYPLTFGGGTKVEIK

SEQ ID NO: 150 - protein, 107 aa, synthetic construct
  REGION 1..107: note = Synthetic
AIRMTQSPSSLSASVGDRVTITCRASQGIRNDLAWYQQKPGKTPKLLIYDASSLESGVPSRFSGSGSGTDFTLTISSLQPEDFATYYCQQFNSYPLTFGGGTKVEIK

SEQ ID NO: 151 - protein, 107 aa, synthetic construct
  REGION 1..107: note = Synthetic
AIQMTQSPSSLSASVGDRVTITCRASQGIRNDLGWYQQKPGKAPKLLIYDASSLESGVPSRFSGSGSGTDFTLTISSLQPEDFATYYCQQFNSYPLTFGGGTKVDIK

SEQ ID NO: 152 - protein, 107 aa, synthetic construct
  REGION 1..107: note = Synthetic
NIQMTQSPSSLSASVGDRVTITCRASQAISDYLAWFQQKPGKAPKLLIYDASNLETGVPSRFSGSGSGTDFTLTISSLQPEDFATYYCQQLNSYPLTFGGGTKVEIK

SEQ ID NO: 153 - protein, 107 aa, synthetic construct
  REGION 1..107: note = Synthetic
AIRMTQSPSSLSASVGDRVIIACRASQGIGGALAWYQQKPGNAPKVLVYDASTLESGVPSRFSGGGSGTDFTLTISSLQPEDFATYYCQQFNSYPLTFGGGTKLEIK

SEQ ID NO: 154 - protein, 107 aa, synthetic construct
  REGION 1..107: note = Synthetic
DIAMTQSPPSLSAFVGDRVTITCRASQGIISSLAWYQQKPGKAPKLLIYDASSLESGVPSRFSGSGSGTDFTLTIRSLQPEDFATYYCQQFNSYPLTFGGGTKLEIK

SEQ ID NO: 155 - protein, 107 aa, synthetic construct
  REGION 1..107: note = Synthetic
DIQMTQSPSSLSASVGDRVTITCRASQGISSALAWYQQKAGKAPKVLISDASSLESGVPSRFSGSGSGTDFTLSISSLQPEDFATYYCQQFNGYPLTFGGGTKVDIK

SEQ ID NO: 156 - protein, 107 aa, synthetic construct
  REGION 1..107: note = Synthetic
AIRMTQSPSSLSASVGDRVTITCQASQGIRNDLGWYQQKPGKAPKLLIYDASNLETGVPSRFSGSGSGTDFTFTISSLQPEDIATYYCQQFNSYPLTFGGGTKLEIK

SEQ ID NO: 157 - protein, 107 aa, synthetic construct
  REGION 1..107: note = Synthetic
NIQMTQSPSSLSTSVGDRVTITCRASQGIGTSLAWYQQKPGKPPKLLIYDASSLESGVPSRLSGSGSGTDFTLTISSLQPEDFATYYCQQSNSYPITFGQGTRLEIK

SEQ ID NO: 158 - protein, 107 aa, synthetic construct
  REGION 1..107: note = Synthetic
AIQLTQSPSSLSASVGDRVTITCRASQSIGDYLTWYQQKPGKAPKVLIYGASSLQSGVPPRFSGSGSGTDFTLTVSSLQPEDFATYYCQQLNSYPLTFGGGTKLEIK

SEQ ID NO: 159 - protein, 107 aa, synthetic construct
  REGION 1..107: note = Synthetic
DIQLTQSPSSLSASVGDRVTITCRASQGVRSTLAWYQQKPGKAPKLLIYDASILESGVPSRFSGSGSGTDFTLTISSLQPEDFATYYCQQFNGYPLTFGQGTRLEIK

SEQ ID NO: 160 - protein, 107 aa, synthetic construct
  REGION 1..107: note = Synthetic
DIVMTQSPSSLSASVGDRVTITCRASQGIRNDLGWYQQKPGKAPKLLIYDASSLESGVPSRFSGSGSGTDFTLTISSLQPEDFATYYCQQFNSYPLTFGGGTKLEIK

SEQ ID NO: 161 - protein, 107 aa, synthetic construct
  REGION 1..107: note = Synthetic
DIQLTQSPSSLSASVGDRVTITCRASQGISSFLAWYQQKPGKAPKLLIYDASTLQSGVPSRFSGSASGTDFTLTISSLQPEDFATYYCQQLNGYPLTFGGGTKVEIK

SEQ ID NO: 162 - protein, 107 aa, synthetic construct
  REGION 1..107: note = Synthetic
AIQLTQSPSSLSASVGDRVTITCRASQGIGSALAWYQQKPGIGPKLLIYDASTLESGVPARFSGSGSRTDFTLTITSLQPEDFATYYCQQFNGYPLTFGGGTKLEIK

SEQ ID NO: 163 - protein, 107 aa, synthetic construct
  REGION 1..107: note = Synthetic
AIQLTQSPSSLSASVGDRVTITCRASQGITSALAWYQEKPGKAPNLLIYDASSLESGVPSRFSGSGYGTDFTLTISSLQPEDFATYYCQQLNSYPLTFGGGTKVDIK

SEQ ID NO: 164 - protein, 121 aa, synthetic construct
  REGION 1..121: note = Synthetic
QIQLVQSGPELRKPGESVKISCKASGYTFTDYAMYWVKQAPGKGLKWMGWINTYTGKPTYADDFKGRFVFSLEASANTANLQISNLKNEDTATYFCARARGLVDDYVMDAWGQGTSVTVSS

SEQ ID NO: 165 - protein, 108 aa, synthetic construct
  REGION 1..108: note = Synthetic
SYELIQPPSASVTLGNTVSLTCVGDELSKRYAQWYQQKPDKTIVSVIYKDSERPSGISDRFSGSSSGTTATLTIHGTLAEDEADYYCLSTYSDDNLPVFGGGTKLTVL

SEQ ID NO: 166 - protein, 117 aa, synthetic construct
  REGION 1..117: note = Synthetic
EVQLQQYGAELGKPGTSVRLSCKVSGYNIRNTYIHWVNQRPGEGLEWIGRIDPTNGNTISAEKFKTKATLTADTSSHTAYLQFSQLKSDDTAIYFCALNYEGYADYWGQGVMVTGSS

SEQ ID NO: 167 - protein, 106 aa, synthetic construct
  REGION 1..106: note = Synthetic
DIQMTQSPSFLSASVGDRVTINCKASQNINKYLNWYQQKVGEAPKRLIFKTNSLQTGIPSRFSGSGSGTDYTLTISSLQTEDVATYFCFQYNIGYTFGAGTKVELK

SEQ ID NO: 168 - protein, 124 aa, synthetic construct
  REGION 1..124: note = Synthetic
EVQLQESGPGLVKPSQSLSLTCSVTGYSISSNYRWNWIRKFPGNKVEWMGYINSAGSTNYNPSLKSRISMTRDTSKNQFFLQVNSVTTEDTATYYCARSLRGYITDYSGFFDYWGQGVMVTVSS

SEQ ID NO: 169 - protein, 107 aa, synthetic construct
  REGION 1..107: note = Synthetic
DIRMTQSPASLSASLGETVNIECLASEDIFSDLAWYQQKPGKSPQLLIYNANSLQNGVPSRFSGSGSGTRYSLKINSLQSEDVATYFCQQYKNYPLTFGSGTKLEIK

SEQ ID NO: 170 - protein, 117 aa, synthetic construct
  REGION 1..117: note = Synthetic
EVQLQQYGAELGKPGTSVRLSCKLSGYKIRNTYIHWVNQRPGKGLEWIGRIDPANGNTIYAEKFKSKVTLTADTSSNTAYMQLSQLKSDDTALYFCAMNYEGYEDYWGQGVMVTVSS

SEQ ID NO: 171 - protein, 106 aa, synthetic construct
  REGION 1..106: note = Synthetic
DIQMTQSPSFLSASVGDSVTINCKASQNINKYLNWYQQKLGEAPKRLIHKTDSLQTGIPSRFSGSGSGTDYTLTISSLQPEDVATYFCFQYKSGFMFGAGTKLELK

SEQ ID NO: 172 - protein, 120 aa, synthetic construct
  REGION 1..120: note = Synthetic
QIQLVQSGPELKKPGESVKISCKASGYTFTDYAVYWVIQAPGKGLKWMGWINTYTGKPTYADDFKGRFVFSLETSASTANLQISNLKNEDTATYFCARGAGMTKDYVMDAWGRGVLVTVS

SEQ ID NO: 173 - protein, 108 aa, synthetic construct
  REGION 1..108: note = Synthetic
SYELIQPPSASVTLGNTVSLTCVGDELSKRYAQWYQQKPDKTIVSVIYKDSERPSDISDRFSGSSSGTTATLTIHGTLAEDEADYYCLSTYSDDNLPVFGGGTKLTVL

SEQ ID NO: 174 - protein, 116 aa, synthetic construct
  REGION 1..116: note = Synthetic
QVQLKESGPGLVQPSQTLSLTCTVSGFSLTSYLVHWVRQPPGKTLEWVGLMWNDGDTSYNSALKSRLSISRDTSKSQVFLKMHSLQAEDTATYYCARESNLGFTYWGHGTLVTVSS

SEQ ID NO: 175 - protein, 107 aa, synthetic construct
  REGION 1..107: note = Synthetic
DIQMTQSPASLSASLEEIVTITCKASQGIDDDLSWYQQKPGKSPQLLIYDVTRLADGVPSRFSGSRSGTQYSLKISRPQVADSGIYYCLQSYSTPYTFGAGTKLELK

SEQ ID NO: 176 - protein, 117 aa, synthetic construct
  REGION 1..117: note = Synthetic
EVQLQQYGAELGKPGTSVRLSCKVSGYNIRNTYIHWVHQRPGEGLEWIGRIDPTNGNTISAEKFKSKATLTADTSSNTAYMQFSQLKSDDTAIYFCAMNYEGYADYWGQGVMVTVSS

SEQ ID NO: 177 - protein, 106 aa, synthetic construct
  REGION 1..106: note = Synthetic
DIQMTQSPSFLSASVGDRLTINCKASQNINKYLNWYQQKLGEAPKRLIFKTNSLQTGIPSRFSGSGSGTDYTLTISSLQPEDVATYFCFQYNIGFTFGAGTKLELK

SEQ ID NO: 178 - protein, 124 aa, synthetic construct
  REGION 1..124: note = Synthetic
EVQLVESGGGLVQSGRSLKLSCAASGFTVSDYYMAWVRQAPTKGLEWVATINYDGSTTYHRDSVKGRFTISRDNAKSTLYLQMDSLRSEDTATYYCARHGDYGYHYGAYYFDYWGQGVMVTVSS

SEQ ID NO: 179 - protein, 109 aa, synthetic construct
  REGION 1..109: note = Synthetic
DIVLTQSPALAVSLGQRATISCRASQTVSLSGYNLIHWYQQRTGQQPKLLIYRASNLAPGIPARFSGSGSGTDFTLTISPVQSDDIATYYCQQSRESWTFGGGTNLEMK

SEQ ID NO: 180 - protein, 116 aa, synthetic construct
  REGION 1..116: note = Synthetic
QIQLVQSGPELKKPGESVKISCKASGYTFTDYAIHWVKQAPGQGLRWMAWINTETGKPTYADDFKGRFVFSLEASASTAHLQISNLKNEDTATFFCAGGSHWFAYWGQGTLVTVSS

SEQ ID NO: 181 - protein, 108 aa, synthetic construct
  REGION 1..108: note = Synthetic
SYELIQPPSASVTLENTVSITCSGDELSNKYAHWYQQKPDKTILEVIYNDSERPSGISDRFSGSSSGTTAILTIRDAQAEDEADYYCLSTFSDDDLPIFGGGTKLTVL

SEQ ID NO: 182 - protein, 108 aa, synthetic construct
  REGION 1..108: note = Synthetic
SYELIQPPSTSVTLGNTVSLTCVGNELPKRYAYWFQQKPDQSIVRLIYDDDRRPSGISDRFSGSSSGTTATLTIRDAQAEDEAYYYCHSTYTDDKVPIFGGGTKLTVL

SEQ ID NO: 183 - protein, 123 aa, synthetic construct
  REGION 1..123: note = Synthetic
EVQLVESGGGLVQPGRSMKLSCKASGFTFSNYDMAWVRQAPTRGLEWVASISYDGITAYYRDSVKGRFTISRENAKSTLYLQLVSLRSEDTATYYCTTEGGYVYSGPHYFDYWGQGVMVTVSS

SEQ ID NO: 184 - protein, 107 aa, synthetic construct
  REGION 1..107: note = Synthetic
DIQMTQSPSSMSVSLGDTVTITCRASQDVGIFVNWFQQKPGRSPRRMIYRATNLADGVPSRFSGSRSGSDYSLTISSLESEDVADYHCLQYDEFPRTFGGGTKLELK

SEQ ID NO: 185 - protein, 117 aa, synthetic construct
  REGION 1..117: note = Synthetic
EVQLQQYGAELGKPGTSVRLSCKVSGYKIRNTYIHWVNQRPGKGLEWIGRIDPANGNTIYAEKFKSKVTLTADTSSNTAYMQLSQLKSDDTALYFCAMNYEGYEDYWGQGVMVTVSS

SEQ ID NO: 186 - protein, 106 aa, synthetic construct
  REGION 1..106: note = Synthetic
DIQMTQSPSFLSASVGDSVTINCKASQNINKYLNWYQQKLGEAPKRLIHKTNSLQPGFPSRFSGSGSGTDYTLTISSLQPEDVAAYFCFQYNSGFTFGAGTKLELK

SEQ ID NO: 187 - protein, 125 aa, synthetic construct
  REGION 1..125: note = Synthetic
QVQLVQSGAEVKKPGASVKVSCKASGYTFTDYYIHWVRQAPGQGLEWMGWMNPHSGDTGYAQKFQGRVTMTRDTSTSTVYMELSSLRSEDTAVYYCARHGRGYNGYEGAFDIWGQGTLVTVSSAS

SEQ ID NO: 188 - protein, 107 aa, synthetic construct
  REGION 1..107: note = Synthetic
DIQMTQSPSSLSASVGDRVTITCRASQGIGNELGWYQQKPGKAPKLLIYAASNLQSGVPSRFSGSGSGTDFTLTISSLQPEDFATYYCQQYDNLPLTFGQGTKVEIK

SEQ ID NO: 189 - protein, 125 aa, synthetic construct
  REGION 1..125: note = Synthetic
QVQLVQSGAEVKKPGASVKVSCKASGYTFTGYYLHWVRQAPGQGLEWMGWINPNSGDTNYAQNFQGRVTMTRDTSTSTVYMELSSLRSEDTAVYYCARHGRGYNGYEGAFDIWGQGTLVTVSSAS

SEQ ID NO: 190 - protein, 107 aa, synthetic construct
  REGION 1..107: note = Synthetic
DIQMTQSPSSLSASVGDRVTITCRASQGIRNDLGWYQQKPGKAPKLLIYDASSLESGVPSRFSGSGSGTDFTLTISSLQPEDFATYYCQQLNGYPLTFGGGTKVEIK

SEQ ID NO: 191 - protein, 125 aa, synthetic construct
  REGION 1..125: note = Synthetic
QVQLVQSGAEVKKPGASVKVSCKASGYTFTGYYLHWVRQAPGQGLEWMGWINPNSGGTNYAQKFQGRVTMTRDTSTSTVYMELSSLRSEDTAVYYCARHGRGYEGYEGAFDIWGQGTLVTVSSAS

SEQ ID NO: 192 - protein, 107 aa, synthetic construct
  REGION 1..107: note = Synthetic
DIQMTQSPSSLSASVGDRVTITCRASQGIRNDLGWYQQKPGKAPKLLIYDASELETGVPSRFSGSGSGTDFTLTISSLQPEDFATYYCQQLNGYPITFGQGTKVEIK

SEQ ID NO: 193 - protein, 125 aa, synthetic construct
  REGION 1..125: note = Synthetic
QVQLVQSGAEVKKPGASVKVSCKASGYTFTSYYIHWVRQAPGQGLEWMGWLNPSGGGTSYAQKFQGRVTMTRDTSTSTVYMELSSLRSEDTAVYYCARHGRGYDGYEGAFDIWGQGTLVTVSSAS

SEQ ID NO: 194 - protein, 107 aa, synthetic construct
  REGION 1..107: note = Synthetic
DIQMTQSPSSLSASVGDRVTITCRASQGIRNDLGWYQQKPGKAPKLLIYDASNLETGVPSRFSGSGSGTDFTLTISSLQPEDFATYYCQQLNGYPLTFGGGTKVEIK

SEQ ID NO: 195 - protein, 125 aa, synthetic construct
  REGION 1..125: note = Synthetic
QVQLVQSGAEVKKPGASVKVSCKASGYTFSTYYMHWVRQAPGQGLEWMGIINPSGGSTSYAQKFQGRVTMTRDTSTSTVYMKLSSLRSEDTAVYYCARHGRGYEGYEGAFDIWGQGTLVTVSSAS

SEQ ID NO: 196 - protein, 107 aa, synthetic construct
  REGION 1..107: note = Synthetic
DIQMTQSPSSLSASVGDRVTITCRASQGIRDDLGWYQQKPGKAPKLLIYDASNLETGVPSRFSGSGSGTDFTLTISSLQPEDFATYYCQQANGFPLTFGGGTKVEIK

SEQ ID NO: 197 - protein, 125 aa, synthetic construct
  REGION 1..125: note = Synthetic
QVQLVQSGAEVKKPGASVKVSCKASGYTFTGYYIHWVRQAPGQGLEWMGIINPSGGNTNYAQNFQGRVTMTRDTSTSTVYMELSSLRSEDTAVYYCARHGRGYNAYEGAFDIWGQGTLVTVSSAS

SEQ ID NO: 198 - protein, 107 aa, synthetic construct
  REGION 1..107: note = Synthetic
DIQMTQSPSSLSASVGDRVTITCRASQGIRNDLGWYQQKPGKAPKLLIYDASNLETGVPSRFSGSGSGTDFTLTISSLQPEDFATYYCQQVNGYPLTFGGGTKVEIK

SEQ ID NO: 199 - protein, 125 aa, synthetic construct
  REGION 1..125: note = Synthetic
QVQLVQSGAEVKKPGASVKVSCKASGGTFSSYAISWVRQAPGQGLEWMGVINPTVGGANYAQKFQGRVTMTRDTSTSTVYMELSSLRSEDTAVYYCARHGRGYNEYEGAFDIWGQGTLVTVSSAS

SEQ ID NO: 200 - protein, 107 aa, synthetic construct
  REGION 1..107: note = Synthetic
DIQMTQSPSSLSASVGDRVTITCQASQDISDYLNWYQQKPGKAPKLLIYDASNLETGVPSRFSGSGSGTDFTLTISSLQPEDFATYYCQQGNSFPLTFGGGTKLEIK

SEQ ID NO: 201 - protein, 125 aa, synthetic construct
  REGION 1..125: note = Synthetic
QVQLVQSGAEVKKLGASVKVSCKASGYTFSSYYMHWVRQAPGQGLEWMGVINPNGAGTNFAQKFQGRVTMTRDTSTSTVYMELSSLRSEDTAVYYCARHGRGYEGYEGAFDIWGQGTLVTVSSAS

SEQ ID NO: 202 - protein, 125 aa, synthetic construct
  REGION 1..125: note = Synthetic
QVQLVQSGAEVKKPGASVKVSCKASGYTFTTYYMHWVRQAPGQGLEWMGWINPTGGGTNYAQNFQGRVTMTRDTSTSTVYMELSSLRSEDTAVYYCARHGRGYEGYEGAFDIWGQGTLVTVSSAS

SEQ ID NO: 203 - protein, 107 aa, synthetic construct
  REGION 1..107: note = Synthetic
DIQMTQSPSSLSASVGDRVTITCRASQGIRNDVSWYQQKPGKAPKLLIYDASNLETGVPSRFSGSGSGTDFTLTISSLQPEDFATYYCQQLSGYPITFGQGTKLEIK

SEQ ID NO: 204 - protein, 125 aa, synthetic construct
  REGION 1..125: note = Synthetic
QVQLVQSGAEVKKPGASVKVSCKASGYTFTSYYIHWVRQAPGQGLEWMGMINPSGGSTNYAQKFQGRVTMTRDTSTSTVYMELSSLRSEDTAVYYCARHGRGYNDYEGAFDIWGQGTLVTVSSAS

SEQ ID NO: 205 - protein, 107 aa, synthetic construct
  REGION 1..107: note = Synthetic
DIQMTQSPSSLSASVGDRVTITCRASQSISDWLAWYQQKPGKAPKLLIYEASNLEGGVPSRFSGSGSGTDFTLTISSLQPEDFATYYCQQANSFPYTFGQGTKVEIK

SEQ ID NO: 206 - protein, 125 aa, synthetic construct
  REGION 1..125: note = Synthetic
QVQLVQSGAEVKKPGASVKVSCKASGYIFSAYYIHWVRQAPGQGLEWMGIINPSGGSTRYAQKFQGRVTMTRDTSTSTVYMELSSLRSEDTAVYYCARHGRGYGGYEGAFDIWDQGTLVTVSSAS

SEQ ID NO: 207 - protein, 107 aa, synthetic construct
  REGION 1..107: note = Synthetic
DIQMTQSPSSLSASVGDRVTITCRASQGIGDYVAWYQQKPGKAPKLLIYDASNLETGVPSRFSGSGSGTDFTLTISSLQPEDFATYYCQQLNGYPITFGQGTRLEIK

SEQ ID NO: 208 - protein, 125 aa, synthetic construct
  REGION 1..125: note = Synthetic
EVQLVQSGAEVKKPGESLKISCKGSGYRFTSYWIGWVRQMPGKGLEWMGIIYPDDSDTRYSPSFQGQVTISVDKSNSTAYLQWSSLKASDTAMYYCARHGRGYNGYEGAFDIWGQGTLVTVSSAS

SEQ ID NO: 209 - protein, 107 aa, synthetic construct
  REGION 1..107: note = Synthetic
DIQMTQSPSSLSASVGDRVTITCRASQGISSYLAWYQQKPGKAPKLLIYDASNLETGVPSRFSGSGSGTYFTLTISSLQPEDFATYYCQQGASFPITFGQGTKVEIK

SEQ ID NO: 210 - protein, 125 aa, synthetic construct
  REGION 1..125: note = Synthetic
EVQLVQSGAEVKKPGESLKISCKGSGSSFPNSWIAWVRQMPGKGLEWMGIIYPSDSDTRYSPSFQGQVTISADKSISTAYLQWSSLEASDTAMYYCARHGRGYNGYEGAFDIWGQGTLVTVSSAS

SEQ ID NO: 211 - protein, 107 aa, synthetic construct
  REGION 1..107: note = Synthetic
DIQMTQSPSSLSASVGDRVTITCRASQGIRNYLAWYQQKPGKAPKLLIYDASSLQSGVPSRFSGSGSGTDFTLTISSLQPEDFATYYCQQLNSYPLTFGGGTKVEIK

SEQ ID NO: 212 - protein, 125 aa, synthetic construct
  REGION 1..125: note = Synthetic
EVQLVQSGAEVKKPGESLKISCKGSGYSFDSYWIGWVRQMPGKGLEWMGIMYPGDSDTRYSPSFQGQVTISADKSISTAYLQWSSLKASDTAMYYCARHGRGYNAYEGAFDIWGQGTLVTVSSAS

SEQ ID NO: 213 - protein, 107 aa, synthetic construct
  REGION 1..107: note = Synthetic
DIQMTQSPSSLSASVGDRVTITCRASQSINNWLAWYQQKPGKAPKLLIYDAFILQSGVPSRFSGSGSGTDFTLTISSLQPEDFATYYCLQLNSYPLTFGPGTKVDIK

SEQ ID NO: 214 - protein, 124 aa, synthetic construct
  REGION 1..124: note = Synthetic
EVQLVQSGAEVKKPGESLKISCKGSGYSFTNWIAWVRQMPGKGLEWMGIIYPGDSETRYSPSFQGQVTISADKSISTAYLQWSSLKASDTAMYYCARHGRGYYGYEGAFDIWGQGTLVTVSSAS

SEQ ID NO: 215 - protein, 107 aa, synthetic construct
  REGION 1..107: note = Synthetic
DIQMTQSPSSLSASVGDRVTITCRASQGISDNLNWYQQKPGKAPKLLIYDASNLETGVPSRFSGSGSGTDFTLTISSLQPEDFATYYCQQAISFPLTFGQGTKVEIK

SEQ ID NO: 216 - protein, 125 aa, synthetic construct
  REGION 1..125: note = Synthetic
EVQLVQSGAEVKKPGESLKISCKGSGYNFTSYWIGWVRQMPGKGLEWMGVIYPDDSETRYSPSFQGQVTISADKSISTAYLQWSSLKASDTAMYYCARHGRGYNGYEGAFDIWGQGTLVTVSSAS

SEQ ID NO: 217 - protein, 107 aa, synthetic construct
  REGION 1..107: note = Synthetic
DIQMTQSPSSLSASVGDRVTITCRASRDIRDDLGWYQQKPGKAPKLLIYDASNLETGVPSRFSGSGSGTDFTLTISSLQPEDFATYYCQQANSFPLTFGGGTKVEIK

SEQ ID NO: 218 - protein, 124 aa, synthetic construct
  REGION 1..124: note = Synthetic
EVQLVQSGAEVKKPGESLKISCKGSGYTFNTYIGWVRQMPGKGLEWMGIIYPGDSGTRYSPSFQGQVTISADKAISTAYLQWSSLKASDTAMYYCARHSRGYNGYEGAFDIWGQGTLVTVSSAS

SEQ ID NO: 219 - protein, 107 aa, synthetic construct
  REGION 1..107: note = Synthetic
DIQMTQSPSSLSASVGDRVTITCRASQGISNYLAWYQQKPGKAPKLLIYDASNLETGVPSRFSGSGSGTDFTLTISSLQPEDFATYYCQQANSFPVTFGQGTKVEIK

SEQ ID NO: 220 - protein, 125 aa, synthetic construct
  REGION 1..125: note = Synthetic
EVQLVQSGAEVKKPGESLKISCKGSGYNFTTYWIGWVRQMPGKGLEWMGIIHPADSDTRYNPSFQGQVTISADKSISTAYLQWSSLKASDTAMYYCARHGRGYNGYEGAFDIWGQGTLVTVSSAS

SEQ ID NO: 221 - protein, 107 aa, synthetic construct
  REGION 1..107: note = Synthetic
DIQMTQSPSSLSASVGDRVTITCRVSQGISSYLAWYQQKPGKAPKLLIYDASNLETGVPSRFSGSGSGTDFTLTISSLQPEDFATYYCQQANSFPLTFGGGTKVEIK

SEQ ID NO: 222 - protein, 125 aa, synthetic construct
  REGION 1..125: note = Synthetic
EVQLVQSGAEVKKPGESLKISCKGSGYRFSNYWIAWVRQMPGKGLEWMGIIYPDNSDTRYSPSFQGQVTISADKSISTAYLQWSSLKASDTAMYYCARHGRGYDGYEGAFDIWGQGTLVTVSSAS

SEQ ID NO: 223 - protein, 107 aa, synthetic construct
  REGION 1..107: note = Synthetic
DIQMTQSPSSLSASVGDRVTITCRASQGIRSDLAWYQQKPGKAPKLLIYGASSLQSGVPSRFSGSGSGTDFTLTISSLQPEDFATYYCQQANSFPLSFGQGTKVEIK

SEQ ID NO: 224 - protein, 124 aa, synthetic construct
  REGION 1..124: note = Synthetic
EVQLVQSGAEVKKPGESLKISCKGSGYRFASYWIGWVRQMPGKGLEWMGITYPGDSETRYNPSQGQVTISADKSISTAYLQWSSLKASDTAMYYCARHGRGYGGYEGAFDIWGQGTLVTVSSAS

SEQ ID NO: 225 - protein, 107 aa, synthetic construct
  REGION 1..107: note = Synthetic
DIQMTQSPSSLSASVGDRVTITCRASQGIRNDLGWYQQKPGKAPKLLIYDASNLETGVPSRFSGSGSGTDFTLTISSLQPEDFATYYCQQANSFPLTFGGGTKVEIK

SEQ ID NO: 226 - protein, 125 aa, synthetic construct
  REGION 1..125: note = Synthetic
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARHGRGYNGYEGAFDIWGQGTLVTVSSAS

SEQ ID NO: 227 - protein, 107 aa, synthetic construct
  REGION 1..107: note = Synthetic
DIQMTQSPSSLSASVGDRVTITCRASQSISNWLAWYQQKPGKAPKLLIYDASNLETGVPSRFSGSGSGTDFTLTISSLQPEDFATYYCQQTNSFPLTFGQGTRLEIK

SEQ ID NO: 228 - protein, 107 aa, synthetic construct
  REGION 1..107: note = Synthetic
DIQLTQSPSSLSASVGDRVTITCRASQGVISALAWYQQKPGKAPKLLIYDASSLESGVPSRFSGSGSGTDFTLTISSLQPEDFATYYCQQFNSYPLTFGGGTKVEIK

SEQ ID NO: 229 - protein, 107 aa, synthetic construct
  REGION 1..107: note = Synthetic
DIQLTQSPSSLSASVGDRVTITCRASQGIRSALAWYQQKPGKAPKLLIYDASSLESGVPSRFSGSGSGTDFTLTISSLQPEDFATYYCQQFNSYPLTFGGGTKVEIK

SEQ ID NO: 230 - protein, 107 aa, synthetic construct
  REGION 1..107: note = Synthetic
DIQLTQSPSSLSASVGDRVTITCRASQGVGSALAWYQQKPGKAPKLLIYDASSLESGVPSRFSGSGSGTDFTLTISSLQPEDFATYYCQQFNSYPLTFGGGTKVEIK

SEQ ID NO: 231 - protein, 107 aa, synthetic construct
  REGION 1..107: note = Synthetic
DIQLTQSPSSLSASVGDRVTITCRASQGVISALAWYQQKPGKAPKLLIYDASILESGVPSRFSGSGSGTDFTLTISSLQPEDFATYYCQQFNSYPLTFGGGTKVEIK

SEQ ID NO: 232 - protein, 107 aa, synthetic construct
  REGION 1..107: note = Synthetic
DIQLTQSPSSLSASVGDRVTITCRASQGIRSALAWYQQKPGKAPKLLIYDASILESGVPSRFSGSGSGTDFTLTISSLQPEDFATYYCQQFNSYPLTFGGGTKVEIK

SEQ ID NO: 233 - protein, 107 aa, synthetic construct
  REGION 1..107: note = Synthetic
DIQLTQSPSSLSASVGDRVTITCRASQGVGSALAWYQQKPGKAPKLLIYDASILESGVPSRFSGSGSGTDFTLTISSLQPEDFATYYCQQFNSYPLTFGGGTKVEIK

SEQ ID NO: 234 - protein, 107 aa, synthetic construct
  REGION 1..107: note = Synthetic
DIQLTQSPSSLSASVGDRVTITCRASQGISSALAWYQQKPGKAPKLLIYDASILESGVPSRFSGSGSGTDFTLTISSLQPEDFATYYCQQFNSYPLTFGGGTKVEIK

SEQ ID NO: 235 - protein, 107 aa, synthetic construct
  REGION 1..107: note = Synthetic
DIQLTQSPSSLSASVGDRVTITCRASQGVISALAWYQQKPGKAPKLLIYDASTLESGVPSRFSGSGSGTDFTLTISSLQPEDFATYYCQQFNSYPLTFGGGTKVEIK

SEQ ID NO: 236 - protein, 107 aa, synthetic construct
  REGION 1..107: note = Synthetic
DIQLTQSPSSLSASVGDRVTITCRASQGIRSALAWYQQKPGKAPKLLIYDASTLESGVPSRFSGSGSGTDFTLTISSLQPEDFATYYCQQFNSYPLTFGGGTKVEIK

SEQ ID NO: 237 - protein, 107 aa, synthetic construct
  REGION 1..107: note = Synthetic
DIQLTQSPSSLSASVGDRVTITCRASQGVGSALAWYQQKPGKAPKLLIYDASTLESGVPSRFSGSGSGTDFTLTISSLQPEDFATYYCQQFNSYPLTFGGGTKVEIK

SEQ ID NO: 238 - protein, 123 aa, synthetic construct
  REGION 1..123: note = Synthetic
EVQLVQSGAEVKKPGESLKISCKGSGYRFTTSWIGWVRQMPGKGLEWMGIIYPGDSDTRYSPSFQGQVTISADKSISTAYLQWSSLKASDTAMYYCARHGLGYNGYEGAFDIWGQGTLVTVSS

SEQ ID NO: 239 - protein, 107 aa, synthetic construct
  REGION 1..107: note = Synthetic
DIQMTQSPSSLSASVGDRVTITCRASQGIGSALAWYQQKPGKAPKLLIYDASTLESGVPSRFSGSGSGTDFTLTISSLQPEDFATYYCQQFNGYPLTFGQGTRLEIK

SEQ ID NO: 240 - protein, 107 aa, synthetic construct
  REGION 1..107: note = Synthetic
DIQMTQSPSSLSASVGDRVTITCRASRGISDYLAWYQQKPGKAPKLLIYDASNLETGVPSRFSGSGSGTDFTLTISSLQPEDFATYYCQQANSFPITFGQGTRLEIK

SEQ ID NO: 241 - protein, 107 aa, synthetic construct
  REGION 1..107: note = Synthetic
DIQMTQSPSSLSASVGDRVTITCRASQGIGSALAWYQQKPGKAPKLLIYDASTLESGVPSRFSGSGSGTDFTLTISSLQPEDFATYYCQQLNGYPLTFGQGTRLEIK

SEQ ID NO: 242 - protein, 107 aa, synthetic construct
  REGION 1..107: note = Synthetic
DIQMTQSPSSLSASVGDRVTITCRASQGIGSALAWYQQKPGKAPKLLIYDASNLETGVPSRFSGSGSGTDFTLTISSLQPEDFATYYCQQLNGYPLTFGQGTRLEIK

SEQ ID NO: 243 - protein, 123 aa, synthetic construct
  REGION 1..123: note = Synthetic
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSS

SEQ ID NO: 244 - protein, 107 aa, synthetic construct
  REGION 1..107: note = Synthetic
DIQMTQSPSSLSASVGDRVTITCRSSQGIRSDLGWYQQKPGKAPKLLIYDASNLETGVPSRFSGSGSGTDFTLTISSLQPEDFATYYCQQANGFPLTFGGGTKVEIK

SEQ ID NO: 245 - protein, 5 aa, synthetic construct
  REGION 1..5: note = Synthetic
NYWIG

SEQ ID NO: 246 - protein, 17 aa, synthetic construct
  REGION 1..17: note = Synthetic
IINPRDSDTRYRPSFQG

SEQ ID NO: 247 - protein, 14 aa, synthetic construct
  REGION 1..14: note = Synthetic
HGRGYEGYEGAFDI

SEQ ID NO: 248 - protein, 11 aa, synthetic construct
  REGION 1..11: note = Synthetic
RSSQGIRSDLG

SEQ ID NO: 249 - protein, 7 aa, synthetic construct
  REGION 1..7: note = Synthetic
DASNLET

SEQ ID NO: 250 - protein, 9 aa, synthetic construct
  REGION 1..9: note = Synthetic
QQANGFPLT

SEQ ID NO: 251 - protein, 123 aa, synthetic construct
  REGION 1..123: note = Synthetic
EVQLVQSGAEVKKPGESLKISCKGSGYSFTNYWIGWVRQMPGKGLEWMGIIYPGDSDIRYSPSLQGQVTISVDTSTSTAYLQWNSLKPSDTAMYYCARHGRGYNGYEGAFDIWGQGTLVTVSS

SEQ ID NO: 252 - protein, 107 aa, synthetic construct
  REGION 1..107: note = Synthetic
DIQMTQSPSSLSASVGDRVTITCRASQGIGDSLAWYQQKPGKAPKLLIYDASNLETGVPSRFSGSGSGTDFTLTISSLQPEDFATYYCQQLNGYPITFGQGTKVEIK

SEQ ID NO: 253 - protein, 17 aa, synthetic construct
  REGION 1..17: note = Synthetic
IIYPGDSDIRYSPSLQG

SEQ ID NO: 254 - protein, 11 aa, synthetic construct
  REGION 1..11: note = Synthetic
RASQGIGDSLA

SEQ ID NO: 255 - protein, 9 aa, synthetic construct
  REGION 1..9: note = Synthetic
QQLNGYPIT

SEQ ID NO: 256 - protein, 107 aa, synthetic construct
  REGION 1..107: note = Synthetic
DIQMTQSPSSLSASVGDRVTITCRASQGIRNDLGWYQQKPGKAPKLLIYDASSLESGVPSRFSGSGSGTDFTLTISSLQPEDFATYYCQQLNGYPITFGQGTKVEIK

SEQ ID NO: 257 - protein, 11 aa, synthetic construct
  REGION 1..11: note = Synthetic
RASQGIRNDLG

SEQ ID NO: 258 - protein, 123 aa, synthetic construct
  REGION 1..123: note = Synthetic
EVQLVQSGAEVKKPGESLKISCKGSGYSFTNYWIGWVRQMPGKGLEWMGIIYPGDSLTRYSPSFQGQVTISADKSISTAYLQWSSLKASDTAMYYCARHGRGYNGYEGAFDIWGQGTLVTVSS

SEQ ID NO: 259 - protein, 17 aa, synthetic construct
  REGION 1..17: note = Synthetic
IIYPGDSLTRYSPSFQG

SEQ ID NO: 260 - protein, 123 aa, synthetic construct
  REGION 1..123: note = Synthetic
EVQLVQSGAEVKKPGESLKISCKGSGYSFTNYWIGWVRQMPGKGLEWMGIIYPGDSDTRYSPSFQGQVTISADKSISTAYLQWSSLKASDTAMYYCARHGRGYNGYEGAFDIWGQGTLVTVSS

SEQ ID NO: 261 - protein, 123 aa, synthetic construct
  REGION 1..123: note = Synthetic
QVQLVQSGAAVKKPGESLKISCKGSGYRFTSYWIGWVRQMPGKGLEWMGIIYPGDSDTRYSPSFQGQVTISAGKSISTAYLQWSSLKASDTAMYYCARHGRGYNGYEGAFDIWGQGTMVTVSS

SEQ ID NO: 262 - protein, 107 aa, synthetic construct [sequence omitted]

SEQ ID NO: 263 - protein, 5 aa, synthetic construct
  REGION 1..5: note = Synthetic
TYWIG

SEQ ID NO: 264 - protein, 11 aa, synthetic construct
  REGION 1..11: note = Synthetic
RASQGVISALA

SEQ ID NO: 265 - protein, 7 aa, synthetic construct
  REGION 1..7: note = Synthetic
DASILES

SEQ ID NO: 266 - protein, 9 aa, synthetic construct
  REGION 1..9: note = Synthetic
QQFNSYPLT

SEQ ID NO: 267 - protein, 11 aa, synthetic construct
  REGION 1..11: note = Synthetic
RASQGVGSALA

SEQ ID NO: 268 - protein, 7 aa, synthetic construct
  REGION 1..7: note = Synthetic
DASTLES

SEQ ID NO: 269 - protein, 330 aa, Homo sapiens [sequence omitted]

SEQ ID NO: 270 - protein, 330 aa, synthetic construct
  REGION 1..330: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 271 - protein, 330 aa, synthetic construct; identical to SEQ ID NO: 123
  REGION 1..330: note = Synthetic

SEQ ID NO: 272 - protein, 330 aa, synthetic construct
  REGION 1..330: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 273 - protein, 330 aa, synthetic construct; identical to SEQ ID NO: 124
  REGION 1..330: note = Synthetic

SEQ ID NO: 274 - protein, 330 aa, synthetic construct; identical to SEQ ID NO: 126
  REGION 1..330: note = Synthetic

SEQ ID NO: 275 - protein, 453 aa, synthetic construct
  REGION 1..453: note = Synthetic
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 276 - protein, 453 aa, synthetic construct
  REGION 1..453: note = Synthetic
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 277 - protein, 453 aa, synthetic construct
  REGION 1..453: note = Synthetic
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 278 - protein, 453 aa, synthetic construct
  REGION 1..453: note = Synthetic
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 279 - protein, 453 aa, synthetic construct
  REGION 1..453: note = Synthetic
EVQLVQSGAEVKKPGESLKISCKGSGYRFTTSWIGWVRQMPGKGLEWMGIIYPGDSDTRYSPSFQGQVTISADKSISTAYLQWSSLKASDTAMYYCARHGLGYNGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 280 - protein, 453 aa, synthetic construct
  REGION 1..453: note = Synthetic
EVQLVQSGAEVKKPGESLKISCKGSGYRFTTSWIGWVRQMPGKGLEWMGIIYPGDSDTRYSPSFQGQVTISADKSISTAYLQWSSLKASDTAMYYCARHGLGYNGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 281 - protein, 453 aa, synthetic construct
  REGION 1..453: note = Synthetic
EVQLVQSGAEVKKPGESLKISCKGSGYRFTTSWIGWVRQMPGKGLEWMGIIYPGDSDTRYSPSFQGQVTISADKSISTAYLQWSSLKASDTAMYYCARHGLGYNGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 282 - protein, 453 aa, synthetic construct
  REGION 1..453: note = Synthetic
EVQLVQSGAEVKKPGESLKISCKGSGYRFTTSWIGWVRQMPGKGLEWMGIIYPGDSDTRYSPSFQGQVTISADKSISTAYLQWSSLKASDTAMYYCARHGLGYNGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 283 - protein, 107 aa, synthetic construct [sequence omitted]

SEQ ID NO: 284 - protein, 214 aa, synthetic construct
  REGION 1..214: note = Synthetic
DIQMTQSPSSLSASVGDRVTITCRSSQGIRSDLGWYQQKPGKAPKLLIYDASNLETGVPSRFSGSGSGTDFTLTISSLQPEDFATYYCQQANGFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 285 - protein, 214 aa, synthetic construct
  REGION 1..214: note = Synthetic
DIQMTQSPSSLSASVGDRVTITCRASQGIGSALAWYQQKPGKAPKLLIYDASNLETGVPSRFSGSGSGTDFTLTISSLQPEDFATYYCQQLNGYPLTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 286 - protein, 5 aa, synthetic construct
  REGION 1..5: note = Synthetic
TSWIG

SEQ ID NO: 287 - protein, 14 aa, synthetic construct
  REGION 1..14: note = Synthetic
HGLGYNGYEGAFDI

SEQ ID NO: 288 - protein, 11 aa, synthetic construct
  REGION 1..11: note = Synthetic
RASQGIGSALA

SEQ ID NO: 289 - protein, 10 aa, synthetic construct
  REGION 1..10: note = Synthetic
CQQLNGYPLT

SEQ ID NO: 290 - protein, 453 aa, synthetic construct
  REGION 1..453: note = Synthetic
QVQLQQPGAELVRPGASVKLSCKASGYTFTSYWINWVKQRPGQGLEWIGNIYPSDSYTNYNQKFKDKATLTVDKSSNTVYMQLNSPTSEDSAVYYCTRNGVEGYPHYYAMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 291 - protein, 214 aa, synthetic construct
  REGION 1..214: note = Synthetic
DIQMTQTTSALSASLGDRVTIGCRASQDLSNHLYWYQQKPDGTVKLLIYYTSRLHSGVPSRFSGSGSGTDYSLTIRNLEQEDVATYFCQQGYTLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 292 - protein, 5 aa, synthetic construct
  REGION 1..5: note = Synthetic
SYWIN

SEQ ID NO: 293 - protein, 17 aa, synthetic construct
  REGION 1..17: note = Synthetic
NIYPSDSYTNYNQKFKD

SEQ ID NO: 294 - protein, 14 aa, synthetic construct
  REGION 1..14: note = Synthetic
NGVEGYPHYYAMEY

SEQ ID NO: 295 - protein, 11 aa, synthetic construct
  REGION 1..11: note = Synthetic
RASQDLSNHLY

SEQ ID NO: 296 - protein, 7 aa, synthetic construct
  REGION 1..7: note = Synthetic
YTSRLHS

SEQ ID NO: 297 - protein, 9 aa, synthetic construct
  REGION 1..9: note = Synthetic
QQGYTLPYT

SEQ ID NO: 298 - protein, 123 aa, synthetic construct
  REGION 1..123: note = Synthetic
QVQLQQPGAELVRPGASVKLSCKASGYTFTSYWINWVKQRPGQGLEWIGNIYPSDSYTNYNQKFKDKATLTVDKSSNTVYMQLNSPTSEDSAVYYCTRNGVEGYPHYYAMEYWGQGTSVTVSS

SEQ ID NO: 299 - protein, 107 aa, synthetic construct
  REGION 1..107: note = Synthetic
DIQMTQTTSALSASLGDRVTIGCRASQDLSNHLYWYQQKPDGTVKLLIYYTSRLHSGVPSRFSGSGSGTDYSLTIRNLEQEDVATYFCQQGYTLPYTFGGGTKLEIK

SEQ ID NO: 300 - protein, 5 aa, synthetic construct
  REGION 1..5: note = Synthetic
EYYMY

SEQ ID NO: 301 - protein, 17 aa, synthetic construct
  REGION 1..17: note = Synthetic
RIDPEDGSIDYVEKFKK

SEQ ID NO: 302 - protein, 9 aa, synthetic construct
  REGION 1..9: note = Synthetic
GKFNYRFAY

SEQ ID NO: 303 - protein, 16 aa, synthetic construct
  REGION 1..16: note = Synthetic
RSSQSLLHSSGNTYLN

SEQ ID NO: 304 - protein, 7 aa, synthetic construct
  REGION 1..7: note = Synthetic
LVSKLES

SEQ ID NO: 305 - protein, 9 aa, synthetic construct
  REGION 1..9: note = Synthetic
MQFTHYPYT

SEQ ID NO: 306 - protein, 118 aa, synthetic construct
  REGION 1..118: note = Synthetic
QVQLVQSGAEVKKPGASVKVSCKASGYTFTEYYMYWVRQAPGQGLELMGRIDPEDGSIDYVEKFKKKVTLTADTSSSTAYMELSSLTSDDTAVYYCARGKFNYRFAYWGQGTLVTVSS

SEQ ID NO: 307 - protein, 112 aa, synthetic construct
  REGION 1..112: note = Synthetic
DVVMTQSPPSLLVTLGQPASISCRSSQSLLHSSGNTYLNWLLQRPGQSPQPLIYLVSKLESGVPDRFSGSGSGTDFTLKISGVEAEDVGVYYCMQFTHYPYTFGQGTKLEIK

SEQ ID NO: 308 - protein, 118 aa, synthetic construct
  REGION 1..118: note = Synthetic
QVQLVQSGAEVQRPGASVKVSCKASGYIFTEYYMYWVRQAPGQGLELVGRIDPEDGSIDYVEKFKKKVTLTADTSSSTAYMELSSLTSDDTAVYYCARGKFNYRFAYWGQGTLVTVSS

SEQ ID NO: 309 - protein, 112 aa, synthetic construct; identical to SEQ ID NO: 307
  REGION 1..112: note = Synthetic

SEQ ID NO: 310 - protein, 120 aa, synthetic construct; identical to SEQ ID NO: 7
  REGION 1..120: note = Synthetic

SEQ ID NO: 311 - protein, 109 aa, synthetic construct; identical to SEQ ID NO: 8
  REGION 1..109: note = Synthetic

SEQ ID NO: 312 - protein, 351 aa, Homo sapiens
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 313 - protein, 7 aa, synthetic construct
  REGION 1..7: note = Synthetic
GFTFSSY

SEQ ID NO: 314 - protein, 5 aa, synthetic construct
  REGION 1..5: note = Synthetic
SGGGF

SEQ ID NO: 315 - protein, 9 aa, synthetic construct
  REGION 1..9: note = Synthetic
SSYGEIMDY

SEQ ID NO: 316 - protein, 9 aa, synthetic construct
  REGION 1..9: note = Synthetic
SSYGELMDY

SEQ ID NO: 317 - protein, 11 aa, synthetic construct
  REGION 1..11: note = Synthetic
RASQRIGTSIH

SEQ ID NO: 318 - protein, 7 aa, synthetic construct
  REGION 1..7: note = Synthetic
YASESIS

SEQ ID NO: 319 - protein, 10 aa, synthetic construct
  REGION 1..10: note = Synthetic
QQSHGWPFTF

SEQ ID NO: 320 - protein, 117 aa, synthetic construct
  REGION 1..117: note = Synthetic
EVKLVESGGGLVKPGGSLKLSCAASGFTFSSYDMSWVRQTPEKRLEWVASISGGGFLYYLDSVKGRFTISRDNARNILYLHMTSLRSEDTAMYYCARSSYGEIMDYWGQGTSVTVSS

SEQ ID NO: 321 - protein, 117 aa, synthetic construct
  REGION 1..117: note = Synthetic
EVKLVESGGGLVKPGGSLKLSCAASGFTFSSYDMSWVRQTPEKRLEWVASISGGGFLYYLDSVKGRFTISRDNARNILYLHMTSLRSEDTAMYYCARSSYGELMDYWGQGTSVTVSS

SEQ ID NO: 322 - protein, 107 aa, synthetic construct
  REGION 1..107: note = Synthetic
DILLTQSPAILSVSPGERVSFSCRASQRIGTSIHWYQQRTTGSPRLLIKYASESISGIPSRFSGSGSGTDFTLSINSVESEDVADYYCQQSHGWPFTFGGGTKLEIE

SEQ ID NO: 323 - protein, 324 aa, synthetic construct
  REGION 1..324: note = Synthetic
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 324 - protein, 107 aa, synthetic construct
  REGION 1..107: note = Synthetic
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 325 - protein, 107 aa, synthetic construct
  REGION 1..107: note = Synthetic
DIQMTQSPSSMSASLGDRVTITCRASQDINSYLSWFQQKPGKSPKTLIYRANRLVDGVPSRFSGSGSGTDYTLTISSLQYEDFGIYYCQQYDESPWTFGGGTKLEIK

SEQ ID NO: 326 - protein, 118 aa, synthetic construct
  REGION 1..118: note = Synthetic
QIQLVQSGPGLKKPGGSVRISCAASGYTFTNYGMNWVKQAPGKGLRWMGWINTHTGEPTYADDFKGRFTFSLDTSKSTAYLQINSLRAEDTATYFCTRRGYDWYFDVWGQGTTVTVSS

SEQ ID NO: 327 - protein, 7 aa, synthetic construct
  REGION 1..7: note = Synthetic
GYTFTNY

SEQ ID NO: 328 - protein, 6 aa, synthetic construct
  REGION 1..6: note = Synthetic
NTHTGE

SEQ ID NO: 329 - protein, 9 aa, synthetic construct
  REGION 1..9: note = Synthetic
RGYDWYFDV

SEQ ID NO: 330 - protein, 11 aa, synthetic construct
  REGION 1..11: note = Synthetic
RASQDINSYLS

SEQ ID NO: 331 - protein, 7 aa, synthetic construct
  REGION 1..7: note = Synthetic
RANRLVD

SEQ ID NO: 332 - protein, 9 aa, synthetic construct
  REGION 1..9: note = Synthetic
QQYDESPWT

SEQ ID NO: 333 - protein, 107 aa, synthetic construct
  REGION 1..107: note = Synthetic
DIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIK

SEQ ID NO: 334 - protein, 119 aa, synthetic construct
  REGION 1..119: note = Synthetic
EIQLVQSGGGLVKPGGSVRISCAASGYTFTNYGMNWVRQAPGKGLEWMGWINTHYGEPTYADSFKGTRTFSLDDSKNTAYLQINSLRAEDTAVYFCTRRGYDWYFDVWGQGGTTVTVSS

SEQ ID NO: 335 - protein, 7 aa, synthetic construct
  REGION 1..7: note = Synthetic
GYTFTNY

SEQ ID NO: 336 - protein, 6 aa, synthetic construct
  REGION 1..6: note = Synthetic
NTHYGE

SEQ ID NO: 337 - protein, 10 aa, synthetic construct
  REGION 1..10: note = Synthetic
RRGYDWYFDV

SEQ ID NO: 338 - protein, 11 aa, synthetic construct
  REGION 1..11: note = Synthetic
RASQDINSYLS

SEQ ID NO: 339 - protein, 7 aa, synthetic construct
  REGION 1..7: note = Synthetic
RANRLES

SEQ ID NO: 340 - protein, 9 aa, synthetic construct
  REGION 1..9: note = Synthetic
QQYDESPWT

SEQ ID NO: 341 - protein, 9 aa, synthetic construct
  REGION 1..9: note = Synthetic
GYSITSGYY

SEQ ID NO: 342 - protein, 7 aa, synthetic construct
  REGION 1..7: note = Synthetic
ISYSGFT

SEQ ID NO: 343 - protein, 11 aa, synthetic construct
  REGION 1..11: note = Synthetic
AGDRTGSWFAY

SEQ ID NO: 344 - protein, 6 aa, synthetic construct
  REGION 1..6: note = Synthetic
QDISNY

SEQ ID NO: 345 - skipped

SEQ ID NO: 346 - protein, 9 aa, synthetic construct
  REGION 1..9: note = Synthetic
LQYASYPFT

SEQ ID NO: 347 - protein, 8 aa, synthetic construct
  REGION 1..8: note = Synthetic
GYIFTNYG

SEQ ID NO: 348 - protein, 8 aa, synthetic construct
  REGION 1..8: note = Synthetic
INTYNGEP

SEQ ID NO: 349 - protein, 11 aa, synthetic construct
  REGION 1..11: note = Synthetic
ARGDYYGYEDY

SEQ ID NO: 350 - protein, 6 aa, synthetic construct
  REGION 1..6: note = Synthetic
QGISNY

SEQ ID NO: 351 - skipped

SEQ ID NO: 352 - protein, 9 aa, synthetic construct
  REGION 1..9: note = Synthetic
QQYSKLPWT

SEQ ID NO: 353 - protein, 9 aa, synthetic construct
  REGION 1..9: note = Synthetic
FSLSTSGMG

SEQ ID NO: 354 - protein, 5 aa, synthetic construct
  REGION 1..5: note = Synthetic
WWDDD

SEQ ID NO: 355 - protein, 10 aa, synthetic construct
  REGION 1..10: note = Synthetic
RRATGTGFDY

SEQ ID NO: 356 - protein, 6 aa, synthetic construct
  REGION 1..6: note = Synthetic
QDVGTA

SEQ ID NO: 357 - protein, 7 aa, synthetic construct
  REGION 1..7: note = Synthetic
WTSTRHT

SEQ ID NO: 358 - protein, 6 aa, synthetic construct
  REGION 1..6: note = Synthetic
YNSYNT

SEQ ID NO: 359 - protein, 120 aa, synthetic construct
  REGION 1..120: note = Synthetic
QVTLKESGPVLVKPTETLTLTCTFSGFSLSTSGMGVGWIRQAPGKGLEWVAHIWWDDDVYYNPSLKSRLTITKDASKDQVSLKLSSVTAADTAVYYCVRRRATGTGFDYWGQGTLVTVSS

SEQ ID NO: 360 - protein, 106 aa, synthetic construct
  REGION 1..106: note = Synthetic
NIVMTQSPSSLSASVGDRVTITCQASQDVGTAVAWYQQKPDQSPKLLIYWTSTRHTGVPDRFTGSGSGTDFTLTISSLQPEDIATYFCHQYNSYNTFGSGTKLEIK

SEQ ID NO: 361 - protein, 120 aa, synthetic construct; identical to SEQ ID NO: 7
  REGION 1..120: note = Synthetic

SEQ ID NO: 362 - protein, 109 aa, synthetic construct; identical to SEQ ID NO: 8
  REGION 1..109: note = Synthetic

SEQ ID NO: 363 - protein, 438 aa, Homo sapiens
MVCSQSWGRSSKQWEDPSQASKVCQRLNCGVPLSLGPFLVTYTPQSSIICYGQLGSFSNCSHSRNDMCHSLGLTCLEPQKTTPPTTRPPPTTTPEPTAPPRLQLVAQSGGQHCAGVVEFYSGSLGGTISYEAQDKTQDLENFLCNNLQCGSFLKHLPETEAGRAQDPGEPREHQPLPIQWKIQNSSCTSLEHCFRKIKPQKSGRVLALLCSGFQPKVQSRLVGGSSICEGTVEVRQGAQWAALCDSSSARSSLRWEEVCREQQCGSVNSYRVLDAGDPTSRGLFCPHQKLSQCHELWERNSYCKKVFVTCQDPNPAGLAAGTVASIILALVLLVVLLVVCGPLAYKKLVKKFRQKKQRQWIGPTGMNQNMSFHRNHTATVRSHAENPTASHVDNEYSQPPRNSHLSAYPALEGALHRSSMQPDNSSDSDYDLHGAQRL

SEQ ID NO: 364 - protein, 450 aa, synthetic construct
  REGION 1..450: note = Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 365 - protein, 450 aa, synthetic construct
  REGION 1..450: note = Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 366 - protein, 450 aa, synthetic construct
  REGION 1..450: note = Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 367 - protein, 450 aa, synthetic construct
  REGION 1..450: note = Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 368 - protein, 120 aa, synthetic construct [sequence omitted]

SEQ ID NO: 369 - protein, 214 aa, synthetic construct
  REGION 1..214: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 370 - protein, 107 aa, synthetic construct [sequence omitted]

SEQ ID NO: 371 - protein, 450 aa, synthetic construct
  REGION 1..450: note = Synthetic
QVQLVQSGAEVKKPGASVKVSCKASGYTFTYFDINWLRQAPGQGLEWMGGISPGDSNVNYAQKFQGRVTLTIDTSTSTAYMELSSLRSEDTAVYYCARDGNFPYYAMDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 372 - protein, 450 aa, synthetic construct
  REGION 1..450: note = Synthetic
QVQLVQSGAEVKKPGASVKVSCKASGYTFTYFDINWLRQAPGQGLEWMGGISPGDSNVNYAQKFQGRVTLTIDTSTSTAYMELSSLRSEDTAVYYCARDGNFPYYAMDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 373 - protein, 450 aa, synthetic construct
  REGION 1..450: note = Synthetic
QVQLVQSGAEVKKPGASVKVSCKASGYTFTYFDINWLRQAPGQGLEWMGGISPGDSNVNYAQKFQGRVTLTIDTSTSTAYMELSSLRSEDTAVYYCARDGNFPYYAMDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 374 - protein, 450 aa, synthetic construct
  REGION 1..450: note = Synthetic
QVQLVQSGAEVKKPGASVKVSCKASGYTFTYFDINWLRQAPGQGLEWMGGISPGDSNVNYAQKFQGRVTLTIDTSTSTAYMELSSLRSEDTAVYYCARDGNFPYYAMDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 375 - protein, 120 aa, synthetic construct
  REGION 1..120: note = Synthetic
QVQLVQSGAEVKKPGASVKVSCKASGYTFTYFDINWLRQAPGQGLEWMGGISPGDSNVNYAQKFQGRVTLTIDTSTSTAYMELSSLRSEDTAVYYCARDGNFPYYAMDSWGQGTLVTVSS

SEQ ID NO: 376 - protein, 218 aa, synthetic construct
  REGION 1..218: note = Synthetic
DIVMTQSPLSLPVTPGEPASISCRSSQSLVHSSGQTYLHWYQQKPGQSPQLLIYTVSNRASGVPDRFSGSGSGTDFTLKISRVEAEDVGTYYCSQSTHVPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 377 - protein, 111 aa, synthetic construct
  REGION 1..111: note = Synthetic
DIVMTQSPLSLPVTPGEPASISCRSSQSLVHSSGQTYLHWYQQKPGQSPQLLIYTVSNRASGVPDRFSGSGSGTDFTLKISRVEAEDVGTYYCSQSTHVPTFGGGTKVEIK

SEQ ID NO: 378 - protein, 7 aa, synthetic construct
  REGION 1..7: note = Synthetic
GYTFTYF

SEQ ID NO: 379 - protein, 17 aa, synthetic construct
  REGION 1..17: note = Synthetic
GISPGDSNVNYAQKFQG

SEQ ID NO: 380 - protein, 11 aa, synthetic construct
  REGION 1..11: note = Synthetic
DGNFPYYAMDS

SEQ ID NO: 381 - protein, 16 aa, synthetic construct
  REGION 1..16: note = Synthetic
RSSQSLVHSSGQTYLH

SEQ ID NO: 382 - protein, 7 aa, synthetic construct
  REGION 1..7: note = Synthetic
TVSNRAS

SEQ ID NO: 383 - protein, 8 aa, synthetic construct
  REGION 1..8: note = Synthetic
SQSTHVPT

SEQ ID NO: 384 - protein, 450 aa, synthetic construct; identical to SEQ ID NO: 364
  REGION 1..450: note = Synthetic

SEQ ID NO: 385 - protein, 450 aa, synthetic construct; identical to SEQ ID NO: 365
  REGION 1..450: note = Synthetic

SEQ ID NO: 386 - protein, 450 aa, synthetic construct; identical to SEQ ID NO: 366
  REGION 1..450: note = Synthetic

SEQ ID NO: 387 - protein, 450 aa, synthetic construct; identical to SEQ ID NO: 367
  REGION 1..450: note = Synthetic

SEQ ID NO: 388 - protein, 120 aa, synthetic construct [sequence omitted]

SEQ ID NO: 389 - protein, 214 aa, synthetic construct; identical to SEQ ID NO: 369
  REGION 1..214: note = Synthetic

SEQ ID NO: 390 - protein, 107 aa, synthetic construct [sequence omitted]

SEQ ID NO: 391 - protein, 450 aa, synthetic construct; identical to SEQ ID NO: 371
  REGION 1..450: note = Synthetic

SEQ ID NO: 392 - protein, 450 aa, synthetic construct; identical to SEQ ID NO: 372
  REGION 1..450: note = Synthetic

SEQ ID NO: 393 - protein, 450 aa, synthetic construct; identical to SEQ ID NO: 373
  REGION 1..450: note = Synthetic

SEQ ID NO: 394 - protein, 450 aa, synthetic construct; identical to SEQ ID NO: 374
  REGION 1..450: note = Synthetic

SEQ ID NO: 395 - protein, 120 aa, synthetic construct; identical to SEQ ID NO: 375
  REGION 1..120: note = Synthetic

SEQ ID NO: 396 - protein, 218 aa, synthetic construct; identical to SEQ ID NO: 376
  REGION 1..218: note = Synthetic

SEQ ID NO: 397 - protein, 111 aa, synthetic construct; identical to SEQ ID NO: 377
  REGION 1..111: note = Synthetic

SEQ ID NO: 398 - protein, 7 aa, synthetic construct
  REGION 1..7: note = Synthetic
GYTFTYF

SEQ ID NO: 399 - protein, 17 aa, synthetic construct
  REGION 1..17: note = Synthetic
GISPGDSNVNYAQKFQG

SEQ ID NO: 400 - protein, 11 aa, synthetic construct
  REGION 1..11: note = Synthetic
DGNFPYYAMDS

SEQ ID NO: 401 - protein, 16 aa, synthetic construct
  REGION 1..16: note = Synthetic
RSSQSLVHSSGQTYLH

SEQ ID NO: 402 - protein, 7 aa, synthetic construct
  REGION 1..7: note = Synthetic
TVSNRAS

SEQ ID NO: 403 - protein, 8 aa, synthetic construct
  REGION 1..8: note = Synthetic
SQSTHVPT

SEQ ID NO: 404 - protein, 15 aa, synthetic construct
  REGION 1..15: note = Synthetic
EPKSSDKTHTSPPSP

SEQ ID NO: 405 - protein, 8 aa, synthetic construct
  REGION 1..8: note = Synthetic
  SITE 1..8: note = /note="Variant residues given in the sequence have no preference with respect to those in the annotations for variant positions"
  VARIANT 7: note = /replace="Asp"
IWGIGCNP

SEQ ID NO: 406 - protein, 10 aa, synthetic construct
  REGION 1..10: note = Synthetic
SGYSFTGYTM

SEQ ID NO: 407 - protein, 10 aa, synthetic construct
  REGION 1..10: note = Synthetic
SGYSFTDYTM

SEQ ID NO: 408 - protein, 10 aa, synthetic construct
  REGION 1..10: note = Synthetic
SGFTFSNYAM

SEQ ID NO: 409 - protein, 10 aa, synthetic construct
  REGION 1..10: note = Synthetic
SGFTFSSYAM

SEQ ID NO: 410 - protein, 10 aa, synthetic construct
  REGION 1..10: note = Synthetic
SGYSFTAYNI

SEQ ID NO: 411 - protein, 10 aa, synthetic construct
  REGION 1..10: note = Synthetic
SGYSFTAYSM

SEQ ID NO: 412 - protein, 10 aa, synthetic construct
  REGION 1..10: note = Synthetic
SGYTFTNFAI

SEQ ID NO: 413 - protein, 10 aa, synthetic construct
  REGION 1..10: note = Synthetic
SGFNIKDTYM

SEQ ID NO: 414 - protein, 10 aa, synthetic construct
  REGION 1..10: note = Synthetic
SGYSFTSYWM

SEQ ID NO: 415 - protein, 10 aa, synthetic construct
  REGION 1..10: note = Synthetic
SGFSLTNYDV

SEQ ID NO: 416 - protein, 10 aa, synthetic construct
  REGION 1..10: note = Synthetic
SGFTFSNYGM

SEQ ID NO: 417 - protein, 10 aa, synthetic construct
  REGION 1..10: note = Synthetic
SGYIFANYGM

SEQ ID NO: 418 - protein, 10 aa, synthetic construct
  REGION 1..10: note = Synthetic
SGYNFTNYGM

SEQ ID NO: 419 - protein, 10 aa, synthetic construct
  REGION 1..10: note = Synthetic
SGYTFTNYGM

SEQ ID NO: 420 - protein, 10 aa, synthetic construct
  REGION 1..10: note = Synthetic
SGYTFTDYYI

SEQ ID NO: 421 - protein, 10 aa, synthetic construct
  REGION 1..10: note = Synthetic
SGNTFTNFYL

SEQ ID NO: 422 - protein, 10 aa, synthetic construct
  REGION 1..10: note = Synthetic
SEFTFSNYAM

SEQ ID NO: 423 - protein, 10 aa, synthetic construct
  REGION 1..10: note = Synthetic
SGYTFTSYRM

SEQ ID NO: 424 - protein, 10 aa, synthetic construct
  REGION 1..10: note = Synthetic
SGYMFTNHGM

SEQ ID NO: 425 - protein, 10 aa, synthetic construct
  REGION 1..10: note = Synthetic
SGYMFTNYGM

SEQ ID NO: 426 - protein, 10 aa, synthetic construct
  REGION 1..10: note = Synthetic
SGYIFTNYGM

SEQ ID NO: 427 - protein, 10 aa, synthetic construct
  REGION 1..10: note = Synthetic
SGFNIKDYYI

SEQ ID NO: 428 - protein, 10 aa, synthetic construct
  REGION 1..10: note = Synthetic
SGYTFINYGM

SEQ ID NO: 429 - protein, 10 aa, synthetic construct
  REGION 1..10: note = Synthetic
SGYTFTDYFI

SEQ ID NO: 430 - protein, 10 aa, synthetic construct
  REGION 1..10: note = Synthetic
SGYIFTGYNI

SEQ ID NO: 431 - protein, 10 aa, synthetic construct
  REGION 1..10: note = Synthetic
LINPYNGGTT

SEQ ID NO: 432 - protein, 10 aa, synthetic construct
  REGION 1..10: note = Synthetic
LINPYNGGTM

SEQ ID NO: 433 - protein, 9 aa, synthetic construct
  REGION 1..9: note = Synthetic
SISSGGNTF

SEQ ID NO: 434 - protein, 9 aa, synthetic construct
  REGION 1..9: note = Synthetic
SISSGGSTY

SEQ ID NO: 435 - protein, 10 aa, synthetic construct
  REGION 1..10: note = Synthetic
SIDPYYGDTK

SEQ ID NO: 436 - protein, 10 aa, synthetic construct
  REGION 1..10: note = Synthetic
LISSNSGDVS

SEQ ID NO: 437 - protein, 10 aa, synthetic construct
  REGION 1..10: note = Synthetic
LISTSSGDVS

SEQ ID NO: 438 - protein, 10 aa, synthetic construct
  REGION 1..10: note = Synthetic
RIDPANGNTK

SEQ ID NO: 439 - protein, 10 aa, synthetic construct
  REGION 1..10: note = Synthetic
MIHPSDSETR

SEQ ID NO: 440 - protein, 9 aa, synthetic construct
  REGION 1..9: note = Synthetic
VIWSGGNTD

SEQ ID NO: 441 - protein, 10 aa, synthetic construct
  REGION 1..10: note = Synthetic
AINSNGDITY

SEQ ID NO: 442 - protein, 10 aa, synthetic construct
  REGION 1..10: note = Synthetic
LINPYNGGTR

SEQ ID NO: 443 - protein, 10 aa, synthetic construct
  REGION 1..10: note = Synthetic
WINTYTGEPT

SEQ ID NO: 444 - protein, 10 aa, synthetic construct
  REGION 1..10: note = Synthetic
WIYPGGGNTR

SEQ ID NO: 445 - protein, 10 aa, synthetic construct
  REGION 1..10: note = Synthetic
CIYPGNVKTK

SEQ ID NO: 446 - protein, 10 aa, synthetic construct
  REGION 1..10: note = Synthetic
TISSGGSYTY

SEQ ID NO: 447 - protein, 10 aa, synthetic construct
  REGION 1..10: note = Synthetic
RIDPYDSGTH

SEQ ID NO: 448 - protein, 10 aa, synthetic construct
  REGION 1..10: note = Synthetic
WIDPENGRTE

SEQ ID NO: 449 - protein, 10 aa, synthetic construct
  REGION 1..10: note = Synthetic
EIYPGSSNTY

SEQ ID NO: 450 - protein, 10 aa, synthetic construct
  REGION 1..10: note = Synthetic
AVYPGNGDTS

SEQ ID NO: 451 - protein, 15 aa, synthetic construct
  REGION 1..15: note = Synthetic
CARDYYGSSPDFDYW

SEQ ID NO: 452 - protein, 15 aa, synthetic construct
  REGION 1..15: note = Synthetic
CARDNYGSSPDFDYW

SEQ ID NO: 453 - protein, 15 aa, synthetic construct
  REGION 1..15: note = Synthetic
CARDNYGSSPYFDYW

SEQ ID NO: 454 - protein, 16 aa, synthetic construct
  REGION 1..16: note = Synthetic
CVRYYYGVTYWYFDVW

SEQ ID NO: 455 - protein, 16 aa, synthetic construct
  REGION 1..16: note = Synthetic
CVRYYYGIRYWYFDVW

SEQ ID NO: 456 - protein, 16 aa, synthetic construct
  REGION 1..16: note = Synthetic
CARRMITMGDWYFDVW

SEQ ID NO: 457 - protein, 16 aa, synthetic construct
  REGION 1..16: note = Synthetic
CARRMITTGDWYFDVW

SEQ ID NO: 458 - protein, 14 aa, synthetic construct
  REGION 1..14: note = Synthetic
CARHYGAHNYFDYW

SEQ ID NO: 459 - protein, 14 aa, synthetic construct
  REGION 1..14: note = Synthetic
CARHYGANNYFDYW

SEQ ID NO: 460 - protein, 16 aa, synthetic construct
  REGION 1..16: note = Synthetic
CAREENYYGTYYFDYW

SEQ ID NO: 461 - protein, 14 aa, synthetic construct
  REGION 1..14: note = Synthetic
CARWGDHDDAMDFW

SEQ ID NO: 462 - protein, 17 aa, synthetic construct
  REGION 1..17: note = Synthetic
CARNHGDGYFNWYFDVW

SEQ ID NO: 463 - protein, 17 aa, synthetic construct
  REGION 1..17: note = Synthetic
CARNHGDGYYNWYFDVW

SEQ ID NO: 464 - protein, 11 aa, synthetic construct
  REGION 1..11: note = Synthetic
CARGTAWFTYW

SEQ ID NO: 465 - protein, 14 aa, synthetic construct
  REGION 1..14: note = Synthetic
CARDGDDGWDIDVW

SEQ ID NO: 466 - protein, 13 aa, synthetic construct
  REGION 1..13: note = Synthetic
CARRGTYWHFDVW

SEQ ID NO: 467 - protein, 13 aa, synthetic construct
  REGION 1..13: note = Synthetic
CARRGSYWHFDVW

SEQ ID NO: 468 - protein, 12 aa, synthetic construct
  REGION 1..12: note = Synthetic
CARRSTLVFDYW

SEQ ID NO: 469 - protein, 12 aa, synthetic construct
  REGION 1..12: note = Synthetic
CARNGYWYFDVW

SEQ ID NO: 470 - protein, 16 aa, synthetic construct
  REGION 1..16: note = Synthetic
CAKEGDYDGTAYFDYW

SEQ ID NO: 471 - protein, 12 aa, synthetic construct
  REGION 1..12: note = Synthetic
CARRRDGNFDYW

SEQ ID NO: 472 - protein, 10 aa, synthetic construct
  REGION 1..10: note = Synthetic
CVRHGYFDVW

SEQ ID NO: 473 - protein, 9 aa, synthetic construct
  REGION 1..9: note = Synthetic
CAFYDGAYW

SEQ ID NO: 474 - protein, 9 aa, synthetic construct
  REGION 1..9: note = Synthetic
CASYDPDYW

SEQ ID NO: 475 - protein, 14 aa, synthetic construct
  REGION 1..14: note = Synthetic
CARDTTATYYFDYW

SEQ ID NO: 476 - protein, 12 aa, synthetic construct
  REGION 1..12: note = Synthetic
CARRVATYFDVW

SEQ ID NO: 477 - protein, 12 aa, synthetic construct
  REGION 1..12: note = Synthetic
CTRRSHITLDYW

SEQ ID NO: 478 - protein, 12 aa, synthetic construct
  REGION 1..12: note = Synthetic
CARRRTTAFDYW

SEQ ID NO: 479 - protein, 15 aa, synthetic construct
  REGION 1..15: note = Synthetic
CNNGNYVRHYYFDYW

SEQ ID NO: 480 - protein, 12 aa, synthetic construct
  REGION 1..12: note = Synthetic
CTRRREITFDYW

SEQ ID NO: 481 - protein, 12 aa, synthetic construct
  REGION 1..12: note = Synthetic
CARSGISPFTYW

SEQ ID NO: 482 - protein, 11 aa, synthetic construct
  REGION 1..11: note = Synthetic
CAKYDRFFASW

SEQ ID NO: 483 - protein, 8 aa, synthetic construct
  REGION 1..8: note = Synthetic
SQGISNHL

SEQ ID NO: 484 - protein, 8 aa, synthetic construct
  REGION 1..8: note = Synthetic
SQGIRNYL

SEQ ID NO: 485 - protein, 8 aa, synthetic construct
  REGION 1..8: note = Synthetic
SQGINNYL

SEQ ID NO: 486 - protein, 12 aa, synthetic construct
  REGION 1..12: note = Synthetic
SQSVDHDGDSYM

SEQ ID NO: 487 - protein, 12 aa, synthetic construct
  REGION 1..12: note = Synthetic
SQSVDYDGDSYM

SEQ ID NO: 488 - protein, 8 aa, synthetic construct
  REGION 1..8: note = Synthetic
SQDISNYL

SEQ ID NO: 489 - protein, 8 aa, synthetic construct
  REGION 1..8: note = Synthetic
SQDISTYL

SEQ ID NO: 490 - protein, 9 aa, synthetic construct
  REGION 1..9: note = Synthetic
TSSISSSYL

SEQ ID NO: 491 - protein, 9 aa, synthetic construct
  REGION 1..9: note = Synthetic
NSSVSSSYL

SEQ ID NO: 492 - protein, 8 aa, synthetic construct
  REGION 1..8: note = Synthetic
SENIYYNL

SEQ ID NO: 493 - protein, 8 aa, synthetic construct
  REGION 1..8: note = Synthetic
SENIYGYF

SEQ ID NO: 494 - protein, 8 aa, synthetic construct
  REGION 1..8: note = Synthetic
SQDINNYI

SEQ ID NO: 495 - protein, 8 aa, synthetic construct
  REGION 1..8: note = Synthetic
SQDINKYI

SEQ ID NO: 496 - protein, 8 aa, synthetic construct
  REGION 1..8: note = Synthetic
SENIYSYL

SEQ ID NO: 497 - protein, 8 aa, synthetic construct
  REGION 1..8: note = Synthetic
SQDVRTDV

SEQ ID NO: 498 - protein, 8 aa, synthetic construct
  REGION 1..8: note = Synthetic
SQDVITAV

SEQ ID NO: 499 - protein, 8 aa, synthetic construct
  REGION 1..8: note = Synthetic
SQSIGTSI

SEQ ID NO: 500 - protein, 12 aa, synthetic construct
  REGION 1..12: note = Synthetic
SSQSLLNQKNYL

SEQ ID NO: 501 - protein, 9 aa, synthetic construct
  REGION 1..9: note = Synthetic
SSSVSSSYL

SEQ ID NO: 502 - protein, 8 aa, synthetic construct
  REGION 1..8: note = Synthetic
SQTIGTSI

SEQ ID NO: 503 - protein, 14 aa, synthetic construct
  REGION 1..14: note = Synthetic
SQSLLYSSDQKNYL

SEQ ID NO: 504 - protein, 7 aa, synthetic construct
  REGION 1..7: note = Synthetic
NSSVSYM

SEQ ID NO: 505 - protein, 7 aa, synthetic construct
  REGION 1..7: note = Synthetic
SSSLSYM

SEQ ID NO: 506 - protein, 8 aa, synthetic construct
  REGION 1..8: note = Synthetic
SQRIGTSM

SEQ ID NO: 507 - protein, 8 aa, synthetic construct
  REGION 1..8: note = Synthetic
SQNIGTSI

SEQ ID NO: 508 - protein, 7 aa, synthetic construct
  REGION 1..7: note = Synthetic
ISSVSYM

SEQ ID NO: 509 - protein, 8 aa, synthetic construct
  REGION 1..8: note = Synthetic
SQTIATSI

SEQ ID NO: 510 - protein, 13 aa, synthetic construct
  REGION 1..13: note = Synthetic
SQSLVHSNGNTYL

SEQ ID NO: 511 - protein, 12 aa, synthetic construct
  REGION 1..12: note = Synthetic
NESVEYSGTSLM

SEQ ID NO: 512 - protein, 5 aa, synthetic construct
  REGION 1..5: note = Synthetic
YFTSS

SEQ ID NO: 513 - protein, 5 aa, synthetic construct
  REGION 1..5: note = Synthetic
YYTSS

SEQ ID NO: 514 - protein, 5 aa, synthetic construct
  REGION 1..5: note = Synthetic
YAASN

SEQ ID NO: 515 - protein, 5 aa, synthetic construct
  REGION 1..5: note = Synthetic
YYTSR

SEQ ID NO: 516 - protein, 5 aa, synthetic construct
  REGION 1..5: note = Synthetic
FYTSR

SEQ ID NO: 517 - protein, 5 aa, synthetic construct
  REGION 1..5: note = Synthetic
YGTSN

SEQ ID NO: 518 - protein, 5 aa, synthetic construct
  REGION 1..5: note = Synthetic
YNANS

SEQ ID NO: 519 - protein, 5 aa, synthetic construct
  REGION 1..5: note = Synthetic
YNAKT

SEQ ID NO: 520 - protein, 5 aa, synthetic construct
  REGION 1..5: note = Synthetic
HYTST

SEQ ID NO: 521 - protein, 5 aa, synthetic construct
  REGION 1..5: note = Synthetic
YHTST

SEQ ID NO: 522 - protein, 5 aa, synthetic construct
  REGION 1..5: note = Synthetic
YSASF

SEQ ID NO: 523 - protein, 5 aa, synthetic construct
  REGION 1..5: note = Synthetic
YSASY

SEQ ID NO: 524 - protein, 5 aa, synthetic construct
  REGION 1..5: note = Synthetic
KSASE

SEQ ID NO: 525 - protein, 5 aa, synthetic construct
  REGION 1..5: note = Synthetic
YWAST

SEQ ID NO: 526 - protein, 5 aa, synthetic construct
  REGION 1..5: note = Synthetic
YSTSN

SEQ ID NO: 527 - protein, 5 aa, synthetic construct
  REGION 1..5: note = Synthetic
KNASE

SEQ ID NO: 528 - protein, 5 aa, synthetic construct
  REGION 1..5: note = Synthetic
YDTSK

SEQ ID NO: 529 - protein, 5 aa, synthetic construct
  REGION 1..5: note = Synthetic
YDTSN

SEQ ID NO: 530 - protein, 5 aa, synthetic construct
  REGION 1..5: note = Synthetic
KDASE

SEQ ID NO: 531 - protein, 5 aa, synthetic construct
  REGION 1..5: note = Synthetic
YATSN

SEQ ID NO: 532 - protein, 5 aa, synthetic construct
  REGION 1..5: note = Synthetic
YKVSN

SEQ ID NO: 533 - protein, 5 aa, synthetic construct
  REGION 1..5: note = Synthetic
SAASN

SEQ ID NO: 534 - protein, 11 aa, synthetic construct
  REGION 1..11: note = Synthetic
CQQYSNLPYTF

SEQ ID NO: 535 - protein, 11 aa, synthetic construct
  REGION 1..11: note = Synthetic
CQQYSKIPYTC

SEQ ID NO: 536 - protein, 10 aa, synthetic construct
  REGION 1..10: note = Synthetic
CQQNYEDPTF

SEQ ID NO: 537 - protein, 10 aa, synthetic construct
  REGION 1..10: note = Synthetic
CQQSNEDPTF

SEQ ID NO: 538 - protein, 11 aa, synthetic construct
  REGION 1..11: note = Synthetic
CQQGDALPWTF

SEQ ID NO: 539 - protein, 11 aa, synthetic construct [sequence omitted]

SEQ ID NO: 540 - protein, 11 aa, synthetic construct
  REGION 1..11: note = Synthetic
CQQWSSRPPTF

SEQ ID NO: 541 - protein, 11 aa, synthetic construct [sequence omitted]

SEQ ID NO: 542 - protein, 11 aa, synthetic construct [sequence omitted]

SEQ ID NO: 543 - protein, 11 aa, synthetic construct
  REGION 1..11: note = Synthetic
CQQRSYFPFTF

SEQ ID NO: 544 - protein, 11 aa, synthetic construct
  REGION 1..11: note = Synthetic
CKQVYDVPFTF

SEQ ID NO: 545 - protein, 11 aa, synthetic construct
  REGION 1..11: note = Synthetic
CQHHYGTPFTF

SEQ ID NO: 546 - protein, 10 aa, synthetic construct
  REGION 1..10: note = Synthetic
CLQYDNLWTF

SEQ ID NO: 547 - protein, 11 aa, synthetic construct
  REGION 1..11: note = Synthetic
CQHHYGYPYTF

SEQ ID NO: 548 - protein, 11 aa, synthetic construct [sequence omitted]

SEQ ID NO: 549 - protein, 11 aa, synthetic construct
  REGION 1..11: note = Synthetic
CQQHYTSPWTF

SEQ ID NO: 550 - protein, 11 aa, synthetic construct [sequence omitted]

SEQ ID NO: 551 - protein, 11 aa, synthetic construct
  REGION 1..11: note = Synthetic
CQQSNRWPLTF

SEQ ID NO: 552 - protein, 11 aa, synthetic construct
  REGION 1..11: note = Synthetic
CQNDYDYPYTF

SEQ ID NO: 553 - protein, 11 aa, synthetic construct
  REGION 1..11: note = Synthetic
CHQYHRSPLTF

SEQ ID NO: 554 - protein, 11 aa, synthetic construct
  REGION 1..11: note = Synthetic
CQQTFDVPWTF

SEQ ID NO: 555 - protein, 11 aa, synthetic construct
  REGION 1..11: note = Synthetic
CQQSNSWPLTY

SEQ ID NO: 556 - protein, 11 aa, synthetic construct [sequence omitted]

SEQ ID NO: 557 - protein, 11 aa, synthetic construct
  REGION 1..11: note = Synthetic
CQQWSSNPFTF

SEQ ID NO: 558 - protein, 11 aa, synthetic construct
  REGION 1..11: note = Synthetic
CKQAYDVPWTF

SEQ ID NO: 559 - protein, 11 aa, synthetic construct
  REGION 1..11: note = Synthetic
CQQWSSFPPTF

SEQ ID NO: 560 - protein, 11 aa, synthetic construct
  REGION 1..11: note = Synthetic
CQQSNSWPLTF

SEQ ID NO: 561 - protein, 11 aa, synthetic construct
  REGION 1..11: note = Synthetic
CQQSDSWPLTF

SEQ ID NO: 562 - protein, 11 aa, synthetic construct
  REGION 1..11: note = Synthetic
CQQWSSNPRTF

SEQ ID NO: 563 - protein, 11 aa, synthetic construct
  REGION 1..11: note = Synthetic
CWQNTHFPQTF

SEQ ID NO: 564 - protein, 11 aa, synthetic construct
  REGION 1..11: note = Synthetic
CQQSRQVPLTF